SEQUENCE LISTING (907 sequences)

SEQ ID NO: 1 - DNA, 51 bp, Homo sapiens
atacattatatttaaacatatctctatagagtcaacaaaataaaataaaca

SEQ ID NO: 2 - DNA, 51 bp, Homo sapiens
atgtaagatcgtttgggaaaatgttaagacagatatcttgctttaattttt

SEQ ID NO: 3 - DNA, 51 bp, Homo sapiens
gcttatgtttatgtgatggcacctgcgagtacatagaggttggatatgtta

SEQ ID NO: 4 - DNA, 51 bp, Homo sapiens
actccaacccacagcattattattattcagtaggttatagaggtgttatac

SEQ ID NO: 5 - DNA, 51 bp, Homo sapiens
accccttaaattttcattttctctcaaagtctcctctaaatttagtatatt

SEQ ID NO: 6 - DNA, 51 bp, Homo sapiens
tgattagccttccatttcataaacctttttttcccctggaattgataatgg

SEQ ID NO: 7 - DNA, 51 bp, Homo sapiens
ataaagttggaatttggagtcatggcctgaaaaatgtgagcaagtaaagaa

SEQ ID NO: 8 - DNA, 51 bp, Homo sapiens
tggaagggtagaaccttaagtagtttttcattctctgactactcaactaga

SEQ ID NO: 9 - DNA, 51 bp, Homo sapiens
gattttgaggccatgtttccgttaatctggaccgagagccctctgggagag

SEQ ID NO: 10 - DNA, 51 bp, Homo sapiens
aatgcgatttgatgattgtaacaggacaaaattttgattctttcgaaattc

SEQ ID NO: 11 - DNA, 51 bp, Homo sapiens
agctaggtggtgttctcgtgtacatgttagagatgaggaaacccaatctct

SEQ ID NO: 12 - DNA, 51 bp, Homo sapiens
tccttgtttcccccagccttttgtcgcttaacatgtttctttatgcttatt

SEQ ID NO: 13 - DNA, 51 bp, Homo sapiens
gttctagaagtgacaaagctgggacacaatacctttatgcatgaaaaggtt

SEQ ID NO: 14 - DNA, 51 bp, Homo sapiens
catctctcttatcatgctgcctcccaacatgcaggggagagtcctggcctt

SEQ ID NO: 15 - DNA, 51 bp, Homo sapiens
ctgaataaatggctcaatgaataacgcacaagtgaacatgtcaaactgaaa

SEQ ID NO: 16 - DNA, 51 bp, Homo sapiens
tttgggcaggtctgtccttggtttccttatcgatgaccatgcagcccttgc

SEQ ID NO: 17 - DNA, 51 bp, Homo sapiens
agcaagaaaagattactgttctggctcccttcagcttctatgtcattgcat

SEQ ID NO: 18 - DNA, 51 bp, Homo sapiens
cttggccttggctttcattttgcattgctcttaaataataagtttgcttct

SEQ ID NO: 19 - DNA, 51 bp, Homo sapiens
ctcagcccctgctctgagtgccatcaatttaactgttttgtggttcttctc

SEQ ID NO: 20 - DNA, 51 bp, Homo sapiens
aaaggtattggacttatatccttgatagaattgtagactgagtcactataa

SEQ ID NO: 21 - DNA, 51 bp, Homo sapiens
tccatccgcttcccaggcagacctatcagccagacagcttccgtcttgcct

SEQ ID NO: 22 - DNA, 51 bp, Homo sapiens
tagaggcagccatcaaatcaccacccgggaatgttcaactgcaagtgtgcc

SEQ ID NO: 23 - DNA, 51 bp, Homo sapiens
ggagatttccttgacttcgtcttccctcttttggtcaaattaaaaaatatc

SEQ ID NO: 24 - DNA, 51 bp, Homo sapiens
acccaggagggcttcttggaggaggcggccagtaagatgaggttgaagata

SEQ ID NO: 25 - DNA, 51 bp, Homo sapiens
caccccacactggacacatccttataggcactgagacacttctgggagcac

SEQ ID NO: 26 - DNA, 51 bp, Homo sapiens
catcttacagagtgaagtgcctgatcctaagatatggtggtcaaagaggat

SEQ ID NO: 27 - DNA, 51 bp, Homo sapiens
gtgcctggcctacgattttaattacggtagatttatattacacttaaacct

SEQ ID NO: 28 - DNA, 51 bp, Homo sapiens
gtattttcttttaactttcaaaactgtttttgctccaaagaacaaaagcaa

SEQ ID NO: 29 - DNA, 51 bp, Homo sapiens
gctggagaggcaaagttgggacaagcatatttctatatttcttagtataag

SEQ ID NO: 30 - DNA, 51 bp, Homo sapiens
tcctaatcagtgggactttaagagtgctttaagggcaatattcctattatg

SEQ ID NO: 31 - DNA, 51 bp, Homo sapiens
agactagagagactaaaacagttgaagtgaagaacctgaaaggagagctta

SEQ ID NO: 32 - DNA, 51 bp, Homo sapiens
caaatgtttaaagctaatgtcttaatggtatgtactaaatcaagctactaa

SEQ ID NO: 33 - DNA, 51 bp, Homo sapiens
aatggggcatatgttccataagtagaccaggggtatcaccaatgagcgtct

SEQ ID NO: 34 - DNA, 51 bp, Homo sapiens
gtctacctcttacctttgccatttcgttctgtcaatcagaattgagtccag

SEQ ID NO: 35 - DNA, 51 bp, Homo sapiens
cctaccttcttcgctccctagctgcgtttatttaacttagttcagaggctt

SEQ ID NO: 36 - DNA, 51 bp, Homo sapiens
cacacacaaagatgaaatgaactcctttgtcgtgtaagaatctctccaacg

SEQ ID NO: 37 - DNA, 51 bp, Homo sapiens
gtggaccaaacagaatccaggctccaagcctctccctggcaagcctcttgc

SEQ ID NO: 38 - DNA, 51 bp, Homo sapiens
cagcctttgtctcctgcacaggtactgttcgctactcttaaggatgcaaaa

SEQ ID NO: 39 - DNA, 51 bp, Homo sapiens
atcagaacttacagaacacttttagagtagatatcagagaaaaattcatag

SEQ ID NO: 40 - DNA, 51 bp, Homo sapiens
cttttcaagtggacttgtaaaaatcgaagagaaaaatgcaaagtttcccaa

SEQ ID NO: 41 - DNA, 51 bp, Homo sapiens
ggttccattgagactgatgctgcattgaattctgatgatacagttcgctgc

SEQ ID NO: 42 - DNA, 51 bp, Homo sapiens
gatttgtatactattgaggtattaacgatccatatttaaccaagtgttttc

SEQ ID NO: 43 - DNA, 51 bp, Homo sapiens
gggggtgaaaggagtccataccattagttcttagcttcaagacctaaatca

SEQ ID NO: 44 - DNA, 51 bp, Homo sapiens
ggtcacctcaaaccatgattcaaataaggcagattttgagtttgtgcataa

SEQ ID NO: 45 - DNA, 51 bp, Homo sapiens
cagggtaaattcctgtaaggcctggatgccctgctgtgaggtcaaaggggg

SEQ ID NO: 46 - DNA, 51 bp, Homo sapiens
agtgcgtggttagctgtgttagaccagtacgctgaatcgctttgtggaact

SEQ ID NO: 47 - DNA, 51 bp, Homo sapiens
cttaagtctctgaattgagctttcttatagcagtagggtcctcatcgtctg

SEQ ID NO: 48 - DNA, 51 bp, Homo sapiens
ataagaaaaagcatgcagagatacatgcagacttgagaaatcgatggcatt

SEQ ID NO: 49 - DNA, 51 bp, Homo sapiens
acctcatataccttgtatccagaagcaaccaggtcatctcttagcattttc

SEQ ID NO: 50 - DNA, 51 bp, Homo sapiens
gcaatcaatggaatttgcaagtgcgagatgaattttccttaattaaaatct

SEQ ID NO: 51 - DNA, 51 bp, Homo sapiens
ggctcactttagggtccaggagaaacaatatggaaaccagtaaagcttttt

SEQ ID NO: 52 - DNA, 51 bp, Homo sapiens
gcctagcctgatcttcaaccttgtctgttctccagctatagatgagagtct

SEQ ID NO: 53 - DNA, 51 bp, Homo sapiens
ggacaaacatcttacatcttatacaccatggttttgttcagaactgggctt

SEQ ID NO: 54 - DNA, 51 bp, Homo sapiens
cttcttatcttactggggtaatccaattatgacttgtacctgctctggctt

SEQ ID NO: 55 - DNA, 51 bp, Homo sapiens
agggggagacctaattatctgggctgtccttcaagatccttctctttcttt

SEQ ID NO: 56 - DNA, 51 bp, Homo sapiens
aataagaacattttgaaaaataggtaagaatttaagcccatcaacataaaa

SEQ ID NO: 57 - DNA, 51 bp, Homo sapiens
tctagggaatgtgatttaggattaagttctcaagtacttttttttgcaata

SEQ ID NO: 58 - DNA, 51 bp, Homo sapiens
ggccaaattgataatatatacaatccagtagctttgttagttattcaagca

SEQ ID NO: 59 - DNA, 51 bp, Homo sapiens
tttgtaacttccttgaaggcagagtttcttcttcgggtttgtattatctat

SEQ ID NO: 60 - DNA, 51 bp, Homo sapiens
attttcccctaattatcacaaataatttaaaattacatgcatgcctcatta

SEQ ID NO: 61 - DNA, 51 bp, Homo sapiens
ttaatatgacaataatctccacagctggtacatatttgccaaatgtggtag

SEQ ID NO: 62 - DNA, 51 bp, Homo sapiens
tgaagaagcaggtagaacactgtgaccttacgatgtgaattctctaatcag

SEQ ID NO: 63 - DNA, 51 bp, Homo sapiens
acaccctttaactttatttacagtaaatgagagcaagttttaaaagccttt

SEQ ID NO: 64 - DNA, 51 bp, Homo sapiens
ttattgtagtacattgaaagaaacacgaataaacaggaatataccattcaa

SEQ ID NO: 65 - DNA, 51 bp, Homo sapiens
tgtgagccccttttgtcattgtcagggcctaagggtccaggaatcactgtc

SEQ ID NO: 66 - DNA, 51 bp, Homo sapiens
gaggggaaattccaagctgcagagtacgtgaaaacagttttttaaagttga

SEQ ID NO: 67 - DNA, 51 bp, Homo sapiens
cattccatgttcgtcctgaggtaaattagcctaagtgattattgtatcaaa

SEQ ID NO: 68 - DNA, 51 bp, Homo sapiens
tgtattgggagtgccctgtagaggatgaaagggagagaagcaaggagagta

SEQ ID NO: 69 - DNA, 51 bp, Homo sapiens
gggagctcacaccttcgagaggccacgctgctgccgaaaagaggggctgct

SEQ ID NO: 70 - DNA, 51 bp, Homo sapiens
tgaaattgtgggtatcacatgggatccttaggagcagagtataagcacaga

SEQ ID NO: 71 - DNA, 51 bp, Homo sapiens
gtcgcccacggtggtcacatgatcagtcctggtctttataccattacttcc

SEQ ID NO: 72 - DNA, 51 bp, Homo sapiens
ccttttggaaaaaaaatcatgtgtcatctccattatactgaactctctgca

SEQ ID NO: 73 - DNA, 51 bp, Homo sapiens
acaagacacatgagggaaaaaaaccgtcttggaggttcttgccccaacaga

SEQ ID NO: 74 - DNA, 51 bp, Homo sapiens
gacatacactttcttccaattctggatatatttgtgtgcacacaggctact

SEQ ID NO: 75 - DNA, 51 bp, Homo sapiens
ggagtatgggaaggtcctgaaagagcccctaagttatgcttgatttactgc

SEQ ID NO: 76 - DNA, 51 bp, Homo sapiens
cagaaaatcatcttttcaggcttaaggtgatcctcaaaatcagaaaaaaac

SEQ ID NO: 77 - DNA, 51 bp, Homo sapiens
ttttacggaagtatttcttttgctcaggacagttctaagtgcagtggcaga

SEQ ID NO: 78 - DNA, 51 bp, Homo sapiens
ttcagtctccaccttttagaattcagaccattatctgcggttttggataat

SEQ ID NO: 79 - DNA, 51 bp, Homo sapiens
tccaaccaactcattcagatgtgccgagacctgggcatggtgctggggata

SEQ ID NO: 80 - DNA, 51 bp, Homo sapiens
ggaagaagccaaaataaacagagatgaacagacataaaaagaaattatagc

SEQ ID NO: 81 - DNA, 51 bp, Homo sapiens
tatatggtggttcttcttcatttcaatctcagaggataactttctaaattc

SEQ ID NO: 82 - DNA, 51 bp, Homo sapiens
ctcagataaattcatttaagtggctgaggaacacagctaataattggcaga

SEQ ID NO: 83 - DNA, 51 bp, Homo sapiens
attattttatttcatgcagttacatttatttattcagcaaatatttattga

SEQ ID NO: 84 - DNA, 51 bp, Homo sapiens
aaaggccagcaggactacgggttaggcatgagtaagccgacgggagaggga

SEQ ID NO: 85 - DNA, 51 bp, Homo sapiens
gccattatgcaattctccctaactgaactacctttcaccttcatctctgct

SEQ ID NO: 86 - DNA, 51 bp, Homo sapiens
gtgcacaactgtcaagtttatgagacgacttgaggtttgctgttgtctcaa

SEQ ID NO: 87 - DNA, 51 bp, Homo sapiens
actggcctcaccttataatgtaacagcccattattgatttaggacaggttt

SEQ ID NO: 88 - DNA, 51 bp, Homo sapiens
atagcacctgttaaatggaaaagtgattagctattataagtttagaaaaaa

SEQ ID NO: 89 - DNA, 51 bp, Homo sapiens
gtgggagttgtgttcagccgcaaaagaaaatgaattagtgtgttatatttg

SEQ ID NO: 90 - DNA, 51 bp, Homo sapiens
agcaagagaagcttctcaaggatacgggcattcaaaaagccaagaataggc

SEQ ID NO: 91 - DNA, 51 bp, Homo sapiens
ggtcagataatatcatccaaaccacatgaatggaacactgagatactgcta

SEQ ID NO: 92 - DNA, 51 bp, Homo sapiens
tactgatagttacagctactatatacaattcaattgacttcctcaggatta

SEQ ID NO: 93 - DNA, 51 bp, Homo sapiens
taacgagaagccttatatactacataaaggagcctaggttttatcctccag

SEQ ID NO: 94 - DNA, 51 bp, Homo sapiens
acataaaacccttcaatggatcctcaagaccaatgaaataaagtgctaacc

SEQ ID NO: 95 - DNA, 51 bp, Homo sapiens
cccagggacctttcaattttatgcttatctttctttatatattaatatcaa

SEQ ID NO: 96 - DNA, 51 bp, Homo sapiens
cctgaaatcaatcttaaattttcaacttgttttcctctacactgtcactac

SEQ ID NO: 97 - DNA, 51 bp, Homo sapiens
tcaaattaaggggatcatcaacaacgttttctacagttcacataggaggcg

SEQ ID NO: 98 - DNA, 51 bp, Homo sapiens
tttctgagaaggccttaaagaaaatcatggtatacttagaattattggaca

SEQ ID NO: 99 - DNA, 51 bp, Homo sapiens
tgcgataccatctccccacttctacgccaagctgctcacggtctcacccag

SEQ ID NO: 100 - DNA, 51 bp, Homo sapiens
caaggccgtctaaattgtccttccagtgtaaagtccattgaatttttctcc

SEQ ID NO: 101 - DNA, 51 bp, Homo sapiens
taaattactaatatgtagcaccttgcaagtgtataagtggtcaactttata

SEQ ID NO: 102 - DNA, 51 bp, Homo sapiens
atgcaaaatccctggcttcaaggaacgcatttccctgatgcaggccacatg

SEQ ID NO: 103 - DNA, 51 bp, Homo sapiens
cgaaccagttgattttgtttgcatgcatgaatttacatgtcctttcaacag

SEQ ID NO: 104 - DNA, 51 bp, Homo sapiens
tgtctttatctgcactataaaatactgcagcctagctggatgagacggtta

SEQ ID NO: 105 - DNA, 51 bp, Homo sapiens
gtgcccagcccctggtgattttatggagaacttactctgtgcccttggata

SEQ ID NO: 106 - DNA, 51 bp, Homo sapiens
tacaagtatgatagcatcaaacacagggcttagtttgcatgccctcttata

SEQ ID NO: 107 - DNA, 51 bp, Homo sapiens
tttctggtttcaatccagtgaaagtgctcttcaattctctaccttttgcca

SEQ ID NO: 108 - DNA, 51 bp, Homo sapiens
ttgagcaatttcaggacagcagtttatcttttggtctgagttgtttggagt

SEQ ID NO: 109 - DNA, 51 bp, Homo sapiens
ataagaatttccctatcagctacagggtctcttgcagggaaattgttttaa

SEQ ID NO: 110 - DNA, 51 bp, Homo sapiens
gtcaacatttttgtctccccaagcattttctgatttgaactattttctcac

SEQ ID NO: 111 - DNA, 51 bp, Homo sapiens
ttaaaatactcagcttaggaagttgctgttaaataacataaagacaattac

SEQ ID NO: 112 - DNA, 51 bp, Homo sapiens
gtatcatcttgtgtttctcctagtgtatcaatatttgtttctgagtatgat

SEQ ID NO: 113 - DNA, 51 bp, Homo sapiens
aaatagccttgataaaatgaaaagatccctaattgtggcagttggaagttt

SEQ ID NO: 114 - DNA, 51 bp, Homo sapiens
agggaaagaggtgtagcaggcctgagcaaacccaaccatgatgggctttag

SEQ ID NO: 115 - DNA, 51 bp, Homo sapiens
tgggtgtctgaggcccttgcccctcgcttatcttctcccagatacataaga

SEQ ID NO: 116 - DNA, 51 bp, Homo sapiens
gttattatatcagggtagtagaataagggattttttttttaatctattttc

SEQ ID NO: 117 - DNA, 51 bp, Homo sapiens
aggacttgtgcagccgggcctgcctctgagtggtgcctctcctggaaggaa

SEQ ID NO: 118 - DNA, 51 bp, Homo sapiens
ggagcttggccctggtggtgagtctataagatgcagtaggtcttagagtga

SEQ ID NO: 119 - DNA, 51 bp, Homo sapiens
gtgcagtccctacaccctgcttcccgcaaccatcctcaacccaacagcccc

SEQ ID NO: 120 - DNA, 51 bp, Homo sapiens
tttctcatacacaattcaatcaaaaattactaggaccaagtgaccaaattc

SEQ ID NO: 121 - DNA, 51 bp, Homo sapiens
tttttttttaaagagaatcaaagatgaataagatttagtaccaagtgagac

SEQ ID NO: 122 - DNA, 51 bp, Homo sapiens
tagttactaaagcttccttcctaaatgaatgatcaccaagaggaccccatg

SEQ ID NO: 123 - DNA, 51 bp, Homo sapiens
cagcagagatgtcccaggctcaacaaatttgcgactgaactcttgttctga

SEQ ID NO: 124 - DNA, 51 bp, Homo sapiens
cactctagaggagtctacttttcagcgatgagcaggagaatgccaataaat

SEQ ID NO: 125 - DNA, 51 bp, Homo sapiens
ccttaccaaatggagggtctgaacttggatgccgggaagttttctggaagg

SEQ ID NO: 126 - DNA, 51 bp, Homo sapiens
tagtgtttttcaggagataatgtgactgtttttgcgctagaaagtcatgaa

SEQ ID NO: 127 - DNA, 51 bp, Homo sapiens
atgcagtgagcaaaatggatttggcagtgacttaaaaagacttcatgtatt

SEQ ID NO: 128 - DNA, 51 bp, Homo sapiens
gaaaaaaaaaactcattcttacccagcttacttattgtcaatacataaata

SEQ ID NO: 129 - DNA, 51 bp, Homo sapiens
cctatttattcacttgagcattctgaatatcattgacattttctttgactg

SEQ ID NO: 130 - DNA, 51 bp, Homo sapiens
ggctgtaatcgaagcagaagtgcctcacaaattaactagcagtcgcccaca

SEQ ID NO: 131 - DNA, 51 bp, Homo sapiens
gttcaaacaccggaaatgtctccagtcatggaaaatgagcaaataatgagc

SEQ ID NO: 132 - DNA, 51 bp, Homo sapiens
gggcccagcctgtgcacctcgactccttccagcctatgctttcttgcccct

SEQ ID NO: 133 - DNA, 51 bp, Homo sapiens
caaggaagaagcaagtccttgcatagtgatcgtttgtgacctaaacaacta

SEQ ID NO: 134 - DNA, 51 bp, Homo sapiens
ccctaatttatttgtgggataaaaacgatattgaatatgagagtggtggga

SEQ ID NO: 135 - DNA, 51 bp, Homo sapiens
ttatgttgatatattttccttttcataacaccctaaaatctagatacaata

SEQ ID NO: 136 - DNA, 51 bp, Homo sapiens
gatgttgtcgtcggggtgaactgtgaccctgtgggacaagtatatagaggg

SEQ ID NO: 137 - DNA, 51 bp, Homo sapiens
gtagcaagtgcattctgtgaaacaaagattttttatcattattattggcca

SEQ ID NO: 138 - DNA, 51 bp, Homo sapiens
agccaaaaaagtatcacttaatgcaccaaagtaataatgagcaaattagta

SEQ ID NO: 139 - DNA, 51 bp, Homo sapiens
gaaaacttcagagaatcttgtgtaacggaacagataattttagtaagttaa

SEQ ID NO: 140 - DNA, 51 bp, Homo sapiens
tcatgtaattggtaaagtaccattggtattttttcctcatattagatatga

SEQ ID NO: 141 - DNA, 51 bp, Homo sapiens
atttgcaacagccattcaactctagtattagcctaatgatatcaaagtcca

SEQ ID NO: 142 - DNA, 51 bp, Homo sapiens
atttgctgaggatataaaggggagactgtccaaatgcaaaccctcaggaag

SEQ ID NO: 143 - DNA, 51 bp, Homo sapiens
aaaatgtattgttactttactacagagaccattacattgtatagaccagtt

SEQ ID NO: 144 - DNA, 51 bp, Homo sapiens
aggaggtaggtttgtccaaaccatgcaaattctaaggacagccgataatac

SEQ ID NO: 145 - DNA, 51 bp, Homo sapiens
gtaaattaatttgaagggtaaagtataattctgatacttttttgcattttt

SEQ ID NO: 146 - DNA, 51 bp, Homo sapiens
aagtggtgttccatgttgcttccttattactaaacatgagaaactgcatta

SEQ ID NO: 147 - DNA, 51 bp, Homo sapiens
gcaaggaactcaagacagcctatctttctggaaaagtaactgtccagtttt

SEQ ID NO: 148 - DNA, 51 bp, Homo sapiens
actccagctgtgtgtgattcctgtcctatctgtctgactacaaagcacata

SEQ ID NO: 149 - DNA, 51 bp, Homo sapiens
tggtcatccattcctcaagccagccggaatctccaagttcatttcttccat

SEQ ID NO: 150 - DNA, 51 bp, Homo sapiens
ccattcattccagatgtctcacaacggatgactggccccagtcacactgcc

SEQ ID NO: 151 - DNA, 51 bp, Homo sapiens
tgctgttcctctgacaatcaattcattgatcttcaccattccactccagga

SEQ ID NO: 152 - DNA, 51 bp, Homo sapiens
ggcactcaattaatacttgaaacaatgtgtgaatgagaagcgtgcagtaat

SEQ ID NO: 153 - DNA, 51 bp, Homo sapiens
actatatctaaacaaacccttaattacagcagtaccacaggacactgttct

SEQ ID NO: 154 - DNA, 51 bp, Homo sapiens
agtgacatttcaagaataccaccttcatccagtcaggcgtatgctgagaaa

SEQ ID NO: 155 - DNA, 51 bp, Homo sapiens
attatagaagagtttggaggaagacgttcagcatttgagctaatatgtgca

SEQ ID NO: 156 - DNA, 51 bp, Homo sapiens
tttcttctctgtatcactgtttctatctctgtccctctcagtagcaccctg

SEQ ID NO: 157 - DNA, 51 bp, Homo sapiens
ttggacgattgcttgggtgagccacgatacatacgttggtgcattcattta

SEQ ID NO: 158 - DNA, 51 bp, Homo sapiens
tccatcaggcccattcttggcatcacgttagttttctgataatttatcaca

SEQ ID NO: 159 - DNA, 51 bp, Homo sapiens
tgccttcccagaagccaaaggataatgatggtctcattcttcacattgtaa

SEQ ID NO: 160 - DNA, 51 bp, Homo sapiens
tattaacagaacaaatgtgcagagggatacacagacaggataaaatctaaa

SEQ ID NO: 161 - DNA, 51 bp, Homo sapiens
gcattcactctctcccctttaatctaggttccctgaggctactggaggcct

SEQ ID NO: 162 - DNA, 51 bp, Homo sapiens
agttgccaaccaggtatttctccccaaatgtccactgttacctgaaactcc

SEQ ID NO: 163 - DNA, 51 bp, Homo sapiens
gaggtttcattaccagtaacactgctgacgcttgagctcagaggtagcaag

SEQ ID NO: 164 - DNA, 51 bp, Homo sapiens
atctcatctgctcccacaaccaccatctaaaataggcagagcaggttttac

SEQ ID NO: 165 - DNA, 51 bp, Homo sapiens
tttcatatcccacactgaataccttgtgatggcactgccactaccactgtt

SEQ ID NO: 166 - DNA, 51 bp, Homo sapiens
cccttcaagagctgactgaccagggctggacagttaactcactcctccagt

SEQ ID NO: 167 - DNA, 51 bp, Homo sapiens
gatgagccctgctccctgggtaaggccggtgtggatgaagaagtggtcaga

SEQ ID NO: 168 - DNA, 51 bp, Homo sapiens
ggtcctgatatactatgataagtgctatatgcagagtgaacacagggcact

SEQ ID NO: 169 - DNA, 51 bp, Homo sapiens
cctaatctctgggaacaagtgaaatcgagagagccggaagcacccaaaacc

SEQ ID NO: 170 - DNA, 51 bp, Homo sapiens
attagataattaaaagcctctgccatcagtcaaaatgaaactttttttgtg

SEQ ID NO: 171 - DNA, 51 bp, Homo sapiens
gataattgatagattggttgagtaactagaaataatcagtgcaattaaatt

SEQ ID NO: 172 - DNA, 51 bp, Homo sapiens
tcttctgtagtcatataagaaatttaaggcaatagcagatatttgtatatt

SEQ ID NO: 173 - DNA, 51 bp, Homo sapiens
tgtgacagtatttaaaatgcaaagggaagacttagagagtagagtatgtct

SEQ ID NO: 174 - DNA, 51 bp, Homo sapiens
gcaatgtctaaccttgccttcttttaagaaaactctgtgtctgtgttgcat

SEQ ID NO: 175 - DNA, 51 bp, Homo sapiens
ctcccatctgtgcctgcaatataagtttgaatcactgaacttatctgattg

SEQ ID NO: 176 - DNA, 51 bp, Homo sapiens
agcctgcgtgctgcaaacgacgcatttgacctccaagatgactccacgttc

SEQ ID NO: 177 - DNA, 51 bp, Homo sapiens
tccaatcaagtctctgctacttagtatcttattaactggaaccctctaaaa

SEQ ID NO: 178 - DNA, 51 bp, Homo sapiens
cccttcaaattcgttaattctctgactgcagttgaagaaaatccagccaag

SEQ ID NO: 179 - DNA, 51 bp, Homo sapiens
tctgcctatgactctggccagtgtgtttttcgctgagctgttggtggaagt

SEQ ID NO: 180 - DNA, 51 bp, Homo sapiens
ttttctagttgattaggcagattaactttctgtttttctgctgacataaat

SEQ ID NO: 181 - DNA, 51 bp, Homo sapiens
aggctgagggaatctgctgccatgaatatccatttacttacagaatttgtt

SEQ ID NO: 182 - DNA, 51 bp, Homo sapiens
ttttcaataattttttccaggttgtctgcattcaaaagagcattctattaa

SEQ ID NO: 183 - DNA, 51 bp, Homo sapiens
cctctttttcaagatgaaatatgcaaagtgaagtatgcagatagcaggacc

SEQ ID NO: 184 - DNA, 51 bp, Homo sapiens
cacaactgtatctgaacagattctcgttacataaaaccgcacacacagtgt

SEQ ID NO: 185 - DNA, 51 bp, Homo sapiens
ataatggcaccaattatttcttcaattatgaaggcaccaatgggatggaaa

SEQ ID NO: 186 - DNA, 51 bp, Homo sapiens
cttcaaacttgtgcaagatggtgccatagaggataaaaacaagatggaggc

SEQ ID NO: 187 - DNA, 51 bp, Homo sapiens
taccttcatataatagcatttgaaaggctcattaaatagaacagagaagaa

SEQ ID NO: 188 - DNA, 51 bp, Homo sapiens
attgtacattcctaatctacacgaattttctccaaattgtataggcctcct

SEQ ID NO: 189 - DNA, 51 bp, Homo sapiens
gtttgactcatttcgggaatattctatctccaccatttttcattgcccttt

SEQ ID NO: 190 - DNA, 51 bp, Homo sapiens
gaacattcaagtttaaagtgaaagcgagtacaaagccaaggtacagaaaag

SEQ ID NO: 191 - DNA, 51 bp, Homo sapiens
aattttaagccaaaattgtttttcacaattgcaaccataattcagtttttc

SEQ ID NO: 192 - DNA, 51 bp, Homo sapiens
catgaaggccaacaagaaatagagatgttgaaagaaaacagagagaaagga

SEQ ID NO: 193 - DNA, 51 bp, Homo sapiens
gtggtcaggttctggacagttctctgtgagaatatttaaatgatacattgc

SEQ ID NO: 194 - DNA, 51 bp, Homo sapiens
cactgatgagtaccagactagaagaagatatttggaataccacagaaggag

SEQ ID NO: 195 - DNA, 51 bp, Homo sapiens
tgccctctgcaccttgggcacaaatcgagattggattcctggggaaactct

SEQ ID NO: 196 - DNA, 51 bp, Homo sapiens
tgcaggtacacggagcgccttcttttcgtttagagcattggtaagtaccta

SEQ ID NO: 197 - DNA, 51 bp, Homo sapiens
tctgttggcgaaagaaaaaatgcacctgtattatgtaaataagaatccatg

SEQ ID NO: 198 - DNA, 51 bp, Homo sapiens
ttgcaaaagcctaaggactgtgtaaatattcataatactgtttttaaatta

SEQ ID NO: 199 - DNA, 51 bp, Homo sapiens
tggggaagggtatgtttgcaggctattgttaacccagatgagtgagatgca

SEQ ID NO: 200 - DNA, 51 bp, Homo sapiens
ttgtaagaaagtgtgtgctatttctgggttatccatgtgggcagggggaaa

SEQ ID NO: 201 - DNA, 51 bp, Homo sapiens
gttgagtcccattatcactccaggaggtacttttgggataggtcctttttt

SEQ ID NO: 202 - DNA, 51 bp, Homo sapiens
tcgtgcaatgtctctgtgtggtagaaagaataagtacggtaatcctccctt

SEQ ID NO: 203 - DNA, 51 bp, Homo sapiens
gtgcctttttcataccttaaatagatgctcataaaccaaagtgttatatgt

SEQ ID NO: 204 - DNA, 51 bp, Homo sapiens
cagtataatttaagattaatattgcttgtgtagtcaatgcatattcagggt

SEQ ID NO: 205 - DNA, 51 bp, Homo sapiens
gatttgttctaagttatctctactcccttgcctggccttttgttaaaaaca

SEQ ID NO: 206 - DNA, 51 bp, Homo sapiens
ccaccagtccataggggtgaaaaaacctatttttatgcactgtgacatttt

SEQ ID NO: 207 - DNA, 51 bp, Homo sapiens
ggccaacatcagagtttttctagccgaagttattttagagggtaatcagga

SEQ ID NO: 208 - DNA, 51 bp, Homo sapiens
agaaaacaatgttacagtgcagttgatgggcccttcatggtgctggccact

SEQ ID NO: 209 - DNA, 51 bp, Homo sapiens
cattcattcttttattctttaatgcactcattcattcaacatttattaagc

SEQ ID NO: 210 - DNA, 51 bp, Homo sapiens
tctaagtgcactgcaaatgtggcataactagttaactttcttcagtgattt

SEQ ID NO: 211 - DNA, 51 bp, Homo sapiens
acctatttcctataagaccatggaggtgagcttacctctctaagcctcaat

SEQ ID NO: 212 - DNA, 51 bp, Homo sapiens
caatcatgttacaaaaagaaaattaacaataccctaaaataaagaattcca

SEQ ID NO: 213 - DNA, 51 bp, Homo sapiens
tagatgtattagaacatcatccttccgctagtaaatagagtactgtgggtt

SEQ ID NO: 214 - DNA, 51 bp, Homo sapiens
aacttcattttccagcctcaccaatcttttagaacagaagatcaagtcaaa

SEQ ID NO: 215 - DNA, 51 bp, Homo sapiens
aagtagaaccattttgagtttttcacaagaaagatatttaaggctgacgtt

SEQ ID NO: 216 - DNA, 51 bp, Homo sapiens
tatactgagtgaatggctattaaacatccatcccaatgtaagtaacaggac

SEQ ID NO: 217 - DNA, 51 bp, Homo sapiens
tatcagaaatgcttgacttagaagcgtctccttcatttgctggaatatgaa

SEQ ID NO: 218 - DNA, 51 bp, Homo sapiens
gttacaagttatttctttggtgtcaacgctcattttgtgtgtgtgactaac

SEQ ID NO: 219 - DNA, 51 bp, Homo sapiens
tggtacaggtaacactcataagtcacgtgtatagtactctccaattgacaa

SEQ ID NO: 220 - DNA, 51 bp, Homo sapiens
gagcagacctgatgacactgaacctggctagagaggtactcaaaaccaagt

SEQ ID NO: 221 - DNA, 51 bp, Homo sapiens
gtgtcaatgcataattttgtaaactatgttactgtcctcaagattacaaac

SEQ ID NO: 222 - DNA, 51 bp, Homo sapiens
actagagcctttattctcagaatctaagttattgtaggtaaaaaatggatg

SEQ ID NO: 223 - DNA, 51 bp, Homo sapiens
agggaatggttagtggcacttaaagagactgaaatacaacatatggggagt

SEQ ID NO: 224 - DNA, 51 bp, Homo sapiens
tgaagcctcaaactccttgcgtgtttaatttactgctgttcatccagaact

SEQ ID NO: 225 - DNA, 51 bp, Homo sapiens
atttatccataactggtatgacagggaatctcaaaaaaggaaatatcctgt

SEQ ID NO: 226 - DNA, 51 bp, Homo sapiens
tgcagaggcaggcttaggtggagttgagctagatgtaacaggactatttct

SEQ ID NO: 227 - DNA, 51 bp, Homo sapiens
attacggatagaacaggggagcctaagtttattctctctgacctcccacct

SEQ ID NO: 228 - DNA, 51 bp, Homo sapiens
gcaggagagaatatttagtctggccagatcagagagggcgaggtggagaat

SEQ ID NO: 229 - DNA, 51 bp, Homo sapiens
tgacattgaagaccagaatggttcacttgatgagagtccccaaagctagtg

SEQ ID NO: 230 - DNA, 51 bp, Homo sapiens
tcaatcttttcacctttacgtttgacctttcaatcctttcgccttattgag

SEQ ID NO: 231 - DNA, 51 bp, Homo sapiens
taaaatttataaggtgatttttgtttctaccagtgtaaagaagacccatgg

SEQ ID NO: 232 - DNA, 51 bp, Homo sapiens
tttggggtgggtgatgggacaggctgttgaatagcagaaacaatggaagaa

SEQ ID NO: 233 - DNA, 51 bp, Homo sapiens
gtgaagcttccttccctgacctttcgcttatcataattgagacctatttaa

SEQ ID NO: 234 - DNA, 51 bp, Homo sapiens
ttcaggagactgggaaacattggattgaacacatgatatgtgagctgggag

SEQ ID NO: 235 - DNA, 51 bp, Homo sapiens
agatgttatccagtttgccaagaaaccctcacctagaggcaccaaattatt

SEQ ID NO: 236 - DNA, 51 bp, Homo sapiens
aagatatgtcctgttcaaaaccaatgttctgagagtttgagagccgtgaaa

SEQ ID NO: 237 - DNA, 51 bp, Homo sapiens
ttataaattactactagtgttaatatttcctggaacagtaaaaagaagtag

SEQ ID NO: 238 - DNA, 51 bp, Homo sapiens
atagatgaacaagtaaaaagtctcaatggggtctgtttcactgtggagcag

SEQ ID NO: 239 - DNA, 51 bp, Homo sapiens
tctcccactcttttcccttctccttcttgcctatgaagcctttcctgacca

SEQ ID NO: 240 - DNA, 51 bp, Homo sapiens
gcaaagaagaaaaactaataagcccataactgacattgagttctagttgag

SEQ ID NO: 241 - DNA, 51 bp, Homo sapiens
agctcactccctgcttcagaagtgaccatggatctcaatggcacaagattt

SEQ ID NO: 242 - DNA, 51 bp, Homo sapiens
aggataataaggctgaccttgccgggtgttgggagaatcagataatcagag

SEQ ID NO: 243 - DNA, 51 bp, Homo sapiens
tacagaacatctgaatagtggaatgttgagccaccattaaataaatttctt

SEQ ID NO: 244 - DNA, 51 bp, Homo sapiens
aaatttgggcaaattaccaaccattgtgggtctcagtttcgactgttaaca

SEQ ID NO: 245 - DNA, 51 bp, Homo sapiens
aaacagtaccatacattgccttttattagatttttttttttttccagagtc

SEQ ID NO: 246 - DNA, 51 bp, Homo sapiens
ggcagacagtgatctccaggatatatcagtaaatttaaaaatggtaattta

SEQ ID NO: 247 - DNA, 51 bp, Homo sapiens
tatgtgtataattgctcattcatggactggtggacaagcctctttcagatg

SEQ ID NO: 248 - DNA, 51 bp, Homo sapiens
ggaatgcatgtgctggtgaatgttacggtggggaaatggttgatgagactc

SEQ ID NO: 249 - DNA, 51 bp, Homo sapiens
gctaatgtgaattcaacacaggaaaactaaaaggtggtcactggttttcat

SEQ ID NO: 250 - DNA, 51 bp, Homo sapiens
aaagtagaaaacagcagagctaaaatacaaaaattttcaaagaaataatct

SEQ ID NO: 251 - DNA, 51 bp, Homo sapiens
gtgtatttagaattatgtctgacacaccgttaagaaccataaagtattagt

SEQ ID NO: 252 - DNA, 51 bp, Homo sapiens
cagtaaagggagaggaagaggatttgagaagcctagggacttaacatgctt

SEQ ID NO: 253 - DNA, 51 bp, Homo sapiens
ttgtttgccattccattttgaaacaagtagtataattctaaaacaaacaaa

SEQ ID NO: 254 - DNA, 51 bp, Homo sapiens
gatctacaattctggacttggcagcaacttcccagtagccattctacaatg

SEQ ID NO: 255 - DNA, 51 bp, Homo sapiens
ggcttggagaatctttgcatgtacagtttatataagcaagcttcataatta

SEQ ID NO: 256 - DNA, 51 bp, Homo sapiens
ttaaatgtgaattgtaactttatacgtgcgctatttttaacgtttctctct

SEQ ID NO: 257 - DNA, 51 bp, Homo sapiens
agaaatagaaataaggtttccagttgttatttactcaggcttccaattatt

SEQ ID NO: 258 - DNA, 51 bp, Homo sapiens
atagtacagaatatggctaaaaggaccaggtgtgaggtgcagattaagtgc

SEQ ID NO: 259 - DNA, 51 bp, Homo sapiens
tacagttcacaagctcaaagctttgttgttctctgtcgtcacatgactatc

SEQ ID NO: 260 - DNA, 51 bp, Homo sapiens
agtcagatcctgcaaaatgatttatctgttcctatgcctttggcctctagt

SEQ ID NO: 261 - DNA, 51 bp, Homo sapiens
gaaagatttctactgtagagaacaacgtggcatttcttcaggggatggaga

SEQ ID NO: 262 - DNA, 51 bp, Homo sapiens
ggcaattaaacaaaaatttaaagtaccaccctgagaggtaaaaagagtaaa

SEQ ID NO: 263 - DNA, 51 bp, Homo sapiens
gtttcaaatcatggaagcatgttatttataaggaatgtataaaatatcttt

SEQ ID NO: 264 - DNA, 51 bp, Homo sapiens
gtgaaaagatataaagcaatgttccttcatttttatgaatttaaagtacct

SEQ ID NO: 265 - DNA, 51 bp, Homo sapiens
ccgacatctgcttattaacaatatggaatgttgagagctttacgaaactga

SEQ ID NO: 266 - DNA, 51 bp, Homo sapiens
ttgggttgggcaacatgtggaggaactgaaagcctggagcctgggcccagg

SEQ ID NO: 267 - DNA, 51 bp, Homo sapiens
acctccccttatcaaagacacacacttgaatagcgtgatcagtgacacgtt

SEQ ID NO: 268 - DNA, 51 bp, Homo sapiens
tgacctgtttcagagcacacctccaaccttacccagctctcacccacctac

SEQ ID NO: 269 - DNA, 51 bp, Homo sapiens
ggaagaaagatacttggagagttaatgatgtaaggacacaagtacagtcat

SEQ ID NO: 270 - DNA, 51 bp, Homo sapiens
ttcctatgcattatgatttgaactcaaaaaacagaaggctatcagaaagac

SEQ ID NO: 271 - DNA, 51 bp, Homo sapiens
gaaggactggacaagacaggagcatttcctaatcaatttatgcaaaacctc

SEQ ID NO: 272 - DNA, 51 bp, Homo sapiens
tgtgtttagaatatggactgtgttctggctgttacaggttcatgtgtgtta

SEQ ID NO: 273 - DNA, 51 bp, Homo sapiens
tgtatactcttgtgttatgatacccgcagcacagcagtgccaggcactcac

SEQ ID NO: 274 - DNA, 51 bp, Homo sapiens
gctgctctttgaagacaatattttcgaatttcatctgttatttcaaggttt

SEQ ID NO: 275 - DNA, 51 bp, Homo sapiens
catgatacctgaggaagtaacaaagattatacaatcatccctaagtattca

SEQ ID NO: 276 - DNA, 51 bp, Homo sapiens
gaacttgcttttatttatctgtcaaggagccttatgtctgtgaaatcacta

SEQ ID NO: 277 - DNA, 51 bp, Homo sapiens
cattgtctcttcttgagaagaatcagtttgcactatatgatatctaagtac

SEQ ID NO: 278 - DNA, 51 bp, Homo sapiens
gtgccctgcagggttgcttaggcctgtaatgtggtccttcacacctctctc

SEQ ID NO: 279 - DNA, 51 bp, Homo sapiens
tttctttgcaaacctgtcttgcctatttttccttaggttgaaaggattctg

SEQ ID NO: 280 - DNA, 51 bp, Homo sapiens
tgatgatttctgagaagatatgacttgtaactttccaataacacttttcta

SEQ ID NO: 281 - DNA, 51 bp, Homo sapiens
gcctgagttttccttagcaacctaattctgtttgccttatgtgcttgactt

SEQ ID NO: 282 - DNA, 51 bp, Homo sapiens
tgcagggaaagaaggccaagaatggccaacactgacttggagaaggatacc

SEQ ID NO: 283 - DNA, 51 bp, Homo sapiens
atattgaatactgatattttggggccgttattaacccagctttcttttaaa

SEQ ID NO: 284 - DNA, 51 bp, Homo sapiens
acagactggtaacatcttcccattattgcatgagaaatttttttcccttag

SEQ ID NO: 285 - DNA, 51 bp, Homo sapiens
tatcctccaaaacacctccatgggatctttagagaacttcagaacatggtt

SEQ ID NO: 286 - DNA, 51 bp, Homo sapiens
gggaaaggccatttcagctcattgacaggtagtagaggattaacgtaattg

SEQ ID NO: 287 - DNA, 51 bp, Homo sapiens
gggtgacaaagagtatcttgagctccctgaactgtagatcacaagcccctt

SEQ ID NO: 288 - DNA, 51 bp, Homo sapiens
gagaactttgtatgcactcacaagtatacatcaaaatctcctggtggtttg

SEQ ID NO: 289 - DNA, 51 bp, Homo sapiens
catagtgagaccctggaacccctactaagctgtttgatttagagactcttg

SEQ ID NO: 290 - DNA, 51 bp, Homo sapiens
tatagtgattaaagtagcacttacacttttgaccctggcaggtattatctg

SEQ ID NO: 291 - DNA, 51 bp, Homo sapiens
gcagcataatgtcccatcagtccctttctgacaaataacctagaactctca

SEQ ID NO: 292 - DNA, 51 bp, Homo sapiens
aaaccaacacattgcaattattttgtattatatacagtataagtacatcat

SEQ ID NO: 293 - DNA, 51 bp, Homo sapiens
ttacagacgcttaacacagtttaacgaatttctctgatacaccaacagtgg

SEQ ID NO: 294 - DNA, 51 bp, Homo sapiens
atcacttgggtaatgcaacccatttatctgctcccactgtataacagcgga

SEQ ID NO: 295 - DNA, 51 bp, Homo sapiens
tattaatctttaaaaattttaaagtaaagcctctacctattcacagcaaga

SEQ ID NO: 296 - DNA, 51 bp, Homo sapiens
agactcatctccatgaaatgataggatgttctcttgtgtgcattccattgg

SEQ ID NO: 297 - DNA, 51 bp, Homo sapiens
aaggagatcaggatagccaatttcaggagcttcccttccttgatctcattg

SEQ ID NO: 298 - DNA, 51 bp, Homo sapiens
aacaggctaaggtaaatactcacgcgaaagaaccaaaaactttgtaagagt

SEQ ID NO: 299 - DNA, 51 bp, Homo sapiens
ctttggggaactgaaagaagccctggagaacacaatatacaatggcacacc

SEQ ID NO: 300 - DNA, 51 bp, Homo sapiens
cccaccaccactatttattgactgggtgactataggtaagatagtgagcct

SEQ ID NO: 301 - DNA, 51 bp, Homo sapiens
tctttgaaagccttgaaaatcctaatgttcttgctaacagtggaaatgctt

SEQ ID NO: 302 - DNA, 51 bp, Homo sapiens
agactgatacattgctcatattcatgttgatcttcattatgtttgtgacac

SEQ ID NO: 303 - DNA, 51 bp, Homo sapiens
tacacaaagacaaatgagaaatattcttacctcaggaggttaatatgaagt

SEQ ID NO: 304 - DNA, 51 bp, Homo sapiens
cttgaaaattgttatctcttgtttagtatcttatttcggaaagcagcatct

SEQ ID NO: 305 - DNA, 51 bp, Homo sapiens
taaatggaatgagcacgtctaaaagtgtatataccatgtgtcctaatttca

SEQ ID NO: 306 - DNA, 51 bp, Homo sapiens
agtaggtcctgacttcatcagtctatagtgttctgttccagcattcttttg

SEQ ID NO: 307 - DNA, 51 bp, Homo sapiens
catgcatacttacatagttcatcagtaagtgaggattcaatggatggatga

SEQ ID NO: 308 - DNA, 51 bp, Homo sapiens
ataggaggactattcagtatttggtagggagcagggtaacagaaaggttac

SEQ ID NO: 309 - DNA, 51 bp, Homo sapiens
gatttttgcacaaaatgggttcttcaatataaacccttcagtatttccagc

SEQ ID NO: 310 - DNA, 51 bp, Homo sapiens
cttttaactcttcctttactgcatttgtgcctttccaatgacaatctcgtt

SEQ ID NO: 311 - DNA, 51 bp, Homo sapiens
ctgaaccataagcactgaattgaccgttctctttccccataactgccatct

SEQ ID NO: 312 - DNA, 51 bp, Homo sapiens
ggagatgataccttcatcgaagacatcctcctattagcattctagagcaga

SEQ ID NO: 313 - DNA, 51 bp, Homo sapiens
tacactaacacagagtgcacataccccatcttttaagccgagttccattac

SEQ ID NO: 314 - DNA, 51 bp, Homo sapiens
ttgaaagcttctgtaaacagttgaacttcaaattaaaaggtaagtaggaac

SEQ ID NO: 315 - DNA, 51 bp, Homo sapiens
gtttatttttcatggttttaacccagcattaagtagcatggtttttagcat

SEQ ID NO: 316 - DNA, 51 bp, Homo sapiens
aatatgaaagagacatgtgaatctctgcctttgaatacttaggatgtgttt

SEQ ID NO: 317 - DNA, 51 bp, Homo sapiens
gttccataacctttggggccaattacaggtcatggatacactgttcctaag

SEQ ID NO: 318 - DNA, 51 bp, Homo sapiens
aacaccagcatggatgacttccacaatgatatgactttcatgcctcccagt

SEQ ID NO: 319 - DNA, 51 bp, Homo sapiens
aagaattattaaagtacctactacacactacataccatatatcaattaaat

SEQ ID NO: 320 - DNA, 51 bp, Homo sapiens
gaaagaagtttcacctgcatgctgcgagctttgtgcctgctgctataataa

SEQ ID NO: 321 - DNA, 51 bp, Homo sapiens
tatgattgtaaagaacattacagctgaaaactcaaaataataagtgttttg

SEQ ID NO: 322 - DNA, 51 bp, Homo sapiens
ctttgagagcacaaagaggtggcaactaacatactccagtgtgggacagag

SEQ ID NO: 323 - DNA, 51 bp, Homo sapiens
caaaaaccttaatttaaaaaaatctgtatcaaagaataaaattttcccaat

SEQ ID NO: 324 - DNA, 51 bp, Homo sapiens
actgtggtggcaaactcatctatttatttaacactggtatttcagccatct

SEQ ID NO: 325 - DNA, 51 bp, Homo sapiens
gcatgtgtggagcttgagccggcgataaattgaggcgctaatcctgatgca

SEQ ID NO: 326 - DNA, 51 bp, Homo sapiens
caagtaattgaatcttctaatggaacaaactggtctctgcttaatgatttg

SEQ ID NO: 327 - DNA, 51 bp, Homo sapiens
tatccagatctctccccaggctacatctctctcatgagctccatacctctg

SEQ ID NO: 328 - DNA, 51 bp, Homo sapiens
tgactctgtgtcagcttgacgagcaagttttttggtagtgaaacatttgtc

SEQ ID NO: 329 - DNA, 51 bp, Homo sapiens
cttatcacactttttcattgagtgtctattttacaaacaggggagatcaaa

SEQ ID NO: 330 - DNA, 51 bp, Homo sapiens
tcatttcgtgcaggtcagaagtatgctgcagtactgattgtgaaagaagta

SEQ ID NO: 331 - DNA, 51 bp, Homo sapiens
cagaggaagctctccatgtaacacaaagtgcagtgaggagtgagaaccaca

SEQ ID NO: 332 - DNA, 51 bp, Homo sapiens
attttcccctctctcaaatgttgtaactcctagtacagtttctttagcatc

SEQ ID NO: 333 - DNA, 51 bp, Homo sapiens
atttttgttagagccattaattctactatgctggactagtcaagagggccc

SEQ ID NO: 334 - DNA, 51 bp, Homo sapiens
catttcatgacatgcatttcaaatattttatacagctgcttccttaaagac

SEQ ID NO: 335 - DNA, 51 bp, Homo sapiens
gcaccaaacgaatatgtccaatgtagggcgaatgttgtcttctcaattctt

SEQ ID NO: 336 - DNA, 51 bp, Homo sapiens
ttcacgaagaaactaacttttcagttgctataagatttttgtagaacccaa

SEQ ID NO: 337 - DNA, 51 bp, Homo sapiens
aacagagatcctactagacatagacgatttaaaaaaaaatttgaaaagtct

SEQ ID NO: 338 - DNA, 51 bp, Homo sapiens
atactcatgacagcccgacacactcaagccgtgctgcagactatgaaagac

SEQ ID NO: 339 - DNA, 51 bp, Homo sapiens
caatttggcactgccaaggacttcagcccataatcccttcttccgtagtaa

SEQ ID NO: 340 - DNA, 51 bp, Homo sapiens
taaccaacaagaaaaacaagatcagcgctatgagagagagtgacagggtgg

SEQ ID NO: 341 - DNA, 51 bp, Homo sapiens
gctcacaaactcaacaattgcttgtcttttttcctagggtataagcctttg

SEQ ID NO: 342 - DNA, 51 bp, Homo sapiens
acctgctggtcccagtggcaggtaacggctgctcttatcagcaggggtaac

SEQ ID NO: 343 - DNA, 51 bp, Homo sapiens
gtcggagagaaacgatggtggctcagactagagtggtgtttgtggaacttg

SEQ ID NO: 344 - DNA, 51 bp, Homo sapiens
ctctacattcccatgcctcagtgacattttatttattttatttatttattt

SEQ ID NO: 345 - DNA, 51 bp, Homo sapiens
ctggagaggctttttttgattccaataacatgattcctagggtaaaattac

SEQ ID NO: 346 - DNA, 51 bp, Homo sapiens
gattaataatgttgctgtccagattcccgttatagcgctaacctgatgtta

SEQ ID NO: 347 - DNA, 51 bp, Homo sapiens
tttcacagtaaggtaaattgcttttataattattggattgcctctctgact

SEQ ID NO: 348 - DNA, 51 bp, Homo sapiens
cgatggccgccatgaaacggtcggcgatgatggacactcccagaaacatgt

SEQ ID NO: 349 - DNA, 51 bp, Homo sapiens
ttatcctacacatccatatcctggaagttgaactgtggtgagctcctggac

SEQ ID NO: 350 - DNA, 51 bp, Homo sapiens
ggagaggcccatgggtactcttcatctcccaaggagctcccaaagtccttt

SEQ ID NO: 351 - DNA, 51 bp, Homo sapiens
agaaacttctctgatcacatatccaattgttaaaaagaattgtgggttact

SEQ ID NO: 352 - DNA, 51 bp, Homo sapiens
gggaaagtgtcacacagattgacaaatgtggagtcacaaagcaaaggaggt

SEQ ID NO: 353 - DNA, 51 bp, Homo sapiens
tggccacattcaggaaccagggaatggaagggatgctaatctgaggacatt

SEQ ID NO: 354 - DNA, 51 bp, Homo sapiens
cacaaccttcaccctgctgtgcctcgctgcaaggctctgttcagtcaatta

SEQ ID NO: 355 - DNA, 51 bp, Homo sapiens
gaagaagtcgggtaggcctagaccagaatcccacctgggtgaccttgggca

SEQ ID NO: 356 - DNA, 51 bp, Homo sapiens
tttgtacaatcaaaaaggagcctacgggatcacttctgagcaagagcgctg

SEQ ID NO: 357 - DNA, 51 bp, Homo sapiens
ggggagtgatttggtcctttacagacggatgaatgaatttctgtatccaaa

SEQ ID NO: 358 - DNA, 51 bp, Homo sapiens
catctttttttaagcttttatcatgttttttgacaatgtagttaaagtcta

SEQ ID NO: 359 - DNA, 51 bp, Homo sapiens
gccttcagcctccacagagtcttctagtagtaaacccttaaagatgcttca

SEQ ID NO: 360 - DNA, 51 bp, Homo sapiens
actatgattattaaaaaggaattgattgggattggaaatcaagagggccag

SEQ ID NO: 361 - DNA, 51 bp, Homo sapiens
acagaattaatgataatggtagtgacggctaacattttcaagcacgtacat

SEQ ID NO: 362 - DNA, 51 bp, Homo sapiens
cttacatagaagggagggtgtttgaaataaaggatagttatattaggtagg

SEQ ID NO: 363 - DNA, 51 bp, Homo sapiens
tgtggttggcactgaaaccagcaggcaagaccagagagtaaagaagcaaaa

SEQ ID NO: 364 - DNA, 51 bp, Homo sapiens
cagctattgcttatgctccacgcaccatttgcccttttggaggatcatcgt

SEQ ID NO: 365 - DNA, 51 bp, Homo sapiens
acttactgaagtccaaaaccaagctaagtacatttgttgtcaagtgagttc

SEQ ID NO: 366 - DNA, 51 bp, Homo sapiens
agagccaccatttccttgacctaatttggactcttctcaaactcaccacaa

SEQ ID NO: 367 - DNA, 51 bp, Homo sapiens
agttgtccatgaatcagtatttgggcaacaaaatcactatacaaccctgct

SEQ ID NO: 368 - DNA, 51 bp, Homo sapiens
aatggatgagaatcttctgcagccatctgaatcaaattctgtgtgcttagc

SEQ ID NO: 369 - DNA, 51 bp, Homo sapiens
tcatcacacggtctcaaatccctacttactcatgtttgtctcttgccagtt

SEQ ID NO: 370 - DNA, 51 bp, Homo sapiens
agtccgcaggagcttaaatgaggctcatggtctggactggctgttttcctc

SEQ ID NO: 371 - DNA, 51 bp, Homo sapiens
tctatacccactgatctcaagaccacaaagtttttgttgttgttgttgttg

SEQ ID NO: 372 - DNA, 51 bp, Homo sapiens
ttcaacccatcctgaaatctgctgactgattaacccacttcaatccagtct

SEQ ID NO: 373 - DNA, 51 bp, Homo sapiens
gaacttctttaacaggttctgagtagaaacatagctcaatgaagaaataaa

SEQ ID NO: 374 - DNA, 51 bp, Homo sapiens
atatgtatgaaaatagattctaaaggtattagatcagcagtccccaacctt

SEQ ID NO: 375 - DNA, 51 bp, Homo sapiens
ctgtttcagttctatggcagtaattcaagtaagagtgaagacacccattgc

SEQ ID NO: 376 - DNA, 51 bp, Homo sapiens
ggtgatcagcatgctgctggccctatgatgataagtagtgggctcttcctt

SEQ ID NO: 377 - DNA, 51 bp, Homo sapiens
gtaaccccgggagctgagtgagagaggctccttcccttacatccacatgcc

SEQ ID NO: 378 - DNA, 51 bp, Homo sapiens
tcctgggccactgaattgccaactgcgtgacccaaggctcctctaaacctg

SEQ ID NO: 379 - DNA, 51 bp, Homo sapiens
cactgagtaagcaggtgcctccaaaggtcttactaagccacaggtaggaag

SEQ ID NO: 380 - DNA, 51 bp, Homo sapiens
caaccctggctgacatgactccttcgattgctaatcagtcctcagtcaccc

SEQ ID NO: 381 - DNA, 51 bp, Homo sapiens
actttagtactctgaatctcccgcagtgtccaatactgtacttttttacat

SEQ ID NO: 382 - DNA, 51 bp, Homo sapiens
gcagtgtgatagcataagtcacttaatcttcacaatgctcctttgattctc

SEQ ID NO: 383 - DNA, 51 bp, Homo sapiens
agttttaggaacctaatttttttcagtggtcaattttggcttacaaaccag

SEQ ID NO: 384 - DNA, 51 bp, Homo sapiens
gcctttagaaacgcaggttctgggccgctcacccccatttctggagctgca

SEQ ID NO: 385 - DNA, 51 bp, Homo sapiens
actgggtgcagccagattctgcttacgttttggttgccccatgaaatcgcc

SEQ ID NO: 386 - DNA, 51 bp, Homo sapiens
cccaagaaggtatttctttgtctagaatcaccatatagcttatcttggtct

SEQ ID NO: 387 - DNA, 51 bp, Homo sapiens
tcactatatgttggccttgattggtgttcctgaagtcttttgggcatttct

SEQ ID NO: 388 - DNA, 51 bp, Homo sapiens
ggtttgccttcttaaatatgttattctaggtcattggtaacataagtttta

SEQ ID NO: 389 - DNA, 51 bp, Homo sapiens
cgttctccccttcttagcttcgactcgcattctcatgaacatctctcctag

SEQ ID NO: 390 - DNA, 51 bp, Homo sapiens
gtctattcatcttcacagcaatctatttcaaaagtgcttatctctgctgct

SEQ ID NO: 391 - DNA, 51 bp, Homo sapiens
cacatattttctcttgcaagttcgtatgatttgcattatttaaacttgcaa

SEQ ID NO: 392 - DNA, 51 bp, Homo sapiens
ctattacaggactcctcataaacgcagtgaagctagagtgaaaaatagata

SEQ ID NO: 393 - DNA, 51 bp, Homo sapiens
acttgctaatgaattataacatcaattgtgtatcaacaaacaatatttaat

SEQ ID NO: 394 - DNA, 51 bp, Homo sapiens
gaagtgataaagtgttgtgagtaacccagctttcttagaattgaaacaagt

SEQ ID NO: 395 - DNA, 51 bp, Homo sapiens
gtctcagtcctgtttaggtttatagttttaatgtagggcagcagggatctt

SEQ ID NO: 396 - DNA, 51 bp, Homo sapiens
caatctgttaagggtaaataagatataaacacgtgaaaacataaatcacaa

SEQ ID NO: 397 - DNA, 51 bp, Homo sapiens
attataaactaggtcaattttctgctactccatgaggccttttttagaagg

SEQ ID NO: 398 - DNA, 51 bp, Homo sapiens
agattcactacacaccttcagccctgaccagtgcgactgaggcagaaggct

SEQ ID NO: 399 - DNA, 51 bp, Homo sapiens
ttatctaaatcctagtagagaatcaatctaagttaacccacactttggtgt

SEQ ID NO: 400 - DNA, 51 bp, Homo sapiens
gactgctagcatctccttcttggctaatgtttatttgggcagatgtcctat

SEQ ID NO: 401 - DNA, 51 bp, Homo sapiens
aactcaaccaccacttattgttttttgaagataatgttggtcgtgcgcctg

SEQ ID NO: 402 - DNA, 51 bp, Homo sapiens
agaaattgtacgaagtagatgcaaatagttcattaggactactaagtatga

SEQ ID NO: 403 - DNA, 51 bp, Homo sapiens
tgatagcatttggattgggctttaaggtatgactaggagcttaccagatag

SEQ ID NO: 404 - DNA, 51 bp, Homo sapiens
agccaatcattttcccacaaagttacgatacagcatcaaccactggtgtct

SEQ ID NO: 405 - DNA, 51 bp, Homo sapiens
aaatattttttacacctcatagtcattcaaataggtacacgtcctttttga

SEQ ID NO: 406 - DNA, 51 bp, Homo sapiens
gctaaacaaagagacagatacctaatcatgggtactttcccaaggagaaat

SEQ ID NO: 407 - DNA, 51 bp, Homo sapiens
tgaagtaggagtcaggagaactgaatgctattgaaatagctgtcactcata

SEQ ID NO: 408 - DNA, 51 bp, Homo sapiens
actttggcatgtactaacgatactatattatgagatattttatgtatcctt

SEQ ID NO: 409 - DNA, 51 bp, Homo sapiens
ctgtaaagaatctagcctactaattgtatatcaccattagtgaacattttt

SEQ ID NO: 410 - DNA, 51 bp, Homo sapiens
gtctgcttttgtaagattgttaattgtataaccccagattttcaagtgaca

SEQ ID NO: 411 - DNA, 51 bp, Homo sapiens
atgcaggagcaaaagcagcacggttcgaggcagcggaagcattaggtatca

SEQ ID NO: 412 - DNA, 51 bp, Homo sapiens
cgtatattgcacacaaaaagatctcaattaacaacatctctcgctctggat

SEQ ID NO: 413 - DNA, 51 bp, Homo sapiens
cattttttttttaagaggtgctgttgttcattctcacatgaattagagaga

SEQ ID NO: 414 - DNA, 51 bp, Homo sapiens
taaatcagttcttgaaaaaatagcttgaggctcatgcagctttggaaactc

SEQ ID NO: 415 - DNA, 51 bp, Homo sapiens
tttcctttccctttttatggtgtttttatggtggttaagagcgggagcttc

SEQ ID NO: 416 - DNA, 51 bp, Homo sapiens
actgagacttttcttcatgctacggcctaactcgtcccctctccttcctat

SEQ ID NO: 417 - DNA, 51 bp, Homo sapiens
ccagtgtatattgatctaatgttcactaatactcttagaaattgatactga

SEQ ID NO: 418 - DNA, 51 bp, Homo sapiens
tgaaaagaattattggccagagcgacccatagttgtttttgctaatgtgag

SEQ ID NO: 419 - DNA, 51 bp, Homo sapiens
tccaggtgtttcatcatttggtagcagataacaattccagcctctgctaac

SEQ ID NO: 420 - DNA, 51 bp, Homo sapiens
aggaaagcagaattcaacacatctttgaaagaaaatccaaatgctgtatta

SEQ ID NO: 421 - DNA, 51 bp, Homo sapiens
gttggacactaggaactgttatacacaagggaagtccaggacctctctccg

SEQ ID NO: 422 - DNA, 51 bp, Homo sapiens
attaactaatacttcatattctttacgctgaaacctaatttgtttttaatt

SEQ ID NO: 423 - DNA, 51 bp, Homo sapiens
aaacagctgtatctaatactttaatggcatgttagagctaagtaaatagta

SEQ ID NO: 424 - DNA, 51 bp, Homo sapiens
aaggtttggaatcaaggtcatgggtagagttggcatgatggaagaacggat

SEQ ID NO: 425 - DNA, 51 bp, Homo sapiens
gaaggataattttatttccagatgattatttccagtttttaacgtgaaatc

SEQ ID NO: 426 - DNA, 51 bp, Homo sapiens
tatgttggctagctgcatgcacagtcttctcttgaagaacgtctacattgg

SEQ ID NO: 427 - DNA, 51 bp, Homo sapiens
taagtttgacaagcactatttgccatatcacattcttggaaattcacagtg

SEQ ID NO: 428 - DNA, 51 bp, Homo sapiens
gataaagatctggatggagaatgcccgacatatgtgcaaatttgtgccaaa

SEQ ID NO: 429 - DNA, 51 bp, Homo sapiens
taggatcctgcagttaataatggccagtggcactgatggtcattaacgttc

SEQ ID NO: 430 - DNA, 51 bp, Homo sapiens
atgtccctttagagcctctctgataagattgctggaatgaataagctactg

SEQ ID NO: 431 - DNA, 51 bp, Homo sapiens
atattactgcctacccacatatttcgtcttctctatttctctataataaaa

SEQ ID NO: 432 - DNA, 51 bp, Homo sapiens
aagtaacactataaaccttggcgttaggggatttttacatgcctaaatttg

SEQ ID NO: 433 - DNA, 51 bp, Homo sapiens
tggttgtaacaaatattgtgccagcattatgaggacttagcatcaatctca

SEQ ID NO: 434 - DNA, 51 bp, Homo sapiens
gctaagacatgagattccattatcagtaatgttattcctgtaagtgtgtgc

SEQ ID NO: 435 - DNA, 51 bp, Homo sapiens
tgccttttggcccctgtaatttagttagtctctgattttatcctaagagta

SEQ ID NO: 436 - DNA, 51 bp, Homo sapiens
aaggccctggacatcccatgacaaaattagcttaaatgtcaggaccagaga

SEQ ID NO: 437 - DNA, 51 bp, Homo sapiens
aaattgatatccttatttccagagtgagactgtacagttattttcttatac

SEQ ID NO: 438 - DNA, 51 bp, Homo sapiens
attttattgtatggtaaagtcaaggtaaacaatttataactataatgtcac

SEQ ID NO: 439 - DNA, 51 bp, Homo sapiens
tatgtagagcttcgaaatcaattaagttccttacaacctctgacataaagc

SEQ ID NO: 440 - DNA, 51 bp, Homo sapiens
actataaaacgttttcatgatccctgttctcatggagtattctattagtga

SEQ ID NO: 441 - DNA, 51 bp, Homo sapiens
aagtaaaccaggtaggggttttcaacagcctttatatttacatatgtatat

SEQ ID NO: 442 - DNA, 51 bp, Homo sapiens
tgatttcagaatggggtttggcatacggccaatgattagctctctgcatat

SEQ ID NO: 443 - DNA, 51 bp, Homo sapiens
caattgtgtggtatgcaatatgttcgtagtaaacaattcaagtagcacaga

SEQ ID NO: 444 - DNA, 51 bp, Homo sapiens
tgctgcccttagccactggcataactgcaagcctctcgggtcatctagctt

SEQ ID NO: 445 - DNA, 51 bp, Homo sapiens
tgatattagatgatggtaatttaaatttagagcaatggctgttctgggaac

SEQ ID NO: 446 - DNA, 51 bp, Homo sapiens
ttcactttttgtcagttcatgccctgtggtaggttccccatatacaatgag

SEQ ID NO: 447 - DNA, 51 bp, Homo sapiens
ctggggaagccccagcagatgcctcgatgtgagtgtttctgggatagtaca

SEQ ID NO: 448 - DNA, 51 bp, Homo sapiens
tgctgtgagtaataatgactctgctggtaatttgtgtccttctgcttggaa

SEQ ID NO: 449 - DNA, 51 bp, Homo sapiens
gtaaactctagttctgtgtcatatccgatggggaaagggttccaccattaa

SEQ ID NO: 450 - DNA, 51 bp, Homo sapiens
aacaaaaatgtgaggaattatcttcgcagggagcagataaactatgctttc

SEQ ID NO: 451 - DNA, 51 bp, Homo sapiens
caacagacaaaatgcactagcaactggagaggtgagttttgttaaattaca

SEQ ID NO: 452 - DNA, 51 bp, Homo sapiens
aggagagataaatatataagcaacaagttatattggaaataaaacccatgg

SEQ ID NO: 453 - DNA, 51 bp, Homo sapiens
cacacctgcacgcctgagcagactttcgagcagggcgcaaagttgggtggc

SEQ ID NO: 454 - DNA, 51 bp, Homo sapiens
tccgtctcttcctcatccttattcttgtcactgggagacattcgtcactga

SEQ ID NO: 455 - DNA, 51 bp, Homo sapiens
aaatgcaaggaatgacaaaagaatgagtgcttcatcctagtttctaggcct

SEQ ID NO: 456 - DNA, 51 bp, Homo sapiens
catattaccttattatcctacaaaacaaaatgcatactcaataacattaat

SEQ ID NO: 457 - DNA, 51 bp, Homo sapiens
ctgcatcccctctttttctcagcaaacactctcaagtctctcccatctatt

SEQ ID NO: 458 - DNA, 51 bp, Homo sapiens
taaacactttcaccattaataacagttcattggcattttagcagatctaca

SEQ ID NO: 459 - DNA, 51 bp, Homo sapiens
tatgtgtgggcgtgtatgtgtgtccattcatttaaggaaacttggaaaata

SEQ ID NO: 460 - DNA, 51 bp, Homo sapiens
tttcaatatacaccagctgctaaatgtagtttctattgaatattcagacag

SEQ ID NO: 461 - DNA, 51 bp, Homo sapiens
taaacatgtaaacatgctgtctctactattctgaaaaactacaacaggatt

SEQ ID NO: 462 - DNA, 51 bp, Homo sapiens
ttttatagttacagaatgtgtagtatatgccaggtgctattgctggcactg

SEQ ID NO: 463 - DNA, 51 bp, Homo sapiens
ggggcaaagatggggattagaatgatgacattgcaaagaaagtatcatcag

SEQ ID NO: 464 - DNA, 51 bp, Homo sapiens
tctacaaccggttcaagggccacaacgacctgatggaatacgcaaagcacc

SEQ ID NO: 465 - DNA, 51 bp, Homo sapiens
gttttgctttacatagccaaattaagtacctgaaataaaatatagaatata

SEQ ID NO: 466 - DNA, 51 bp, Homo sapiens
ttgaggagaatgaggatatggtgagcgttagcatagaatgtaacctgaaac

SEQ ID NO: 467 - DNA, 51 bp, Homo sapiens
tctcagttgcttttaatctacttcttttgggatagagagagatgagttgca

SEQ ID NO: 468 - DNA, 51 bp, Homo sapiens
tcccgtcctcctgtgctgtaagctcatcttcataacataatgtttattttt

SEQ ID NO: 469 - DNA, 51 bp, Homo sapiens
gacatagcagagggccagagggccacaaagaataaaagatgtgaggccttg

SEQ ID NO: 470 - DNA, 51 bp, Homo sapiens
ctttgtatcctttcagtttcatatcttcctctcttgtgctcagacccttac

SEQ ID NO: 471 - DNA, 51 bp, Homo sapiens
tgggatggaggaatgaatgaagaaacggtgaggctgggtgaggtggctcac

SEQ ID NO: 472 - DNA, 51 bp, Homo sapiens
taaagtaccaaatttcatgaataacttctgcaaaggagaaccatttattta

SEQ ID NO: 473 - DNA, 51 bp, Homo sapiens
agaggtgcaagtagtgaacgcctgacgccccgaccactgtgctctccattc

SEQ ID NO: 474 - DNA, 51 bp, Homo sapiens
aggtcaccaaggaagggttttgatttgtttatatatgtttctttggacaag

SEQ ID NO: 475 - DNA, 51 bp, Homo sapiens
ggaagaattaaccaaaagccattcatgagtgactggaacccaggattctgg

SEQ ID NO: 476 - DNA, 51 bp, Homo sapiens
gtaatttaactgaactagtctcctattggacacttgcattctgttcagtgt

SEQ ID NO: 477 - DNA, 51 bp, Homo sapiens
atggagagattgaacctaaagatctgtaaagtctctgcaactctcaagtcc

SEQ ID NO: 478 - DNA, 51 bp, Homo sapiens
catattattaaaactcttgatgtaggccaccagttgctttctagaaagatt

SEQ ID NO: 479 - DNA, 51 bp, Homo sapiens
gttagttcagcatggtaaaaataaccctgagtgggctgtaattttggtgac

SEQ ID NO: 480 - DNA, 51 bp, Homo sapiens
atgtttatgaaagcacatacaggcaataacagtgctaaccttcagaggtta

SEQ ID NO: 481 - DNA, 51 bp, Homo sapiens
ccttccctggctgtatctgcctccttgccacaggttatcattattctaaat

SEQ ID NO: 482 - DNA, 51 bp, Homo sapiens
taaatagaataacatggatatcaagaggttcttaattttcatatttcagat

SEQ ID NO: 483 - DNA, 51 bp, Homo sapiens
gagaagctgtccttaaatgggggtgtccgggagtcctgacgaaagatgagc

SEQ ID NO: 484 - DNA, 51 bp, Homo sapiens
gatttctcaatgttgttgtaggtctcggaggaacatgtgtgaatgtgggtt

SEQ ID NO: 485 - DNA, 51 bp, Homo sapiens
gtccaaacataccgatacaaaaacagttacattatttcatctcatactggt

SEQ ID NO: 486 - DNA, 51 bp, Homo sapiens
aataaatgactttgcacataattatccttggtttacttcttctcagctgca

SEQ ID NO: 487 - DNA, 51 bp, Homo sapiens
ttcactcttttggaagtgtttcttcgtgcatgcccccacttaaaaaaaaac

SEQ ID NO: 488 - DNA, 51 bp, Homo sapiens
tgctgagggtacacatatgactagatacaaagctgtccacttgatctacta

SEQ ID NO: 489 - DNA, 51 bp, Homo sapiens
aagaaacaatttgtaaaactgtctcggcgaattcacttaaccaaaacttgt

SEQ ID NO: 490 - DNA, 51 bp, Homo sapiens
tgctgttgtacatactctgaagcaggattttgagtcactgatttgacatgg

SEQ ID NO: 491 - DNA, 51 bp, Homo sapiens
ttcataatcagtaattgtttctcagacgtgcttgattctggaatctctttg

SEQ ID NO: 492 - DNA, 51 bp, Homo sapiens
tcagtcaccagcatgccaaaaccattcttaggaattgcccaacagaccctg

SEQ ID NO: 493 - DNA, 51 bp, Homo sapiens
accagctatgctaccatttctactctgtttatgcctactaagagggcttga

SEQ ID NO: 494 - DNA, 51 bp, Homo sapiens
gatctttagtacacagtctattaatccactcaaaaccccatgattatccta

SEQ ID NO: 495 - DNA, 51 bp, Homo sapiens
ccctgaaaagttggtattttcttccgtatcggtccttcatcattgaacatg

SEQ ID NO: 496 - DNA, 51 bp, Homo sapiens
cattcacctattgttccttggagaccagtgacattgatagggaaaatactc

SEQ ID NO: 497 - DNA, 51 bp, Homo sapiens
agcaaaaggcaaaatcgactaagtgccttgccttcctcttaagttggtaga

SEQ ID NO: 498 - DNA, 51 bp, Homo sapiens
tatgccaccttcaaaagcctacctcgtaaaaattcttcctctttagcctgc

SEQ ID NO: 499 - DNA, 51 bp, Homo sapiens
tataatgatagtgattgtttcctatatatcacaatttggagataaataatc

SEQ ID NO: 500 - DNA, 51 bp, Homo sapiens
actgtaatcattccagtgaacagtgtgggtttttctcagagactgatgggc

SEQ ID NO: 501 - DNA, 51 bp, Homo sapiens
aatgttttaaagtacagtatttgctcgatgaaaatatgatgttgtgacttg

SEQ ID NO: 502 - DNA, 51 bp, Homo sapiens
gtaaatgcaagaaagtgagagtatcagttatgaaaggcacctttaatacca

SEQ ID NO: 503 - DNA, 51 bp, Homo sapiens
agaaggacaggaatttccaccagcagagccccttaagaaggttatctggga

SEQ ID NO: 504 - DNA, 51 bp, Homo sapiens
cttatcatccttagcattttttagacggccagatttggagtttaatactag

SEQ ID NO: 505 - DNA, 51 bp, Homo sapiens
tattctcaacagatctctcctgaccatgccgttcattgcccttgctgtctt

SEQ ID NO: 506 - DNA, 51 bp, Homo sapiens
aaaaggcagagattacaaccgaagtgttttccttgacccgggaagtgatcg

SEQ ID NO: 507 - DNA, 51 bp, Homo sapiens
taaatactgtctcaaggttggttagcgctttcagatgaagcatctcatttg

SEQ ID NO: 508 - DNA, 51 bp, Homo sapiens
cagaactggtaactaaggcggtgatcaaacaggaatgcttttcttctcagt

SEQ ID NO: 509 - DNA, 51 bp, Homo sapiens
agaccttgagaaacgcttccaaaaactgcaggaatgatttctgaaggctgc

SEQ ID NO: 510 - DNA, 51 bp, Homo sapiens
taagctaatcatacctcccactctgcatctgagcagggtatctgagactcc

SEQ ID NO: 511 - DNA, 51 bp, Homo sapiens
atcactatttgtttgaaaacaatgtcctgataccctgctgtcttcccagta

SEQ ID NO: 512 - DNA, 51 bp, Homo sapiens
ccctgatgacctggtcaatagaaacaaatgaacacgtgacttgcatttgag

SEQ ID NO: 513 - DNA, 51 bp, Homo sapiens
aagaccatcaacatcagacaataaagggcagtgaccactgagagacgggaa

SEQ ID NO: 514 - DNA, 51 bp, Homo sapiens
gttgggttgttcccgtgggttgacagtcagcatgtttttatttattaaatg

SEQ ID NO: 515 - DNA, 51 bp, Homo sapiens
actttgaggcagtaaatgtgaggcagtgacatacatacctccttgtaatag

SEQ ID NO: 516 - DNA, 51 bp, Homo sapiens
gctgagtttgagactatgttacaaatgtttatcctcaatgacatatgagaa

SEQ ID NO: 517 - DNA, 51 bp, Homo sapiens
agaaagagagtatttggttttccagcctcttcaggaggaggttactgggga

SEQ ID NO: 518 - DNA, 51 bp, Homo sapiens
tgtggattaactgtgctcacaataagtatttgtatcatcaattgcatgact

SEQ ID NO: 519 - DNA, 51 bp, Homo sapiens
gattttgtaagaagcaaaagtaatatgagaacatgatcagctgatggctta

SEQ ID NO: 520 - DNA, 51 bp, Homo sapiens
gatcctcattagtcccaaggttgacatatgaatctcttctccctacaccat

SEQ ID NO: 521 - DNA, 51 bp, Homo sapiens
caagaatattcaatggccaacatatttttaggcaaaaaatgcaaactacaa

SEQ ID NO: 522 - DNA, 51 bp, Homo sapiens
tggaaaacattagaagagggttcctgtgtcatagtttcaaaattcttttca

SEQ ID NO: 523 - DNA, 51 bp, Homo sapiens
cctttcacctgagcaaggcatagacgattcattctaaaaagtgacactggg

SEQ ID NO: 524 - DNA, 51 bp, Homo sapiens
cactaaagataactttatagagaggggactttcaaatatttctataactag

SEQ ID NO: 525 - DNA, 51 bp, Homo sapiens
aattaggaaaggaagaggtcaattaaaattactaagtaaggcattaagaca

SEQ ID NO: 526 - DNA, 51 bp, Homo sapiens
agaatacactttttatttcaaagacaagtaaggcaacaccctcctcccagc

SEQ ID NO: 527 - DNA, 51 bp, Homo sapiens
caactggcaggttaatccctgttaatgagccccaaatttaattatcttacc

SEQ ID NO: 528 - DNA, 51 bp, Homo sapiens
tcaaggaaggtgtttcaaaccaagactgatcaagcagcaggaactgcaagg

SEQ ID NO: 529 - DNA, 51 bp, Homo sapiens
tcataggttgagactaagccatcaactccaagtttgaaaagaaaaatagca

SEQ ID NO: 530 - DNA, 51 bp, Homo sapiens
tgtgaacttgtaaggatatttgtttcgggcaaacaccacagttgaatgata

SEQ ID NO: 531 - DNA, 51 bp, Homo sapiens
aactactcacctattatccataaagcgcccatgtattctacttctaattgt

SEQ ID NO: 532 - DNA, 51 bp, Homo sapiens
taaatcagggtaattctcaagttcccagtctacagaggctacaaacagttt

SEQ ID NO: 533 - DNA, 51 bp, Homo sapiens
gacatagagaaatgttcaagagctcgtttggaaaaggagatagatagttca

SEQ ID NO: 534 - DNA, 51 bp, Homo sapiens
atggagttggaagcccacagtattacggccttgcagttctaagagcgggcc

SEQ ID NO: 535 - DNA, 51 bp, Homo sapiens
gtggtgggtggcggatgggacagattacttagacagggttgctgtagactt

SEQ ID NO: 536 - DNA, 51 bp, Homo sapiens
ctcatgcttgagttttaatggtggacaaattatgggagggacatttcccca

SEQ ID NO: 537 - DNA, 51 bp, Homo sapiens
aaagcaccgcaacagtgtgttttgatctgaagtcaattacttgtatcagaa

SEQ ID NO: 538 - DNA, 51 bp, Homo sapiens
tagatgttggagctatcagataacctcataattggtataataaaatgttaa

SEQ ID NO: 539 - DNA, 51 bp, Homo sapiens
accagcaagtccccctaacaaggccaacaggtatttgccataagaacagac

SEQ ID NO: 540 - DNA, 51 bp, Homo sapiens
tgatcatacccagtactcgctggtttttatttaatgtagctttgaaaaaaa

SEQ ID NO: 541 - DNA, 51 bp, Homo sapiens
ctctgaggccagggccaggcctgtgtcccagtgaccaggttcagactttag

SEQ ID NO: 542 - DNA, 51 bp, Homo sapiens
ggcacgtatgctcacaaagtaccagatgttctacatatgccattctctgaa

SEQ ID NO: 543 - DNA, 51 bp, Homo sapiens
aagttccagtgacatgccctaatgcctcttgccattttatacatcgctgcc

SEQ ID NO: 544 - DNA, 51 bp, Homo sapiens
taatgactgtataatattctatctgggtagaccaggcacggtggctcatac

SEQ ID NO: 545 - DNA, 51 bp, Homo sapiens
agctacccaagtacctgtcagctctgttttcaatcttacacttttctttcc

SEQ ID NO: 546 - DNA, 51 bp, Homo sapiens
gtgtaatacccttagctttatatctctcagttttcacacaatgtgttgtat

SEQ ID NO: 547 - DNA, 51 bp, Homo sapiens
ttaaatttttatttgcatatttgttttctatcctaattccctactgatctt

SEQ ID NO: 548 - DNA, 51 bp, Homo sapiens
ctgtgtttctccaaagaaatgatcaccttgatagttggtgatttatatgtg

SEQ ID NO: 549 - DNA, 51 bp, Homo sapiens
ctgaggaccctgaagccaccagtgtcgtattaatcgaaaccagggacttgt

SEQ ID NO: 550 - DNA, 51 bp, Homo sapiens
agaccaaattcatgacattgtttcaaccagtgcagctcctttcatattagt

SEQ ID NO: 551 - DNA, 51 bp, Homo sapiens
gggcagttggttattacacagaaaacatgtgcaacacttaattgactcaaa

SEQ ID NO: 552 - DNA, 51 bp, Homo sapiens
acaccttttattcatgcagagaatcgtgttcctctatcaaagacccacaac

SEQ ID NO: 553 - DNA, 51 bp, Homo sapiens
gtgcaggctgagtcaaactgctgtcatcaccagaagcctccatcggaaggc

SEQ ID NO: 554 - DNA, 51 bp, Homo sapiens
tcattcacagatttatttatggccaagccacacacctttgtgttcaacttc

SEQ ID NO: 555 - DNA, 51 bp, Homo sapiens
tcagggcatggttagtttcaaatcaattaagctccttatgactttatgatt

SEQ ID NO: 556 - DNA, 51 bp, Homo sapiens
agtggtatataaaacacagttgttgaccacaatataactaagttacagagc

SEQ ID NO: 557 - DNA, 51 bp, Homo sapiens
ctctgcattaaaataataatcatggcgagcaacagataaaataatgttaaa

SEQ ID NO: 558 - DNA, 51 bp, Homo sapiens
gtattatacttcagtttacgtaatcgggaaaataagagtggtctagagaaa

SEQ ID NO: 559 - DNA, 51 bp, Homo sapiens
aaacagtaacaacaactgtatttgcataagcaccccataatccacacccac

SEQ ID NO: 560 - DNA, 51 bp, Homo sapiens
ctgctttctatgctggggtggcaacctaatccaaaattcctattgcaggtt

SEQ ID NO: 561 - DNA, 51 bp, Homo sapiens
ccttctctcgaagtttctatatgcagatcatgactgaatattgttgtttaa

SEQ ID NO: 562 - DNA, 51 bp, Homo sapiens
ccatagtggttagttctacattcagcgagtggcttaaaatttatgccaatg

SEQ ID NO: 563 - DNA, 51 bp, Homo sapiens
caaagagttcttaaagctgctttcttggtaattataactgtttcaaggaat

SEQ ID NO: 564 - DNA, 51 bp, Homo sapiens
aagaacaaccatcttgttgctccagggcataggtgaagatccgctgcagcc

SEQ ID NO: 565 - DNA, 51 bp, Homo sapiens
tgagattcttccctgccttcttccataaattcattatattctagccctaat

SEQ ID NO: 566 - DNA, 51 bp, Homo sapiens
ctagataacacacagctactaaatggtaggtcaaactttttgatctattta

SEQ ID NO: 567 - DNA, 51 bp, Homo sapiens
ccttacatgtatttctttaaatggcgtaactccaccaaagatggttttgat

SEQ ID NO: 568 - DNA, 51 bp, Homo sapiens
ttctaaaaaatcacaagctgtgacaatcttgactataagtattaaattcaa

SEQ ID NO: 569 - DNA, 51 bp, Homo sapiens
tttctactttttcaacatcagaaggatagatgtaggacctgctgctttttg

SEQ ID NO: 570 - DNA, 51 bp, Homo sapiens
cccttattctgtcttcacggaaaattattatgcatccctcagttaatactg

SEQ ID NO: 571 - DNA, 51 bp, Homo sapiens
tgtagaggccaggaaaagcttgctgaatttaaaagtcaagtttattccccc

SEQ ID NO: 572 - DNA, 51 bp, Homo sapiens
aggccctaactctacttgaaggtagattcatggagggctatgcagagatga

SEQ ID NO: 573 - DNA, 51 bp, Homo sapiens
gaaacttacagcatattaacagacagttatcaatgacaaaaacattaaaat

SEQ ID NO: 574 - DNA, 51 bp, Homo sapiens
aaaagattagtttacttagtctctacagggtctattgtagccctctattat

SEQ ID NO: 575 - DNA, 51 bp, Homo sapiens
aaaataatttttccaaagagttgattatcttagactaaactaatgactcaa

SEQ ID NO: 576 - DNA, 51 bp, Homo sapiens
ttcccacagtgggccctgcagaaccctcagatacgaaaagtcagcccttcg

SEQ ID NO: 577 - DNA, 51 bp, Homo sapiens
aagcagctgggatctaagttcatttgcatcagggccactccagctcagttc

SEQ ID NO: 578 - DNA, 51 bp, Homo sapiens
ttcacccacaagctcctgttaaaatgccctcttgttagactccatacctct

SEQ ID NO: 579 - DNA, 51 bp, Homo sapiens
tgaaattaaattatttttctagtttgtagaggctatatccatgaccattat

SEQ ID NO: 580 - DNA, 51 bp, Homo sapiens
aggacctattggcgtgaattaacttttgcattgcgttagaaaaactttcta

SEQ ID NO: 581 - DNA, 51 bp, Homo sapiens
ggccaagagctggtggctgactagtgctgtgtgagcatgggtttataaatg

SEQ ID NO: 582 - DNA, 51 bp, Homo sapiens
atgaataagctattaactaaactgagtgtgagtatagcaatgtataatctc

SEQ ID NO: 583 - DNA, 51 bp, Homo sapiens
tcctcctctccttctctcaactctcgctggcctagatgtctctcacatagt

SEQ ID NO: 584 - DNA, 51 bp, Homo sapiens
ctacctctactaccttgtctgaggtcttaacctgcccagtcctgaattcct

SEQ ID NO: 585 - DNA, 51 bp, Homo sapiens
tctggcctatctcatttccaaaagttgtgagacctgatatcgtacctatag

SEQ ID NO: 586 - DNA, 51 bp, Homo sapiens
tagaccccgaccagcctacatattgtttgctctaaggtctagtgccctctc

SEQ ID NO: 587 - DNA, 51 bp, Homo sapiens
ttgagatataaactaaaattccctccctataggttttgtatctcttcgtca

SEQ ID NO: 588 - DNA, 51 bp, Homo sapiens
gttcccagtctgtgcccattcacaattgactccagcacatgaaagaattag

SEQ ID NO: 589 - DNA, 51 bp, Homo sapiens
aaactgtctattcatgatttatgcactgagtattgcagtcctccaaacact

SEQ ID NO: 590 - DNA, 51 bp, Homo sapiens
gtccttccattaggaggtctgcagaacagagataaaaatggaagccagaga

SEQ ID NO: 591 - DNA, 51 bp, Homo sapiens
aggcaattcagacaaaaagttgtgatagcatagaaagctcactggacagac

SEQ ID NO: 592 - DNA, 51 bp, Homo sapiens
ctattttcctcccatagataattttgctcatcgagctgaagttcggaaagc

SEQ ID NO: 593 - DNA, 51 bp, Homo sapiens
attttaggatgttggctataaacacgaatccttcctctatttgcatgctga

SEQ ID NO: 594 - DNA, 51 bp, Homo sapiens
ggaggaaaagatgaaccaactcccacaaagactgaatagaaaaagactcac

SEQ ID NO: 595 - DNA, 51 bp, Homo sapiens
tccaggaagagaagcacttggtctcatatcattatatttaaggagctccaa

SEQ ID NO: 596 - DNA, 51 bp, Homo sapiens
aaagatgacagtggcaatcaacatatgaggaaaaaacactgagagtcaata

SEQ ID NO: 597 - DNA, 51 bp, Homo sapiens
tgcatccagtgcagcccaagaggcgtcatgcattctcttccccttgtttgc

SEQ ID NO: 598 - DNA, 51 bp, Homo sapiens
ccagactacacaagcaccctgagaaatgtgaaacctctcccttggccccct

SEQ ID NO: 599 - DNA, 51 bp, Homo sapiens
gatatctgaaaacaatttgtgaactataaagcattgttaaaataaaagcca

SEQ ID NO: 600 - DNA, 51 bp, Homo sapiens
gccccctttctgcctttgaccctcatggtggctttgagcaggcatctgaat

SEQ ID NO: 601 - DNA, 51 bp, Homo sapiens
ttgtttcctccttggtcttcagttttctcacttgacagttgaggggttgga

SEQ ID NO: 602 - DNA, 51 bp, Homo sapiens
tgtgtcagttggtaaagaactggaacgaaatgtcctggtttgacgttttga

SEQ ID NO: 603 - DNA, 51 bp, Homo sapiens
aatcaaatccatttgcatgccgctagtattctgtggattcttattataact

SEQ ID NO: 604 - DNA, 51 bp, Homo sapiens
acattactttaagaaagaaatcaccgatcagttcctctaatgccttgttct

SEQ ID NO: 605 - DNA, 51 bp, Homo sapiens
attctggtcataagaaaggggaaaattgaggcagattttttcgccctacta

SEQ ID NO: 606 - DNA, 51 bp, Homo sapiens
accctgctctttgaaagtaggaaccccatcttttcatagttcttggcatat

SEQ ID NO: 607 - DNA, 51 bp, Homo sapiens
tccttccacttcttcatcaatgaccggcacctgcagggtgtgagtcactgg

SEQ ID NO: 608 - DNA, 51 bp, Homo sapiens
ttcctggtttgtgcagttcaacaacggtggttttggactgtatctacttgt

SEQ ID NO: 609 - DNA, 51 bp, Homo sapiens
aacttgctgacttagtaccatgaaatgaatagctatgaaggaagtgagaga

SEQ ID NO: 610 - DNA, 51 bp, Homo sapiens
taaattcttaccaaactatgcccaacatgttttcatttcattaagcagtta

SEQ ID NO: 611 - DNA, 51 bp, Homo sapiens
taaagcaatattttgctagtgctagcgtgtgtccaaatggaattctttccc

SEQ ID NO: 612 - DNA, 51 bp, Homo sapiens
gtatcccaattcctgaacggtgggttattaaatctgaaacttgaaggagga

SEQ ID NO: 613 - DNA, 51 bp, Homo sapiens
taagatttcactttggttagttaacacttgaaagtaaatactcagatacta

SEQ ID NO: 614 - DNA, 51 bp, Homo sapiens
cctattttcttgttgttgacaagaaagaaacatacagcgtttcaatgacag

SEQ ID NO: 615 - DNA, 51 bp, Homo sapiens
aaataaatacataaataaataaaactaagagggagttgagaatgaccagaa

SEQ ID NO: 616 - DNA, 51 bp, Homo sapiens
ttttctgccaatgactttcttcaccatctctttcctgtgtctcatttggca

SEQ ID NO: 617 - DNA, 51 bp, Homo sapiens
gcatggagaatattggtcatatttgtttacagcatgtgtgacttaataaat

SEQ ID NO: 618 - DNA, 51 bp, Homo sapiens
gcttttcttcaaacatttactagattcttatttgtcaaatctttatgtgtg

SEQ ID NO: 619 - DNA, 51 bp, Homo sapiens
atccaatcccttcccacccgcatgatgacactttgccagaactggcagcaa

SEQ ID NO: 620 - DNA, 51 bp, Homo sapiens
actaagtcaggtgggtaacacagcagtgagggctgagatcttgcagcttag

SEQ ID NO: 621 - DNA, 51 bp, Homo sapiens
caaatgatataacggcagaaataccgtatctcgtatctctattgactgtga

SEQ ID NO: 622 - DNA, 51 bp, Homo sapiens
gggaaaaattaagatgttggccacaattgctattaatttggcctgagctgc

SEQ ID NO: 623 - DNA, 51 bp, Homo sapiens
ggagctggccttggaaagctgattggtcgggagaagtggaagaaggagatg

SEQ ID NO: 624 - DNA, 51 bp, Homo sapiens
aagtccctgagattaagaacacggcagaggatttgtgactctacttgtgtt

SEQ ID NO: 625 - DNA, 51 bp, Homo sapiens
agatgagatcaagtaaaatctccttagaagcagaatggttgatcaaatgta

SEQ ID NO: 626 - DNA, 51 bp, Homo sapiens
gactatttatgtgctgtttgctaagacagttcctgagtagaaccctgtccc

SEQ ID NO: 627 - DNA, 51 bp, Homo sapiens
actaaatagggaaaatgtcagctgagtcccacatgtagattccttgtcatc

SEQ ID NO: 628 - DNA, 51 bp, Homo sapiens
tcttgctgatgggaactatgacaacatgacattgaagaattatgagctaaa

SEQ ID NO: 629 - DNA, 51 bp, Homo sapiens
ccattttagatttattagcagttagttgaaacaacagattttgtgattttt

SEQ ID NO: 630 - DNA, 51 bp, Homo sapiens
ttcacaatctttgaatttaaactcaatcggaaggaacctcacatgagatta

SEQ ID NO: 631 - DNA, 51 bp, Homo sapiens
gagctgtgttttgcatacatggtctgtggtctgttttgtgtcctaggtctg

SEQ ID NO: 632 - DNA, 51 bp, Homo sapiens
aagtgaattttactttctccatgaagctttctaatctcacagagttacccc

SEQ ID NO: 633 - DNA, 51 bp, Homo sapiens
aaatgtatgtggtcattaggttttcagcattattcagtcatcccctctttt

SEQ ID NO: 634 - DNA, 51 bp, Homo sapiens
acagcattttacacccctagcgtaactgtgattcagatgaggattgcaaca

SEQ ID NO: 635 - DNA, 51 bp, Homo sapiens
aaccgaggacacagacgcagggctcgggcagagttcccacggtgggcacag

SEQ ID NO: 636 - DNA, 51 bp, Homo sapiens
cacaagctcatcatctattcgagtcgagagtattcagttatcagcctcaga

SEQ ID NO: 637 - DNA, 51 bp, Homo sapiens
taaattcctagtgccttcgagtaaagctgttcatggagcatatttaagaca

SEQ ID NO: 638 - DNA, 51 bp, Homo sapiens
cttcttgtggtcatgtgagactcattccttttactgtttctgtgcatacct

SEQ ID NO: 639 - DNA, 51 bp, Homo sapiens
tctccagttgtttcttttgtgtgaagtaacttccagaatccatgactcaca

SEQ ID NO: 640 - DNA, 51 bp, Homo sapiens
tttacttgattctcccagttgctggatatttaggtagtttgattttgtctc

SEQ ID NO: 641 - DNA, 51 bp, Homo sapiens
tctgtctcaactctatatcacctgtatgcttgtagactggcaaagaaagtc

SEQ ID NO: 642 - DNA, 51 bp, Homo sapiens
ctcaactcagagtcagggtttgaactatatccccggctggaatgggggcca

SEQ ID NO: 643 - DNA, 51 bp, Homo sapiens
tctccaaacaaaacctctctctttccgactctccaccctatgcttttccaa

SEQ ID NO: 644 - DNA, 51 bp, Homo sapiens
aattctcccaactttgttattggtcgttgagatgatacacattcagtacca

SEQ ID NO: 645 - DNA, 51 bp, Homo sapiens
tacctagaaactcctttaaatttgggtttctgagagtagtaatgacttgta

SEQ ID NO: 646 - DNA, 51 bp, Homo sapiens
tgtttttggagggcagagataaactatggaaagatggaatgctgtattttt

SEQ ID NO: 647 - DNA, 51 bp, Homo sapiens
caaagcacatgtgaggcccagaaggtggtttttcatgcctttaataaccat

SEQ ID NO: 648 - DNA, 51 bp, Homo sapiens
cgagtttttaagtgcccagagaagatgtactcaacagggagagttaagagg

SEQ ID NO: 649 - DNA, 51 bp, Homo sapiens
ggtttccagctatcatccctgttttacctcttcttattcctgggtttgaga

SEQ ID NO: 650 - DNA, 51 bp, Homo sapiens
taaattaaagcatgaactacgaggaacctgtatctaagtaaagggactgac

SEQ ID NO: 651 - DNA, 51 bp, Homo sapiens
gatgatggtcatataattaagtttgacatctttactggagagcacatcagt

SEQ ID NO: 652 - DNA, 51 bp, Homo sapiens
cacattctttgtatctataacacatgcttctgaaatatcagatacccatca

SEQ ID NO: 653 - DNA, 51 bp, Homo sapiens
ctcattagaaaggactttgatgcacatttttatgcaagaggtgaattcttc

SEQ ID NO: 654 - DNA, 51 bp, Homo sapiens
tggagcattcttaaagctagtaatgatctttgcaaatttaaattttccata

SEQ ID NO: 655 - DNA, 51 bp, Homo sapiens
gataagagagtaaaggtaagctttcggggccctttttccacatgtcatatt

SEQ ID NO: 656 - DNA, 51 bp, Homo sapiens
accaccctataccatctccttaggcgcagcactgttttgcttcagatgctt

SEQ ID NO: 657 - DNA, 51 bp, Homo sapiens
taatctgagtttctgaatgtacatgtagatgagagtacacatgtgtgagag

SEQ ID NO: 658 - DNA, 51 bp, Homo sapiens
aaacagagaagtcaatggcaagttgaattttgtatggtgactttaccagaa

SEQ ID NO: 659 - DNA, 51 bp, Homo sapiens
tgatatttcaaagttgttttcgctcacattcctttcattactgtcaagaat

SEQ ID NO: 660 - DNA, 51 bp, Homo sapiens
tgggaagaagatccaaaccacatcaccaggattcagttaaccatccccctc

SEQ ID NO: 661 - DNA, 51 bp, Homo sapiens
tgagactttctctggctggctgccaaccaaagacttcccacaatggttcta

SEQ ID NO: 662 - DNA, 51 bp, Homo sapiens
ggcttgatcctctaatgacccatggacttgaattagaacatcatcctggat

SEQ ID NO: 663 - DNA, 51 bp, Homo sapiens
gcaaagacttcagggccaggctccaacttttacaaacactcaagataggaa

SEQ ID NO: 664 - DNA, 51 bp, Homo sapiens
actaacgctgacaagagcagacatcgtacaagtgggacaggggaaaaaaat

SEQ ID NO: 665 - DNA, 51 bp, Homo sapiens
atgttgcatttcagatttttttgaaaaaaagccagagccccaacaatttaa

SEQ ID NO: 666 - DNA, 51 bp, Homo sapiens
gaaatcctccttcctttttaacaagacaagtagaaaacgaaaacaacatca

SEQ ID NO: 667 - DNA, 51 bp, Homo sapiens
tgcaaatcagcaaagtgaagctggtaaatctcacttcctattccctcttag

SEQ ID NO: 668 - DNA, 51 bp, Homo sapiens
gccacacacaagcctattctaaacccgtctttcttccttaagtgtgccaat

SEQ ID NO: 669 - DNA, 51 bp, Homo sapiens
gtcagatgtagatacgtttttgaaggactgaaaactaaataattccatttc

SEQ ID NO: 670 - DNA, 51 bp, Homo sapiens
tcttatccatgaaatatttttgaatgaattatctgaatgaatatgcacatt

SEQ ID NO: 671 - DNA, 51 bp, Homo sapiens
cagattggcttcattctttaatttcgatatggactcacttacaaatggacc

SEQ ID NO: 672 - DNA, 51 bp, Homo sapiens
tatattagtttgtgagcattgagttgcctagaaaagtttggaagtcctaca

SEQ ID NO: 673 - DNA, 51 bp, Homo sapiens
ggaaaaaaaaaagactgaggccgtgatatgtgtaacatgttattagatatg

SEQ ID NO: 674 - DNA, 51 bp, Homo sapiens
ttcttgactgttggtgagggagttgtatcaaccccaggttcttaaacttgt

SEQ ID NO: 675 - DNA, 51 bp, Homo sapiens
atcaaactcagtcttctgactggctcctttggtcttccctatttctcttaa

SEQ ID NO: 676 - DNA, 51 bp, Homo sapiens
acaaatgcaagagtagaagtacacacggagcagtggcaactcaagagagaa

SEQ ID NO: 677 - DNA, 51 bp, Homo sapiens
aactcattttccattttccatgcataggatagtaggtccacctcaccttta

SEQ ID NO: 678 - DNA, 51 bp, Homo sapiens
cccacctcagtgcatgtgtgtatacatggattgttttagtatcttaatcac

SEQ ID NO: 679 - DNA, 51 bp, Homo sapiens
ttgtgactgtcaccgggagacagaataagtgaggtggcacaggtgcagtat

SEQ ID NO: 680 - DNA, 51 bp, Homo sapiens
gagtgtgggaaattagtgcaggcccaaggaaaggaagtgtgaaactgcttc

SEQ ID NO: 681 - DNA, 51 bp, Homo sapiens
gtgtggacttttactgttgagctaaggtttatgtttatatatgttttattc

SEQ ID NO: 682 - DNA, 51 bp, Homo sapiens
gctgcttcacgcctatagctcagttttacaaatagctatagtttggacgct

SEQ ID NO: 683 - DNA, 51 bp, Homo sapiens
agctagaactcaaatctggccttggtatgcaatcttgactattttaagctc

SEQ ID NO: 684 - DNA, 51 bp, Homo sapiens
ataactctaatgactttgtagtttacgtgcaaaggcagaaagagacctcaa

SEQ ID NO: 685 - DNA, 51 bp, Homo sapiens
aagtggcaacgaaataaatggtgtatggacctaaagttgttaataactcaa

SEQ ID NO: 686 - DNA, 51 bp, Homo sapiens
ctacatttgcagctgcccatttagaatcaacccacgctgcccagagcagag

SEQ ID NO: 687 - DNA, 51 bp, Homo sapiens
gggcataccagggctgtggaaaaccgacacctcaatgattccctttaccct

SEQ ID NO: 688 - DNA, 51 bp, Homo sapiens
cagaaaaaatttagtgctgtgggagtcatcatgatatcccggaaaggcaca

SEQ ID NO: 689 - DNA, 51 bp, Homo sapiens
taagtaaacattaaatgctcatgaggaaatattttacacccaaatattaat

SEQ ID NO: 690 - DNA, 51 bp, Homo sapiens
tgtaggaaattggatgttactattccttcaattcggggcttctaatgggta

SEQ ID NO: 691 - DNA, 51 bp, Homo sapiens
aatgcctgggaaaatgatctaaagcaataatcctaagaaataaccaagaga

SEQ ID NO: 692 - DNA, 51 bp, Homo sapiens
gaaaatttgagggaaatacagctgattccttatggtgaacctggcccagcc

SEQ ID NO: 693 - DNA, 51 bp, Homo sapiens
gccatctctcctccaggctggagtcggtgcttcccacagttacttctcacg

SEQ ID NO: 694 - DNA, 51 bp, Homo sapiens
cagcagcaactgggacggagcagggcccactgagtcaaccacccaggtgtg

SEQ ID NO: 695 - DNA, 51 bp, Homo sapiens
tcatagacaaaacacatgtaacccacgaagagtttgtttaaacccaaggaa

SEQ ID NO: 696 - DNA, 51 bp, Homo sapiens
accccaatccaaaaatccctttaactgcaactctgaaaaaaatgccaacga

SEQ ID NO: 697 - DNA, 51 bp, Homo sapiens
aaatgtaaatggagcatacctctggatcagaagattttaggtattattatt

SEQ ID NO: 698 - DNA, 51 bp, Homo sapiens
cttggtagaaaatctggaagcactcgtcgcaaatgcctttacaacactatg

SEQ ID NO: 699 - DNA, 51 bp, Homo sapiens
ggcacagccttgatgaaccttacaaactgctctcagtgagtggttctcatc

SEQ ID NO: 700 - DNA, 51 bp, Homo sapiens
gaacattaacagcatctctctcctatagatccagttacgacaaaggttttc

SEQ ID NO: 701 - DNA, 51 bp, Homo sapiens
cataattctcaaggacttttatttaataggactggagagggatctggacat

SEQ ID NO: 702 - DNA, 51 bp, Homo sapiens
ccacttctatattctcttcttccttcatttttcaaatgtttgcatttaaaa

SEQ ID NO: 703 - DNA, 51 bp, Homo sapiens
ctttttgtaagaggatacaataaaagtatgagtcaaagaatatattgggga

SEQ ID NO: 704 - DNA, 51 bp, Homo sapiens
gaggagtcagttatctcggcgactctatttccttttctgaatatacgatca

SEQ ID NO: 705 - DNA, 51 bp, Homo sapiens
ctccaggatcactgtgatgataggttcagaattcctcccttcaatgcaact

SEQ ID NO: 706 - DNA, 51 bp, Homo sapiens
atcaacaccatcccttctgcagtcatttcttcaccatcccttccacgcatg

SEQ ID NO: 707 - DNA, 51 bp, Homo sapiens
aaaggccatatagaagacaaaaataccaactgtatctatctcttacatgaa

SEQ ID NO: 708 - DNA, 51 bp, Homo sapiens
ggacttttagcttttgaaatacaggttttttttttggtaatcacattagaa

SEQ ID NO: 709 - DNA, 51 bp, Homo sapiens
ataacaaaattgtaagtgttactacggaaaattcctttttactactcttag

SEQ ID NO: 710 - DNA, 51 bp, Homo sapiens
gaaattaccatatctctgtcttcttcgctgagtcatggaatcccttcaaaa

SEQ ID NO: 711 - DNA, 51 bp, Homo sapiens
taatggcctgggagagagaaatactttggacttaagcattatggtgtgtag

SEQ ID NO: 712 - DNA, 51 bp, Homo sapiens
tatttttctgagcgacaaaatgtaggaggcatcataaaatatgaggaatgg

SEQ ID NO: 713 - DNA, 51 bp, Homo sapiens
aggtgggtaggacagatgtcaccctctttcaggcaggaatctgagaggctg

SEQ ID NO: 714 - DNA, 51 bp, Homo sapiens
tgcgtatgatctcaatactaagaaagatcagaactgcttatacgttacagt

SEQ ID NO: 715 - DNA, 51 bp, Homo sapiens
tacgtaggaaaatatctatgttacacgatcttgtgcagtcactgctattaa

SEQ ID NO: 716 - DNA, 51 bp, Homo sapiens
tcacagagacttgcagaaaagagattaaatttaccataggatatccttagt

SEQ ID NO: 717 - DNA, 51 bp, Homo sapiens
gctgaacattattgttcctgagactgacttcttagccatttattttacttc

SEQ ID NO: 718 - DNA, 51 bp, Homo sapiens
ggacttgtacaacccaggattcctagtcattcctgtcaaacatacactttc

SEQ ID NO: 719 - DNA, 51 bp, Homo sapiens
gctttgggaatagggcttaccaaaactggggcatttggtagggatgtggta

SEQ ID NO: 720 - DNA, 51 bp, Homo sapiens
ccatcaggatgcaatacataagcaagtgaacccacccataattacttaata

SEQ ID NO: 721 - DNA, 51 bp, Homo sapiens
ttcatctcctgtcatctcatcttgtattctcactgaacctgtatgtttaaa

SEQ ID NO: 722 - DNA, 51 bp, Homo sapiens
gttttgtcctgctgaattggcagagcagtcccaaaagaactgtaactaagg

SEQ ID NO: 723 - DNA, 51 bp, Homo sapiens
aatcactgccaacagtggtgcctgcgtttgcaagggcagaggggctctcag

SEQ ID NO: 724 - DNA, 51 bp, Homo sapiens
agcagagctattttgtagattgttaagaagcttttgaggcctttaaagtag

SEQ ID NO: 725 - DNA, 51 bp, Homo sapiens
ctaggcagaggttgttctttctcgatagctgttccaagagtgctatcctgg

SEQ ID NO: 726 - DNA, 51 bp, Homo sapiens
tattttaagtttttttgtctgatggacttctgagctctgctgtctgtcaat

SEQ ID NO: 727 - DNA, 51 bp, Homo sapiens
tgcagtcggtgtgtaattaagaggatgtaattaaggattattgctggatgt

SEQ ID NO: 728 - DNA, 51 bp, Homo sapiens
cagaattaacgtgcttccaaactgacagcacccattaacaactcactcaca

SEQ ID NO: 729 - DNA, 51 bp, Homo sapiens
ggacagttcaaatgattctgggtctcataattgctacatgatgtaaggtga

SEQ ID NO: 730 - DNA, 51 bp, Homo sapiens
ttaaggctccattcaatcaaaaaaacatttcatatcatcaataccactgtt

SEQ ID NO: 731 - DNA, 51 bp, Homo sapiens
atggtaccagaagacatgctagaggggattggaataggtgtggagagccaa

SEQ ID NO: 732 - DNA, 51 bp, Homo sapiens
ctgtgctgaatgccaggtaattgtaaccaaactcaggttcaactgctcact

SEQ ID NO: 733 - DNA, 51 bp, Homo sapiens
gatgtgtaaaacgtacgtgcctaagatcaatgactgtgtcatcacagaaga

SEQ ID NO: 734 - DNA, 51 bp, Homo sapiens
agacccttctctggccttttcactagaatacaacaaagattaaatcagctt

SEQ ID NO: 735 - DNA, 51 bp, Homo sapiens
gcagagttttcacgaacaatgaaaacgcctgggagtgttcattgccagtta

SEQ ID NO: 736 - DNA, 51 bp, Homo sapiens
gtgtaatcctgtctcaacatcttgcgtaagaactcattctaggaaaaagat

SEQ ID NO: 737 - DNA, 51 bp, Homo sapiens
ggtctgtggccaaaggcatatcccagtcacatgaccactccagcagcaagg

SEQ ID NO: 738 - DNA, 51 bp, Homo sapiens
agcaaagaagtttgtgccatttcaaataagtgacatgtcagacatggagtc

SEQ ID NO: 739 - DNA, 51 bp, Homo sapiens
gcacaaaaatgcctcaaaaagtgtcgtagcctatatagaccatatgatctc

SEQ ID NO: 740 - DNA, 51 bp, Homo sapiens
gttttgatgccattaggttcaaggggctcaacatctggtagaagagataaa

SEQ ID NO: 741 - DNA, 51 bp, Homo sapiens
aagagcttggaacaaaattgccactggggaccataagactaactgctgcgc

SEQ ID NO: 742 - DNA, 51 bp, Homo sapiens
aagactgctacattaataatttggtataagtgtggatgtgtgtgggggtgt

SEQ ID NO: 743 - DNA, 51 bp, Homo sapiens
caaccactatgccctcaccatctgcacaactgctttggagctgcagatcag

SEQ ID NO: 744 - DNA, 51 bp, Homo sapiens
atctcttcatattgggttttttgaacgtggggaaatgtctttttctttcta

SEQ ID NO: 745 - DNA, 51 bp, Homo sapiens
aactaacgatatcatagaagatgacgctcctgatttgtgggttaatctttc

SEQ ID NO: 746 - DNA, 51 bp, Homo sapiens
tagttatctcaatatccacagaatgctaccctgacagcaaaatgccttgca

SEQ ID NO: 747 - DNA, 51 bp, Homo sapiens
gggtgacaactagtggagtcctaatgtgcaccaggtgtgctctttgtacca

SEQ ID NO: 748 - DNA, 51 bp, Homo sapiens
gaaactacagttgctgcttactatttgaaagactgtcaaaggagtggccat

SEQ ID NO: 749 - DNA, 51 bp, Homo sapiens
tggtcttgtataacaaaaaaccaacgcgttcaaaatggaatgatatcctct

SEQ ID NO: 750 - DNA, 51 bp, Homo sapiens
ctcaacaattactgataaccaaattggcataagaaacttacttgcagttaa

SEQ ID NO: 751 - DNA, 51 bp, Homo sapiens
tgcaggtgccacataacaggtagtacggatttccactcccacattcaccgc

SEQ ID NO: 752 - DNA, 51 bp, Homo sapiens
acaaaatgtaataaaaagcttttgctggtataaagttttgttgttctaacc

SEQ ID NO: 753 - DNA, 51 bp, Homo sapiens
aaccctatcagcaaagcagaatgaacgtgtgcttccaggagtttggagtat

SEQ ID NO: 754 - DNA, 51 bp, Homo sapiens
gttaacttcaaatcagtcaatgacacggagttgttcaacaaaattataaac

SEQ ID NO: 755 - DNA, 51 bp, Homo sapiens
ccctacgctcaccaacacagatttactcacttcctcctctaactctcttac

SEQ ID NO: 756 - DNA, 51 bp, Homo sapiens
tatacaatttgtgcatcatggtaccacaacgatgaaagtcaattctttctc

SEQ ID NO: 757 - DNA, 51 bp, Homo sapiens
tagtcacttaaatttggctgactagagcctaatagaccaggagaattaaaa

SEQ ID NO: 758 - DNA, 51 bp, Homo sapiens
catctctatgatttttgttattacagtaggtcactgagctgttatttattt

SEQ ID NO: 759 - DNA, 51 bp, Homo sapiens
agggagcttcatcagtactttgttcgtacctccaaatggcaactacaaggg

SEQ ID NO: 760 - DNA, 51 bp, Homo sapiens
ctttaaatcaattgaattagggttagtgtgatccccagatcatgaggaagc

SEQ ID NO: 761 - DNA, 51 bp, Homo sapiens
attccaacatagtttaaatatcaatgtgcaatgcgtgcaaaagaaatcaaa

SEQ ID NO: 762 - DNA, 51 bp, Homo sapiens
ggcaggaagcagggagctctaagggtctaaagaatcagagtgaaaacagga

SEQ ID NO: 763 - DNA, 51 bp, Homo sapiens
ttcaccggaaatggtgtaccaattaacaacattcacattatagtttgggac

SEQ ID NO: 764 - DNA, 51 bp, Homo sapiens
aagactagatctttttggtaaactgcctactgaagggatcagctaaggtcc

SEQ ID NO: 765 - DNA, 51 bp, Homo sapiens
ccagtaacttttaaaatggaggcggaattaataacagcagcaacaaccaca

SEQ ID NO: 766 - DNA, 51 bp, Homo sapiens
gcaggtgttcacttgcgacccttttcgtcccttccaaatccggcaggttct

SEQ ID NO: 767 - DNA, 51 bp, Homo sapiens
aatgctttattgacattatacgactatgctaagaactgtcaggcctctgag

SEQ ID NO: 768 - DNA, 51 bp, Homo sapiens
caggcataaatgaaatcagatgcccgcagagaaagctgtgtgatcactcca

SEQ ID NO: 769 - DNA, 51 bp, Homo sapiens
acatccaggagcactcaatatgtgttgaatcaattgttcaaaaacagaaat

SEQ ID NO: 770 - DNA, 51 bp, Homo sapiens
agaaccattcatcttcttaatcccatgcctcctgcttggtttatttgtatg

SEQ ID NO: 771 - DNA, 51 bp, Homo sapiens
cacgtctgtttttgttttcaggaatttcccgagtctgttggtataaaaaga

SEQ ID NO: 772 - DNA, 51 bp, Homo sapiens
ttaaccctgttatagaaagaatgcaaacagaaagaaaagacttaatgtaat

SEQ ID NO: 773 - DNA, 51 bp, Homo sapiens
tttcttccatccctttggaaaatgctgtttaaccttaacaattgatatacc

SEQ ID NO: 774 - DNA, 51 bp, Homo sapiens
ggggacaggaatcaaggcgggcccagggtcgagatagaaggagctatttga

SEQ ID NO: 775 - DNA, 51 bp, Homo sapiens
ggacacagagacgagtataaattcaaaggtcctccctcctttaataacaca

SEQ ID NO: 776 - DNA, 51 bp, Homo sapiens
gtatcctgaccattcacaaagtgtcgtgccacagcacttgcatcattaggg

SEQ ID NO: 777 - DNA, 51 bp, Homo sapiens
cacgaaggctactttctccgagaagccctccttacccccatgttaaatcag

SEQ ID NO: 778 - DNA, 51 bp, Homo sapiens
ttccttcaaacaatcaccaactccagtttcctgattttaactcaattgtca

SEQ ID NO: 779 - DNA, 51 bp, Homo sapiens
caaaaaaaattaccaaattgatcactctgaagaatttaaactaagatcact

SEQ ID NO: 780 - DNA, 51 bp, Homo sapiens
gaagcaaaatatatacaagttaaagatatgtgttcagcttcagtccagtct

SEQ ID NO: 781 - DNA, 51 bp, Homo sapiens
atttcccatttgtacacatgcaatatgattaaaatagatctctaaagaaga

SEQ ID NO: 782 - DNA, 51 bp, Homo sapiens
gcttcacttttctgcctttactttgctattggaaattcctataatttgcct

SEQ ID NO: 783 - DNA, 51 bp, Homo sapiens
ttttcatcatctcctttcctggggtgttttcacctcaccattggaggcagc

SEQ ID NO: 784 - DNA, 51 bp, Homo sapiens
tttgcgaagatgtttcctattgccttaaatacttgccttgcacagtagctt

SEQ ID NO: 785 - DNA, 51 bp, Homo sapiens
ttgatggaattggaaaggcaatttcagctctaaatcaccacaaatcttcag

SEQ ID NO: 786 - DNA, 51 bp, Homo sapiens
acagctgcagcagctgcatatcagataatggaatctgctgagacactggga

SEQ ID NO: 787 - DNA, 51 bp, Homo sapiens
tagaaccagaaatgggtgccagagatatgcctgcactaatcttaagtgggg

SEQ ID NO: 788 - DNA, 51 bp, Homo sapiens
gctccaggtgaaagaacaaagccacatgtccttttctccactcacccctga

SEQ ID NO: 789 - DNA, 51 bp, Homo sapiens
gtctttaacatgtaactcgatcttccacagattaagtgacaaagtcattca

SEQ ID NO: 790 - DNA, 51 bp, Homo sapiens
cagctccatggatgactgggagagacagccccttgcagagcacccacactt

SEQ ID NO: 791 - DNA, 51 bp, Homo sapiens
tcccagttcctacaggttgttcctcaatgatgcctggatctcacttcaggg

SEQ ID NO: 792 - DNA, 51 bp, Homo sapiens
cacccatatctgcactaagattttccgtaggactttggggtcttgtttaat

SEQ ID NO: 793 - DNA, 51 bp, Homo sapiens
gataacggatagcggctcaggagaatattaagagcatcgactctggagcca

SEQ ID NO: 794 - DNA, 51 bp, Homo sapiens
gttggacagcttggcctctgattcatgtaagcttaagaaggtctgctacta

SEQ ID NO: 795 - DNA, 51 bp, Homo sapiens
tctgctctccttgggccttagtgtcagctctaaagatttataaaggttgca

SEQ ID NO: 796 - DNA, 51 bp, Homo sapiens
gtacagtgtacaaacgtcaacagaagacaaagatatgcctagaactatcta

SEQ ID NO: 797 - DNA, 51 bp, Homo sapiens
tgattgccgaccccgtatattggttcgaggagctagatttccttactgtta

SEQ ID NO: 798 - DNA, 51 bp, Homo sapiens
gtaaaatgtttattttgctctactacaccagactaattaaacaaatcagaa

SEQ ID NO: 799 - DNA, 51 bp, Homo sapiens
ttatatgaagcattgccccatctgctcaatccacagacacacttgcttctt

SEQ ID NO: 800 - DNA, 51 bp, Homo sapiens
cccagaaaacattagtagttgccccaagttattcaaatacagttttcctga

SEQ ID NO: 801 - DNA, 51 bp, Homo sapiens
cttagtgggtaacatctttcaacccaggtaagatttctaattaagtttaga

SEQ ID NO: 802 - DNA, 51 bp, Homo sapiens
cttaaggggaggatagctgggacccgtctatttccgtggatctgggcacag

SEQ ID NO: 803 - DNA, 51 bp, Homo sapiens
attctctgctcttggagacctgaaaggccagagaactccaaaagacaaaag

SEQ ID NO: 804 - DNA, 51 bp, Homo sapiens
actaaacaactgaaatgttgcactggtgaccaatgggctggctgtcaccag

SEQ ID NO: 805 - DNA, 51 bp, Homo sapiens
gccctagaaggatcttttacttgctttcatttttccacagtcaagttatat

SEQ ID NO: 806 - DNA, 51 bp, Homo sapiens
tttcctctttcctttctcacacttatgcagttggttatagttcatctattg

SEQ ID NO: 807 - DNA, 51 bp, Homo sapiens
taaccagatttcccattgactcctacgatgagatttacataacttgatatt

SEQ ID NO: 808 - DNA, 51 bp, Homo sapiens
aatttataaaatggataggcaatgcctacttgacatgattggtgaggtgaa

SEQ ID NO: 809 - DNA, 51 bp, Homo sapiens
ttctatttaccactggacaaagaccgagctagaggcaaatcttgagaataa

SEQ ID NO: 810 - DNA, 51 bp, Homo sapiens
cccttcatggaggaccttcagttttggggagctgacctgtgctgacttagc

SEQ ID NO: 811 - DNA, 51 bp, Homo sapiens
cagcctttatcctcagagcaataacgatgatagtgacagttcttgactttt

SEQ ID NO: 812 - DNA, 51 bp, Homo sapiens
gaaaaatggtctcctaatattaacattcctagcataccactgtccccttct

SEQ ID NO: 813 - DNA, 51 bp, Homo sapiens
aagccatctagtcagctgaggatgactaaacaattcaaaagactaaaaaat

SEQ ID NO: 814 - DNA, 51 bp, Homo sapiens
atactctacagcacatctcattccccttatgaacctgcctctagtcagtca

SEQ ID NO: 815 - DNA, 51 bp, Homo sapiens
tgtattttatcagactgtaaataggcctgtttagaagatttgctgcatttt

SEQ ID NO: 816 - DNA, 51 bp, Homo sapiens
gtaagagaaagacttaagaacctgtaacaatattgaatccacaatcagata

SEQ ID NO: 817 - DNA, 51 bp, Homo sapiens
gtccaattttctatatcagttgttagaattgaccattaactctataatatt

SEQ ID NO: 818 - DNA, 51 bp, Homo sapiens
tataaccatttctgtgggttcagcaatcaccaaccagagagaggtaaattg

SEQ ID NO: 819 - DNA, 51 bp, Homo sapiens
cactgtaaggccttctgtgttaaaacagcagccttgcctccagcacacata

SEQ ID NO: 820 - DNA, 51 bp, Homo sapiens
ggtcttgtctagggaggaatgcacccaatccaaacaaatcatgacatgcca

SEQ ID NO: 821 - DNA, 51 bp, Homo sapiens
ctggaatgcagctgaaatgacagaaatgttccgagcacttttttttttttt

SEQ ID NO: 822 - DNA, 51 bp, Homo sapiens
gtgggctgctgtctcctgatgctgcgtggtgtgccccatggacatttgaaa

SEQ ID NO: 823 - DNA, 51 bp, Homo sapiens
tcacaaaaaagctggttttcatcaagtcatgcactgatttgcatctattct

SEQ ID NO: 824 - DNA, 51 bp, Homo sapiens
tatgctaatgatctctattccaggcgaacaaatgtcctctgaatttccttt

SEQ ID NO: 825 - DNA, 51 bp, Homo sapiens
gacatacagcaaaacacttctaagctttttttttaatagcaagtttaagtt

SEQ ID NO: 826 - DNA, 51 bp, Homo sapiens
cttttattttttaagtgaagtgaaacggtaacctgtgctaacaggaagcaa

SEQ ID NO: 827 - DNA, 51 bp, Homo sapiens
tggggcaggcccagaatggccctgaatccagtaacaccaactgtccctgga

SEQ ID NO: 828 - DNA, 51 bp, Homo sapiens
tatggtatttgggggactagaattacaaataggctctgtggtgcttttttc

SEQ ID NO: 829 - DNA, 51 bp, Homo sapiens
agaatctgggttcaagtccaggtggcatggcttccaggctggcatgtgatg

SEQ ID NO: 830 - DNA, 51 bp, Homo sapiens
tgataggaaatggggcttcaggttgaaatcagatgagagctggcctctgag

SEQ ID NO: 831 - DNA, 51 bp, Homo sapiens
tgctgttcagagcattgctgacaccagggcatgaaactagtgacacagtta

SEQ ID NO: 832 - DNA, 51 bp, Homo sapiens
ctttttttgatattatacaaagagtgagatacaaaggcatttgttttatta

SEQ ID NO: 833 - DNA, 51 bp, Homo sapiens
caggaaagtagtgaattgtggcacaaagctggaatatgctgtcttttcctt

SEQ ID NO: 834 - DNA, 51 bp, Homo sapiens
agttttcctggggaagccgaggccgcctagaggcagaaagctgtcatcccg

SEQ ID NO: 835 - DNA, 51 bp, Homo sapiens
atcatttccttgactgctgtcctaacgttggatgtttgaataggggttttg

SEQ ID NO: 836 - DNA, 51 bp, Homo sapiens
atattttggtggcttggctctggctcggaccctggctgtgtaggcctggtc

SEQ ID NO: 837 - DNA, 51 bp, Homo sapiens
cattcttctcaggacctttgctctttgcctttctctgtctgaaggttcact

SEQ ID NO: 838 - DNA, 51 bp, Homo sapiens
ccatgcttactcactctatgacccactgagtaaaagctagtttggaataaa

SEQ ID NO: 839 - DNA, 51 bp, Homo sapiens
tgtggtcttaggtgagagaagccgcgcaggccctgcttctggcggaactga

SEQ ID NO: 840 - DNA, 51 bp, Homo sapiens
ttataaaatgatgattcactttgtctaatatctcagtttttataattaatc

SEQ ID NO: 841 - DNA, 51 bp, Homo sapiens
aaaatactaaattcatttttcccgctagaatttcaacaaggtatcaagttc

SEQ ID NO: 842 - DNA, 51 bp, Homo sapiens
ctggagggatccgagacccactctcttatttttccttctgaacaaaagcct

SEQ ID NO: 843 - DNA, 51 bp, Homo sapiens
tccagacttacaagattacttcatggtgaaagtttggattgatcaataatt

SEQ ID NO: 844 - DNA, 51 bp, Homo sapiens
cagttccagcagggttgggtgagacctcagggtgatgcatgggcactgttt

SEQ ID NO: 845 - DNA, 51 bp, Homo sapiens
agagctgaaggaaagggtatttatatataagctgttcttggtcctaaaaaa

SEQ ID NO: 846 - DNA, 51 bp, Homo sapiens
gcttgtaccttgcttactaacattgtgctgcaatcacatggccaagtccaa

SEQ ID NO: 847 - DNA, 51 bp, Homo sapiens
taaccaccaccttggctacatcttcactttatactttcacaggagattttg

SEQ ID NO: 848 - DNA, 51 bp, Homo sapiens
gccccagactttggggggcccatctacaaacctaggctcacccagaatcct

SEQ ID NO: 849 - DNA, 51 bp, Homo sapiens
atgaatgaaactgatgtggcagtatttataaacagatgtaaatgggagatt

SEQ ID NO: 850 - DNA, 51 bp, Homo sapiens
ctaaaggcaagcaacataaagataagtaagcccagtgtcagagctcaggct

SEQ ID NO: 851 - DNA, 51 bp, Homo sapiens
ctgtattcagtaaaggagtattcggtgagggggaagttcatgatcaaatgt

SEQ ID NO: 852 - DNA, 51 bp, Homo sapiens
ttaagaccagtctttctaactaacccagtcagacaaaaataaagaaaaagt

SEQ ID NO: 853 - DNA, 51 bp, Homo sapiens
acaggaatagctgaggaaaaaggctgcttatcatgatcttcctctgctgag

SEQ ID NO: 854 - DNA, 51 bp, Homo sapiens
gtcaaatccatatgagtataaactcattgtaccaatatccacgtgaagcta

SEQ ID NO: 855 - DNA, 51 bp, Homo sapiens
tccacttctccatcgcactgtctgccttcacacaactcattcttggtcctg

SEQ ID NO: 856 - DNA, 51 bp, Homo sapiens
tcaaatcgaacaggtagtatttgtaagtgactccacctcttcttttacaca

SEQ ID NO: 857 - DNA, 51 bp, Homo sapiens
ggtaagagatactggttattatgatataacaagtggtgggattcttatgct

SEQ ID NO: 858 - DNA, 51 bp, Homo sapiens
caggcagggctaatgtgcagctcccacttggatgggaagaacagtgtgtgg

SEQ ID NO: 859 - DNA, 51 bp, Homo sapiens
tggagccctgccatctctcgctatcgtcttctgcctctcaactaggggaat

SEQ ID NO: 860 - DNA, 51 bp, Homo sapiens
tagagactagaggaggaggtaattggtttacatagggtccaggggattggt

SEQ ID NO: 861 - DNA, 51 bp, Homo sapiens
aagacataattgtataatttattccaattgcttttttttaaacaaaaatct

SEQ ID NO: 862 - DNA, 51 bp, Homo sapiens
gctgctggtcactgactctgaactgggagcctgatcttcttctccagctct

SEQ ID NO: 863 - DNA, 51 bp, Homo sapiens
acagcgattctgtaaagatttccagggcatatggtccctaaaggatcatcc

SEQ ID NO: 864 - DNA, 51 bp, Homo sapiens
ctggcaatatttttccagctgcaagaccagtgtcaccccaggaaactgtcc

SEQ ID NO: 865 - DNA, 51 bp, Homo sapiens
tagtaggggaattttctgttgtcttgtgtggttaacttctagactgtattt

SEQ ID NO: 866 - DNA, 51 bp, Homo sapiens
taatcattgggtctacagaaaccacgtgcttgagaaatggaagccctggag

SEQ ID NO: 867 - DNA, 51 bp, Homo sapiens
tgtccacataagacaacctctgttcggagcaattaaaggcgaatctggacc

SEQ ID NO: 868 - DNA, 51 bp, Homo sapiens
tattggccatgtaactttagtggaatgttggataaaggtcactaatcttat

SEQ ID NO: 869 - DNA, 51 bp, Homo sapiens
ctttccatgttcctgactcctaagctgaacacattttaatttattatatat

SEQ ID NO: 870 - DNA, 51 bp, Homo sapiens
gtttaatttccatgtatcttgtaccgtttccagagttcctctcattattgt

SEQ ID NO: 871 - DNA, 51 bp, Homo sapiens; identical to SEQ ID NO: 1

SEQ ID NO: 872 - DNA, 51 bp, Homo sapiens; identical to SEQ ID NO: 2

SEQ ID NO: 873 - DNA, 51 bp, Homo sapiens; identical to SEQ ID NO: 3

SEQ ID NO: 874 - DNA, 51 bp, Homo sapiens; identical to SEQ ID NO: 4

SEQ ID NO: 875 - DNA, 51 bp, Homo sapiens; identical to SEQ ID NO: 5

SEQ ID NO: 876 - DNA, 51 bp, Homo sapiens; identical to SEQ ID NO: 6

SEQ ID NO: 877 - DNA, 51 bp, Homo sapiens; identical to SEQ ID NO: 7

SEQ ID NO: 878 - DNA, 51 bp, Homo sapiens; identical to SEQ ID NO: 8

SEQ ID NO: 879 - DNA, 51 bp, Homo sapiens; identical to SEQ ID NO: 9

SEQ ID NO: 880 - DNA, 51 bp, Homo sapiens; identical to SEQ ID NO: 10

SEQ ID NO: 881 - DNA, 51 bp, Homo sapiens; identical to SEQ ID NO: 11

SEQ ID NO: 882 - DNA, 51 bp, Homo sapiens; identical to SEQ ID NO: 12

SEQ ID NO: 883 - DNA, 51 bp, Homo sapiens; identical to SEQ ID NO: 13

SEQ ID NO: 884 - DNA, 51 bp, Homo sapiens; identical to SEQ ID NO: 14

SEQ ID NO: 885 - DNA, 51 bp, Homo sapiens; identical to SEQ ID NO: 15

SEQ ID NO: 886 - DNA, 51 bp, Homo sapiens; identical to SEQ ID NO: 16

SEQ ID NO: 887 - DNA, 51 bp, Homo sapiens; identical to SEQ ID NO: 17

SEQ ID NO: 888 - DNA, 51 bp, Homo sapiens; identical to SEQ ID NO: 18

SEQ ID NO: 889 - DNA, 51 bp, Homo sapiens; identical to SEQ ID NO: 19

SEQ ID NO: 890 - DNA, 51 bp, Homo sapiens; identical to SEQ ID NO: 20

SEQ ID NO: 891 - DNA, 51 bp, Homo sapiens; identical to SEQ ID NO: 21

SEQ ID NO: 892 - DNA, 51 bp, Homo sapiens; identical to SEQ ID NO: 22

SEQ ID NO: 893 - DNA, 51 bp, Homo sapiens; identical to SEQ ID NO: 23

SEQ ID NO: 894 - DNA, 51 bp, Homo sapiens; identical to SEQ ID NO: 24

SEQ ID NO: 895 - DNA, 51 bp, Homo sapiens; identical to SEQ ID NO: 25

SEQ ID NO: 896 - DNA, 51 bp, Homo sapiens; identical to SEQ ID NO: 26

SEQ ID NO: 897 - DNA, 51 bp, Homo sapiens; identical to SEQ ID NO: 27

SEQ ID NO: 898 - DNA, 51 bp, Homo sapiens; identical to SEQ ID NO: 28

SEQ ID NO: 899 - protein, 21 aa, Homo sapiens
  VARIANT (0)...(0): Xaa = Any amino acid
CXXCXXXXXXXXXXXXHXXXH

SEQ ID NO: 900 - protein, 22 aa, Homo sapiens
  VARIANT (0)...(0): Xaa = Any amino acid
CXXXCXXXXXXXXXXXXHXXXH

SEQ ID NO: 901 - protein, 23 aa, Homo sapiens
  VARIANT (0)...(0): Xaa = Any amino acid
CXXXXCXXXXXXXXXXXXHXXXH

SEQ ID NO: 902 - protein, 22 aa, Homo sapiens
  VARIANT (0)...(0): Xaa = Any amino acid
CXXCXXXXXXXXXXXXHXXXXH

SEQ ID NO: 903 - protein, 23 aa, Homo sapiens
  VARIANT (0)...(0): Xaa = Any amino acid
CXXCXXXXXXXXXXXXHXXXXXH

SEQ ID NO: 904 - protein, 23 aa, Homo sapiens
  VARIANT (0)...(0): Xaa = Any amino acid
CXXXCXXXXXXXXXXXXHXXXXH

SEQ ID NO: 905 - protein, 24 aa, Homo sapiens
  VARIANT (0)...(0): Xaa = Any amino acid
CXXXCXXXXXXXXXXXXHXXXXXH

SEQ ID NO: 906 - protein, 24 aa, Homo sapiens
  VARIANT (0)...(0): Xaa = Any amino acid
CXXXXCXXXXXXXXXXXXHXXXXH

SEQ ID NO: 907 - protein, 25 aa, Homo sapiens
  VARIANT (0)...(0): Xaa = Any amino acid
CXXXXCXXXXXXXXXXXXHXXXXXH

(19) United States
(12) Patent Application Publication
(10) Pub. No.: US 2008/0299125 A1
(43) Pub. Date: Dec. 4, 2008

(54) Genetic basis of treatment response in depression patients

(75) Inventors: David A. Hinds, Mountain View, CA (US); David R. Cox, Belmont, CA (US); Craig L. Hyde, Old Lyme, CT (US); Hakan Sakul, Old Lyme, CT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Perlegen Sciences, Inc., Mountain View, CA (US)

(21) Appl. No.: 11/810,694

(22) Filed: Jun. 5, 2007

Related U.S. Application Data
(60) Provisional application No. 60/811,465, filed on Jun. 5, 2006.

(51) Int. Cl. C12Q 1/68; A61K 39/395; A61K 38/16; A01K 67/027; A61P 25/24; A61K 31/7088; G01N 33/53

(52) U.S. Cl. 424/139.1; 435/6; 436/501; 514/12; 514/44; 800/13

(57) ABSTRACT

The invention provides a collection of polymorphic sites associated with response to treatment by an SSRI or placebo in depression patients. The polymorphic sites and others in linkage disequilibrium with them are useful in determining whether to treat a patient with an SSRI or include a patient in a clinical trial to test an SSRI.

### CROSS-REFERENCE TO RELATED APPLICATION

The present application is a nonprovisional and claims the benefit of 60/811,465 including CD filed Jun. 5, 2006, which is incorporated by reference in its entirety for all purposes.

### BACKGROUND OF THE INVENTION

Major depressive disorder (MDD) is a serious medical illness affecting about 10 million American adults. In a given year, about 5-7% adults in the developed countries suffer from mood disorders, a cluster of mental disorders best recognized by depression or mania. Unlike normal emotional experiences of sadness, loss, or passing mood states, major depression is persistent and can significantly interfere with an individual's thoughts, behavior, mood, activity, and physical health. Among all medical illnesses, major depression is the leading cause of disability in the U.S. and many other developed countries. The occurrence rate for MDD is two times higher among women than among men (Blehar et al., Medscape Women's Health 2:3 (1997)). Major depression can occur at any age including childhood, the teenage years and adulthood. All ethnic, racial and socioeconomic groups suffer from depression. About three-fourths of those who experience a first episode of depression will have at least one other episode in their lives. Some individuals may have several episodes in the course of a year. If untreated, episodes commonly last anywhere from six months to a year. Left untreated, depression can lead to suicide.

Several different treatment options are available for patients with depression as well as psychiatric counseling. The therapeutic effects of antidepressants are believed to be related to an effect on neurotransmitters, particularly by inhibiting the monoamine transporter proteins of serotonin and norepinephrine. Selective serotonin reuptake inhibitors (SSRIs) specifically prevent the reuptake of serotonin (thereby increasing the level of serotonin in synapses of the brain), whereas earlier monoamine oxidase inhibitors (MAOIs) blocked the destruction of neurotransmitters by enzymes which normally break them down. Tricyclic antidepressants (TCAs) prevent the reuptake of various neurotransmitters, including serotonin, norepinephrine, and dopamine.

At present no specific genetic or biochemical tests are available for the positive diagnosis of depression. Diagnosis and treatment is presently based solely on patient self-reporting and symptom description. The clinical heterogeneity associated with depression has complicated patient reporting as well as the diagnosis and treatment of the disorder. As a result, no clear modality of treatment for all individuals with depression has emerged, and treatment as well as diagnosis varies greatly not only from patient to patient but from physician to physician. Thus, many sufferers of depression are not effectively treated.

### SUMMARY OF THE CLAIMED INVENTION

The invention provides a method of polymorphic profiling an individual. The method comprises determining a polymorphic profile in at least two but no more than 1000 polymorphic sites, the polymorphic sites including at least two sites shown in Table 1 or in linkage disequilibrium therewith. Optionally, the polymorphic profile is determined in at least two polymorphic sites shown in Table 3. Optionally, the polymorphic profile is determined in at least 2 and no more than 50 different polymorphic sites shown in Table 3. Optionally, the polymorphic profile is determined in at least 5 polymorphic sites shown in Table 1 or 3. Optionally, the polymorphic profile is determined in at least 10 polymorphic sites shown in Table 1 or 3. Optionally, the polymorphic profile is determined in at least two polymorphic sites in or within 10 kb of the at least two genes shown in Table 1. Optionally, the polymorphic profile is determined in at least two polymorphic sites in or within 10 kb of at least two genes shown in Table 2. Optionally, the polymorphic profile is determined in at least two polymorphic sites in at least two genes shown in Table 1 or Table 2. Optionally, the polymorphic profile is determined at polymorphic sites in at least 5 genes shown in Table 1 or Table 2. Optionally, the polymorphic profile is determined in at least two polymorphic sites shown in Table 1 or 3. Optionally, the polymorphic profile is determined in at least five polymorphic sites shown in Table 1 or 3. Optionally, one of the polymorphic sites is in the TTC12 gene or in linkage disequilibrium therewith. Optionally, one of the polymorphic sites is SNP No. 1752273.

The invention further provides a method of determining whether a patient with depression is suitable for treatment with an SSRI or inclusion in a clinical trial for testing an SSRI. The method comprises determining presence of a polymorphic profile in at least one polymorphic site shown in Table 1 or 3 or in linkage disequilibrium therewith; and determining whether to treat the patient with the SSRI or include the patient in a clinical trial based on the polymorphic profile. Optionally, the method further comprises determining the total number of alleles in the polymorphic profile associated with a positive response to SSRIs and the total number of alleles in the polymorphic profile associated with a negative (or lack of) response to SSRIs, whereby a higher number of alleles associated with the positive response than alleles associated with a negative response is an indication of whether a patient with depression is amenable to treatment with SSRIs or should be included in a clinical trial for testing an SSRI. Optionally, the method further comprises determining the total number of alleles in the polymorphic profile associated with a positive response to placebo and the total number of alleles in the polymorphic profile associated with a negative response (or lack of) to placebo, whereby a higher number of alleles associated with the positive response than alleles associated with a negative response is an indication of whether a patient is susceptible to a placebo effect or should be excluded from a clinical trial for testing an SSRI. Optionally, the method determines which polymorphic forms are present in at least 10 polymorphic sites shown in Table 1 or Table 3. Optionally, the method further comprises treating the patient with an SSRI. Optionally, the method further comprises treating the patient with a treatment for depression other than with an SSRI. Optionally, the method further comprises further comprises performing a clinical trial to test an SSRI on a population including the patient. Optionally, the method further comprises performing a clinical trial to test the SSRI on a population not including the patient. Optionally, one of the polymorphic sites is in the gene TTC12 or in linkage disequilibrium therewith. Optionally, the polymorphism is SNP No. 1752273.

The invention further provides a method of expression profiling. The method comprises determining expression levels of at least 2 and no more than 10,000 genes in a subject, wherein at least two of the genes are from Table 1 or 2, the expression levels forming an expression profile. Optionally, the subject has depression. Optionally, the method further comprises determining expression levels of the genes in an individual not having depression to determine genes differentially expressed in depression. Optionally, the method further comprises determining the expression levels of the genes in a positive control subject having depression and amenable to treatment with SSRIs and a negative control subject having depression and not amenable to treatment with SSRIs, and comparing the expression levels of the genes in the subject with expression levels of the genes in the positive control and negative control, wherein similarity of expression profiles in the subject and the positive control is an indication the subject is amenable to treatment with an SSRI, and similarity of the expression profiles in the subject and the negative is an indication that the subject is not amenable to treatment with an SSRI. Optionally, the expression levels of at least five genes shown in Table 1 or 2 are determined. Optionally, the determining step determines the expression level of at least 2 and no more than 100 genes, wherein the at least two genes are shown in Table 1 or 2. Optionally, the determining step determines the expression levels of at least 5 genes shown in Table 1 or 2. Optionally, the determining step determines the expression levels of at least 10 genes shown in Table 1 or 2.

The invention further provides a method of screening a compound activity in modulating depression. The method comprises determining whether a compound binds to, modulates expression of, or modulates the activity of a polypeptide encoded by a gene shown in Table 1 or Table 2. Optionally the determining comprises contacting the compound with the polypeptide and detecting specific binding between the compound and the polypeptide. Optionally, the determining comprises contacting the compound with the polypeptide and detecting a modulation of activity of the polypeptide. Optionally, the determining comprises contacting the gene or other nucleic acid encoding the polypeptide with the compound and detecting a modulation of expression of the polypeptide.

The invention further provides a method of effecting treatment or prophylaxis of depression. The method comprises administering to a subject having or at risk of depression a compound that modulates expression or activity of a gene shown in Table 1 or 2. Optionally, the compound is selected from the group consisting of an antibody that specifically binds to a protein encoded by a gene shown in Table 1 or 2; a zinc finger protein that modulates expression of a gene shown in Table 1 or 2; an siRNA, antisense RNA, RNA complementary to a regulatory sequence, or ribozyme that inhibits expression of a gene shown in Table 1 or 2. Optionally, the gene is shown in Table 1 or 2.

The invention further provides a transgenic nonhuman animal having a genome comprising an exogenous gene shown in Table 1 or 2.

The invention further provides a transgenic nonhuman animal having a genome with a disrupted endogenous gene that is a species variant of a gene shown in Table 1 or 2.

### DEFINITIONS

A polymorphic site is a locus of genetic variation in a genome. A polymorphic site is occupied by two or more polymorphic forms (also known as variant forms or alleles). A single nucleotide polymorphic site (SNP) is a variation at a single nucleotide.

The term “haplotype block” refers to a region of a chromosome that contains one or more polymorphic sites (e.g., 1-10) that tend to be inherited together (i.e., are in linkage disequilibrium) (see Patil et al., Science, 294:1719-1723 (2001); US 20030186244)). Combinations of polymorphic forms at the polymorphic sites within a block cosegregate in a population more frequently than combinations of polymorphic sites that occur in different haplotype blocks.

The term “haplotype pattern” refers to a combination of polymorphic forms that occupy polymorphic sites, usually SNPs, in a haplotype block on a single DNA strand. For example, the combination of variant forms that occupy all the polymorphisms within a particular haplotype block on a single strand of nucleic acid is collectively referred to as a haplotype pattern of that particular haplotype block. Many haplotype blocks are characterized by four or fewer haplotype patterns in at least 80% of individuals. The identity of a haplotype pattern can often be determined from one or more haplotype determining polymorphic sites (e.g., “tag SNPs”) without analyzing all polymorphic sites constituting the pattern.

The term “linkage disequilibrium” refers to the preferential segregation of a particular polymorphic form at one polymorphic site with another polymorphic form at a different polymorphic site more frequently than expected by chance. Such polymorphic forms, polymorphic sites at which the polymorphic forms occur, and genes including the polymorphic sites are said to be in linkage disequilibrium with each other. Linkage disequilibrium can also refer to a situation in which a phenotypic trait displays preferential segregation with a particular polymorphic form or another phenotypic trait more frequently than expected by chance.

A polymorphic site is proximal to a gene if it occurs within the intergenic region between the transcribed region of the gene and that of an adjacent gene. Usually, proximal implies that the polymorphic site occurs closer to the transcribed region of the particular gene than that of an adjacent gene. Typically, proximal implies that a polymorphic site is within 50 kb, and preferably within 10 kb of the transcribed region. Polymorphic sites not occurring in proximal regions as defined above are said to occur in regions that are distal to the gene.

Specific binding between two entities means a mutual affinity of at least 10⁶ M⁻¹, and usually at least 10⁷ or 10 M⁻¹. The two entities also usually have at least 10-fold greater affinity for each other than the affinity of either entity for an irrelevant control.

A nonhuman homolog of a human gene is the gene in a nonhuman species, such as a mouse, that shows greatest sequence identity at the nucleic acid and encoded protein level, and higher order structure and function of the protein product to that of the human gene or encoded product.

Modulation means a change in the function of a gene product. For example, such change may be related to an increase or decrease in activity or expression, or altered timing of expression or activity.

The terms “isolated” and “purified” refer to a material that is substantially or essentially removed from or concentrated in its natural environment. For example, an isolated nucleic acid is one that is separated from the nucleic acids that normally flank it or from other biological materials (e.g., other nucleic acids, proteins, lipids, cellular components, etc.) in a sample. In another example, a polypeptide is purified if it is substantially removed from or concentrated in its natural environment.

“Statistically significant” means significant at a p value ≦0.05.

The term “comprising” indicates that other elements can be present besides those explicitly stated.

### DETAILED DESCRIPTION OF THE INVENTION

### I. General

The invention provides a collection of polymorphic sites associated with variation in outcome from treatment of patients suffering from depression with a selective serotonin reuptake inhibitor (SSRI) or a placebo. Some polymorphic sites are occupied by variant forms associated with a positive response or negative response to SSRI's. That is, at a given site, one of the alleles is associated with a positive response and the other with a negative response or lack of response. Other polymorphic sites are occupied by variant forms associated with a positive or negative (lack of) response to a placebo. Likewise, this means that at one polymorphic site, one allele is associated with a positive response and the other with a negative response or lack of response. In general, the polymorphic sites associated with response to an SSRI are different from the polymorphic sites associated with response to a placebo.

The collection of polymorphic sites and genes has a variety of uses. Depression patients identified with a variant form or predominance of variant forms associated with a positive outcome to treatment with SSRI's are identified as being suitable for treatment with SSRI's and for inclusion in clinical trials intended to test SSRI's. Conversely, depression patients identified with a variant form or a predominance of variant forms associated with a negative (lack of) response to treatment with SSR's are identified as being less suitable or not suitable for treatment with SSRI's or inclusion in clinical trials to test SSRI's. Individuals identified with a variant form or a predominance of variant forms associated with a positive outcome from placebo (i.e., in the absence of treatment) are indicated as being less suitable or unsuitable for treatment with SSRI's and for inclusion in clinical trials. Individuals identified with a variant form or a predominance of variant forms associated with a negative outcome from placebo are indicated as being suitable for treatment with SSRI's and inclusion in clinical trials.

The genes containing, or in linkage disequilibrium, with the polymorphic sites and their encoded proteins can be used to identify compounds that modulate the expression or activity of the encoded proteins. Such compounds are useful for treating depression, optionally in combination with other treatments, particularly SSRIs. The collection of genes is also useful for generating transgenic animal models of depression. These models are useful for screening compounds to determine presence of pharmacological activity useful for treating depression.

### II. Measurement of Response to Treatment

A depression patient's response to an SRRI or a placebo can be measured in either a quantitative or binary fashion. A quantitative analysis means that each patient is associated with a value indicating the magnitude of the response (i.e., improvement in the condition of the patient), if any. A binary response means that each patient is classified as responding (i.e., improving in condition) or not responding based on whether the patient achieves a predefined threshold response value. Irrespective whether the analysis is quantitative or binary, the response can be evaluated on several different scales of depression including HAM-D, or its subscales: insomnia, anxiety and Core Lilly.

An allele is associated with a positive response to treatment with an SRRI or a placebo if the presence of the allele correlates positively and significantly with the magnitude of the response or rate of response (inverse of time) on any quantitative scale of severity of depression or its component phenotypes in a population of patients so treated. An allele is also associated with a positive response to treatment with an SSRI or placebo if the allele is present significantly more frequently in a population of patients achieving a threshold value of response on any quantitative scale than not a achieving a threshold in a binary analysis. Conversely, an allele is associated with a negative (or lack of) response to treatment with an SSRI or placebo if the presence of the allele correlates negatively and significantly with the magnitude or rate of the response in a population of patients. An allele is also associated with a negative (lack of) response to treatment with an SSRI or a placebo if the allele is present significantly less frequently in a population of patients achieving a threshold than in a population not achieving a threshold value of response on any quantitative scale in a binary analysis. In general, each polymorphic site of the invention can be occupied by two variant alleles, one of which associates with a positive response to treatment with an SSRI or a placebo and the other a negative (lack of) response to treatment with an SSRI or a placebo.

### III. Polmorphic Sites and Genes

The invention provides a large collection of polymorphic sites associated with response to SSRIs and/or a placebo as shown in Table 1. The first and second columns provide identification numbers for each SNP. The first column is an internal Perlegen number. The second column is the reference number according to dbSNP database established and maintained by NCBI of the National Library of Medicine at the National Institute of Health, Build 34). If a SNP does not have an rs_ID, this means that Perlegen Sciences has not submitted this SNP to dbSNP, but that this is an existing SNP in dbSNP mapped (in the Perlegen alignment process) to the same location as the Perlegen SNP. The third column of the table indicates the chromosome on which the polymorphic site is found. The fourth column provides the accession number for the genomic region containing the SNP. The fifth column provides the location of the SNP in the genomic region identified by the accession number in the fourth column (NCBI, Build 34 of the human genome map). The sixth and seventh columns provide the alternative bases occupying the polymorphic sites. The assignment as ref or alternative does not indicate whether an allele correlates positively or negatively with a placebo or an SRRI response. The eighth column indicates 51 bases of nucleotide sequence centered about a polymorphic site. The ninth column provides the frequency of the reference allele in all tested populations (irrespective of treatment regime). The tenth column lists the genes flanking a polymorphic site with the polymorphic site indicated by square brackets. If the square brackets enclose a gene, the polymorphic site is within the gene. The gene names are those defined by the authorities in the field such as HUGO, or conventionally used in the art to describe the genes. Further information as to whether each polymorphic site associates with an SRRI treatment or placebo response by a variety of scales and measurements on each scale, together with statistical parameters is provided in Tables 5-10 and in the Examples.

Table 2 shows a preferred collection of about 27 genes shown in Table 1, all of which have been identified as “CNS-relevant” based on a search of the published literature and public databases (e.g., some are known to be expressed in the CNS). The first three columns of the Table list the genes, GeneID from the NCBI Gene database, and their functions known to date. The remaining six columns indicate the type of response associated with each gene. A total of 24 different responses were analyzed for a polymorphic site in each gene. Each polymorphic site was analyzed for associations with outcome to treatment with placebo and an SSRI. These analyses are collectively referred to as “by genoytpe” and “by interaction” respectively in Table 2. Both placebo and SSRI responses were analyzed using HAM-D and its three subscales of depression. HAM-D is an overall measure of depression. Insomnia, Core Lilly, and anxiety are measures of included aspects of depression, as discussed in the Examples. Each scale was in turn analyzed by three measures of the response on that scale (time to response, binary, i.e., subject either meets or does not meet an endpoint, or quantitative measure of response). The last six columns in Table 2 are grouped in three pairs. Each pair shows placebo and SSRI responses, and the three pairs show the three different measures of response. If a particular column is occupied by a scale, it signifies that the gene in the same row as the scale contained a polymorphic site for which one allele showed a positive response on the scale and the other allele showed a negative (or lack of) response. Thus, for example, a polymorphic site in the AUTS2 gene contains variant alleles, one of which showed a positive response and the other a negative (or lack of) response to placebo as determined by binary measurement of insomnia and linear (i.e., quantitative) measures of Core Lilly and HAM-D. Likewise, a polymorphic site in the GRM8 gene contains variant alleles, one of which showed a positive response and the other a negative (or lack of) response to placebo as determined by time to respond on the CLilly scale. Likewise a polymorphism in the gene HTR2C contains variant alleles, one of which showed a positive response and the other negative response to SSRI treatment determined by a linear measurement on the CLilly scale.

Table 3 shows polymorphic sites within the genes of Table 2. Some genes contain more than one polymorphic site. The columns of Table 3 correspond to those of Table 1 as discussed above, except that the ninth column provides the identity of a single gene containing the polymorphic site of that row of the table, and the tenth column provides information regarding the analysis or analyses that showed the SNP to be significantly associated. For the three letter designations, the first letter indicates whether the analysis was binary (B), linear (L) or time (T); the second letter indicates whether the analysis was by genotype (G) or by interaction (I); and the third letter indicates which measure was used (e.g., anxiety (A), HAM-D (H), Core Lilly (C), and insomnia (I)). Each of the polymorphic sites shown in Table 3 has one variant form positively associated with either a placebo or an SSRI response, and one variant form negatively associated with either a placebo or an SSRI response.

Table 4 shows additional SNPs in CNS relevant genes that have been associated with a placebo or SSRI effect. The first column indicates the model (e.g., “linearinteract” means association with SSRI effect by a linear measurement). The second column indicates the scale of depression used. Table 4 provides a reference for the SNP used. Further information regarding the SNP can be obtained from Table 1. Columns 5 and 6 provide statistical information regarding the association as further defined below.

Tables 5-10 shows additional SNPs in genes not known to have CNS roles. The first column shows the SNP number. Further information regarding the SNP can be obtained from Table 1. The second and third columns provide statistical information regarding the association as further defined below.

### IV. Depression

Depression is a mood disorder characterized by persistent feelings of sadness for several weeks or more. There are several subtypes of depression. Major Depressive Disorder (MDD) impairs a person's ability to work, sleep, eat, and function as he or she normally would. It keeps subjects from enjoying activities that were once pleasurable, and causes them to think about themselves and the world in negative ways. MDD is often disabling and may occur several times in a person's lifetime. Dysthymic Disorder (DD) is a milder yet more enduring type of major depression. People with DD may appear to be chronically mildly depressed to the point that it seems to be a part of their personality. When a subject seeks treatment for dysthymia, it is not uncommon that he/she has struggled with this condition for a number of years. Bipolar Disorder also known as manic-depression or manic-depressive disorder is characterized by mood swings that alternates between periods of depression and periods of elation and excitable behavior known as mania. For people who have bipolar disorder, the depressions can be severe and the mania can seriously impair one's normal judgment. When manic, a person is prone towards reckless and inappropriate behavior. Cyclothymic Disorder is a milder yet more enduring type of bipolar disorder. A person's mood alternates between a less severe mania (known as hypomania) and a less severe depression.

Presence of depression can be determined by questionnaire according to the Diagnostic and Statistical Manual of Mental Disorders—Fourth Edition (American Psychiatric Association, 1994) patients. HAM-D is a commonly used scale to assess the severity of depression. The scale was developed for use primarily on patients who have already been diagnosed as suffering from affective disorders. Questions are related to symptoms such as, for example, depressed mood, guilty feelings, suicide, sleep disturbances, anxiety levels and weight loss (Hamilton, J. Neurology Neurosurgery Psychiatry 23:56-62 (1960). Subsets of questions on the HAM-D scale can also be used to calculate subscores for depression, anxiety and insomnia as described in the Examples. Another scale is the Montgomery-Åsberg Depression Rating Scale (MADRS). This scale has been designed to measure the treatment changes of depression. It measures the severity of many symptoms of depression such as, for example, mood and sadness, tension, sleep, appetite, energy, concentration, suicide and restlessness.

Most forms of depression can be treated by psychiatric counseling and a variety of drugs. The most commonly prescribed drugs for depression are SSRIs. Other available classes of drugs are monoamine oxidase inhibitors (MAOIs), tricyclic antidepressants, serotonin-norepinephrine reuptake inhibitors (SNRIs), norepinephrine/noradrenaline reuptake inhibitors (NRIs aka NERIs/NARIs), dopamine reuptake inhibitors (DRIs), opioids, selective serotonin reuptake enhancers (SSREs), and tetracyclic antidepressants. Within each class there are numerous different drugs. Examples of SSRIs include fluoxetine, paroxetine, citalopram, escitalopram and sertraline. Venlafaxine and duolxetine are examples of SNRIs, Fluvoxamine of an SSRI, and Bupropion of a DRI and NRI.

### V. Methods of Polymorphic Profiling

The invention provides methods of profiling individuals at one or more SNPs of the invention. A polymorphic profile refers to the matrix of variant forms occupying one or more polymorphic sites. The profile can be determined on at least 1, 2, 5, 10, 25, 35, 50, 100, 500, 1000 or all of polymorphic sites shown in any one of Tables 1, 2, 3, 4, 5A-D, 6A-D, 7A-D, 8A-D, 9A-D and 10A-D, all of these tables, or any combinations thereof and optionally other polymorphisms in linkage disequilibrium with them. The profile can include polymorphic sites from CNS relevant genes (Tables 2-4) or other genes (Tables 5A-D, 6A-D, 7A-D, 8A-D, 9A-D and 10A-D) or a combination thereof. The polymorphic profile is preferably determined in at least 1, 2, 5, 10, 25 or all of the polymorphic sites shown in any of Tables 3, 4, 5A-D, 6A-D, 7A-D, 8A-D, 9A-D and 10A-D, all of these tables or any combination thereof. For polymorphic sites in linkage disequilibrium with a polymorphic site shown in Table 1 or 3, polymorphic sites occurring in the same gene as shown in Table 1 or 3 or proximal thereto are preferred. The polymorphic profile preferably includes polymorphic sites from at least 2, 5, 10, 15, 25 or all of the genes shown in Table 1, 2 and/or 3. The polymorphic profile can alternatively or additionally including polymorphic sites from at least 2, 5, 10, 15, 25 or all the genes containing a polymorphic site shown in any of Tables 4, 5A-D, 6A-D, 7A-D, 8A-D, 9A-D, or 10A-D. The polymorphic sites of the invention can be analyzed in combination with other polymorphic sites. However, the total number of polymorphic sites analyzed is usually less than 10,000, 1000, 100, 50 or 25.

The number of alleles associated positively or negatively with a given response present in a particular individual can be combined additively or as ratio to provide an overall score for the individual's genetic propensity to the response (see US 2005-0196770 A1). For example, alleles associated with a positive response to an SSRI can be arbitrarily each scored as +1 and alleles associated with a negative response as −1 (or vice versa). For example, if an individual is typed at 30 polymorphic sites of the invention and is homozygous for alleles associated with a positive response to an SSRI at all of them, he or she could be assigned a score of 100% genetic amenability to treatment with an SSRI. The reverse applies if the individual is homozygous for all alleles associated with a negative (or lack of) response to an SSRI. More typically, an individual is homozygous for positively associated alleles at some loci, homozygous for negatively associated alleles at some loci, and heterozygous for positively and negatively associated alleles at other loci. Such an individual's genetic amenability to treatment with an SSRI can be scored by assigning all positively associated alleles a score of +1, and all negatively associated alleles a score of −1 (or vice versa) and combining the scores. For example, if an individual has 40 positively associated alleles and 20 negatively associated alleles, the individual can be scored as having a 67% genetic amenability to treatment with an SSRI. Alternatively, homozygous positively associated alleles can be assigned a score of +1, heterozygous alleles a score of zero and homozygous negatively associated alleles a score of −1. The relative numbers of resistance alleles and susceptibility alleles can also be expressed as a percentage. Thus, an individual who is homozygous for positively associated alleles at 20 polymorphic sites, homozygous for negatively associated alleles at 40 polymorphic sites, and heterozygous at 10 sites is assigned a genetic amenability of 33% for treatment with an SSRI. As a further alternative, homozygosity for positively associated alleles can be scored as +2, heterozygosity, as +1 and homozygosity for negatively associated alleles as 0.

Similar calculations can be performed to assess the individual's genetic susceptibility to a placebo response. In general the polymorphic sites associating with a placebo response are different from those associating with an SSRI response, so any given polymorphic site is used in only one of the two calculations.

The nature of the polymorphic profile of an individual and the scores calculated from it are useful in determining how to treat a patient and/or whether to include the patient in a clinical trial to test a new SSRI. If a patient has a genetic amenability to treatment with an SSRI, the test indicates that treatment of the patient with an SSRI should be begun or continued. Alternatively, if the treatment has proved or proves to be unsuccessful, such an outcome signals that a different SSRI should be tried. The test also signifies that the patient is suitable for inclusion in a clinical trial to test a new SSRI. Alternatively, if the patient has a low genetic amenability to treatment with an SSRI, the test indicates that treatment with an SSRI should not be initiated or should be discontinued. The test also provides an indication that the patient should preferably not be included in a clinical trial to test an SSRI.

If the analysis indicates a patient has a high genetic amenability to respond positively to a placebo, the test provides an indication that the individual should not be treated with an SSRI because the patient has a propensity to recover without treatment. However, the test does not distinguish between whether the patient recovers without treatment due to the psychological placebo effect or due to the subtype of depression affecting the patient. Accordingly, the patient can be prescribed a placebo. The test also provides an indication that the patient should be excluded from clinical trials to test an SSRI. If the analysis indicates a patient has a low genetic amenability to a placebo effect, the test provides an indication that some treatment is desirable but does not distinguish whether an SSRI or other treatment is preferred. However, such can be indicated by analysis of polymorphisms associated with the SSRI response. Similarly, a low genetic amenability to a placebo effect provides an indication that the patient is suitable for inclusion in a clinical trial to treat depression but does not indicate whether the patient is amenable to treatment with SSRIs or other treatment. Again, this information can be obtained from analysis of polymorphic sites associated with the SSRI response.

Polymorphic profiling is useful for stratifying individuals in clinical trials of compounds being tested for capacity to treat depression, particularly of SSRIs. Such trials are performed on treated or control populations having similar or identical polymorphic profiles (see WO0033161). Use of genetically matched populations (i.e., statistically significant similarity of polymorphic profile at a defined set of polymorphic sites of the invention relative to similarity of polymorphic profile at these sites in the general population) eliminates or reduces variation in treatment outcome due to genetic factors, leading to a more accurate assessment of the efficacy of a potential drug. This also provides for maximum treatment difference when response to SSRI treatment is assessed against response to placebo treatment in a clinical trial.

Polymorphic profiles can also be used after the completion of a clinical trial to elucidated differences in response to a given treatment. For example, the set of polymorphisms can be used to stratify the enrolled patients into disease sub-types or classes. It is also possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphisms can be used to identify the genetic factors involved in adverse response to treatment (adverse events). For example, patients who show adverse response may have more similar polymorphic profiles than would be expected by chance. This allows the early identification and modification or protocol or exclusion of such individuals from treatment. It also provides information that can be used to understand the biological causes of adverse events and to modify the treatment to avoid such outcomes.

Polymorphic profiles can also be used for other purposes, including paternity testing and forensic analysis, such as described by U.S. Pat. No. 6,525,185. In forensic analysis, the polymorphic profile from a sample at the scene of a crime is compared with that of a suspect. A match between the two is evidence that the suspect in fact committed the crime, whereas lack of a match excludes the suspect.

Polymorphic profiles can be used in further association studies of traits related to depression. Such traits include presence of depression and its subtypes, related diseases, amenability to treatment of depression with agents other than SSRIs or with combinations of agents, amenability to recovery without treatment or placebo. Polymorphic forms can also be further characterized for their effect on the activity of a gene or its expression levels. Polymorphic forms occurring within a protein coding sequence are likely to effect activity of the encoded protein particularly if the change between forms is nonsynonymous. Polymorphic forms occurring between genes are more likely to affect expression levels. Polymorphic forms occurring in introns can affect expression levels or splice variation.

Although polymorphic profiling can be done at the level of individual polymorphic sites as described above, a more sophisticated analysis can be performed by analyzing haplotype blocks containing SNPs of the invention and/or others in linkage disequilibrium with them (see, e.g., US 20040220750). Each haplotype block can be characterized by two or more haplotype patterns (i.e., combinations of polymeric forms). In some instances, a haplotype pattern can be determined by detecting a single haplotype-determining polymorphic form within a haplotype block. In other instances, multiple polymorphic forms are determined within the block (see Patil et al., Science 294, 1719-23 (2001)). The haplotype pattern at each of the haplotype blocks containing SNPs of the invention in an individual is a factor in determining response to an SRRI or a placebo, and can be characterized as associating positively or negatively with an SSRI or placebo response as can individual polymorphic forms. The number of haplotype blocks occupied by haplotype patterns associated with a positive response and the number associated with a negative response in a particular individual can be combined additively as for individual polymorphic forms to arrive at a percentage representing genetic propensity to positive or negative response. The measure is more accurate than simply combining individual polymorphic forms because it gives the same weight to haplotype blocks containing multiple polymorphic sites as haplotype blocks within a single polymorphic site. The multiple polymorphic forms within the same block are associated with the same propensity to positive or negative response, and should not be given the same weight as multiple polymorphic forms in different haplotype blocks, which indicate independent propensity for positive or negative response.

The methods of the invention detect haplotype patterns in at least 1, 2, 5, 10, 25, 100, 500, 1000 or all of the haplotype blocks of the invention. Preferably, the haplotype patterns include at least 1, 2, 5, 10 or 25 or all of the genes shown in Table 1, 2 or 3. Alternatively or additional, the haplotype patterns can include at least 1, 2, 5, 10 or 25 or all of the genes including a polymorphic site shown in any of Tables 4, 5A-D, 6, A-D, 7A-D, 8A-D, 9A-D, 10A-D. The haplotype patterns can be detected in combination with haplotype patterns at haplotype blocks other than those of the invention. However, the number of haplotype blocks is typically fewer than 10,000, 1000 and often fewer than 100 or 50.

Polymorphic forms can be detected at polymorphic sites by a variety of methods. The design and use of allele-specific probes for analyzing polymorphisms is described by e.g., Saiki et al., Nature 324, 163-166 (1986); EP 235,726; WO 89/11548. Allele-specific probes can be designed that hybridize to a segment of target DNA from one individual but that do not hybridize to the corresponding segment from another individual due to the presence of different polymorphic forms in the respective segments from the two individuals.

The polymorphisms can also be identified by hybridization to nucleic acid arrays, some example of which are described by WO 95/11995. Polymorphic forms can also be detected using allele-specific primers, which hybridize to a site on target DNA overlapping a polymorphism and only primes amplification of an allelic form to which the primer exhibits perfect complementarily. See Gibbs, Nucleic Acid Res. 17, 2427-2448 (1989). Polymorphic forms can also be detected by direct sequences, denaturing gradient gel electrophoresis (Erlich, ed., PCR Technology, Principles and Applications for DNA Amplification, (W.H. Freeman and Co, New York, 1992, Chapter 7), and single stranded polymorphisms analysis (Orita et al., Proc. Nat. Acad. Sci. 86, 2766-2770 (1989)). Polymorphic forms can also be detected by single-base extension methods as described by e.g., U.S. Pat. No. 5,846,710, U.S. Pat. No. 6,004,744, U.S. Pat. No. 5,888,819 and U.S. Pat. No. 5,856,092. The methods hybridize a primer that is complementary to a target sequence such that the 3′ end of the primer is immediately adjacent to but does not span a site of potential variation in the target sequence. That is, the primer comprises a subsequence from the complement of a target polynucleotide terminating at the base that is immediately adjacent and 5′ to the polymorphic site. The hybridization is performed in the presence of one or more labeled nucleotides complementary to base(s) that may occupy the site of potential variation. Some polymorphic forms resulting in a corresponding change in encoded proteins can also be detected at the protein level by immunoassay using antibodies known to be specific for particular variants, or by direct peptide sequencing.

### VI. Expression Monitoring

The invention also provides methods of expression profiling by determining levels of expression of one or more genes shown in Table 1. The methods preferably determine expression levels of at least 2, 5, 10, 15, 20, 25, 100, 200, 500 or all of the genes shown in Table 1, 2 or 3. Alternatively or additionally, the methods determine expression levels in at least 2, 5, 10, 15, 20, 25, 100, 200, 500 or all of the genes containing a polymorphism shown in any of Tables 4, 5A-D, 6A-D, 7A-D, 8A-D, 9A-D, or 10A-D. Preferably, the expression levels are determined of at least 2, 5, 10, 15, 20, 25 or all of the genes shown in Table 2 or 3. Alternatively or additionally, the expression levels are determined in at least 2, 5, 10, 15, 20, of all of the genes shown in any of Tables 4, 5A-D, 6A-D, 7A-D, 8A-D, 9A-D, or 10A-D. Optionally, expression levels of other genes other than those associated with response to an SSRI or placebo as described in this application are also determined. However, the expression profile is preferably not determined at more than 1000, 5000, or 10,000 genes.

The expression levels of one or more genes in a discrete sample (e.g., from a particular individual or cell line) are referred to as an expression profile. Typically, the expression profile is compared with an expression profile of the same genes in a control sample to determine genes differentially expressed between the two samples. If the test sample is a depression patient, the control can be a subject not having depression. Alternatively, if the test subject is a depression patient being treated with an SSRI, the control can be a depression patient being treated with a placebo, another class of drug, psychotherapy or receiving no treatment. In other methods, the amenability of a test subject to treatment with an SSRI is unknown and the object is to determine the same. In such methods, the expression profile of the test subject is compared with the expression profile of positive and negative control subjects. The positive control subject is an individual known to be amenable to treatment with SSRI. Such an individual at minimum shows a significant benefit from treatment with at least one SSRI and preferably scores in the top ten percentile of depressed individuals in responding to the SSRI. Such an individual can also be recognized by a predominance of alleles positively associated with a response to an SSRI as discussed above. The negative control subject is an individual known to have an insignificant response to at least one SSRI (e.g., scorring in the bottom ten percentile of depressed individuals in responding to the SSRI), and can also be recognized by a predominance of alleles negatively associated with a response to an SSRI, as discussed above. The controls can be contemporaneous or historical. Individual expression levels in both the test and control samples can be normalized before comparison, e.g., by reference to the levels of a housekeeping gene to avoid differences unrelated to the disease.

If the expression profile of the test subject is more similar to that of the positive control than the negative control, the analysis provides an indication that the test subject is amenable to treatment with an SSRI. Conversely if the expression profile of the test subject is more similar to that of the negative control than the positive control, the analysis provides an indication that the test subject is not amenable to treatment with an SSRI. For example, if an expression profile is determined for ten genes of the invention, and the expression levels in the test subject are more similar to the positive control than the negative control for nine of the genes, one can conclude that the test individual is amenable to treatment with an SSRI. The analysis can be performed at a more sophisticated level by weighting expression level according to where they lie between negative and positive controls. For example, if there is a large difference between negative and positive controls, and an expression level of a particular gene in a test individual lies close to the positive control that expression level is accorded greater weight than an expression level in a gene in which there is a smaller difference in expression levels between negative and positive controls, and the expression level of the test individual lies only slightly above the midpoint of the negative and positive control expression levels.

### VII. Compounds to Modulate Depression or Response to Treatment Thereof

A variety of compounds can be screened for capacity to modulate expression or activity of genes associated with response to treatment of depression with an SSRI or placebo, i.e., the genes shown in Tables 1, 2 and/or 3 or genes containing a polymorphic site shown in any of tables 4, 5A-D, 6A-D, 7A-D, 8A-D, 9A-D, and 10A-D. Compounds can be obtained from natural sources, such as, e.g., marine microorganisms, algae, plants, and fungi. Alternatively, compounds can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, organic compounds, lipids, glucocorticoids, antibiotics, peptides, proteins, sugars, carbohydrates, and chimeric molecules.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods. See, e.g., WO91/19818. Compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, or the like. For genes encoding transporters, the compounds include substrates of the transporters, and analogs of the same.

Many compounds currently in use for treating depression can be screened for capacity to modulate the above proteins. The compounds include antibodies, both intact and binding fragments thereof, such as Fabs, Fvs, which specifically bind to a protein encoded by a gene of the invention. Usually the antibody is a monoclonal antibody although polyclonal antibodies can also be expressed recombinantly (see, e.g., U.S. Pat. No. 6,555,310). Examples of antibodies that can be expressed include mouse antibodies, chimeric antibodies, humanized antibodies, veneered antibodies and human antibodies. Chimeric antibodies are antibodies whose light and heavy chain genes have been constructed, typically by genetic engineering, from immunoglobulin gene segments belonging to different species (see, e.g., Boyce et al., Annals of Oncology 14:520-535 (2003)). For example, the variable (V) segments of the genes from a mouse monoclonal antibody may be joined to human constant (C) segments. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions substantially from a mouse-antibody, (referred to as the donor immunoglobulin). See Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539. The constant region(s), if present, are also substantially or entirely from a human immunoglobulin. Antibodies can be obtained by conventional hybridoma approaches, phage display (see, e.g., Dower et al., WO 91/17271 and McCafferty et al., WO 92/01047), use of transgenic mice with human immune systems (Lonberg et al., WO93/12227 (1993)), among other sources. Nucleic acids encoding immunoglobulin chains can be obtained from hybridomas or cell lines producing antibodies, or based on immunoglobulin nucleic acid or amino acid sequences in the published literature.

The compounds also include several categories of molecules known to regulate gene expression, such as zinc finger proteins, ribozymes, siRNAs and antisense RNAs. Zinc finger proteins can be engineered or selected to bind to any desired target site within a gene of the invention. An exemplary motif characterizing one class of these proteins (C₂H₂ class) is -Cys-(X)₂₋₄-Cys-(X)₁₂-His-(X)₃₋₅-His (where X is any amino acid). A single finger domain is about 30 amino acids in length, and several structural studies have demonstrated that it contains an alpha helix containing the two invariant histidine residues and two invariant cysteine residues in a beta turn co-ordinated through zinc. In some methods, the target site is within a promoter or enhancer. In other methods, the target site is within the structural gene. In some methods, the zinc finger protein is linked to a transcriptional repressor, such as the KRAB repression domain from the human KOX-1 protein (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994)). In some methods, the zinc finger protein is linked to a transcriptional activator, such as VIP16. Methods for selecting target sites suitable for targeting by zinc finger proteins, and methods for design zinc finger proteins to bind to selected target sites are described in WO 00/00388. Methods for selecting zinc finger proteins to bind to a target using phage display are described by EP.95908614.1. The target site used for design of a zinc finger protein is typically of the order of 9-19 nucleotides.

Ribozymes are RNA molecules that act as enzymes and can be engineered to cleave other RNA molecules at specific sites. The ribozyme itself is not consumed in this process, and can act catalytically to cleave multiple copies of mRNA target molecules. General rules for the design of ribozymes that cleave target RNA in trans are described in Haseloff & Gerlach, (1988) Nature 334:585-591 and Hollenbeck, (1987) Nature 328:596-603 and U.S. Pat. No. 5,496,698. Ribozymes typically include two flanking segments that show complementarity to and bind to two sites on a transcript (target subsites) of one of the genes of the invention and a catalytic region between the flanking segments. The flanking segments are typically 5-9 nucleotides long and optimally 6 to 8 nucleotides long. The catalytic region of the ribozyme is generally about 22 nucleotides in length. The mRNA target contains a consensus cleavage site between the target subsites having the general formula NUN, and preferably GUC. (Kashani-Sabet and Scanlon, (1995) Cancer Gene Therapy 2:213-223; Perriman, et al., (1992) Gene (Amst.) 113:157-163; Ruffner, et al., (1990) Biochemistry 29: 10695-10702); Birikh, et al., (1997) Eur. J. Biochem. 245:1-16; and perrealt, et al., (1991) Biochemistry 30:4020-4025). The specificity of a ribozyme can be controlled by selection of the target subsites and thus the flanking segments of the ribozyme that are complementary to such subsites. Ribozymes can be delivered either as RNA molecules, or in the form of DNA encoding the ribozyme as a component of a replicable vector, or in nonreplicable form as described below.

Endogenous expression of a target gene can also be reduced by delivering nucleic acids having sequences complementary to the regulatory region of the target gene (i.e., the target gene promoter and/or enhancers) to form triple helical structures which prevent transcription of the target gene in target cells in the body. See generally, Helene, (1991), Anticancer Drug Des., 6(6):569-584; Helene, et al., (1992), Ann. N.Y. Acad. Sci., 60:27-36; and Maher, (1992), Bioassays 14(12):807-815.

Antisense polynucleotides can cause suppression by binding to, and interfering with the translation of sense mRNA, interfering with transcription, interfering with processing or localization of RNA precursors, repressing transcription of mRNA or acting through some other mechanism (see, e.g., Sallenger et al. Nature 418, 252 (2002). The particular mechanism by which the antisense molecule reduces expression is not critical. Typically antisense polynucleotides comprise a single-stranded antisense sequence of at least 7 to 10 to typically 20 or more nucleotides that specifically hybridize to a sequence from mRNA of a gene of the invention. Some antisense polynucleotides are from about 10 to about 50 nucleotides in length or from about 14 to about 35 nucleotides in length. Some antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors.

siRNAs are relatively short, at least partly double stranded, RNA molecules that serve to inhibit expression of a complementary mRNA transcript. Although an understanding of mechanism is not required for practice of the invention, it is believed that siRNAs act by inducing degradation of a complementary mRNA transcript. Principles for design and use of siRNAs generally are described by WO 99/32619, Elbashir, EMB J. 20, 6877-6888 (2001) and Nykanen et al., Cell 107, 309-321 (2001); WO 01/29058. siRNAs are formed from two strands of at least partly complementary RNA, each strand preferably of 10-30, 15-25, or 17-23 or 19-21 nucleotides long. The strands can be perfectly complementary to each other throughout their length or can have single stranded 3′-overhangs at one or both ends of an otherwise double stranded molecule. Single stranded overhangs, if present, are usually of 1-6 bases with 1 or 2 bases being preferred. The antisense strand of an siRNA is selected to be substantially complementary (e.g., at least 80, 90, 95% and preferably 100%) complementary to a segment of a transcript from a gene of the invention. Any mismatched based preferably occur at or near the ends of the strands of the siRNA. Mismatched bases at the ends can be deoxyribonucleotides. The sense strand of an siRNA shows an analogous relationship with the complement of the segment of the gene transcript of interest. siRNAs having two strands, each having 19 bases of perfect complementarity, and having two unmatched bases at the 3′ end of the sense strand and one at the 3′ end of the antisense strand are particularly suitable.

If an siRNA is to be administered as such, as distinct from in the form of DNA encoding the siRNA, then the strands of an siRNA can contain one or more nucleotide analogs. The nucleotide analogs are located at positions at which inhibitor activity is not substantially affected, e.g., in a region at the 5′-end and/or the 3′-end, particularly single stranded overhang regions. Preferred nucleotide analogues are sugar- or backbone-modified ribonucleotides. Nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8 position, e.g. 8-bromo guanosine; deaza nucleotides, e.g. 7-deaza-adenosine; O- and N-alkylated nucleotides, e.g. N6-methyl adenosine are also suitable. In preferred sugar-modified ribonucleotides, the 2′ OH-group is replaced by a group selected from H, OR, R, halo, SH, SR, NH2, NHR, NR2 or CN, wherein R is C1-C6 alkyl, alkenyl or alkynyl and halo is F, CI, Br or I. In preferred backbone-modified ribonucleotides the phosphoester group connecting to adjacent ribonucleotides is replaced by a modified group, e.g. of phosphothioate group. A further preferred modification is to introduce a phosphate group on the 5′ hydroxide residue of an siRNA. Such a group can be introduced by treatment of an siRNA with ATP and T4 kinase. The phosphodiester linkages of natural RNA can also be modified to include at least one of a nitrogen or sulfur heteroatom. Modifications in RNA structure can be tailored to allow specific genetic inhibition while avoiding a general panic response in some organisms which is generated by dsRNA. Likewise, bases can be modified to block the activity of adenosine deaminase.

### VIII. Assays to Detect Modulation

Compounds are tested for their capacity to modulate expression or activity of one of the genes of the invention (i.e., the genes shown in Tables 1, 2 and/or 3). Expression assays are usually performed in cell culture, but can also be performed in animal models or in an in vitro transcription/translation system. The cell culture can be of primary cells, particularly those known or suspected to have a role in depression, such as cells of the CNS transfected with a gene of the invention. In the latter case, the coding portion of the gene is typically transfected with its naturally associated regulatory sequences, so as to permit expression of the gene in the transfected cell. However, the coding portion of the gene can also be operably linked to regulatory sequences from other (i.e., heterologous) genes. Optionally, the protein encoded by the gene is expressed fused to a tag or marker to facilitate its detection. The compound to be screened is introduced into the cell. The compound can be introduced directly (e.g., as an RNA or protein) or in the form of a DNA molecule that can be expressed. Expression of the gene can be detected either at the mRNA or protein level. Expression at the mRNA level can be detected by e.g., a hybridization assay, and at the protein level by e.g., an immunoassay. Detection of the protein level is facilitated by the presence of a tag. Similar screens can be performed in an animal, either natural or transgenic, or in vitro. Expression levels in the presence of a test compound are compared with those in a control assay in the absence of test compound, an increase or decrease in expression indicating that the compound modulates activity of the gene.

Assays to detect modulation of a protein encoded by a gene of the invention can also be performed. In some instances, a preliminary assay is performed to detect specific binding between a compound and a protein encoded by a gene of the invention. A binding assay can be performed between the compound and a purified protein, of if the protein is expressed extracellularly, between the compound and the protein expressed from a cell. Optionally, either the compound or protein can be immobilized before or during the assay. Such an assay reduces the pool of candidate compounds for an activity assay. The nature of the activity assay depends on the activity of the gene.

Transporters can be assayed by transfecting a cell, such as an oocyte, with DNA encoding the transporter, such that the transporter is expressed in the outer membrane of the cell. The cell is then contacted with a known substrate of the transporter, optionally labeled. Uptake of the substrate can be detected by measuring intracellular label, or ionic or pH gradients across the membrane. Compounds are screened for capacity to inhibit or stimulate transport relative to a control assay lacking the substrate being tested (see, e.g., WO0120331, US2005170394, US2005170390).

Compounds that modulate expression or activity of the genes of the invention can then be tested in animal models of depression (see Willner, Trends Pharmacol Sci. 12, 131-6 (1991)) for modulation of depression or response to treatment thereof. The animal models can be transgenic (as described below) or nontransgenic. Compounds are tested in comparison with otherwise similar control assays except for the absence of the compound being tested. An SRRI can be administered together with a compound under test to assess combinative or synergistic effects. A change in extent of depression of the animal relative to the control indicates a compound modulates depression or response to treatment thereof.

Compounds that modulate expression or activity of the genes of the invention can also be screened in similar fashion in animal models of other neuropsychiatric diseases

### IX. Transgenic Animals

The invention provides transgenic animals having a genome comprising a transgene comprising one of the genes of the invention (i.e., the genes shown in Tables 1, 2, or 3 or any of the genes containing a polymorphic site shown in Table 4, 5A-D, 6A-D, 7A-D, 8A-D, 9A-D, or 10A-D), or corresponding cDNA or mini-gene nucleic acid. The coding sequence of the gene is in operable linkage with regulatory element(s) required for its expression. Such regulatory elements can include a promoter, enhancer, one or more introns, ribosome binding site, signal sequence, polyadenylation sequence, 5′ or 3′ UTR and 5′ or 3′ flanking sequences. The regulatory sequence can be from the gene being expressed or can be heterologous. If heterologous, the regulatory sequences are usually obtained from a gene known to be expressed in the intended tissue in which the gene of the invention is to be expressed (e.g., the skin).

The invention also provides transgenic animals in which a nonhuman homolog (i.e., species variant) of one of the human genes of the invention is disrupted so as to reduce or eliminate its expression relative to a nontransgenic animal of the same species. Disruption can be achieved either by genetic modification of the nonhuman homolog or by functional disruption by introducing an inhibitor of expression of the gene into the nonhuman animal.

Some transgenic animals have a plurality of transgenes respectively comprising a plurality of genes of the invention. Some transgenic animals have a plurality of disrupted nonhuman homologs of genes of the invention. Some transgenic animals combine both the presence of transgenes expressing one or more genes of the invention and one or more disruptions of nonhuman homologs of other genes of the invention.

Transgenic animals of the invention are preferably rodents, such as mice or rats, or insects, such as Drosophila. Other transgenic animals such as primates, ovines, porcines, caprines and bovines can also be used. The transgene in such animals is integrated into the genome of the animal. The transgene can be integrated in single or multiple copies. Multiple copies are generally preferred for higher expression levels. In a typical transgenic animal all germline and somatic cells include the transgene in the genome with the possible exception of a few cells that have lost the transgene as a result of spontaneous mutation or rearrangement.

For some animals, such as mice and rabbits, fertilization is performed in vivo and fertilized ova are surgically removed. In other animals, particularly bovines, it is preferable to remove ova and fertilize the ova in vitro. See DeBoer et al., WO 91/08216. Methods for culturing fertilized oocytes to the pre-implantation stage are described by Gordon et al., Methods Enzymol. 101, 414 (1984); Hogan et al., Manipulation of the Mouse Embryo: A Laboratory Manual, C.S.H.L. N.Y. (1986) (mouse embryo); Hammer et al., Nature 315, 680 (1985) (rabbit and porcine embryos); Gandolfi et al. J. Reprod. Fert. 81, 23-28 (1987); Rexroad et al., J. Anim. Sci. 66, 947-953 (1988) (ovine embryos) and Eyestone et al. J. Reprod. Fert. 85, 715-720 (1989); Camous et al., J. Reprod. Fert. 72, 779-785 (1984); and Heyman et al. Theriogenology 27, 5968 (1987) (bovine embryos). Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al., Nature 309, 255-258 (1984). Transgenes can be introduced into such cells by electroporation or microinjection. ES cells are suitable for introducing transgenes at specific chromosomal locations via homologous recombination. Transformed ES cells are combined with blastocysts from a non-human animal. The ES cells colonize the embryo and in some embryos form or contribute to the germline of the resulting chimeric animal. See Jaenisch, Science, 240, 1468-1474 (1988) (incorporated by reference in its entirety for all purposes).

Alternatively, transgenic animals can be produced by methods involving nuclear transfer. Donor nuclei are obtained from cells cultured in vitro into which a human alpha synuclein transgene is introduced using conventional methods such as Ca-phosphate transfection, microinjection or lipofection. The cells are subsequently been selected or screened for the presence of a transgene or a specific integration of a transgene (see, e.g., WO 98/37183 and WO 98/39416). Donor nuclei are introduced into oocytes by means of fusion, induced electrically or chemically (see, e.g., WO 97/07669, WO 98/30683 and WO 98/39416), or by microinjection (see WO 99/37143). Transplanted oocytes are subsequently cultured to develop into embryos which are subsequently implanted in the oviducts of pseudopregnant female animals, resulting in birth of transgenic offspring (see, e.g., WO 97/07669, WO 98/30683 and WO 98/39416).

For production of transgenic animals containing two or more transgenes, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal. Optionally, transgenes whose length would otherwise exceed about 50 kb, are constructed as overlapping fragments. Such overlapping fragments are introduced into a fertilized oocyte or embryonic stem cell simultaneously and undergo homologous recombination in vivo. See WO 92/03917.

Nonhuman homologs of human genes of the invention can be disrupted by gene targeting. Gene targeting is a method of using homologous recombination to modify a mammalian genome, can be used to introduce changes into cultured cells. By targeting a gene of interest in embryonic stem (ES) cells, these changes can be introduced into the germline of laboratory animals. The gene targeting procedure is accomplished by introducing into tissue culture cells a DNA targeting construct that has a segment that can undergo homologous combination with a target locus and which also comprises an intended sequence modification (e.g., insertion, deletion, point mutation). The treated cells are then screened for accurate targeting to identify and isolate those which have been properly targeted. A common scheme to disrupt gene function by gene targeting in ES cells is to construct a targeting construct which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. Similar procedures can also be performed on other cell types in combination with nuclear transfer. Nuclear transfer is particularly useful for creating knockouts in species other than mice for which ES cells may not be available Polejaeva et al., Nature 407, 86-90 (2000). Breeding of nonhuman animals which are heterozygous for a null allele may be performed to produce nonhuman animals homozygous for said null allele, so-called “knockout” animals (Donehower et al. Nature 256:215 (1992)).

### X. Variant Proteins

Some of the polymorphic sites of the invention are characterized by the presence of polymorphic forms encoding different amino acids. Such polymorphisms are referred to as non-synonymous indicating that the different polymorphic forms are translated into different protein variants. The invention further provides such variant proteins or fragments thereof retaining the activity of the full length protein in isolated form.

### XI. Methods of Treatment

Compounds having activity in modulating a gene of the invention can be used in methods of treatment or prophylaxis of depression optionally in combination with other treatments, particularly an SSRI.

A compound can be administered to a patient for prophylactic and/or therapeutic treatments. A therapeutic amount is an amount sufficient to remedy a disease state or symptoms in a patient presently having symptoms of a disease, or otherwise prevent, hinder, retard, or reverse the progression of disease or any other undesirable symptoms. In prophylactic applications, a compound is administered to a patient susceptible to or otherwise at risk of a particular disease or infection but not currently having symptoms of the disease. Hence, a “prophylactically effective” amount is an amount sufficient to prevent, hinder or retard a disease state or its symptoms. In either instance, the precise amount of compound contained in the composition depends on the patient's state of health and weight.

An appropriate dosage of the pharmaceutical composition is determined, for example, using animal studies (e.g., mice, rats) to determine the maximal tolerable dose of the bioactive agent per kilogram of weight. In general, at least one of the animal species tested is mammalian. The results from the animal studies can be extrapolated to determine doses for use in other species, such as human beings for example.

The pharmaceutical compositions can be administered in a variety of different ways. Compounds can also be administered as a composition containing a pharmaceutically acceptable carrier via oral, intranasal, rectal, topical, intraperitoneal, intravenous, intramuscular, subcutaneous, subdermal, transdermal, intrathecal, and intracranial methods. The route of administration depends in part on the chemical composition of the active compound and any carriers.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions. Compositions for oral administration need not be sterile or substantially isotonic but are usually made under GMP conditions.

### EXAMPLES

The Examples describe association studies to identify polymorphic sites having alleles associated with response to treatment with an SSRI or placebo.

### 1. Samples

The DNA samples used in the study were collected from 1,024 Caucasian subjects across eight MDD Phase II, III and IV clinical trials. Among the total cohort of samples, 511 were SSRI treated and 513 were placebo treated. The study sample comprised 652 females and 372 males. These samples were equally divided into two sets by matching based on treatment group, gender, clinical study, and investigator site. The two sets of samples are designated as primary analysis and replication analysis set, respectively.

The primary scales used to diagnose MDD patients and to measure response to treatment are the HAM-D scale (Williams, Archives of General Psychiatry, American Medical Association, August 1988, Vol. 45, Num. 8, pp. 742-74) and three sub-scales that capture different aspect of depression (Table 1), as well as the Clinical Global Impression of Improvement (CGI-I) for binary definition of responder and non-responder.

TABLE 11
Individual items for each subscale considered

| | Questions |
|---|---|
| Ham-D subscale | (i.e. item #s in HAMD scale) |
| Depression (Core Lilly) | 1, 2, 3, 7, 8 |
| Anxiety | 10, 11, 12, 13, 15 |
| Insomnia | 4, 5, 6 |

### 2. Genotyping

The subjects of both sets were each genotyped for about 250,000 tag SNPs in a whole genome scan study (Hinds et al., Science, Vol. 307, 1072-1079 (2005)). These SNPs were selected based on the linkage disequilibrium structure of the human genome. The resulting genotype data quality checking was performed using the standard quality control (QC) procedures (Maraganore, Am. J. Hum. Genet. 77:000-000 (2005).

### 3. Analysis

A variety of statistical models were employed to investigate associations between SNPs and both binary and quantitative response variables involved in MDD phenotypes, anti-depressant SSRI and placebo response, as well as time-to-response. In the primary analysis of the whole genome scan, linear regression and logistic regression were used. In the replication analysis, analysis of covariance (ANCOVA) model, as well as Fisher test and Bonferroni correction and False Discovery Rate (FDR) were also calculated. In these analyses, statistical significance was assessed using the q-value approach (Storey et la., (2003) Proc. Natl. Acad. Sci. USA 100 (16): 9440-9445.6), a method based on an assessment of the overall false discovery rate of the experiment.

In addition, FDR analysis was performed on 11 CNS genes that were chosen a priori based on literature reports.

The statistical procedure, model and covariates, and response variables for the depression and subscale association analysis are briefly summarized in Table 12.

TABLE 12
Analysis procedure to find marks associated with MDD or a subscale phenotype

| Population | Model | Response | Covariate | Test |
|---|---|---|---|---|
| Male, female, | ANCOVA | Quantitative | Age, | Compare full (with |
| SSRI treated, | | measurement of | gender, | genotypes and |
| placebo treated groups | | Total HAM-D | study | interaction terms |
| combined | | or subscale | | included) and reduced |
| | | scores at | | (without genotypes) |
| | | baseline | | model to determine |
| | | | | SNPs involved in |
| | | | | MDD phenotype |
| | | | | Follow-up significant |
| | | | | SNPs to determine |
| | | | | interaction with gender |

The statistical procedure, model and covariates, and response variables for SSRI and placebo treatment analysis are summarized in Table 13.

TABLE 13
Analysis procedure to find markers that are associated with SSRI treatment
response or placebo effect

| Population | Model | Response | Covariate | Test |
|---|---|---|---|---|
| Male, female, | ANCOVA | HAM-D total and | Baseline | Type III test of |
| SSRI treated, | | Subscale score | total, | genotype by |
| placebo treated | | delta changes | Study, | treatment interaction |
| groups combined | | from baseline | Age, | Contrast tests to |
| | | | Gender, | estimate the separate |
| | | | Treatment | marker effects for |
| | | Binary response: | Study, | SSRI and placebo. |
| | | Responder or | Treatment | |
| | | non-responder | | |
| | | (based on CGI-I 1 or 2) | | |

### Results

In the whole genome association analysis, SNPs with significant associations in both primary and replication data sets were observed. These SNPs were annotated as CNS-relevant or novel (i.e., not previously known to be expressed in the CNS) based on review of literature and various databases. Table 14 lists the numbers of SNPs that were assessed as potentially associated.

TABLE 14
Significant SNPs associated with MDD and/or SSRI/placebo responses

| | Placebo Response | | SSRI Response | |
|---|---|---|---|---|
| Response variable | CNS relevant | Novel | CNS relevant | Novel |
| Binary | 3 | 168 | 10 | 168 |
| Quantitative | 6 | 146 | 10 | 142 |
| Time to response | 1 | 153 | 8 | 129 |

The data from the analysis are summarized in the Tables that follow. In the whole genome association analysis, SNPs with significant associations in both primary and replication data sets were observed. These SNPs were annotated as CNS-relevant or novel based on review of literature and various databases.

In addition to the whole genome analysis, a priori hypothesis testing was performed on both the primary and replication sets of samples on a list of 11 CNS genes (BDNF, COMT, DRD2, DRD3, DRD4, HTR1A, HTR2A, SLC6A2, SLC6A3, SLC6A4, TPH2) that were reported in the medical literature. The significance is based on the multiple comparisons to only the SNPs within the 11 CNS genes tested. Among the SNPs located in these 11 genes, the most significant result was for the Total HAMD end-point for SSRI treatment response for SNP 1752273 located within the gene TTC12 on chromosome 11, within 50 kb of the well-known dopamine receptor D2 (DRD2). This SNP is a part of a 3 SNP haplo-block that are in high LD This SNP which is located in gene TTC12 adjacent to DRD2, and two other adjacent SNPs sharing the LD bins are listed in the table “CNS_relevant_CNS.txt”.

The SNPs meeting the significance level and FDR level are listed in the tables described below.

### A. CNS a Priori and CNS-Relevant Category

For the a priori hypothesis testing and the CNS-relevant genes categories of SNPs, the results are provided in Tables 2-4. In these tables, the SNP association results are organized by the primary objective categories and types of analysis into separate work sheets. The first table (CNS_relevant_CNS.txt) contains results from a priori hypothesis testing on the 11 CNS candidate genes, and the other six tables contain results from the whole genome association study for only those SNPs that could be annotated to CNS-relevant genes based on public literature and databases.

### B. Novel SNP Category

For the novel categories of SNPs, there are 6 tables, Tables 5-10. Table 5 relates to linear genotype (i.e., SNPs associated with placebo effect by a linear measurement). Table 6 relates to binary genotype (i.e., SNPs associated with placebo effect by a binary measurement). Table 7 relates to binary interactions (i.e., SNPs associated with SSRI effect by a binary measurement); Table 8 relates to linear interactions (i.e., SNPs associated with SSRI effect by a linear measurement). Table 9 relates to time genotype (i.e., SNPs associated with placebo effect by a time measurement). Table 10 relates to time interaction (i.e., SNPs associated with SSRI effect by a time measurement). Each of the tables is divided into four subparts (A, B, C, D) corresponding to the four scales of the HAM-D phenotypes HMDT: Total HAM-D, CLILLY: Core depression, ANX: Anxiety, and INSOM: Insomnia. Tables 5-10 contain the associated SNPs that were not annotated to CNS-relevant genes as described above. Fisher Pval is Fisher's method for combining the main p-values from the two sets and FisherQval is the estimated False

Various embodiments and modifications can be made to the invention disclosed in this application without departing from the scope and spirit of the invention. Unless otherwise apparent from the context any embodiment, feature or element of the invention can be used in combination with any other. All references including patents, patent publications, applications, SNP or sequence identifiers or the like and journal articles cited herein are incorporated by reference in their entireties for all purposes to the same extent as if each were so individually denoted.

TABLE 1

| | | chro- | | | | | | | |
|---|---|---|---|---|---|---|---|---|---|
| | | mo- | | | ref_{—} | alt_{—} | | ref_{—} | |
| snp_id | dbsnp_id | some | accession | position | base | base | flank | freq | genes_near |
| 10940 | rs2823731 | 21 | NC_000021.4 | 16566315 | A | G | ATACATTATATTTAAACATATCTCTATAGAGTCAACAAAATAAAATAAACA | 0.5986 | VDAC2P-[LOC388815]-LOC391270 |
| 12394 | rs2823937 | 21 | NC_000021.4 | 16947265 | A | C | ATGTAAGATCGTTTGGGAAAATGTTAAGACAGATATCTTGCTTTAATTTTT | 0.6183 | LOC388815 [ ]-LOC391270 |
| 17207 | rs2824839 | 21 | NC_000021.4 | 18746350 | C | A | GCTTATGTTTATGTGATGGCACCTGCGAGTACATAGAGGTTGGATATGTTA | 0.5494 | PRSS7 [ ]-LOC388816 |
| 17255 | rs2824848 | 21 | NC_000021.4 | 18775552 | T | C | ACTCCAACCCACAGCATTATTATTATTCAGTAGGTTATAGAGGTGTTATAC | 0.5109 | PRSS7-[ ]-LOC388816 |
| 20802 | rs2825756 | 21 | NC_000021.4 | 19999393 | A | G | ACCCCTTAAATTTTCATTTTCTCTCAAAGTCTCCTCTAAATTTAGTATATT | 0.8111 | SLC6A6P-[ ]-C1QBPP |
| 39641 | rs928261 | 21 | NC_000021.4 | 25378214 | T | C | TGATTAGCCTTCCATTTCATAAACCTTTTTTTCCCCTGGAATTGATAATGG | 0.3077 | LOC400860-[ ]-LOC284821 |
| 40257 | rs2829674 | 21 | NC_000021.4 | 25557670 | C | T | ATAAAGTTGGAATTTGGAGTCATGGCCTGAAAAATGTGAGCAAGTAAAGAA | 0.5755 | LOC400860-[ ]-LOC284821 |
| 54303 | rs2832046 | 21 | NC_000021.4 | 29038677 | T | C | TGGAAGGGTAGAACCTTAAGTAGTTTTTCATTCTCTGACTACTCAACTAGA | 0.8541 | C21orf100-[ ]-C21orf127 |
| 75560 | rs2226829 | 21 | NC_000021.4 | 36987160 | T | G | GATTTTGAGGCCATGTTTCCGTTAATCTGGACCGAGAGCCCTCTGGGAGAG | 0.311 | LOC388823-[ ] SIM2 |
| 76470 | rs2835628 | 21 | NC_000021.4 | 37439923 | A | G | AATGCGATTTGATGATTGTAACAGGACAAAATTTTGATTCTTTCGAAATTC | 0.2715 | DSCR5-[TTC3]-DSCR9 |
| 82434 | rs2836671 | 21 | NC_000021.4 | 39052714 | G | A | AGCTAGGTGGTGTTCTCGTGTACATGTTAGAGATGAGGAAACCCAATCTCT | 0.6101 | ERG-[LOC400866] ETS2 |
| 104324 | rs1296754 | 22 | NC_000022.5 | 16360114 | G | A | TCCTTGTTTCCCCCAGCCTTTTGTCGCTTAACATGTTTCTTTATGCTTATT | 0.3001 | CLCP1-[CECR2]-SLC25A18 |
| 120178 | rs465736 | 22 | NC_000022.5 | 28159320 | A | G | GTTCTAGAAGTGACAAAGCTGGGACACAATACCTTTATGCATGAAAAGGTT | 0.8434 | AP1B1-[RFPL1] NEFH |
| 120634 | rs740041 | 22 | NC_000022.5 | 28524653 | A | G | CATCTCTCTTATCATGCTGCCTCCCAACATGCAGGGGAGAGTCCTGGCCTT | 0.601 | HSPC051, LOC55954 [ASC1p100]- |
| | | | | | | | | | MTMR3 |
| 120658 | rs2074707 | 22 | NC_000022.5 | 28534910 | G | A | CTGAATAAATGGCTCAATGAATAACGCACAAGTGAACATGTCAAACTGAAA | 0.6 | HSPC051 [ASC1p100]-MTMR3 |
| 120666 | rs17711377 | 22 | NC_000022.5 | 28537540 | C | T | TTTGGGCAGGTCTGTCCTTGGTTTCCTTATCGATGACCATGCAGCCCTTGC | 0.5875 | HSPC051 [ASC1p100]-MTMR3 |
| 120843 | rs2285667 | 22 | NC_000022.5 | 28708658 | T | A | AGCAAGAAAAGATTACTGTTCTGGCTCCCTTCAGCTTCTATGTCATTGCAT | 0.6244 | ASC1p100-[MTMR3] LOC400924 |
| 120880 | rs41157 | 22 | NC_000022.5 | 28729705 | T | C | CTTGGCCTTGGCTTTCATTTTGCATTGCTCTTAAATAATAAGTTTGCTTCT | 0.3814 | ASC1p100-[MTMR3] LOC391326, |
| | | | | | | | | | LOC400924 |
| 120906 | rs41168 | 22 | NC_000022.5 | 28742715 | A | C | CTCAGCCCCTGCTCTGAGTGCCATCAATTTAACTGTTTTGTGGTTCTTCTC | 0.3829 | ASC1p100-[MTMR3] LOC391326, |
| | | | | | | | | | LOC400924 |
| 121018 | rs1548389 | 22 | NC_000022.5 | 28898106 | T | C | AAAGGTATTGGACTTATATCCTTGATAGAATTGTAGACTGAGTCACTATAA | 0.5895 | MGC26710 [ ]-LIF |
| 133091 | rs34770535 | 22 | NC_000022.5 | 36928983 | T | A | TCCATCCGCTTCCCAGGCAGACCTATCAGCCAGACAGCTTCCGTCTTGCCT | 0.9712 | C22orf5 [ ] CSNK1E, LOC400927 |
| 137315 | rs926350 | 22 | NC_000022.5 | 41702889 | C | T | TAGAGGCAGCCATCAAATCACCACCCGGGAATGTTCAACTGCAAGTGTGCC | 0.7844 | PACSIN2-[TTLL1] BIK |
| 137829 | rs5996341 | 22 | NC_000022.5 | 42090418 | C | T | GGAGATTTCCTTGACTTCGTCTTCCCTCTTTTGGTCAAATTAAAAAATATC | 0.5363 | SCUBE1-[C22orf1]-FLJ23588 |
| 138564 | rs16991431 | 22 | NC_000022.5 | 42742499 | C | T | ACCCAGGAGGGCTTCTTGGAGGAGGCGGCCAGTAAGATGAGGTTGAAGATA | 0.7787 | CGI-51-[PARVB]-TRSPP1 |
| 145360 | rs6007770 | 22 | NC_000022.5 | 46571750 | A | G | CACCCCACACTGGACACATCCTTATAGGCACTGAGACACTTCTGGGAGCAC | 0.8837 | LOC400932-[ ]-LOC388914 |
| 159809 | rs4127784 | 14 | NC_000014.4 | 26997334 | C | T | CATCTTACAGAGTGAAGTGCCTGATCCTAAGATATGGTGGTCAAAGAGGAT | 0.5709 | RPL26P3 [ ]-BTF3P2 |
| 159815 | rs12882372 | 14 | NC_000014.4 | 27002793 | G | A | GTGCCTGGCCTACGATTTTAATTACGGTAGATTTATATTACACTTAAACCT | 0.6128 | RPL26P3-[ ]-BTF3P2 |
| 160343 | rs17114346 | 14 | NC_000014.4 | 27343901 | G | C | GTATTTTCTTTTAACTTTCAAAACTGTTTTTGCTCCAAAGAACAAAAGCAA | 0.9947 | LOC387978-[ ]-PRKCM |
| 162110 | rs2819780 | 14 | NC_000014.4 | 29261783 | T | C | GCTGGAGAGGCAAAGTTGGGACAAGCATATTTCTATATTTCTTAGTATAAG | 0.973 | RPL12P5 [ ] RPL27P1 |
| 163068 | rs225940 | 14 | NC_000014.4 | 28516976 | G | A | TCCTAATCAGTGGGACTTTAAGAGTGCTTTAAGGGCAATATTCCTATTATG | 0.4135 | PRKCM-[ ]-CBPINP |
| 169827 | rs10129645 | 14 | NC_000014.4 | 35848871 | A | C | AGACTAGAGAGACTAAAACAGTTGAAGTGAAGAACCTGAAAGGAGAGCTTA | 0.7576 | SLC25A21-[MIPOL1]-FOXA1 |
| 178191 | rs10483614 | 14 | NC_000014.4 | 51141466 | T | C | CAAATGTTTAAAGCTAATGTCTTAATGGTATGTACTAAATCAAGCTACTAA | 0.8227 | DKFZp762F0713 [ERO1L] STYX, |
| | | | | | | | | | PSMC6 |
| 195161 | rs10143275 | 14 | NC_000014.4 | 67131529 | A | G | AATGGGGCATATGTTCCATAAGTAGACCAGGGGTATCACCAATGAGCGTCT | 0.2865 | RAD51L1-[ ] RPL12P7 |
| 208445 | rs880373 | 14 | NC_000014.4 | 76732331 | G | A | GTCTACCTCTTACCTTTGCCATTTCGTTCTGTCAATCAGAATTGAGTCCAG | 0.8337 | FRDAP-[ ]-NRXN3 |
| 214385 | rs1955427 | 14 | NC_000014.4 | 83914088 | G | A | CCTACCTTCTTCGCTCCCTAGCTGCGTTTATTTAACTTAGTTCAGAGGCTT | 0.7949 | RNU3P3-[ ]-LOC283583 |
| 222093 | rs732171 | 14 | NC_000014.4 | 88905407 | T | C | CACACACAAAGATGAAATGAACTCCTTTGTCGTGTAAGAATCTCTCCAACG | 0.8442 | CALM1 [ ] LOC400238 |
| 238656 | rs11625351 | 14 | NC_000014.4 | 101746762 | A | G | GTGGACCAAACAGAATCCAGGCTCCAAGCCTCTCCCTGGCAAGCCTCTTGC | 0.4456 | RPL21P13, RPL17P4 [ ] LOC388019, |
| | | | | | | | | | EIF5 |
| 256781 | rs9368147 | 6 | NC_000006.6 | 10345221 | T | C | CAGCCTTTGTCTCCTGCACAGGTACTGTTCGCTACTCTTAAGGATGCAAAA | 0.8468 | OFCC1-[ ]-TFAP2A |
| 269181 | rs17320151 | 6 | NC_000006.6 | 18844754 | A | G | ATCAGAACTTACAGAACACTTTTAGAGTAGATATCAGAGAAAAATTCATAG | 0.4911 | IBRDC2-[ ]-ID4 |
| 272259 | rs851601 | 6 | NC_000006.6 | 66889451 | G | A | CTTTTCAAGTGGACTTGTAAAAATCGAAGAGAAAAATGCAAAGTTTCCCAA | 0.2565 | NUFIP1P-[ ]-BAI3 |
| 279774 | rs6920211 | 6 | NC_000006.6 | 135411888 | T | C | GGTTCCATTGAGACTGATGCTGCATTGAATTCTGATGATACAGTTCGCTGC | 0.7901 | HBS1L-[ ]-MYB |
| 280097 | rs728030 | 6 | NC_000006.6 | 135226864 | C | A | GATTTGTATACTATTGAGGTATTAACGATCCATATTTAACCAAGTGTTTTC | 0.3688 | LOC154094 [ALDH8A1] HBS1L |
| 280907 | rs567049 | 6 | NC_000006.6 | 149602520 | A | G | GGGGGTGAAAGGAGTCCATACCATTAGTTCTTAGCTTCAAGACCTAAATCA | 0.615 | UST-[ ] MAP3K7IP2 |
| 283793 | rs1099652 | 6 | NC_000006.6 | 167139812 | G | A | GGTCACCTCAAACCATGATTCAAATAAGGCAGATTTTGAGTTTGTGCATAA | 0.8295 | RPS6KA2-[ ]-RNASET2 |
| 289615 | rs9260980 | 6 | NC_000006.6 | 30067845 | A | G | CAGGGTAAATTCCTGTAAGGCCTGGATGCCCTGCTGTGAGGTCAAAGGGGG | 0.867 | HLA-A, MICD, HCG9, HLA- |
| | | | | | | | | | 80, HCP5P3, HCG2P6, HCG4P4 [ ] |
| | | | | | | | | | HLA-J, C6orf12, ETF1P1, HCP5P2, |
| | | | | | | | | | 3.8-1.2 |
| 295720 | rs9364688 | 6 | NC_000006.6 | 163810679 | A | C | AGTGCGTGGTTAGCTGTGTTAGACCAGTACGCTGAATCGCTTTGTGGAACT | 0.4757 | LOC401283-[QKI]-C6orf118 |
| 302732 | rs581785 | 6 | NC_000006.6 | 54001550 | T | C | CTTAAGTCTCTGAATTGAGCTTTCTTATAGCAGTAGGGTCCTCATCGTCTG | 0.0946 | LRRC1-[C6orf142]-TINAG |
| 303552 | rs12055820 | 6 | NC_000006.6 | 24077292 | T | G | ATAAGAAAAAGCATGCAGAGATACATGCAGACTTGAGAAATCGATGGCATT | 0.751 | LOC401238-[ ]-VMP |
| 313699 | rs9373149 | 6 | NC_000006.6 | 136025972 | C | A | ACCTCATATACCTTGTATCCAGAAGCAACCAGGTCATCTCTTAGCATTTTC | 0.6896 | AH1-[ ]-PDE7B |
| 313717 | rs1475069 | 6 | NC_000006.6 | 136036804 | A | C | GCAATCAATGGAATTTGCAAGTGCGAGATGAATTTTCCTTAATTAAAATCT | 0.6985 | AH1-[ ]-PDE7B |
| 320863 | rs1572674 | 6 | NC_000006.6 | 140244804 | C | A | GGCTCACTTTAGGGTCCAGGAGAAACAATATGGAAACCAGTAAAGCTTTTT | 0.88 | LOC340148-[ ] LOC401276 |
| 331584 | rs6458829 | 6 | NC_000006.6 | 52238093 | T | C | GCCTAGCCTGATCTTCAACCTTGTCTGTTCTCCAGCTATAGATGAGAGTCT | 0.9502 | MCM3 C6orf33 |
| 334643 | rs9486657 | 6 | NC_000006.6 | 107974946 | C | T | GGACAAACATCTTACATCTTATACACCATGGTTTTGTTCAGAACTGGGCTT | 0.7184 | C6orf210-[ ] FLJ10159 |
| 340241 | rs4945651 | 6 | NC_000006.6 | 120164665 | A | G | CTTCTTATCTTACTGGGGTAATCCAATTATGACTTGTACCTGCTCTGGCTT | 0.7778 | MAN1A1-[ ]-C6orf170 |
| 342860 | rs199638 | 6 | NC_000006.6 | 72232763 | G | C | AGGGGGAGACCTAATTATCTGGGCTGTCCTTCAAGATCCTTCTCTTTCTTT | 0.2344 | C6orf155-[ ]-RIMS1 |
| 343816 | rs9360051 | 6 | NC_000006.6 | 94782633 | A | G | AATAAGAACATTTTGAAAAATAGGTAAGAATTTAAGCCCATCAACATAAAA | 0.9105 | EPHA7-[ ]-HCP17 |
| 348386 | rs17717044 | 6 | NC_000006.6 | 82737724 | G | C | TCTAGGGAATGTGATTTAGGATTAAGTTCTCAAGTACTTTTTTTTGCAATA | 0.9595 | LOC389413-[ ]-IBTK |
| 350246 | rs9320552 | 6 | NC_000006.6 | 116355906 | C | T | GGCCAAATTGATAATATATACAATCCAGTAGCTTTGTTAGTTATTCAAGCA | 0.6471 | HS3ST5-[FRK]-NT5C2L1 |
| 355828 | rs9375582 | 6 | NC_000006.6 | 128938976 | T | G | TTTGTAACTTCCTTGAAGGCAGAGTTTCTTCTTCGGGTTTGTATTATCTAT | 0.8004 | PTPRK-[ ]-LAMA2 |
| 358303 | rs2806719 | 6 | NC_000006.6 | 116865194 | T | C | ATTTTCCCCTAATTATCACAAATAATTTAAAATTACATGGATGCCTCATTA | 0.0413 | SART2-[ ] C6orf188, C6orf78 |
| 358756 | rs615199 | 6 | NC_000006.6 | 117159223 | T | C | TTAATATGACAATAATCTCCACAGCTGGTACATATTTGCCAAATGTGGTAG | 0.684 | KPNA5-[GPRC6A]-RFXDC1 |
| 363483 | rs10949074 | 6 | NC_000000.6 | 57381100 | C | T | TGAAGAAGCAGGTAGAACACTGTGACCTTACGATGTGAATTCTCTAATCAG | 0.7415 | RAB23-[PRIM2A]-LOC389401 |
| 365290 | rs3799070 | 6 | NC_000006.6 | 69959617 | A | C | ACACCCTTTAACTTTATTTACAGTAAATGAGAGCAAGTTTTAAAAGCCTTT | 0.786 | NUFIP1P-[BAI3]-C6orf209 |
| 368144 | rs4840208 | 6 | NC_000006.6 | 102686804 | C | T | TTATTGTAGTACATTGAAAGAAACACGAATAAACAGGAATATACCATTCAA | 0.4766 | GRIK2-[ ]-LOC389419 |
| 383165 | rs1883324 | 6 | NC_000006.6 | 11914419 | G | T | TGTGAGCCCCTTTTGTCATTGTCAGGGCCTAAGGGTCCAGGAATCACTGTC | 0.6534 | C6orf105-[ ] LOC389369 |
| 383212 | rs1569731 | 6 | NC_000006.6 | 11933400 | A | G | GAGGGGAAATTCCAAGCTGCAGAGTACGTGAAAACAGTTTTTTAAAGTTGA | 0.7302 | C6orf105 [LOC389369]-HIVEP1 |
| 383214 | rs2143715 | 6 | NC_000006.6 | 11934194 | T | C | CATTCCATGTTCGTCCTGAGGTAAATTAGCCTAAGTGATTATTGTATCAAA | 0.7308 | C6orf105 [LOC389369]-HIVEP1 |
| 389068 | rs1571913 | 20 | NC_000020.6 | 46813492 | T | C | TGTATTGGGAGTGCCCTGTAGAGGATGAAAGGGAGAGAAGCAAGGAGAGTA | 0.0054 | RPL35AP [ ] NCOA3 |
| 396080 | rs17123518 | 20 | NC_000020.6 | 31993265 | C | T | GGGAGCTCACACCTTCGAGAGGCCACGCTGCTGCCGAAAAGAGGGGCTGCT | 0.9593 | FLJ33706 [ ] COMMD7 |
| 406464 | rs4396776 | 20 | NC_000020.6 | 17048777 | C | A | TGAAATTGTGGGTATCACATGGGATCCTTAGGAGCAGAGTATAAGCACAGA | 0.5296 | OTOR-[ ]-PCSK2 |
| 407964 | rs16823 | 20 | NC_000020.6 | 17786064 | G | A | GTCGCCCACGGTGGTCACATGATCAGTCCTGGTCTTTATACCATTACTTCC | 0.8016 | C20orf179-[ ]-SNX5 |
| 409576 | rs6035200 | 20 | NC_000020.6 | 19017894 | A | T | CCTTTTGGAAAAAAAATCATGTGTCATCTCCATTATACTGAACTCTCTGCA | 0.6532 | C20orf79-[ ]-SLC24A3 |
| 411305 | rs6081854 | 20 | NC_000020.6 | 20037883 | G | A | ACAAGACACATGAGGGAAAAAAACCGTCTTGGAGGTTCTTGCCCCAACAGA | 0.4435 | NAT5, CRNKL1 [C20orf26]-RPL17P1 |
| 414780 | rs2301020 | 20 | NC_000020.6 | 49389941 | A | G | GACATACACTTTCTTCCAATTCTGGATATATTTGTGTGCACACAGGCTACT | 0.5338 | LOC149738 [UBE2V1, Kua-UEV] Kua |
| 428043 | rs2425501 | 20 | NC_000020.6 | 41963754 | C | A | GGAGTATGGGAAGGTCCTGAAAGAGCCCCTAAGTTATGCTTGATTTACTGC | 0.5802 | LOC391249-[PTPRT]-PPIAL |
| 437310 | rs6040221 | 20 | NC_000020.6 | 10861996 | G | A | CAGAAAATCATCTTTTCAGGCTTAAGGTGATCCTCAAAATCAGAAAAAAAC | 0.4245 | JAG1-[ ] FAT1P1 |
| 441242 | rs6109927 | 20 | NC_000020.6 | 13508267 | A | G | TTTTACGGAAGTATTTCTTTTGCTCAGGACAGTTCTAAGTGCAGTGGCAGA | 0.6658 | MRPS36P6-[C20orf13]-C20orf6 |
| 446808 | rs2870357 | 20 | NC_000020.6 | 54006326 | G | A | TTCAGTCTCCACCTTTTAGAATTCAGACCATTATCTGCGGTTTTGGATAAT | 0.5479 | DOK5-[ ]-RPL12P4 |
| 456564 | rs728265 | 20 | NC_000020.6 | 881379 | G | A | TCCAACCAACTCATTCAGATGTGCCGAGACCTGGGCATGGTGCTGGGGATA | 0.8092 | C20orf55-[ANGPT4]-LOC343637 |
| 470618 | rs17155560 | 7 | NC_000007.8 | 81078793 | G | A | GGAAGAAGCCAAAATAAACAGAGATGAACAGACATAAAAAGAAATTATAGC | 0.6565 | HGF-[ ]-CACNA2D1 |
| 472520 | rs6943619 | 7 | NC_000007.8 | 105765830 | A | G | TATATGGTGGTTCTTCTTCATTTCAATCTCAGAGGATAACTTTCTAAATTC | 0.3746 | PBEF1-[ ]-FLJ36031 |
| 481459 | rs7801675 | 7 | NC_000007.8 | 18882744 | G | C | CTCAGATAAATTCATTTAAGTGGCTGAGGAACACAGCTAATAATTGGCAGA | 0.8295 | HDAC9-[ ] TWIST1, FERD3L |
| 486447 | rs13222512 | 7 | NC_000007.8 | 25263122 | T | A | ATTATTTTATTTCATGCAGTTACATTTATTTATTCAGCAAATATTTATTGA | 0.7984 | C7orf9-[ ]-UBA52P1 |
| 492423 | rs9718390 | 7 | NC_000007.8 | 29071360 | G | C | AAAGGCCAGCAGGACTACGGGTTAGGCATGAGTAAGCCGACGGGAGAGGGA | 0.6501 | LOC401318-[CHN2]-LOC222171 |
| 497179 | rs2214882 | 7 | NC_000007.8 | 40767209 | A | G | GCCATTATGCAATTCTCCCTAACTGAACTACCTTTCACCTTCATCTCTGCT | 0.2157 | C7orf10-[ ]-INHBA |
| 499954 | rs17134115 | 7 | NC_000007.8 | 50783028 | C | T | GTGCACAACTGTCAAGTTTATGAGACGACTTGAGGTTTGCTGTTGTCTCAA | 0.9462 | GRB10-[ ] COBL |
| 502021 | rs17169536 | 7 | NC_000007.8 | 16318284 | G | A | ACTGGCCTCACCTTATAATGTAACAGCCCATTATTGATTTAGGACAGGTTT | 0.6804 | SOSTDC1-[ ] LOC317727 |
| 502183 | rs2301971 | 7 | NC_000007.8 | 16255401 | A | G | ATAGCACCTGTTAAATGGAAAAGTGATTAGCTATTATAAGTTTAGAAAAAA | 0.7038 | SOSTDC1-[ ] LOC317727 |
| 507206 | rs11344954 | 7 | NC_000007.8 | 81180842 | G | A | GTGGGAGTTGTGTTCAGCCGCAAAAGAAAATGAATTAGTGTGTTATATTTG | 0.5191 | HGF-[ ] CACNA2D1 |
| 507284 | rs37089 | 7 | NC_000007.8 | 81222775 | G | A | AGCAAGAGAAGCTTCTCAAGGATACGGGCATTCAAAAAGCCAAGAATAGGC | 0.7225 | HGF-[CACNA2D1]-PCLO |
| 509722 | rs36068462 | 7 | NC_000007.8 | 87862275 | A | G | GGTCAGATAATATCATCCAAACCACATGAATGGAACACTGAGATACTGCTA | 0.3996 | LOC392069-[ ] LOC219558 |
| 516338 | rs4729988 | 7 | NC_000007.8 | 103468701 | C | A | TACTGATAGTTACAGCTACTATATACAATTCAATTGACTTCCTCAGGATTA | 0.7117 | ORC5L-[ ]-LHFPL3 |
| 519293 | rs4730253 | 7 | NC_000007.8 | 106921967 | A | G | TAACGAGAAGCCTTATATACTACATAAAGGAGCCTAGGTTTTATCCTCCAG | 0.2889 | SLC26A4 [ ] SLC26A3, CBLL1 |
| 522335 | rs9649349 | 7 | NC_000007.8 | 111226234 | A | C | ACATAAAACCCTTCAATGGATCCTCAAGACCAATGAAATAAAGTGCTAACC | 0.5962 | LOC402295-[DOCK4]-ZNF277 |
| 523650 | rs39311 | 7 | NC_000007.8 | 116508620 | T | G | CCCAGGGACCTTTCAATTTTATGCTTATCTTTCTTTATATATTAATATCAA | 0.7317 | LOC402296 [WNT2] GASZ |
| 526026 | rs34624434 | 7 | NC_000007.8 | 119115798 | C | A | CCTGAAATCAATCTTAAATTTTCAACTTGTTTTCCTCTACACTGTCACTAC | 0.8411 | ANKRD7-[ ]-KCND2 |
| 528496 | rs2237794 | 7 | NC_000007.8 | 126346076 | G | C | TCAAATTAAGGGGATCATCAACAACGTTTTCTACAGTTCACATAGGAGGCG | 0.7806 | LOC401398-[GRM8]-LOC346646 |
| 532395 | rs2301896 | 7 | NC_000007.8 | 143076729 | C | T | TTTCTGAGAAGGCCTTAAAGAAAATCATGGTATACTTAGAATTATTGGACA | 0.6507 | OR2F1, LOC346524 [ ] LOC135944, |
| | | | | | | | | | LOC135946 |
| 539841 | rs2267706 | 7 | NC_000007.8 | 139103545 | G | A | TGCGATACCATCTCCCCACTTCTACGCCAAGCTGCTCACGGTCTCACCCAG | 0.7513 | LOC389562-[TBXAS1] ZC3HDC1 |
| 542193 | rs3901848 | 7 | NC_000007.8 | 30548185 | G | A | CAAGGCCGTCTAAATTGTCCTTCCAGTGTAAAGTCCATTGAATTTTTCTCC | 0.4635 | INMT, LOC402254 [ ] FLJ22374 |
| 552812 | rs17286418 | 7 | NC_000007.8 | 15881856 | C | G | TAAATTACTAATATGTAGCACCTTGCAAGTGTATAAGTGGTCAACTTTATA | 0.632 | MEOX2-[LOC402250]-SOSTDC1 |
| 552869 | rs35150436 | 7 | NC_000007.8 | 15931275 | C | T | ATGCAAAATCCCTGGCTTCAAGGAACGCATTTCCCTGATGCAGGCCACATG | 0.6557 | MEOX2-[LOC402250]-SOSTDC1 |
| 552991 | rs17153388 | 7 | NC_000007.8 | 105909694 | C | A | CGAACCAGTTGATTTTGTTTGCATGCATGAATTTACATGTCCTTTCAACAG | 0.6769 | FLJ36031 [ ]-LOC340340 |
| 555251 | rs1003404 | 7 | NC_000007.8 | 68467077 | T | C | TGTCTTTATCTGCACTATAAAATACTGCAGCCTAGCTGGATGAGACGGTTA | 0.154 | FLJ13195-[ ] AUTS2 |
| 555297 | rs10487947 | 7 | NC_000007.8 | 68416498 | G | A | GTGCCCAGCCCCTGGTGATTTTATGGAGAACTTACTCTGTGCCCTTGGATA | 0.8703 | FLJ13195-[ ]-AUTS2 |
| 555302 | rs17140651 | 7 | NC_000007.8 | 68407681 | G | A | TACAAGTATGATAGCATCAAACACAGGGCTTAGTTTGCATGCCCTCTTATA | 0.8691 | FLJ13195-[ ]-AUTS2 |
| 555380 | rs6967772 | 7 | NC_000007.8 | 122605855 | G | T | TTTCTGGTTTCAATCCAGTGAAAGTGCTCTTCAATTCTCTACCTTTTGCCA | 0.8383 | SLC13A1-[ ] FLJ35834 |
| 561917 | rs2041357 | 7 | NC_000007.8 | 22840820 | A | G | TTGAGCAATTTCAGGACAGCAGTTTATCTTTTGGTCTGAGTTGTTTGGAGT | 0.7383 | DRCTNNB1A [ ] SBB126 |
| 583132 | rs17167392 | 7 | NC_000007.8 | 133121089 | G | C | ATAAGAATTTCCCTATCAGCTACAGGGTCTCTTGCAGGGAAATTGTTTTAA | 0.8063 | FAM10A7-[SEC8L1]-FLJ32786 |
| 583166 | rs12669887 | 7 | NC_000007.8 | 133145749 | T | C | GTCAACATTTTTGTCTCCCCAAGCATTTTCTGATTTGAACTATTTTCTCAC | 0.7991 | FAM10A7-[SEC8L1]-FLJ32786 |
| 594176 | rs12154667 | 7 | NC_000007.8 | 92790831 | C | T | TTAAAATACTCAGCTTAGGAAGTTGCTGTTAAATAACATAAAGACAATTAC | 0.5109 | CALCR-[ ]-LOC346588 |
| 594210 | rs13237782 | 7 | NC_000007.8 | 92799057 | T | C | GTATCATCTTGTGTTTCTCCTAGTGTATCAATATTTGTTTCTGAGTATGAT | 0.6046 | CALCR-[ ]-LOC346588 |
| 603200 | rs2033047 | 7 | NC_000007.8 | 37270115 | T | G | AAATAGCCTTGATAAAATGAAAAGATCCCTAATTGTGGCAGTTGGAAGTTT | 0.4847 | ELMO1 [ ]-LOC340285 |
| 607843 | rs713504 | 11 | NC_000011.5 | 16964299 | G | C | AGGGAAAGAGGTGTAGCAGGCCTGAGCAAACCCAACCATGATGGGCTTTAG | 0.8566 | LOC390096 [ ] LOC399872 |
| 612631 | rs1107162 | 11 | NC_000011.5 | 112826688 | A | G | TGGGTGTCTGAGGCCCTTGCCCCTCGCTTATCTTCTCCCAGATACATAAGA | 0.598 | TTC12, ANKK1 [DRD2]-TMPRSS5 |
| 617225 | rs560097 | 11 | NC_000011.5 | 64817890 | A | C | GTTATTATATCAGGGTAGTAGAATAAGGGATTTTTTTTTTAATCTATTTTC | 0.8621 | LOC387780-[POLA2] CDC42EP2 |
| 617266 | rs8638 | 11 | NC_000011.5 | 64840278 | C | G | AGGACTTGTGCAGCCGGGCCTGCCTCTGAGTGGTGCCTCTCCTGGAAGGAA | 0.8548 | LOC387780-[POLA2] DPF2, CDC42EP2 |
| 617297 | rs630055 | 11 | NC_000011.5 | 64862893 | A | C | GGAGCTTGGCCCTGGTGGTGAGTCTATAAGATGCAGTAGGTCTTAGAGTGA | 0.8614 | POLA2 [CDC42EP2] DPF2, TIGD3 |
| 617524 | rs7940108 | 11 | NC_000011.5 | 64041560 | G | A | GTGCAGTCCCTACACCCTGCTTCCCGCAACCATCCTCAACCCAACAGCCCC | 0.5662 | FLJ37045 [ ]-SLC22A11 |
| 619434 | rs11032807 | 11 | NC_000011.5 | 34658644 | A | C | TTTCTCATACACAATTCAATCAAAAATTACTAGGACCAAGTGACCAAATTC | 0.8406 | EHF [ ]-MMRP19 |
| 622215 | rs655875 | 11 | NC_000011.5 | 79506694 | G | C | TTTTTTTTTAAAGAGAATCAAAGATGAATAAGATTTAGTACCAAGTGAGAC | 0.6546 | LOC390227-[ ]-MGC33846 |
| 625834 | rs2295756 | 11 | NC_000011.5 | 35205538 | T | C | TAGTTACTAAAGCTTCCTTCCTAAATGAATGATCACCAAGAGGACCCCATG | 0.6259 | PDHX-[CD44] SLC1A2 |
| 627822 | rs3135056 | 4 | NC_000004.6 | 2068475 | C | A | CAGCAGAGATGTCCCAGGCTCAACAAATTTGCGACTGAACTCTTGTTCTGA | 0.9281 | LOC401115, FLJ37478 [POLN]- |
| | | | | | | | | | MGC4701 |
| 632286 | rs243978 | 4 | NC_000004.6 | 111740752 | C | T | CACTCTAGAGGAGTCTACTTTTCAGCGATGAGCAGGAGAATGCCAATAAAT | 0.3735 | LOC132707-[ ] LOC391684, |
| | | | | | | | | | LOC391685 |
| 652898 | rs10489158 | 1 | NC_000001.5 | 21969871 | T | C | CCTTACCAAATGGAGGGTCTGAACTTGGATGCCGGGAAGTTTTCTGGAAGG | 0.9487 | WNT4 [ ]-LOC343384 |
| 653852 | rs591858 | 1 | NC_000001.5 | 30393689 | C | T | TAGTGTTTTTCAGGAGATAATGTGACTGTTTTTGCGCTAGAAAGTCATGAA | 0.8238 | LOC388614 [LOC388615]-MATN1 |
| 663947 | rs12090000 | 1 | NC_000001.5 | 174391736 | A | G | ATGCAGTGAGCAAAATGGATTTGGCAGTGACTTAAAAAGACTTCATGTATT | 0.9749 | PLAC3-[ASTN, LOC57795]-LOC400796 |
| 664783 | rs859413 | 1 | NC_000001.5 | 172684309 | G | A | GAAAAAAAAAACTCATTCTTACCCAGCTTACTTATTGTCAATACATAAATA | 0.4077 | TNR -[ ]-LOC388716 |
| 668040 | rs2420178 | 1 | NC_000001.5 | 68541785 | A | G | CCTATTTATTCACTTGAGCATTCTGAATATCATTGACATTTTCTTTGACTG | 0.1122 | AF357533-[ ]-LOC388639 |
| 670526 | rs845451 | 1 | NC_000001.5 | 207508491 | C | T | GGCTGTAATCGAAGCAGAAGTGCCTCACAAATTAACTAGCAGTCGCCCACA | 0.5548 | LOC400802, SERTAD4 [ ] LOC339397, |
| | | | | | | | | | LOC199827 |
| 674238 | rs12750241 | 1 | NC_000001.5 | 37252821 | T | C | GTTCAAACACCGGAAATGTCTCCAGTCATGGAAAATGAGCAAATAATGAGC | 0.4822 | GRIK3-[ ]-FLJ23231 |
| 679867 | rs11203277 | 1 | NC_000001.5 | 16680808 | C | T | GGGCCCAGCCTGTGCACCTCGACTCCTTCCAGCCTATGCTTTCTTGCCCCT | 0.2183 | CROCC, LOC400742 [ ] MFAP2, SDHB, |
| | | | | | | | | | HSA9947 |
| 690495 | rs2474291 | 1 | NC_000001.5 | 27352326 | G | T | CAAGGAAGAAGCAAGTCCTTGCATAGTGATCGTTTGTGACCTAAACAACTA | 0.8979 | GPR3, FCN3, LOC388611 [ASF2]- |
| | | | | | | | | | DJ159A19.3 |
| 697891 | rs3766983 | 1 | NC_000001.5 | 178886415 | C | G | CCCTAATTTATTTGTGGGATAAAAACGATATTGAATATGAGAGTGGTGGGA | 0.4278 | LOC284646-[CACNA1E]-LOC127665 |
| 704088 | rs9425465 | 1 | NC_000001.5 | 169118706 | T | C | TTATGTTGATATATTTTCCTTTTCATAACACCCTAAAATCTAGATACAATA | 0.5234 | LOC127099-[KIAA0820]-LOC92346 |
| 707407 | rs2295633 | 1 | NC_000001.5 | 46244300 | A | G | GATGTTGTCGTCGGGGTGAACTGTGACCCTGTGGGACAAGTATATAGAGGG | 0.3721 | MGC22960 [FAAH] LOC388628, |
| | | | | | | | | | LOC391036 |
| 727193 | rs6436839 | 2 | NC_000002.6 | 230172337 | A | G | GTAGCAAGTGCATTCTGTGAAACAAAGATTTTTTATCATTATTATTGGCCA | 0.75 | SKIP-[FLJ20701]-DNER |
| 736610 | rs3732051 | 2 | NC_000002.6 | 51401708 | C | T | AGCCAAAAAAGTATCACTTAATGCACCAAAGTAATAATGAGCAAATTAGTA | 0.8612 | NRXN1-[ ]-CRYGGP1 |
| 739904 | rs2192720 | 2 | NC_000002.6 | 40221508 | C | T | GAAAACTTCAGAGAATCTTGTGTAACGGAACAGATAATTTTAGTAAGTTAA | 0.7268 | LOC391368-[ ]-SLC8A1 |
| 755186 | rs12993541 | 2 | NC_000002.6 | 41213992 | G | A | TCATGTAATTGGTAAAGTACCATTGGTATTTTTTCCTCATATTAGATATGA | 0.6772 | SLC8A1-[ ]-LOC388941 |
| 761347 | rs17014806 | 2 | NC_000002.6 | 34368781 | T | C | ATTTGCAACAGCCATTCAACTCTAGTATTAGCCTAATGATATCAAAGTCCA | 0.8685 | LOC344371-[ ]-MRPL50P1 |
| 761361 | rs17014794 | 2 | NC_000002.6 | 34363274 | C | A | ATTTGCTGAGGATATAAAGGGGAGACTGTCCAAATGCAAACCCTCAGGAAG | 0.868 | LOC344371-[ ]-MRPL50P1 |
| 764102 | rs3770403 | 2 | NC_000002.6 | 53897359 | A | T | AAAATGTATTGTTACTTTACTACAGAGACCATTACATTGTATAGACCAGTT | 0.5631 | LOC388949-[ASB3]-XTP3TPB |
| 765032 | rs11904632 | 2 | NC_000002.6 | 15427783 | C | A | AGGAGGTAGGTTTGTCCAAACCATGCAAATTCTAAGGACAGCCGATAATAC | 0.545 | LOC400944-[NAG]-DDX1 |
| 766107 | rs12990693 | 2 | NC_000002.6 | 215338370 | T | C | GTAAATTAATTTGAAGGGTAAAGTATAATTCTGATACTTTTTTGCATTTTT | 0.3988 | ZNFN1A2-[PF20]-LOC402117 |
| 769719 | rs1424677 | 2 | NC_000002.6 | 155129332 | A | G | AAGTGGTGTTCCATGTTGCTTCCTTATTACTAAACATGAGAAACTGCATTA | 0.117 | REPRIMO-[GALNT13]-KCNJ3 |
| 770100 | rs4669889 | 2 | NC_000002.6 | 13002330 | T | C | GCAAGGAACTCAAGACAGCCTATCTTTCTGGAAAAGTAACTGTCCAGTTTT | 0.4341 | TRB2-[ ]-NSE1 |
| 770116 | rs4669885 | 2 | NC_000002.6 | 12994758 | C | G | ACTCCAGCTGTGTGTGATTCCTGTCCTATCTGTCTGACTACAAAGCACATA | 0.4391 | TRB2-[ ]-NSE1 |
| 770139 | rs10164960 | 2 | NC_000002.6 | 12986898 | G | A | TGGTCATCCATTCCTCAAGCCAGCCGGAATCTCCAAGTTCATTTCTTCCAT | 0.4335 | TRB2-[ ]-NSE1 |
| 772518 | rs13015492 | 2 | NC_000002.6 | 15527455 | G | A | CCATTCATTCCAGATGTCTCACAACGGATGACTGGCCCCAGTCACACTGCC | 0.3029 | LOC400944-[NAG]-DDX1 |
| 773759 | rs17202778 | 2 | NC_000002.6 | 208437329 | T | C | TGCTGTTCCTCTGACAATCAATTCATTGATCTTCACCATTCCACTCCAGGA | 0.8093 | LOC389071 [ ]-CREB1 |
| 784172 | rs7562607 | 2 | NC_000002.6 | 169740134 | T | C | GGCACTCAATTAATACTTGAAACAATGTGTGAATGAGAAGCGTGCAGTAAT | 0.5558 | STK39-[LOC253782]-NOSTRIN |
| 797205 | rs3804701 | 3 | NC_000003.6 | 110034946 | A | G | ACTATATCTAAACAAACCCTTAATTACAGCAGTACCACAGGACACTGTTCT | 0.6342 | GUCA1C [MORC]-LOC401081 |
| 799054 | rs16851191 | 3 | NC_000003.6 | 142283107 | C | T | AGTGACATTTCAAGAATACCACCTTCATCCAGTCAGGCGTATGCTGAGAAA | 0.8408 | SSB4-[FLJ23751]-FLJ35036 |
| 800376 | rs562511 | 3 | NC_000003.6 | 62736442 | G | A | ATTATAGAAGAGTTTGGAGGAAGACGTTCAGCATTTGAGCTAATATGTGCA | 0.0534 | FEZL-[CADPS]-LOC389127 |
| 803273 | rs961155 | 10 | NC_000010.5 | 117243752 | T | A | TTTCTTCTCTGTATCACTGTTTCTATCTCTGTCCCTCTCAGTAGCACCCTG | 0.4533 | TRUB1-[KIAA0534]-GFRA1 |
| 805214 | rs3814218 | 10 | NC_000010.5 | 105253204 | G | A | TTGGACGATTGCTTGGGTGAGCCACGATACATACGTTGGTGCATTCATTTA | 0.8981 | SH3MD1-[ ]-FLJ22559 |
| 806436 | rs11010228 | 10 | NC_000010.5 | 35856021 | C | T | TCCATCAGGCCCATTCTTGGCATCACGTTAGTTTTCTGATAATTTATCACA | 0.6246 | CREM-[C10orf9] CX40.1 |
| 811022 | rs7069805 | 10 | NC_000010.5 | 21062602 | T | C | TGCCTTCCCAGAAGCCAAAGGATAATGATGGTCTCATTCTTCACATTGTAA | 0.7095 | LOC220998-[ ] NEBL |
| 813112 | rs3865353 | 17 | NC_000017.6 | 5561640 | G | T | TATTAACAGAACAAATGTGCAGAGGGATACACAGACAGGATAAAATCTAAA | 0.5718 | C1QBP, MGC4189, NUP88 |
| | | | | | | | | | [DHX33]F-LANa, MIS12, LOC388326 |
| 814339 | rs17747162 | 17 | NC_000017.6 | 10080545 | A | G | GCATTCACTCTCTCCCCTTTAATCTAGGTTCCCTGAGGCTACTGGAGGCCT | 0.6573 | RCV1-[GAS7]-RPS27AP1 |
| 814347 | rs3826528 | 17 | NC_000017.6 | 10086172 | A | G | AGTTGCCAACCAGGTATTTCTCCCCAAATGTCCACTGTTACCTGAAACTCC | 0.6873 | RCV1-[GAS7]-RPS27AP1 |
| 814713 | rs12150523 | 17 | NC_000017.6 | 11045062 | T | A | GAGGTTTCATTACCAGTAACACTGCTGACGCTTGAGCTCAGAGGTAGCAAG | 0.721 | LOC400573-[ ]-LOC388336 |
| 821871 | rs11869222 | 17 | NC_000017.6 | 49468518 | T | C | ATCTCATCTGCTCCCACAACCACCATCTAAAATAGGCAGAGCAGGTTTTAC | 0.8306 | LOC400604 [ ] SPAG9 |
| 829556 | rs3822787 | 5 | NC_000005.5 | 9345951 | A | G | TTTCATATCCCACACTGAATACCTTGTGATGGCACTGCCACTACCACTGTT | 0.8257 | MTRR-[SEMA5A]-TAS2R1 |
| 829565 | rs6874451 | 5 | NC_000005.5 | 9339456 | C | A | CCCTTCAAGAGCTGACTGACCAGGGCTGGACAGTTAACTCACTCCTCCAGT | 0.2043 | MTRR-[SEMA5A]-TAS2R1 |
| 842261 | rs16887892 | 5 | NC_000005.5 | 57453557 | C | T | GATGAGCCCTGCTCCCTGGGTAAGGCCGGTGTGGATGAAGAAGTGGTCAGA | 0.8404 | LOC401188-[ ]-PLK2 |
| 848514 | rs12350573 | 9 | NC_000009.6 | 117713981 | T | C | GGTCCTGATATACTATGATAAGTGCTATATGCAGAGTGAACACAGGGCACT | 0.4887 | DBC1-[ ]-CDK5RAP2 |
| 849011 | rs10984496 | 9 | NC_000009.6 | 117483406 | C | T | CCTAATCTCTGGGAACAAGTGAAATCGAGAGAGCCGGAAGCACCCAAAACC | 0.8859 | LOC347165-[DBC1]-CDK5RAP2 |
| 863475 | rs6476875 | 9 | NC_000009.6 | 4519671 | T | C | ATTAGATAATTAAAAGCCTCTGCCATCAGTCAAAATGAAACTTTTTTTGTG | 0.6466 | ZNF515-[SLC1A1]-C9orf68 |
| 872816 | rs7855014 | 9 | NC_000009.6 | 75386914 | C | A | GATAATTGATAGATTGGTTGAGTAACTAGAAATAATCAGTGCAATTAAATT | 0.5079 | C9orf65-[VPS13A]-GNA14 |
| 872845 | rs10781429 | 9 | NC_000009.6 | 75346051 | A | C | TCTTCTGTAGTCATATAAGAAATTTAAGGCAATAGCAGATATTTGTATATT | 0.4739 | C9orf65-[VPS13A]-GNA14 |
| 882230 | rs10959932 | 9 | NC_000009.6 | 11552051 | G | A | TGTGACAGTATTTAAAATGCAAAGGGAAGACTTAGAGAGTAGAGTATGTCT | 0.7533 | LOC340479-[ ]-TYRP1 |
| 882560 | rs10125743 | 9 | NC_000009.6 | 11367279 | A | C | GCAATGTCTAACCTTGCCTTCTTTTAAGAAAACTCTGTGTCTGTGTTGCAT | 0.0731 | LOC340479-[ ]-TYRP1 |
| 882595 | rs1029187 | 9 | NC_000009.6 | 11347578 | T | C | CTCCCATCTGTGCCTGCAATATAAGTTTGAATCACTGAACTTATCTGATTG | 0.9284 | LOC340479-[ ]-TYRP1 |
| 883568 | rs7967032 | 12 | NC_000012.6 | 1916360 | T | G | AGCCTGCGTGCTGCAAACGACGCATTTGACCTCCAAGATGACTCCACGTTC | 0.493 | LOC399984, CACNA2D4 [ ] DCP1B |
| 885372 | rs4765746 | 12 | NC_000012.6 | 3600144 | A | G | TCCAATCAAGTCTCTGCTACTTAGTATCTTATTAACTGGPACCCTCTAAAA | 0.3636 | HRMT1L3 [ ] MGC4266 |
| 891440 | rs7959121 | 12 | NC_000012.6 | 125182304 | C | T | CCCTTCAAATTCGTTAATTCTCTGACTGCAGTTGAAGAAAATCCAGCCAAG | 0.9911 | LOC387894-[ ]-LOC144678 |
| 894518 | rs11063482 | 12 | NC_000012.6 | 5029947 | T | C | TCTGCCTATGACTCTGGCCAGTGTGTTTTTCGCTGAGCTGTTGGTGGAAGT | 0.7316 | LOC390282, KCNA5 [ ]-LOC387826 |
| 897732 | rs2110099 | 12 | NC_000012.6 | 10921228 | C | A | TTTTCTAGTTGATTAGGCAGATTAACTTTCTGTTTTTCTGCTGACATAAAT | 0.3716 | PROL4 [ ] PRH1, TAS2R13, PRH2 |
| 898321 | rs2889626 | 12 | NC_000012.6 | 8131284 | A | G | AGGCTGAGGGAATCTGCTGCCATGAATATCCATTTACTTACAGAATTTGTT | 0.4393 | C3AR1, FHX [DKFZPS66B183] |
| | | | | | | | | | CLECSF6 |
| 903569 | rs3764022 | 12 | NC_000012.6 | 9724791 | C | G | TTTTCAATAATTTTTTCCAGGTTGTCTGCATTCAAAAGAGCATTCTATTAA | 0.6714 | LOC374443 [LLT1] LOC400000, |
| | | | | | | | | | DCAL1 |
| 904811 | rs12424340 | 12 | NC_000012.6 | 96108690 | A | G | CCTCTTTTTCAAGATGAAATATGCAAAGTGAAGTATGCAGATAGCAGGACC | 0.7507 | NEDD1-[ ]-NCRMS |
| 906272 | rs7304507 | 12 | NC_000012.6 | 16545580 | G | A | CACAACTGTATCTGAACAGATTCTCGTTACATAAAACCGCACACACAGTGT | 0.2683 | LOC400011-[ ] DAT1, LOC121520 |
| 908741 | rs10492234 | 12 | NC_000012.6 | 4457860 | T | G | ATAATGGCACCAATTATTTCTTCAATTATGAAGGCACCAATGGGATGGAAA | 0.9472 | FGF6 [ ] C12orf4 |
| 910353 | rs7968801 | 12 | NC_000012.6 | 12928074 | A | G | CTTCAAACTTGTGCAAGATGGTGCCATAGAGGATAAAAACAAGATGGAGGC | 0.3915 | LOC387841, LOC341465 [ ] RAI3 |
| 918677 | rs16932246 | 12 | NC_000012.6 | 27675896 | G | A | TACCTTCATATAATAGCATTTGAAAGGCTCATTAAATAGAACAGAGAAGAA | 0.7807 | LOC341346-[PPFIBP1]-LOC387849 |
| 918719 | rs7959164 | 12 | NC_000012.6 | 27691713 | T | C | ATTGTACATTCCTAATCTACACGAATTTTCTCCAAATTGTATAGGCCTCCT | 0.24 | LOC341346-[PPFIBP1] LOC387849 |
| 920234 | rs1029398 | 13 | NC_000013.6 | 30597288 | A | G | GTTTGACTCATTTCGGGAATATTCTATCTCCACCATTTTTCATTGCCCTTT | 0.7692 | LOC196549-[13CDNA73]-BRCA2 |
| 921310 | rs2858826 | 13 | NC_000013.6 | 31504291 | A | G | GAACATTCAAGTTTAAAGTGAAAGCGAGTACAAAGCCAAGGTACAGAAAAG | 0.4179 | LOC387918 [STARD13]-RFC3 |
| 944388 | rs9558696 | 13 | NC_000013.6 | 104519581 | C | T | AATTTTAAGCCAAAATTGTTTTTCACAATTGCAACCATAATTCAGTTTTTC | 0.9159 | LOC144920-[ ]-LOC341604 |
| 945100 | rs9783551 | 13 | NC_000013.6 | 96167159 | T | C | CATGAAGGCCAACAAGAAATAGAGATGTTGAAAGAAAACAGAGAGAAAGGA | 0.4442 | LOC387943-[ ]-KPNB3 |
| 962652 | rs9931136 | 16 | NC_000016.5 | 25504539 | G | A | GTGGTCAGGTTCTGGACAGTTCTCTGTGAGAATATTTAAATGATACATTGC | 0.5767 | HCP39-[ ]-HS3ST4 |
| 972242 | rs39573 | 16 | NC_000016.5 | 8401150 | A | T | CACTGATGAGTACCAGACTAGAAGAAGATATTTGGAATACCACAGAAGGAG | 0.7096 | A2BP1-[ ]-LOC401830 |
| 979327 | rs6668830 | 1 | NC_000001.5 | 4892273 | C | G | TGCCCTCTGCACCTTGGGCACAAATCGAGATTGGATTCCTGGGGAAACTCT | 0.7515 | LOC388589-[ ]-LOC126772 |
| 982183 | rs7522080 | 1 | NC_000001.5 | 4145831 | T | C | TGCAGGTACACGGAGCGCCTTCTTTTCGTTTAGAGCATTGGTAAGTACCTA | 0.9751 | LOC401937-[LOC284661]-SHREW1 |
| 1001406 | rs6693871 | 1 | NC_000001.5 | 27391611 | C | T | TCTGTTGGCGAAAGAAAAAATGCACCTGTATTATGTAAATAAGAATCCATG | 0.1008 | WASF2 [ ]- DJ159A19.3 |
| 1004017 | rs4265476 | 1 | NC_000001.5 | 30531562 | A | G | TTGCAAAAGCCTAAGGACTGTGTAAATATTCATAATACTGTTTTTAAATTA | 0.007 | LOC388615-[ ]-MATN1 |
| 1009275 | rs12078977 | 1 | NC_000001.5 | 34814977 | T | C | TGGGGAAGGGTATGTTTGCAGGCTATTGTTAACCCAGATGAGTGAGATGCA | 0.9556 | SAPAP3 [LOC388617]-MGC14276 |
| 1016164 | rs17362424 | 1 | NC_000001.5 | 41207697 | G | T | TTGTAAGAAAGTGTGTGCTATTTCTGGGTTATCCATGTGGGCAGGGGGAAA | 0.7777 | SCMH1-[ ]-LOC391030 |
| 1043573 | rs1496012 | 1 | NC_000001.5 | 64177746 | G | A | GTTGAGTCCCATTATCACTCCAGGAGGTACTTTTGGGATAGGTCCTTTTTT | 0.5974 | MGC35130-[ ]-KIAA1573 |
| 1046450 | rs17486706 | 1 | NC_000001.5 | 66377596 | A | G | TCGTGCAATGTCTCTGTGTGGTAGAAAGAATAAGTACGGTAATCCTCCCTT | 0.8692 | PDE4B-[DKFZp761D221]-FLJ40873 |
| 1054104 | rs11162856 | 1 | NC_000001.5 | 75120079 | T | C | GTGCCTTTTTCATACCTTAAATAGATGCTCATAAACCAAAGTGTTATATGT | 0.7249 | LOC148864 [MGC34032]-LOC388641 |
| 1054144 | rs666397 | 1 | NC_000001.5 | 75132657 | 1 | C | CAGTATAATTTAAGATTAATATTGCTTGTGTAGTCAATGCATATTCAGGGT | 0.2729 | LOC148864-[MGC34032]-LOC388641 |
| 1057517 | rs626492 | 15 | NC_000015.5 | 46559654 | T | C | GATTTGTTCTAAGTTATCTCTACTCCCTTGCCTGGCCTTTTGTTAAAAACA | 0.2488 | DUT-[FBN1]-LOC400370 |
| 1059228 | rs1933328 | 1 | NC_000001.5 | 80781549 | C | A | CCACCAGTCCATAGGGGTGAAAAAACCTATTTTTATGCACTGTGACATTTT | 0.3202 | COX6A1P-[ ]-LOC391050 |
| 1059350 | rs1195772 | 1 | NC_000001.5 | 80957737 | A | G | GGCCAACATCAGAGTTTTTCTAGCCGAAGTTATTTTAGAGGGTAATCAGGA | 0.8526 | COX6A1P-[ ] LOC391050 |
| 1062376 | rs12133778 | 1 | NC_000001.5 | 83790219 | A | G | AGAAAACAATGTTACAGTGCAGTTGATGGGCCCTTCATGGTGCTGGCCACT | 0.4847 | LPHN2-[FLJ23033]-PRKACB |
| 1064263 | rs12725350 | 1 | NC_000001.5 | 85766024 | A | G | CATTCATTCTTTTATTCTTTAATGCACTCATTCATTCAACATTTATTAAGC | 0.7568 | FLJ20729-[COL24A1]-K1AA1229 |
| 1064904 | rs7543542 | 1 | NC_000001.5 | 86957632 | A | G | TCTAAGTGCACTGCAAATGTGGCATAACTAGTTAACTTTCTTCAGTGATTT | 0.1962 | SEP15-[HS2ST1]-LOC339524 |
| 1066286 | rs551904 | 1 | NC_000001.5 | 89148422 | C | G | ACCTATTTCCTATAAGACCATGGAGGTGAGCTTACCTCTCTAAGCCTCAAT | 0.6953 | GBP4, LOC388646 [ ] GBP5 |
| 1067713 | rs17131120 | 1 | NC_000001.5 | 90580175 | A | G | CAATCATGTTACAAAAAGAAAATTAACAATACCCTAAAATAAAGAATTCCA | 0.9274 | FLJ20403-[ ]-BARHL2 |
| 1067961 | rs17131433 | 1 | NC_000001.5 | 91348930 | C | T | TAGATGTATTAGAACATCATCCTTCCGCTAGTAAATAGAGTACTGTGGGTT | 0.7929 | LOC164045 [ ]-CDC7 |
| 1068351 | rs10493858 | 1 | NC_000001.5 | 91759738 | C | G | AACTTCATTTTCCAGCCTCACCAATCTTTTAGAACAGAAGATCAAGTCAAA | 0.7742 | TRAP2-[TGFBR3]-BRDT |
| 1071700 | rs11165895 | 1 | NC_000001.5 | 97498176 | C | A | AAGTAGAACCATITTGAGTTTTTCACAAGAAAGATATTTAAGGCTGACGTT | 0.1506 | PTBP2-[DPYD]-LOC400765 |
| 1071708 | rs6663357 | 1 | NC_000001.5 | 97507862 | A | C | TATACTGAGTGAATGGCTATTAAACATCCATCCCAATGTAAGTAACAGGAC | 0.1436 | PTBP2-[DPYD]-LOC400765 |
| 1084766 | rs17563390 | 1 | NC_000001.5 | 108741590 | G | A | TATCAGAAATGCTTGACTTAGAAGCGTCTCCTTCATTTGCTGGAATATGAA | 0.8716 | STXBP3 [MGC26989] GPSM2 |
| 1085178 | rs2297757 | 1 | NC_000001.5 | 109013646 | A | G | GTTACAAGTTATTTCTTTGGTGTCAACGCTCATTTTGTGTGTGTGACTAAC | 0.6066 | LOC127003-[KIAA1324] SARS |
| 1085506 | rs682288 | 1 | NC_000001.5 | 109022367 | C | T | TGGTACAGGTAACACTCATAAGTCACGTGTATAGTACTCTCCAATTGACAA | 0.3918 | LOC127003-[KIAA1324] SARS |
| 1088326 | rs4838884 | 1 | NC_000001.5 | 110827297 | G | A | GAGCAGACCTGATGACACTGAACCTGGCTAGAGAGGTACTCAAAACCAAGT | 0.19 | RIF1 [ ]-MGC54289 |
| 1095559 | rs17185373 | 1 | NC_000001.5 | 117629056 | A | G | GTGTCAATGCATAATTTTGTAAACTATGTTACTGTCCTCAAGATTACAAAC | 0.5434 | LOC401959-[ ]-GDAP2 |
| 1096046 | rs6671887 | 1 | NC_000001.5 | 118213764 | A | G | ACTAGAGCCTTTATTCTCAGAATCTAAGTTATTGTAGGTAAAAAATGGATG | 0.2368 | WDR3-[ ]-LOC391072 |
| 1097038 | rs12030458 | 1 | NC_000001.5 | 119199457 | A | C | AGGGAATGGTTAGTGGCACTTAAAGAGACTGAAATACAACATATGGGGAGT | 0.924 | LOC343495-[ ]-HAO2 |
| 1102760 | rs4950328 | 1 | NC_000001.5 | 144511888 | T | C | TGAAGCCTCAAACTCCTTGCGTGTTTAATTTACTGCTGTTCATCCAGAACT | 0.2817 | CHD1L-[ ] BCL9 |
| 1110134 | rs564211 | 1 | NC_000001.5 | 150330659 | G | T | ATTTATCCATAACTGGTATGACAGGGAATCTCAAAAAAGGAAATATCCTGT | 0.8768 | SPRR2C [ ]-LOC149018 |
| 1119230 | rs1875766 | 1 | NC_000001.5 | 157767072 | G | A | TGCAGAGGCAGGCTTAGGTGGAGTTGAGCTAGATGTAACAGGACTATTTCT | 0.103 | CD84 [ ] SLAMF1 |
| 1124752 | rs12034856 | 1 | NC_000001.5 | 160731951 | A | G | ATTACGGATAGAACAGGGGAGCCTAAGTTTATTCTCTCTGACCTCCCACCT | 0.5336 | COCA1-[ ]-LOC388711 |
| 1125283 | rs11577916 | 1 | NC_000001.5 | 161037481 | A | C | GCAGGAGAGAATATTTAGTCTGGCCAGATCAGAGAGGGCGAGGTGGAGAAT | 0.7547 | CDCA1-[ ]-LOC388711 |
| 1128559 | rs4578194 | 1 | NC_000001.5 | 162837993 | C | T | TGACATTGAAGACCAGAATGGTTCACTTGATGAGAGTCCCCAAAGCTAGTG | 0.673 | MGST3, LOC400795 [ALDH9A1] |
| | | | | | | | | | LOC54499 |
| 1132867 | [NULL] | 1 | NC_000001.5 | 169327931 | C | T | TCAATCTTTTCACCTTTACGTTTGACCTTTCAATCCTTTCGCCTTATTGAG | 0.7652 | LOC127099-[KIAA0820]-LOC92346 |
| 1136710 | rs1498123 | 1 | NC_000001.5 | 176921360 | T | G | TAAAATTTATAAGGTGATTTTTGTTTCTACCAGTGTAAAGAAGACCCATGG | 0.6995 | TDRDS [ ] MGC16664 |
| 1136732 | rs2454196 | 1 | NC_000001.5 | 176955962 | G | C | TTTGGGGTGGGTGATGGGACAGGCTGTTGAATAGCAGAAACAATGGAAGAA | 0.4391 | TDRDS-[MGC16664]-LOC163590 |
| 1139305 | rs7534913 | 1 | NC_000001.5 | 178777653 | G | A | GTGAAGCTTCCTTCCCTGACCTTTCGCTTATCATAATTGAGACCTATTTAA | 0.4891 | LOC284646-[CACNA1E]-LOC127665 |
| 1139328 | rs10494540 | 1 | NC_000001.5 | 178788928 | T | G | TTCAGGAGACTGGGAAACATTGGATTGAACACATGATATGTGAGCTGGGAG | 0.4931 | LOC284646-[CACNA1E]-LOC127665 |
| 1142006 | rs17574056 | 1 | NC_000001.5 | 181421075 | C | T | AGATGTTATCCAGTTTGCCAAGAAACCCTCACCTAGAGGCACCAAATTATT | 0.5719 | C1orf19-[ ]-LOC391144 |
| 1148525 | rs1535868 | 1 | NC_000001.5 | 190721875 | G | A | AAGATATGTCCTGTTCAAAACCAATGTTCTGAGAGTTTGAGAGCCGTGAAA | 0.7708 | HRPT2-[ ]-LOC401978 |
| 1150093 | rs1929218 | 1 | NC_000001.5 | 192025798 | T | C | TTATAAATTACTACTAGTGTTAATATTTCCTGGAACAGTAAAAAGAAGTAG | 0.3091 | LOC401978-[ ]-SLICK |
| 1162438 | rs7551756 | 1 | NC_000001.5 | 201263042 | A | G | ATAGATGAACAAGTAAAAAGTCTCAATGGGGTCTGTTTCACTGTGGAGCAG | 0.643 | FLJ40343-[S0X13] REN, |
| | | | | | | | | | FLJ10761 |
| 1166312 | rs2036100 | 1 | NC_000001.5 | 203085841 | C | G | TCTCCCACTCTTTTCCCTTCTCCTTCTTGCCTATGAAGCCTTTCCTGACCA | 0.6087 | LOC284581 [SLC26A9]-LOC391156 |
| 1178707 | rs2147477 | 1 | NC_000001.5 | 213328552 | A | G | GCAAAGAAGAAAAACTAATAAGCCCATAACTGACATTGAGTTCTAGTTGAG | 0.3312 | LOC391164, LOC200125 [ ]-USH2A |
| 1203600 | rs271735 | 1 | NC_000001.5 | 231746617 | A | C | AGCTCACTCCCTGCTTCAGAAGTGACCATGGATCTCAATGGCACAAGATTT | 0.3847 | TARBP1 [ ]-IRF2BP2 |
| 1203638 | rs271771 | 1 | NC_000001.5 | 231761537 | C | G | AGGATAATAAGGCTGACCTTGCCGGGTGTTGGGAGAATCAGATAATCAGAG | 0.3927 | TARBP1-[ ]-IRF2BP2 |
| 1205110 | rs4659838 | 1 | NC_000001.5 | 232606464 | T | C | TACAGAACATCTGAATAGTGGAATGTTGAGCCACCATTAAATAAATTTCTT | 0.3449 | GGPS1-[TBCE] MGC39558 |
| 1205283 | rs6429197 | 1 | NC_000001.5 | 232816166 | G | C | AAATTTGGGCAAATTACCAACCATTGTGGGTCTCAGTTTCGACTGTTAACA | 0.7073 | MGC39558-[GNG4] CHS1 |
| 1206778 | rs35712364 | 1 | NC_000001.5 | 233804984 | T | C | AAACAGTACCATACATTGCCTTTTATTAGATTTTTTTTTTTTTCCAGAGTC | 0.9569 | LGALS8-[FLJ10359]-ACTN2 |
| 1208382 | rs2808221 | 1 | NC_000001.5 | 234529712 | T | A | GGCAGACAGTGATCTCCAGGATATATCAGTAAATTTAAAAATGGTAATTTA | 0.6403 | LOC388754-[RYR2]-ZP4 |
| 1208424 | rs7545575 | 1 | NC_000001.5 | 234565210 | A | G | TATGTGTATAATTGCTCATTCATGGACTGGTGGACAAGCCTCTTTCAGATG | 0.6917 | LOC388754-[RYR2]-ZP4 |
| 1215253 | rs17713396 | 2 | NC_000002.6 | 217201 | C | T | GGAATGCATGTGCTGGTGAATGTTACGGTGGGGAAATGGTTGATGAGACTC | 0.6475 | LOC400937 [SH3YL1] ACP1 |
| 1219347 | rs2580871 | 2 | NC_000002.6 | 4501226 | A | G | GCTAATGTGAATTCAACACAGGAAAACTAAAAGGTGGTCACTGGTTTTCAT | 0.1308 | LOC253662-[ ]-LOC200475 |
| 1221183 | rs1554740 | 2 | NC_000002.6 | 6251182 | T | C | AAAGTAGAAAACAGCAGAGCTAAAATACAAAAATTTTCAAAGAAATAATCT | 0.6613 | LOC400940-[ ]-LOC391349 |
| 1224387 | rs4233868 | 2 | NC_000002.6 | 9523902 | A | G | GTGTATTTAGAATTATGTCTGACACACCGTTAAGAACCATAAAGTATTAGT | 0.7102 | LOC129642-[DDEF2] ITGB1BP1 |
| 1226296 | rs13016049 | 2 | NC_000002.6 | 12121165 | G | A | CAGTAAAGGGAGAGGAAGAGGATTTGAGAAGCCTAGGGACTTAACATGCTT | 0.5979 | LPIN1-[ ]-FAM10A3 |
| 1228577 | rs1349842 | 2 | NC_000002.6 | 13985779 | A | G | TTGTTTGCCATTCCATTTTGAAACAAGTAGTATAATTCTAAAACAAACAAA | 0.9133 | TRB2-[ ]-NSE1 |
| 1232320 | rs1983376 | 2 | NC_000002.6 | 17410547 | A | C | GATCTACAATTCTGGACTTGGCAGCAACTTCCCAGTAGCCATTCTACAATG | 0.8243 | DKFZP566A1524-[LOC391354]- |
| | | | | | | | | | LOC388925 |
| 1234410 | rs4832602 | 2 | NC_000002.6 | 18697968 | G | A | GGCTTGGAGAATCTTTGCATGTACAGTTTATATAAGCAAGCTTCATAATTA | 0.3249 | KCNS3-[ ] RDH14, NT5C1B |
| 1246865 | rs12623550 | 2 | NC_000002.6 | 34574064 | G | A | TTAAATGTGAATTGTAACTTTATACGTGCGCTATTTTTAACGTTTCTCTCT | 0.8355 | LOC344371-[ ]-MRPL50P1 |
| 1255229 | rs11124986 | 2 | NC_000002.6 | 44483370 | G | A | AGAAATAGAAATAAGGTTTCCAGTTGTTATTTACTCAGGCTTCCAATTATT | 0.3088 | PPM1B, LOC391371 [SLC3A1]- |
| | | | | | | | | | FLJ23451 |
| 1256280 | rs11693792 | 2 | NC_000002.6 | 45235692 | C | G | ATAGTACAGAATATGGCTAAPAGGACCAGGTGTGAGGTGCAGATTAAGTGC | 0.8445 | LOC151111, SIX2 [ ]-LOC400952 |
| 1257281 | rs13001566 | 2 | NC_000002.6 | 46804021 | T | G | TACAGTTCACAAGCTCAAAGCTTTGTTGTTCTCTGTCGTCACATGACTATC | 0.4778 | ARHQ [PIGF] CRIPT |
| 1260720 | rs10495984 | 2 | NC_000002.6 | 50054137 | C | G | AGTCAGATCCTGCAAAATGATTTATCTGTTCCTATGCCITTGGCCTCTAGT | 0.6132 | LOC339793-[ ] LOC130728 |
| 1266440 | rs777593 | 2 | NC_000002.6 | 61393637 | C | A | GAAAGATTTCTACTGTAGAGAACAACGTGGCATTTCTTCAGGGGATGGAGA | 0.3749 | AHSA2, LOC339803, LOC339804 |
| | | | | | | | | | [USP34]-XPO1 |
| 1266507 | rs10496092 | 2 | NC_000002.6 | 61499213 | C | T | GGCAATTAAACAAAAATTTAAAGTACCACCCTGAGAGGTAAAAAGAGTAAA | 0.6278 | AHSA2-[USP34]-XPO1 |
| 1266518 | rs1838978 | 2 | NC_000002.6 | 61515457 | T | C | GTTTCAAATCATGGAAGCATGTTATTTATAAGGAATGTATAAAATATCTTT | 0.6219 | AHSA2-[USP34]-XPO1 |
| 1266537 | rs2694632 | 2 | NC_000002.6 | 61533216 | T | C | GTGAAAAGATATAAAGCAATGTTCCTTCATTTTTATGAATTTAAAGTACCT | 0.6313 | AHSA2-[USP34]-XPO1 |
| 1266544 | rs2463102 | 2 | NC_000002.6 | 61545829 | G | C | CCGACATCTGCTTATTAACAATATGGAATGTTGAGAGCTTTACGAAACTGA | 0.6375 | AHSA2-[USP34]-XPO1 |
| 1266553 | rs778143 | 2 | NC_000002.6 | 61556424 | C | A | TTGGGTTGGGCAACATGTGGAGGAACTGAAAGCCTGGAGCCTGGGCCCAGG | 0.633 | AHSA2-[USP34]-XPO1 |
| 1268020 | rs11885480 | 2 | NC_000002.6 | 65310704 | T | C | ACCTCCCCTTATCAAAGACACACACTTGAATAGCGTGATCAGTGACACGTT | 0.181 | KIAA0582 [RAB1A]-LOC150984 |
| 1268046 | rs11684110 | 2 | NC_000002.6 | 65342824 | A | G | TGACCTGTTTCAGAGCACACCTCCAACCTTACCCAGCTCTCACCCACCTAC | 0.3055 | RAB1A [ ]-LOC150984 |
| 1273590 | rs13384240 | 2 | NC_000002.6 | 75074409 | T | C | GGAAGAAAGATACTTGGAGAGTTAATGATGTAAGGACACAAGTACAGTCAT | 0.8218 | SEMA4F-[HK2]-POLE4 |
| 1276506 | rs10170918 | 2 | NC_000002.6 | 80241437 | A | G | TTCCTATGCATTATGATTTGAACTCAAAAAACAGAAGGCTATCAGAAAGAC | 0.7298 | PAP-[CTNNA2]-LRRTM1 |
| 1276558 | rs1867806 | 2 | NC_000002.6 | 80293514 | T | A | GAAGGACTGGACAAGACAGGAGCATTTCCTAATCAATTTATGCAAAACCTC | 0.6288 | PAP-[CTNNA2]-LRRTM1 |
| 1276573 | rs1900265 | 2 | NC_000002.6 | 80317959 | T | C | TGTGTTTAGAATATGGACTGTGTTCTGGCTGTTACAGGTTCATGTGTGTTA | 0.6234 | PAP-[CTNNA2]-LRRTM1 |
| 1276632 | rs4852559 | 2 | NC_000002.6 | 80376037 | G | A | TGTATACTCTTGTGTTATGATACCCGCAGCACAGCAGTGCCAGGCACTCAC | 0.502 | PAP-[CTNNA2]-LRRTM1 |
| 1276639 | rs4142758 | 2 | NC_000002.6 | 80389782 | G | A | GCTGCTCTTTGAAGACAATATTTTCGAATTTCATCTGTTATTTCAAGGTTT | 0.502 | PAP-[CTNNA2]-LRRTM1 |
| 1276648 | rs7589687 | 2 | NC_000002.6 | 80402602 | A | T | CATGATACCTGAGGAAGTAACAAAGATTATACAATCATCCCTAAGTATTCA | 0.4956 | PAP-[CTNNA2]-LRRTM1 |
| 1276683 | rs2587148 | 2 | NC_000002.6 | 80438390 | G | A | GAACTTGCTTTTATTTATCTGTCAAGGAGCCTTATGTCTGTGAAATCACTA | 0.3232 | PAP-[CTNNA2]-LRRTM1 |
| 1276697 | rs188836 | 2 | NC_000002.6 | 80454928 | G | A | CATTGTCTCTTCTTGAGAAGAATCAGTTTGCACTATATGATATCTAAGTAC | 0.3908 | PAP-[CTNNA2] LRRTM1 |
| 1276713 | rs318366 | 2 | NC_000002.6 | 80464250 | G | A | GTGCCCTGCAGGGTTGCTTAGGCCTGTAATGTGGTCCTTCACACCTCTCTC | 0.3745 | PAP-[CTNNA2] LRRTM1 |
| 1293364 | rs2630505 | 2 | NC_000002.6 | 108260966 | C | T | TTTCTTTGCAAACCTGTCTTGCCTATTTTTCCTTAGGTTGAAAGGATTCTG | 0.1429 | SLC5A7 [ ]-LOC391418 |
| 1311548 | rs167164 | 2 | NC_000002.6 | 123660072 | T | C | TGATGATTTCTGAGAAGATATGACTTGTAACTTTCCAATAACACTTTTCTA | 0.1742 | LOC389028-[ ]-caspr5 |
| 1325111 | rs4560177 | 2 | NC_000002.6 | 133419267 | T | C | GCCTGAGTTTTCCTTAGCAACCTAATTCTGTTTGCCTTATGTGCTTGACTT | 0.1329 | LOC401012-[GPR39]-MGC29643 |
| 1327662 | rs7803969 | 7 | NC_000007.8 | 95114075 | C | G | TGCAGGGAAAGAAGGCCAAGAATGGCCAACACTGACTTGGAGAAGGATACC | 0.8493 | LOC389533-[DNCI1]-SLC25A13 |
| 1329983 | rs12470730 | 2 | NC_000002.6 | 135900830 | C | T | ATATTGAATACTGATATTTTGGGGCCGTTATTAACCCAGCTTTCTTTTAAA | 0.7975 | ACMSD [CCNT2] FLJ23074 |
| 1334452 | rs16840302 | 2 | NC_000002.6 | 139064740 | T | C | ACAGACTGGTAACATCTTCCCATTATTGCATGAGAAATTTTTTTCCCTTAG | 0.8954 | HNMT-[ ]-LOC150498 |
| 1337252 | rs7563559 | 2 | NC_000002.6 | 140992751 | T | A | TATCCTCCAAAACACCTCCATGGGATCTTTAGAGAACTTCAGAACATGGTT | 0.5725 | MRPS18BP2-[ ]-LRP1B |
| 1356550 | rs10497140 | 2 | NC_000002.6 | 155700080 | C | T | GGGAAAGGCCATTTCAGCTCATTGACAGGTAGTAGAGGATTAACGTAATTG | 0.9374 | GALNT13-[ ]-KCNJ3 |
| 1363739 | rs1834216 | 2 | NC_000002.6 | 163941427 | C | T | GGGTGACAAAGAGTATCTTGAGCTCCCTGAACTGTAGATCACAAGCCCCTT | 0.4037 | KCNH7 [ ]-FIGN |
| 1369909 | rs17568204 | 2 | NC_000002.6 | 168739502 | A | G | GAGAACTTTGTATGCACTCACAAGTATACATCAAAATCTCCTGGTGGTTTG | 0.7759 | CMYA3-[ ] LOC401018 |
| 1385352 | rs1518408 | 2 | NC_000002.6 | 181160044 | T | C | CATAGTGAGACCCTGGAACCCCTACTAAGCTGTTTGATTTAGAGACTCTTG | 0.0602 | KIAA1604-[ ]-UBE2E3 |
| 1386738 | rs6755680 | 2 | NC_000002.6 | 182431790 | C | T | TATAGTGATTAAAGTAGCACTTACACTTTTGACCCTGGCAGGTATTATCTG | 0.7009 | UBE2E3-[ ]-ITGA4 |
| 1387272 | rs12621807 | 2 | NC_000002.6 | 183058617 | T | G | GCAGCATAATGTCCCATCAGTCCCTTTCTGACAAATAACCTAGAACTCTCA | 0.7842 | LOC344318 [LOC151242]-PDE1A |
| 1390626 | rs2170203 | 2 | NC_000002.6 | 185811547 | T | C | AAACCAACACATTGCAATTATTTTGTATTATATACAGTATAAGTACATCAT | 0.3647 | PRO2964-[LOC91752]-LOC389066 |
| 1401365 | rs34481285 | 2 | NC_000002.6 | 197542278 | G | A | TTACAGACGCTTAACACAGTTTAACGAATTTCTCTGATACACCAACAGTGG | 0.9059 | STK17B-[NEDL2]-FLJ39660 |
| 1405790 | rs4393736 | 2 | NC_000002.6 | 201334094 | A | C | ATCACTTGGGTAATGCAACCCATTTATCTGCTCCCACTGTATAACAGCGGA | 0.8052 | FLJ22555-[ ] DNAPTP6 |
| 1405831 | rs17592517 | 2 | NC_000002.6 | 201359478 | A | G | TATTAATCTTTAAAAATTTTAAAGTAAAGCCTCTACCTATTCACAGCAAGA | 0.8298 | FLJ22555-[ ] DNAPTP6 |
| 1406391 | rs6745304 | 2 | NC_000002.6 | 201692033 | A | G | AGACTCATCTCCATGAAATGATAGGATGTTCTCTTGTGTGCATTCCATTGG | 0.3409 | TRIPIN [AOX1]-AOX2 |
| 1410980 | rs12694177 | 2 | NC_000002.6 | 210391688 | G | A | AAGGAGATCAGGATAGCCAATTTCAGGAGCTTCCCTTCCTTGATCTCATTG | 0.3397 | LOC402116-[ ]-MAP2 |
| 1431533 | rs4487084 | 2 | NC_000002.6 | 228915718 | G | A | AACAGGCTAAGGTAAATACTCACGCGAAAGAACCAAAAACTTTGTAAGAGT | 0.2197 | CCL20 [ ] FLJ25955 |
| 1442327 | rs946630 | 14 | NC_000014.4 | 32431904 | G | A | CTTTGGGGAACTGAAAGAAGCCCTGGAGAACACAATATACAATGGCACACC | 0.1732 | EGLN3 [ ]-C14orf147 |
| 1443605 | rs8003136 | 14 | NC_000014.4 | 33665950 | G | A | CCCACCACCACTATTTATTGACTGGGTGACTATAGGTAAGATAGTGAGCCT | 0.309 | CDC10P [KIAA0391] MRP63P8 |
| 1443883 | rs10145821 | 14 | NC_000014.4 | 34362385 | T | C | TCTTTGAAAGCCTTGAAAATCCTAATGTTCTTGCTAACAGTGGAAATGCTT | 0.997 | BRMS1L [ ] LOC390468 |
| 1445906 | rs1112504 | 14 | NC_000014.4 | 39699749 | T | G | AGACTGATACATTGCTCATATTCATGTTGATCTTCATTATGTTTGTGACAC | 0.2219 | FBXO33-[ ]-LRFN5 |
| 1447578 | rs7142438 | 14 | NC_000014.4 | 41754122 | C | G | TACACAAAGACAAATGAGAAATATTCTTACCTCAGGAGGTTAATATGAAGT | 0.7503 | TUBBP3 [ ] HNRPUP |
| 1456212 | rs880193 | 14 | NC_000014.4 | 54281613 | G | C | CTTGAAAATTGTTATCTCTTGTTTAGTATCTTATTTCGGAAAGCAGCATCT | 0.1567 | LOC387989-[ ]-PELI2 |
| 1468451 | rs17261246 | 14 | NC_000014.4 | 90069805 | T | C | TAAATGGAATGAGCACGTCTAAAAGTGTATATACCATGTGTCCTAATTTCA | 0.966 | KIAA2010-[C14orf161]-MTAC2D1 |
| 1476779 | rs6916070 | 6 | NC_000006.6 | 12179270 | T | C | AGTAGGTCCTGACTTCATCAGTCTATAGTGTTCTGTTCCAGCATTCTTTTG | 0.7347 | LOC389369-[HIVEP1]-EDN1 |
| 1487452 | rs6901756 | 6 | NC_000006.6 | 41872445 | T | C | CATGCATACTTACATAGTTCATCAGTAAGTGAGGATTCAATGGATGGATGA | 0.8661 | C6orf49-[MGC20741] USP49 |
| 1506595 | rs480295 | 6 | NC_000006.6 | 81229472 | G | A | ATAGGAGGACTATTCAGTATTTGGTAGGGAGCAGGGTAACAGAAAGGTTAC | 0.3183 | LOC340171-[ ]-C6orf37 |
| 1507256 | rs16894046 | 6 | NC_000006.6 | 83002753 | A | G | GATTTTTGCACAAAATGGGTTCTTCAATATAAACCCTTCAGTATTTCCAGC | 0.987 | IBTK [ ]-TPBG |
| 1507420 | rs2983879 | 6 | NC_000006.6 | 83149474 | T | C | CTTTTAACTCTTCCTTTACTGCATTTGTGCCTTTCCAATGACAATCTCGTT | 0.3868 | TPBG-[ ]-C6orf157 |
| 1508781 | rs9359604 | 6 | NC_000006.6 | 85362249 | G | A | CTGAACCATAAGCACTGAATTGACCGTTCTCTTTCCCCATAACTGCCATCT | 0.8774 | C6orf84-[ ]-LOC401269 |
| 1525920 | rs6937080 | 6 | NC_000006.6 | 121796228 | T | A | GGAGATGATACCTTCATCGAAGACATCCTCCTATTAGCATTCTAGAGCAGA | 0.8254 | GJA1, LOC260339 [ ]-HSF2 |
| 1527003 | rs17686735 | 6 | NC_000006.6 | 123692693 | C | G | TACACTAACACAGAGTGCACATACCCCATCTTTTAAGCCGAGTTCCATTAC | 0.5841 | C6orf213-[TRDN]-TCBA1 |
| 1529845 | rs17056873 | 6 | NC_000006.6 | 129456505 | C | G | TTGAAAGCTTCTGTAAACAGTTGAACTTCAAATTAAAAGGTAAGTAGGAAC | 0.9897 | LOC338470-[LAMA2]-ARHGAP18 |
| 1529998 | rs265326 | 6 | NC_000006.6 | 129570746 | C | G | GTTTATTTTTCATGGTTTTAACCCAGCATTAAGTAGCATGGTTTTTAGCAT | 0.8492 | LOC338470-[LAMA2]-ARHGAP18 |
| 1530007 | rs265392 | 6 | NC_000006.6 | 129576597 | A | T | AATATGAAAGAGACATGTGAATCTCTGCCTTTGAATACTTAGGATGTGTTT | 0.8412 | LOC338470-[LAMA2]-ARHGAP18 |
| 1532522 | rs6924201 | 6 | NC_000006.6 | 132877599 | C | T | GTTCCATAACCTTTGGGGCCAATTACAGGTCATGGATACACTGTTCCTAAG | 0.8415 | TAR3, GPR102, TA4 [ ] PNR, |
| | | | | | | | | | GPR57, GPR58 |
| 1534840 | rs10499200 | 6 | NC_000006.6 | 138752522 | A | G | AACACCAGCATGGATGACTTCCACAATGATATGACTTTCATGCCTCCCAGT | 0.8369 | HEBP2 [ ]-LOC401275 |
| 1534880 | rs7765940 | 6 | NC_000006.6 | 138775313 | C | T | AAGAATTATTAAAGTACCTACTACACACTACATACCATATATCAATTAAAT | 0.8395 | HEBP2-[ ]-LOC401275 |
| 1538591 | rs485434 | 6 | NC_000006.6 | 147492291 | G | A | GAAAGAAGTTTCACCTGCATGCTGCGAGCTTTGTGCCTGCTGCTATAATAA | 0.5208 | LOC389431-[ ] STXBP5 |
| 1542474 | rs11156106 | 6 | NC_000006.6 | 156742533 | G | A | TATGATTGTAAAGAACATTACAGCTGAAAACTCAAAATAATAAGTGTTTTG | 0.4102 | NOX3-[ ]-LOC389437 |
| 1542978 | rs6909234 | 6 | NC_000006.6 | 156384857 | C | T | CTTTGAGAGCACAAAGAGGTGGCAACTAACATACTCCAGTGTGGGACAGAG | 0.5256 | NOX3-[ ]-LOC389437 |
| 1552540 | rs6947755 | 7 | NC_000007.8 | 15983390 | G | C | CAAAAACCTTAATTTAAAAAAATCTGTATCAAAGAATAAAATTTTCCCAAT | 0.7081 | MEOX2-[LOC402250]-SOSTDC1 |
| 1553131 | rs1404963 | 7 | NC_000007.8 | 16906648 | A | G | ACTGTGGTGGCAAACTCATCTATTTATTTAACACTGGTATTTCAGCCATCT | 0.7549 | BCMP11-[ ]-AHR |
| 1555949 | rs17152880 | 7 | NC_000007.8 | 25666356 | T | C | GCATGTGTGGAGCTTGAGCCGGCGATAAATTGAGGCGCTAATCCTGATGCA | 0.9615 | UBA52P1-[ ]-NFE2L3 |
| 1572691 | rs17762851 | 7 | NC_000007.8 | 69630695 | C | T | CAAGTAATTGAATCTTCTAATGGAACAAACTGGTCTCTGCTTAATGATTTG | 0.9142 | FLJ13195-[AUTS2]-WBSCR17 |
| 1579845 | rs6967782 | 7 | NC_000007.8 | 96444809 | T | C | TATCCAGATCTCTCCCCAGGCTACATCTCTCTCATGAGCTCCATACCTCTG | 0.3198 | ACN9, LOC392076 [ ]-TAC1 |
| 1581736 | rs6977560 | 7 | NC_000007.8 | 104649224 | A | C | TGACTCTGTGTCAGCTTGACGAGCAAGTTTTTTGGTAGTGAAACATTTGTC | 0.1901 | SRPK2-[ ] FLJ20485 |
| 1585729 | rs2590597 | 7 | NC_000007.8 | 117961356 | C | G | CTTATCACACTTITTCATTGAGTGTCTATTTTACAAACAGGGGAGATCAAA | 0.3262 | ANKRD7-[ ]-KCND2 |
| 1585755 | rs17318046 | 7 | NC_000007.8 | 117942519 | C | T | TCATTTCGTGCAGGTCAGAAGTATGCTGCAGTACTGATTGTGAAAGAAGTA | 0.4291 | ANKRD7-[ ]-KCND2 |
| 1586038 | rs2402460 | 7 | NC_000007.8 | 118329604 | A | C | CAGAGGAAGCTCTCCATGTAACACAAAGTGCAGTGAGGAGTGAGAACCACA | 0.4936 | ANKRD7-[ ]-KCND2 |
| 1586042 | rs10258702 | 7 | NC_000007.8 | 118335931 | A | T | ATTTTCCCCTCTCTCAAATGTTGTAACTCCTAGTACAGTTTCTTTAGCATC | 0.4881 | ANKRD7-[ ]-KCND2 |
| 1586171 | rs10240560 | 7 | NC_000007.8 | 118668391 | C | T | ATTTTTGTTAGAGCCATTAATTCTACTATGCTGGACTAGTCAAGAGGGCCC | 0.8276 | ANKRD7-[ ]-KCND2 |
| 1586214 | rs1916861 | 7 | NC_000007.8 | 118927945 | T | G | CATTTCATGACATGCATTTCAAATATTTTATACAGCTGCTTCCTTAAAGAC | 0.8192 | ANKRD7-[ ]-KCND2 |
| 1586226 | rs17279126 | 7 | NC_000007.8 | 118933389 | G | A | GCACCAAACGAATATGTCCAATGTAGGGCGAATGTTGTCTTCTCAATTCTT | 0.8113 | ANKRD7-[ ]-KCND2 |
| 1586261 | rs17279645 | 7 | NC_000007.8 | 118961544 | T | C | TTCACGAAGAAACTAACTTTTCAGTTGCTATAAGATTTTTGTAGAACCCAA | 0.8198 | ANKRD7-[ ]-KCND2 |
| 1586311 | rs17280657 | 7 | NC_000007.8 | 119008753 | G | A | AACAGAGATCCTACTAGACATAGACGATTTAAAAAAAAATTTGAAAAGTCT | 0.8226 | ANKRD7-[ ]-KCND2 |
| 1596162 | rs3852883 | 19 | NC_000019.6 | 6434465 | A | C | ATACTCATGACAGCCCGACACACTCAAGCCGTGCTGCAGACTATGAAAGAC | 0.5908 | FAM31CCRB3, MGC2615, |
| | | | | | | | | | MGC34725 [ ] TUBB5, TNFSF9 |
| 1597461 | rs2431792 | 19 | NC 000019.6 | 8939545 | A | G | CAATTTGGCACTGCCAAGGACTTCAGCCCATAATCCCTTCTTCCGTAGTAA | 0.2792 | MBD3L1-[LOC400676]-LOC125963 |
| 1609848 | rs7254744 | 19 | NC_000019.6 | 40702499 | C | G | TAACCAACAAGAAAAACAAGATCAGCGCTATGAGAGAGAGTGACAGGGTGG | 0.268 | LOC388533, Z052F10 [ ] |
| | | | | | | | | | UNQ698, ATP4A, NIFIE14, GAPDS |
| 1611770 | rs3843043 | 19 | NC_000019.6 | 46125771 | C | T | GCTCACAAACTCAACAATTGCTTGTCTTTTTTCCTAGGGTATAAGCCTTTG | 0.2645 | CYP2A7, CYP2G1 [CYP2A7P1, |
| | | | | | | | | | CYP2B7]-CYP2B6 |
| 1613798 | rs3826861 | 19 | NC_000019.6 | 50748460 | C | T | ACCTGCTGGTCCCAGTGGCAGGTAACGGCTGCTCTTATCAGCAGGGGTAAC | 0.3634 | VASP [OPA3, LOC401922] GPR4 |
| 1614288 | rs8101491 | 19 | NC_000019.6 | 52334620 | G | A | GTCGGAGAGAAACGATGGTGGCTCAGACTAGAGTGGTGTTTGTGGAACTTG | 0.4561 | C19orf7 [SAE1]-BBC3 |
| 1614290 | rs12611429 | 19 | NC_000019.6 | 52340669 | A | G | CTCTACATTCCCATGCCTCAGTGACATTTTATTTATTTTATTTATTTATTT | 0.8264 | C19orf7 [SAE1]-BBC3 |
| 1614294 | rs11666272 | 19 | NC_000019.6 | 52350160 | T | C | CTGGAGAGGCTTTTTTTGATTCCAATAACATGATTCCTAGGGTAAAATTAC | 0.8221 | C19orf7 [SAE1]-BBC3 |
| 1614316 | rs3760765 | 19 | NC_000019.6 | 52385001 | C | T | GATTAATAATGTTGCTGTCCAGATTCCCGTTATAGCGCTAACCTGATGTTA | 0.8132 | C19orf7-[SAE1] BBC3 |
| 1614322 | rs11669489 | 19 | NC_000019.6 | 52391945 | A | T | TTTCACAGTAAGGTAAATTGCTTTTATAATTATTGGATTGCCTCTCTGACT | 0.8088 | C19orf7-[SAE1] BBC3 |
| 1614525 | rs12459087 | 19 | NC_000019.6 | 52661206 | G | A | CGATGGCCGCCATGAAACGGTCGGCGATGATGGACACTCCCAGAAACATGT | 0.8824 | MEIS3 [SLC8A2, NAPA, KPTN]- |
| | | | | | | | | | ZNF541 |
| 1616405 | rs2547321 | 19 | NC_000019.6 | 56574475 | A | C | TTATCCTACACATCCATATCCTGGAAGTTGAACTGTGGTGAGCTCCTGGAC | 0.5439 | LOC147645, ETFB, NKG7, |
| | | | | | | | | | MGC33839 [ ] LOC147646, |
| | | | | | | | | | LOC400712, LIM2, SIGLEC10 |
| 1618767 | rs2288519 | 19 | NC_000019.6 | 60401740 | C | T | GGAGAGGCCCATGGGTACTCTTCATCTCCCAAGGAGCTCCCAAAGTCCTTT | 0.5849 | SYT5, TNNT1, TNNI3, |
| | | | | | | | | | LOC352909 [PTPRH] KIAA1115, |
| | | | | | | | | | LOC388563, MGC30208 |
| 1626238 | rs209845 | 16 | NC_000016.5 | 12382754 | A | G | AGAAACTTCTCTGATCACATATCCAATTGTTAAAAAGAATTGTGGGTTACT | 0.4697 | FLJ12363-[LOC92017]-FLJ11151 |
| 1637827 | rs2042364 | 16 | NC_000016.5 | 65031268 | A | G | GGGAAAGTGTCACACAGATTGACAAATGTGGAGTCACAAAGCAAAGGAGGT | 0.4731 | CDH11-[ ]-LOC283867 |
| 1645177 | rs9923084 | 16 | NC_000016.5 | 74134676 | G | T | TGGCCACATTCAGGAACCAGGGAATGGAAGGGATGCTAATCTGAGGACATT | 0.8546 | LOC388291, PSMD7 [ ] LOC283922 |
| 1646094 | rs7106188 | 11 | NC_000011.5 | 3184457 | A | G | CACAACCTTCACCCTGCTGTGCCTCGCTGCAAGGCTCTGTTCAGTCAATTA | 0.5992 | OSBPL5 [ ] FLJ36102, MRGE |
| 1654367 | rs9633862 | 11 | NC_000011.5 | 10628447 | G | C | GAAGAAGTCGGGTAGGCCTAGACCAGAATCCCACCTGGGTGACCTTGGGCA | 0.8024 | LOC399865-[MRVI1]-SH2BP1 |
| 1660150 | rs2593590 | 11 | NC_000011.5 | 15805635 | G | A | TTTGTACAATCAAAAAGGAGCCTACGGGATCACTTCTGAGCAAGAGCGCTG | 0.2801 | LOC387756-[ ]-SOX6 |
| 1661334 | rs1520886 | 11 | NC_000011.5 | 18229047 | C | T | GGGGAGTGATTTGGTCCTTTACAGACGGATGAATGAATTTCTGTATCCAAA | 0.2371 | SAA4 [ ] SAA1, HPS5, FAM10A5, |
| | | | | | | | | | SM2 |
| 1667424 | rs10948827 | 6 | NC_000006.6 | 54430097 | T | G | CATCTTTTTTTAAGCTTTTATCATGTTTTTTGACAATGTAGTTAAAGTCTA | 0.7545 | CLNS1B [ ]-LOC221344 |
| 1674227 | rs2585813 | 11 | NC_000011.5 | 28578799 | A | T | GCCTTCAGCCTCCACAGAGTCTTCTAGTAGTAAACCCTTAAAGATGCTTCA | 0.6198 | FLJ33979-[ ]-LOC401677 |
| 1676930 | rs11032833 | 11 | NC_000011.5 | 34718056 | T | C | ACTATGATTATTAAAAAGGAATTGATTGGGATTGGAAATCAAGAGGGCCAG | 0.8406 | EHF-[ ]-MMRP19 |
| 1678333 | rs7107720 | 11 | NC_000011.5 | 36410525 | C | T | ACAGAATTAATGATAATGGTAGTGACGGCTAACATTTTCAAGCACGTACAT | 0.427 | COMMD9-[FLJ14213]-TRAF6 |
| 1686926 | rs10838121 | 11 | NC_000011.5 | 43514379 | A | G | CTTACATAGAAGGGAGGGTGTTTGAAATAAAGGATAGTTATATTAGGTAGG | 0.7438 | TTC17, LOC120449 [ ] LOC143970, |
| | | | | | | | | | LOC387762 |
| 1689616 | rs17725617 | 11 | NC_000011.5 | 46168067 | C | G | TGTGGTTGGCACTGAAACCAGCAGGCAAGACCAGAGAGTAAAGAAGCAAAA | 0.712 | LOC401679 [ ]-CREB3L1 |
| 1703484 | rs11236931 | 11 | NC_000011.5 | 70195793 | C | G | CAGCTATTGCTTATGCTCCACGCACCATTTGCCCTTTTGGAGGATCATCGT | 0.5501 | EMS1-[SHANK2] LOC399921 |
| 1705437 | rs1670543 | 11 | NC_000011.5 | 73316618 | A | G | ACTTACTGAAGTCCAAAACCAAGCTAAGTACATTTGTTGTCAAGTGAGTTC | 0.5779 | E2IG2, MRPL48 [FLJ11848]-TSARG6 |
| 1709425 | rs17755728 | 11 | NC_000011.5 | 78740181 | T | C | AGAGCCACCATTTCCTTGACCTAATTTGGACTCTTCTCAAACTCACCACAA | 0.8514 | ODZ4-[LOC387795]-LOC390227 |
| 1711184 | rs2448281 | 11 | NC_000011.5 | 79851900 | A | C | AGTTGTCCATGAATCAGTATTTGGGCAACAAAATCACTATACAACCCTGCT | 0.7048 | LOC390227-[ ]-MGC33846 |
| 1718318 | rs580459 | 11 | NC_000011.5 | 85162808 | T | C | AATGGATGAGAATCTTCTGCAGCCATCTGAATCAAATTCTGTGTGCTTAGC | 0.337 | FLJ38159 [SYTL2]-MGC34732 |
| 1718389 | rs537604 | 11 | NC_000011.5 | 85200191 | T | C | TCATCACACGGTCTCAAATCCCTACTTACTCATGTTTGTCTCTTGCCAGTT | 0.2944 | FLJ38159-[SYTL2]-MGC34732 |
| 1718430 | rs11234410 | 11 | NC_000011.5 | 85223048 | C | T | AGTCCGCAGGAGCTTAAATGAGGCTCATGGTCTGGACTGGCTGTTTTCCTC | 0.711 | FLJ38159-[SYTL2]-MGC34732 |
| 1718464 | rs930592 | 11 | NC_000011.5 | 85235182 | C | G | TCTATACCCACTGATCTCAAGACCACAAAGTTTTTGTTGTTGTTGTTGTTG | 0.6121 | FLJ38159-[SYTL2]-MGC34732 |
| 1737980 | rs9326283 | 11 | NC_000011.5 | 98994639 | C | A | TTCAACCCATCCTGAAATCTGCTGACTGATTAACCCACTTCAATCCAGTCT | 0.9498 | LOC390246-[CNTN5]-LOC401706 |
| 1744345 | rs11226307 | 11 | NC_000011.5 | 103746211 | G | C | GAACTTCTTTAACAGGTTCTGAGTAGAAACATAGCTCAATGAAGAAATAAA | 0.6622 | PDGFD-[ ]-CASP12P1 |
| 1749997 | rs17655140 | 11 | NC_000011.5 | 109740440 | G | T | ATATGTATGAAAATAGATTCTAAAGGTATTAGATCAGCAGTCCCCAACCTT | 0.6493 | RDX [ ]-FOX1 |
| 1751158 | rs2177745 | 11 | NC_000011.5 | 111777753 | C | T | CTGTTTCAGTTCTATGGCAGTAATTCAAGTAAGAGTGAAGACACCCATTGC | 0.8679 | LOC399951-[ ]-LOC387810 |
| 1752125 | rs605843 | 11 | NC_000011.5 | 112662883 | T | C | GGTGATCAGCATGCTGCTGGCCCTATGATGATAAGTAGTGGGCTCTTCCTT | 0.7336 | LOC387810-[NCAM1]-TTC12 |
| 1752273 | rs10891539 | 11 | NC_000011.5 | 112774141 | G | C | GTAACCCCGGGAGCTGAGTGAGAGAGGCTCCTTCCCTTACATCCACATGCC | 0.4557 | NCAM1-[TTC12] DRD2, ANKK1 |
| 1752293 | rs754672 | 11 | NC_000011.5 | 112786785 | C | T | TCCTGGGCCACTGAATTGCCAACTGCGTGACCCAAGGCTCCTCTAAACCTG | 0.4574 | TTC12 [ ] DRD2, ANKK1 |
| 1752882 | rs17626940 | 11 | NC_000011.5 | 113430360 | G | A | CACTGAGTAAGCAGGTGCCTCCAAAGGTCTTACTAAGCCACAGGTAGGAAG | 0.7852 | HTR3A [ ] ZNF145 |
| 1754883 | rs11602880 | 11 | NC_000011.5 | 115860421 | G | A | CAACCCTGGCTGACATGACTCCTTCGATTGCTAATCAGTCCTCAGTCACCC | 0.8232 | LOC283143-[ ]-MGC13125 |
| 1756543 | rs572126 | 11 | NC_000011.5 | 117896813 | G | A | ACTTTAGTACTCTGAATCTCCCGCAGTGTCCAATACTGTACTTTTTTACAT | 0.1946 | FLJ11783 [MLL] FLJ14399, |
| | | | | | | | | | LOC143941 |
| 1760288 | rs1228402 | 11 | NC_000011.5 | 121289130 | G | A | GCAGTGTGATAGCATAAGTCACTTAATCTTCACAATGCTCCTTTGATTCTC | 0.6621 | SORL1-[ ]-LOC255849 |
| 1760335 | rs481562 | 11 | NC_000011.5 | 121223675 | G | C | AGTTTTAGGAACCTAATTTTTTTCAGTGGTCAATTTTGGCTTACAAACCAG | 0.6477 | SORL1-[ ]-LOC255849 |
| 1771665 | rs16951805 | 18 | NC_000018.5 | 7448205 | C | T | GCCTTTAGAAACGCAGGTTCTGGGCCGCTCACCCCCATTTCTGGAGCTGCA | 0.9609 | LOC339291-[ ]-PTPRM |
| 1778519 | rs12327306 | 18 | NC_000018.5 | 13120681 | C | T | ACTGGGTGCAGCCAGATTCTGCTTACGTTTTGGTTGCCCCATGAAATCGCC | 0.6591 | SEC13L-[ ] LOC401892 |
| 1790542 | rs1532234 | 18 | NC_000018.5 | 25435619 | C | A | CCCAAGAAGGTATTTCTTTGTCTAGAATCACCATATAGCTTATCTTGGTCT | 0.0819 | LOC390844-[ ]-DSC3 |
| 1793653 | rs985492 | 18 | NC_000018.5 | 27563021 | G | A | TCACTATATGTTGGCCTTGATTGGTGTTCCTGAAGTCTTTTGGGCATTTCT | 0.4697 | LOC390845, LOC390846, |
| | | | | | | | | | B4GALT6 [ ] MCART2 |
| 1798659 | rs17740100 | 18 | NC_000018.5 | 32042471 | C | G | GGTTTGCCTTCTTAAATATGTTATTCTAGGTCATTGGTAACATAAGTTTTA | 0.9203 | STATIP1 [MOCOS]-RNU4P3 |
| 1800398 | rs9955296 | 18 | NC_000018.5 | 33408524 | C | A | CGTTCTCCCCTTCTTAGCTTCGACTCGCATTCTCATGAACATCTCTCCTAG | 0.2339 | BRUNOL4 [ ]-LOC388474 |
| 1801860 | rs1866524 | 18 | NC_000018.5 | 34538449 | T | C | GTCTATTCATCTTCACAGCAATCTATTTCAAAAGTGCTTATCTCTGCTGCT | 0.6251 | BRUNOL4-[ ]-LOC388474 |
| 1803884 | rs9960298 | 18 | NC_000018.5 | 35781021 | A | G | CACATATTTTCTCTTGCAAGTTCGTATGATTTGCATTATTTAAACTTGCAA | 0.9862 | LOC388474-[ ]-NPM1P1 |
| 1804744 | rs16973101 | 18 | NC_000018.5 | 36172105 | A | G | CTATTACAGGACTCCTCATAAACGCAGTGAAGCTAGAGTGAAAAATAGATA | 0.9591 | LOC388474-[ ]-NPM1P1 |
| 1811387 | rs9675482 | 18 | NC_000018.5 | 40075524 | T | C | ACTTGCTAATGAATTATAACATCAATTGTGTATCAACAAACAATATTTAAT | 0.7345 | SYT4-[ ]-LOC342732 |
| 1811405 | rs12957915 | 18 | NC_000018.5 | 40091672 | T | C | GAAGTGATAAAGTGTTGTGAGTAACCCAGCTTTCTTAGAATTGAAACAAGT | 0.7391 | SYT4-[ ]-LOC342732 |
| 1811416 | rs1456608 | 18 | NC_000018.5 | 40104086 | G | T | GTCTCAGTCCTGTTTAGGTTTATAGTTTTAATGTAGGGCAGCAGGGATCTT | 0.7247 | SYT4-[ ]-LOC342732 |
| 1812391 | rs17727782 | 18 | NC_000018.5 | 40843071 | T | C | CAATCTGTTAAGGGTAAATAAGATATAAACACGTGAAAACATAAATCACAA | 0.9769 | LOC400548-[SETBP1]- |
| | | | | | | | | | LOC400649 |
| 1812844 | rs12326596 | 18 | NC_000018.5 | 41022861 | T | A | ATTATAAACTAGGTCAATTTTCTGCTACTCCATGAGGCCTTTTTTAGAAGG | 0.69 | SETBP1-[ ]-LOC400649 |
| 1813965 | rs3760585 | 18 | NC_000018.5 | 41554413 | G | A | AGATTCACTACACACCTTCAGCCCTGACCAGTGCGACTGAGGCAGAAGGCT | 0.7989 | SLC14A2 [ ] SLC14A1 |
| 1816467 | rs16948949 | 18 | NC_000018.5 | 44014628 | A | G | TTATCTAAATCCTAGTAGAGAATCAATCTAAGTTAACCCACACTTTGGTGT | 0.9881 | LOC201501-[ ]-K1AA0427 |
| 1829453 | rs12958775 | 18 | NC_000018.5 | 55268427 | A | G | GACTGCTAGCATCTCCTTCTTGGCTAATGTTTATTTGGGCAGATGTCCTAT | 0.5852 | LMAN1-[FLJ30681]-LOC219542 |
| 1833522 | rs7234317 | 18 | NC_000018.5 | 59955049 | T | C | AACTCAACCACCACTTATTGTTTTTTGAAGATAATGTTGGTCGTGCGCCTG | 0.3892 | SERPINB8-[MGC39571]-LOC400654 |
| 1835296 | rs1942822 | 18 | NC_000018.5 | 61473241 | T | A | AGAAATTGTACGAAGTAGATGCAAATAGTTCATTAGGACTACTAAGTATGA | 0.8468 | LOC400654-[ ]-CDH7 |
| 1839162 | rs11240777 | 1 | NC_000001.5 | 838822 | G | A | TGATAGCATTTGGATTGGGCTTTAAGGTATGACTAGGAGCTTACCAGATAG | 0.7772 | LOC284591, LOC400728, |
| | | | | | | | | | FLJ22639 [LOC388579]-LOC388580 |
| 1863938 | rs11779336 | 8 | NC_000008.6 | 10711588 | C | T | AGCCAATCATTTTCCCACAAAGTTACGATACAGCATCAACCACTGGTGTCT | 0.9195 | SOX7-[PINX1] LOC389624 |
| 1868867 | rs12679640 | 8 | NC_000008.6 | 14038640 | T | C | AAATATTTTTTACACCTCATAGTCATTCAAATAGGTACACGTCCTTTTTGA | 0.9251 | FLJ25402-[SGCZ]-TUSC3 |
| 1871356 | rs6530875 | 8 | NC_000008.6 | 15353466 | T | C | GCTAAACAAAGAGACAGATACCTAATCATGGGTACTTTCCCAAGGAGAAAT | 0.4542 | SGCZ-[ ]-TUSC3 |
| 1871489 | rs17657878 | 8 | NC_000008.6 | 15454569 | T | C | TGAAGTAGGAGTCAGGAGAACTGAATGCTATTGAAATAGCTGTCACTCATA | 0.768 | SGCZ-[TUSC3]-LOC137012 |
| 1871506 | rs17657927 | 8 | NC_000008.6 | 15461887 | T | C | ACTTTGGCATGTACTAACGATACTATATTATGAGATATTTTATGTATCCTT | 0.7668 | SGCZ-[TUSC3]-LOC137012 |
| 1871599 | rs13279752 | 8 | NC_000008.6 | 15520159 | G | C | CTGTAAAGAATCTAGCCTACTAATTGTATATCACCATTAGTGAACATTTTT | 0.8129 | SGCZ-[TUSC3]-LOC137012 |
| 1871685 | rs17121887 | 8 | NC_000008.6 | 15610929 | G | T | GTCTGCTTTTGTAAGATTGTTAATTGTATAACCCCAGATTTTCAAGTGACA | 0.8203 | SGCZ-[TUSC3]-LOC137012 |
| 1872350 | rs7012344 | 8 | NC_000008.6 | 15950245 | C | T | ATGCAGGAGCAAAAGCAGCACGGTTCGAGGCAGCGGAAGCATTAGGTATCA | 0.419 | LOC392202-[ ] MSR1 |
| 1899724 | rs2589757 | 8 | NC_000008.6 | 35391245 | T | A | CGTATATTGCACACAAAAAGATCTCAATTAACAACATCTCTCGCTCTGGAT | 0.2072 | LOC389646-[ ]-UNC5D |
| 1899741 | rs2579884 | 8 | NC_000008.6 | 35398315 | C | G | CATTTTTTTTTTAAGAGGTGCTGTTGTTCATTCTCACATGAATTAGAGAGA | 0.2062 | LOC389646-[ ]-UNC5D |
| 1899780 | rs2589349 | 8 | NC_000008.6 | 35420609 | G | T | TAAATCAGTTCTTGAAAAAATAGCTTGAGGCTCATGCAGCTTTGGAAACTC | 0.205 | LOC389646-[ ] UNC5D |
| 1905516 | rs2920126 | 8 | NC_000008.6 | 41513375 | T | C | TTTCCTTTCCCTTTTTATGGTGTTTTTATGGTGGTTAAGAGCGGGAGCTTC | 0.7262 | DKFZp586M1819 [ ], ANK1, |
| | | | | | | | | | FLJ25169 |
| 1912331 | rs6982235 | 8 | NC_000008.6 | 53442022 | C | T | ACTGAGACTTTTCTTCATGCTACGGCCTAACTCGTCCCCTCTCCTTCCTAT | 0.7981 | ST18-[ ]-LOC389658 |
| 1912900 | rs6473754 | 8 | NC_000008.6 | 53797031 | C | G | CCAGTGTATATTGATCTAATGTTCACTAATACTCTTAGAAATTGATACTGA | 0.2608 | RB1CC1-[ ]-GPR7 |
| 1918804 | rs16923384 | 8 | NC_000008.6 | 59317982 | C | T | TGAAAAGAATTATTGGCCAGAGCGACCCATAGTTGTTTTTGCTAATGTGAG | 0.7225 | MGC39325-[ ]-LOC137886 |
| 1918812 | rs4413762 | 8 | NC_000008.6 | 59320126 | A | T | TCCAGGTGTTTCATCATTTGGTAGCAGATAACAATTCCAGCCTCTGCTAAC | 0.7634 | MGC39325-[ ]-LOC137886 |
| 1919024 | rs10110111 | 8 | NC_000008.6 | 59507136 | T | C | AGGAAAGCAGAATTCAACACATCTTTGAAAGAAAATCCAAATGCTGTATTA | 0.6858 | CYP7A1, LOC137885 [ ] |
| | | | | | | | | | SDCBP, NSMAF |
| 1921315 | rs2242156 | 8 | NC_000008.6 | 61151123 | C | T | GTTGGACACTAGGAACTGTTATACACAAGGGAAGTCCAGGACCTCTCTCCG | 0.6684 | TOX-[ ]-CA8 |
| 1923923 | rs1384683 | 8 | NC_000008.6 | 63219918 | C | G | ATTAACTAATACTTCATATTCTTTACGCTGAAACCTAATTTGTTTTTAATT | 0.3534 | LOC392226-[FLJ39630]-GGH |
| 1929382 | rs11781537 | 8 | NC_000008.6 | 69633631 | G | A | AAACAGCTGTATCTAATACTTTAATGGCATGTTAGAGCTAAGTAAATAGTA | 0.8355 | FLJ12987-[VEST1]-LOC389667 |
| 1933844 | rs6984682 | 8 | NC_000008.6 | 73109526 | A | G | AAGGTTTGGAATCAAGGTCATGGGTAGAGTTGGCATGATGGAAGAACGGAT | 0.8253 | TRPA1-[ ]- LOC392232 |
| 1943237 | rs1530050 | 8 | NC_000008.6 | 81945830 | T | G | GAAGGATAATTTTATTTCCAGATGATTATTTCCAGTTTTTAACGTGAAATC | 0.6467 | CKS1A-[PAG]-LOC392238 |
| 1953067 | rs17733523 | 8 | NC_000008.6 | 92846817 | C | T | TATGTTGGCTAGCTGCATGCACAGTCTTCTCTTGAAGAACGTCTACATTGG | 0.7695 | SLC26A7-[ ] MRPS16P1 |
| 1953110 | rs1838184 | 8 | NC_000008.6 | 92903068 | T | C | TAAGTTTGACAAGCACTATTTGCCATATCACATTCTTGGAAATTCACAGTG | 0.8058 | MRPS16P1 [ ] CBFA2T1 |
| 1953274 | rs17748153 | 8 | NC_000008.6 | 93241264 | C | T | GATAAAGATCTGGATGGAGAATGCCCGACATATGTGCAAATTTGTGCCAAA | 0.7306 | LOC401470-[ ]-LOC286144 |
| 1953297 | rs11775813 | 8 | NC_000008.6 | 93255461 | A | G | TAGGATCCTGCAGTTAATAATGGCCAGTGGCACTGATGGTCATTAACGTTC | 0.2657 | LOC401470-[ ]-LOC286144 |
| 1962816 | rs34179481 | 8 | NC_000008.6 | 103201536 | A | C | ATGTCCCTT1AGAGCCTCTCTGATAAGATTGCTGGAATGAATAAGCTACTG | 0.4743 | NCALD-[RRM2B] DD5 |
| 1966340 | rs10093110 | 8 | NC_000008.6 | 106521997 | G | A | ATATTACTGCCTACCCACATATTTCGTCTTCTCTATTTCTCTATAATAAAA | 0.5745 | LOC402348-[ZFPM2]-LOC346887 |
| 1966361 | rs4734877 | 8 | NC_000008.6 | 106534615 | A | T | AAGTAACACTATAAACCTTGGCGTTAGGGGATTTTTACATGCCTAAATTTG | 0.3899 | LOC402348-[ZFPM2]-LOC346887 |
| 1966373 | rs1470684 | 8 | NC_000008.6 | 106540455 | A | G | TGGTTGTAACAAATATTGTGCCAGCATTATGAGGACTTAGCATCAATCTCA | 0.41 | LOC402348-[ZFPM2]-LOC346887 |
| 1970899 | rs2703389 | 8 | NC_000008.6 | 110513466 | C | G | GCTAAGACATGAGATTCCATTATCAGTAATGTTATTCCTGTAAGTGTGTGC | 0.2073 | PKHD1L1 [EBAG9] FLJ20366 |
| 1971980 | rs11995209 | 8 | NC_000008.6 | 111664622 | T | C | TGCCTTTTGGCCCCTGTAATTTAGTTAGTCTCTGATTTTATCCTAAGAGTA | 0.2157 | LOC392262-[ ]-CSMD3 |
| 1974303 | rs1566835 | 8 | NC_000008.6 | 113975723 | A | G | AAGGCCCTGGACATCCCATGACAAAATTAGCTTAAATGTCAGGACCAGAGA | 0.4432 | LOC392262-[CSMD3]-TRPS1 |
| 1974329 | rs17660742 | 8 | NC_000008.6 | 113988522 | G | C | AAATTGATATCCTTATT1CCAGAGTGAGACTGTACAGTTATTTTCTTATAC | 0.7984 | LOC392262-[CSMD3]-TRPS1 |
| 1974401 | rs9297488 | 8 | NC_000008.6 | 114045624 | T | C | ATTTTATTGTATGGTAAAGTCAAGGTAAACAATTTATAACTATAATGTCAC | 0.4412 | LOC392262-[CSMD3]-TRPS1 |
| 1974421 | rs7840935 | 8 | NC_000008.6 | 114060938 | G | A | TATGTAGAGCTTCGAAATCAATTAAGTTCCTTACAACCTCTGACATAAAGC | 0.4442 | LOC392262-[CSMD3]-TRPS1 |
| 1974444 | rs6999828 | 8 | NC_000008.6 | 114071268 | G | C | ACTATAAAACGTTTTCATGATCCCTGTTCTCATGGAGTATTCTATTAGTGA | 0.4442 | LOC392262-[CSMD3]-TRPS1 |
| 1974476 | rs2356050 | 8 | NC_000008.6 | 114103495 | C | T | AAGTAAACCAGGTAGGGGTTTTCAACAGCCTTTATATTTACATATGTATAT | 0.443 | LOC392262-[CSMD3]-TRPS1 |
| 1974527 | rs17607120 | 8 | NC_000008.6 | 114176761 | C | T | TGATTTCAGAATGGGGTTTGGCATACGGCCAATGATTAGCTCTCTGCATAT | 0.4546 | LOC392262-[CSMD3]-TRPS1 |
| 1975561 | rs17641691 | 8 | NC_000008.6 | 115433400 | G | A | CAATTGTGTGGTATGCAATATGTTCGTAGTAAACAATTCAAGTAGCACAGA | 0.8546 | CSMD3-[ ]-TRPS1 |
| 1982441 | rs17818446 | 8 | NC_000008.6 | 120790094 | T | C | TGCTGCCCTTAGCCACTGGCATAACTGCAAGCCTCTCGGGTCATCTAGCTT | 0.911 | ENPP2-[TAF2] MGC5528 |
| 1988046 | rs17395997 | 8 | NC_000008.6 | 125889640 | T | C | TGATATTAGATGATGGTAATTTAAATTTAGAGCAATGGCTGTTCTGGGAAC | 0.3968 | MTSS1-[LOC392270]-ZNF572 |
| 1988073 | rs1427083 | 8 | NC_000008.6 | 125901530 | G | A | TTCACTTTTTGTCAGTTCATGCCCTGTGGTAGGTTCCCCATATACAATGAG | 0.3838 | LOC392270 [ ] ZNF572 |
| 1992022 | rs2648834 | 8 | NC_000008.6 | 129128085 | G | A | CTGGGGAAGCCCCAGCAGATGCCTCGATGTGAGTGTTTCTGGGATAGTACA | 0.4415 | LOC389686-[ ]-LOC401476 |
| 1992318 | rs3815871 | 8 | NC_000008.6 | 128965167 | G | C | TGCTGTGAGTAATAATGACTCTGCTGGTAATTTGTGTCCTTCTGCTTGGAA | 0.6515 | MYC-[PVT1]-LOC389686 |
| 1994975 | rs10956535 | 8 | NC_000008.6 | 131511350 | T | C | GTAAACTCTAGTTCTGTGTCATATCCGATGGGGAAAGGGTTCCACCATTAA | 0.619 | HSPC054-[ ]-ADCY8 |
| 1999459 | rs10956699 | 8 | NC_000008.6 | 134245172 | G | A | AACAAAAATGTGAGGAATTATCTTCGCAGGGAGCAGATAAACTATGCTTTC | 0.5174 | WISP1 [NDRG1] LOC392271 |
| 2001950 | rs6577889 | 8 | NC_000008.6 | 135821603 | G | A | CAACAGACAAAATGCACTAGCAACTGGAGAGGTGAGTTTTGTTAAATTACA | 0.6749 | ZNF406-[ ]-LOC286094 |
| 2001971 | rs10112307 | 8 | NC_000008.6 | 135835535 | A | G | AGGAGAGATAAATATATAAGCAACAAGTTATATTGGAAATAAAACCCATGG | 0.6905 | ZNF406-[ ]-LOC286094 |
| 2009288 | rs7295775 | 12 | NC_000012.6 | 2647666 | T | C | CACACCTGCACGCCTGAGCAGACTTTCGAGCAGGGCGCAAAGTTGGGTGGC | 0.9461 | FLJ11117-[CACNA1C] LOC283439 |
| 2009302 | rs4765968 | 12 | NC_000012.6 | 2658111 | T | C | TCCGTCTCTTCCTCATCCTTATTCTTGTCACTGGGAGACATTCGTCACTGA | 0.9504 | FLJ11117-[CACNA1C] LOC283439 |
| 2011187 | rs7980515 | 12 | NC_000012.6 | 22995946 | A | G | AAATGCAAGGAATGACAAAAGAATGAGTGCTTCATCCTAGTTTCTAGGCCT | 0.2039 | EKI1-[ ]-SOX5 |
| 2013955 | rs12230476 | 12 | NC_000012.6 | 17716456 | C | A | CATATTACCTTATTATCCTACAAAACAAAATGCATACTCAATAACATTAAT | 0.6949 | LOC390298-[ ]-FLJ22655 |
| 2016686 | rs4762694 | 12 | NC_000012.6 | 21068254 | A | G | CTGCATCCCCTCTTTTTCTCAGCAAACACTCTCAAGTCTCTCCCATCTATT | 0.3262 | SLCO1B3-[LST-3]-SLCO1B1 |
| 2020226 | rs7296211 | 12 | NC_000012.6 | 24864227 | T | A | TAAACACTTTCACCATTAATAACAGTTCATTGGCATTTTAGCAGATCTACA | 0.2869 | LOC387846 [BCAT1]-LOC196415 |
| 2021615 | rs16929857 | 12 | NC_000012.6 | 26042998 | A | G | TATGTGTGGGCGTGTATGTGTGTCCATTCATTTAAGGAAACTTGGAAAATA | 0.8188 | LOC283347-[C12orf2]-BHLHB3 |
| 2022897 | rs4304878 | 12 | NC_000012.6 | 26942203 | G | T | TTTCAATATACACCAGCTGCTAAATGTAGTTTCTATTGAATATTCAGACAG | 0.2215 | LOC341370 [ ] DKFZp564O1863, |
| | | | | | | | | | FLJ10637 |
| 2022921 | rs2029309 | 12 | NC_000012.6 | 26972833 | C | G | TAAACATGTAAACATGCTGTCTCTACTATTCTGAAAAACTACAACAGGATT | 0.2214 | LOC341370 [FLJ10637] |
| | | | | | | | | | DKFZp564O1863, TM7SF3 |
| 2022927 | rs708158 | 12 | NC_000012.6 | 26978970 | T | C | TTTTATAGTTACAGAATGTGTAGTATATGCCAGGTGCTATTGCTGGCACTG | 0.2237 | LOC341370 [FLJ10637] |
| | | | | | | | | | DKFZp564O1863, TM7SF3 |
| 2024965 | rs4362217 | 12 | NC_000012.6 | 30367273 | T | C | GGGGCAAAGATGGGGATTAGAATGATGACATTGCAAAGAAAGTATCATCAG | 0.8593 | ARG99-[ ]-IPO8 |
| 2027435 | rs7306057 | 12 | NC_000012.6 | 33030513 | C | T | TCTACAACCGGTTCAAGGGCCACAACGACCTGATGGAATACGCAAAGCACC | 0.4815 | PKP2-[LOC283343]-SYT10 |
| 2027772 | rs10506102 | 12 | NC_000012.6 | 33105304 | G | C | GTTTTGCTTTACATAGCCAAATTAAGTACCTGAAATAAAATATAGAATATA | 0.5491 | LOC283343-[ ]-SYT10 |
| 2030091 | rs11171720 | 12 | NC_000012.6 | 37996740 | C | T | TTGAGGAGAATGAGGATATGGTGAGCGTTAGCATAGAATGTAACCTGAAAC | 0.8193 | LOC121216-[KIF21A]-LOC390306 |
| 2032297 | rs1997192 | 12 | NC_000012.6 | 40025711 | T | G | TCTCAGTTGCTTTTAATCTACTTCTTTTGGGATAGAGAGAGATGAGTTGCA | 0.08 | CNTN1-[ ]-DKFZp434B0417 |
| 2034022 | rs11837562 | 12 | NC_000012.6 | 41580383 | A | T | TCCCGTCCTCCTGTGCTGTAAGCTCATCTTCATAACATAATGTTTATTTTT | 0.7507 | LOC390308-[ ]-MRPS36P5 |
| 2037647 | rs2731099 | 12 | NC_000012.6 | 47170193 | C | T | GACATAGCAGAGGGCCAGAGGGCCACAAAGAATAAAAGATGTGAGGCCTTG | 0.6249 | LOC390312, ANP32D [MGC35033] |
| | | | | | | | | | LOC341568 |
| 2038957 | rs4761981 | 12 | NC_000012.6 | 49960768 | T | C | CTTTGTATCCTTTCAGTTTCATATCTTCCTCTCTTGTGCTCAGACCCTTAC | 0.1545 | DAZAP2, LOC57228 [ ] ELA1, |
| | | | | | | | | | BIN2 |
| 2039518 | rs17126450 | 12 | NC_000012.6 | 50894979 | C | T | TGGGATGGAGGAATGAATGAAGAAACGGTGAGGCTGGGTGAGGTGGCTCAC | 0.7735 | LOC144501, LOC283403 |
| | | | | | | | | | [LOC4017219 KRT7, KRTHB@ |
| 2039526 | rs7314387 | 12 | NC_000012.6 | 50900229 | T | A | TAAAGTACCAAATTTCATGAATAACTTCTGCAAAGGAGAACCATTTATTTA | 0.7822 | LOC144501, LOC283403 |
| | | | | | | | | | [LOC401721] KRT7, KRTHB@ |
| 2040404 | rs1894033 | 12 | NC_000012.6 | 50981897 | T | C | AGAGGTGCAAGTAGTGAACGCCTGACGCCCCGACCACTGTGCTCTCCATTC | 0.148 | KRTHB@, LOC387859 [ ] KRTHB@ |
| 2041414 | rs7297602 | 12 | NC_000012.6 | 52359171 | T | G | AGGTCACCAAGGAAGGGTTTTGATTTGTTTATATATGTTTCTTTGGACAAG | 0.5091 | ATP5G2, ATF7, LOC341412 [ ] |
| | | | | | | | | | KIAA1536 |
| 2041419 | rs11611695 | 12 | NC_000012.6 | 52340028 | T | G | GGAAGAATTAACCAAAAGCCATTCATGAGTGACTGGAACCCAGGATTCTGG | 0.849 | LOC341412 [ATF7] ATP5G2 |
| 2048484 | rs10877864 | 12 | NC_000012.6 | 61179466 | T | C | GTAATTTAACTGAACTAGTCTCCTATTGGACACTTGCATTCTGTTCAGTGT | 0.8421 | USP15-[KIAA1040]-FLJ25590 |
| 2049385 | rs7973936 | 12 | NC_000012.6 | 62619912 | G | A | ATGGAGAGATTGAACCTAAAGATCTGTAAAGTCTCTGCAACTCTCAAGTCC | 0.6824 | LOC341315-[SRGAP1]-FLJ32549 |
| 2054953 | rs7135345 | 12 | NC_000012.6 | 68573193 | G | A | CATATTATTAAAACTCTTGATGTAGGCCACCAGTTGCTTTCTAGAAAGATT | 0.8842 | RAB3IP-[ ]-FLJ25056 |
| 2056311 | rs11178491 | 12 | NC_000012.6 | 69603195 | C | G | GTTAGTTCAGCATGGTAAAAATAACCCTGAGTGGGCTGTAATTTTGGTGAC | 0.7763 | PTPRR-[ ]-TM4SF3 |
| 2056363 | rs17814619 | 12 | NC_000012.6 | 69659996 | A | G | ATGTTTATGAAAGCACATACAGGCAATAACAGTGCTAACCTTCAGAGGTTA | 0.7863 | PTPRR-[ ]-TM4SF3 |
| 2060561 | rs17112159 | 12 | NC_000012.6 | 71903355 | T | C | CCTTCCCTGGCTGTATCTGCCTCCTTGCCACAGGTTATCATTATTCTAAAT | 0.9729 | TRHDE-[ ]-VENTX2P3 |
| 2063822 | rs10778227 | 12 | NC_000012.6 | 77734860 | A | G | TAAATAGAATAACATGGATATCAAGAGGTTCTTAATTTTCATATTTCAGAT | 0.4645 | LOC390347 [ ]-SYT1 |
| 2083902 | rs7972172 | 12 | NC_000012.6 | 103076762 | T | C | GAGAAGCTGTCCTTAAATGGGGGTGTCCGGGAGTCCTGACGAAAGATGAGC | 0.2649 | NFYB [ ]-LOC400068 |
| 2084108 | rs4964287 | 12 | NC_000012.6 | 103212026 | C | T | GATTTCTCAATGTTGTTGTAGGTCTCGGAGGAACATGTGTGAATGTGGGTT | 0.6959 | LOC400068 [TXNRD1]-CHST11 |
| 2097268 | rs1023229 | 13 | NC_000013.6 | 18068925 | G | A | GTCCAAACATACCGATACAAAAACAGTTACATTATTTCATCTCATACTGGT | 0.1527 | HSMPP8 [PSPC1]-LOC390377 |
| 2099207 | rs7325032 | 13 | NC_000013.6 | 20545054 | C | A | AATAAATGACTTTGCACATAATTATCCTTGGTTTACTTCTTCTCAGCTGCA | 0.5647 | FGF9-[ ]-LOC387908 |
| 2116144 | rs9532890 | 13 | NC_000013.6 | 39914031 | G | A | TTCACTCTTTTGGAAGTGTTTCTTCGTGCATGCCCCCACTTAAAAAAAAAC | 0.395 | RGC32 [ ] KIAA0564 |
| 2118459 | rs35665055 | 13 | NC_000013.6 | 42892501 | T | C | TGCTGAGGGTACACATATGACTAGATACAAAGCTGTCCACTTGATCTACTA | 0.8688 | LOC400128-[TSC22]-LOC400129 |
| 2118498 | rs9533910 | 13 | NC_000013.6 | 42935937 | C | A | AAGAAACAATTTGTAAAACTGTCTCGGCGAATTCACTTAACCAAAACTTGT | 0.8718 | LOC400128-[TSC22] LOC400129 |
| 2118500 | rs9525983 | 13 | NC_000013.6 | 42946257 | C | A | TGCTGTTGTACATACTCTGAAGCAGGATTTTGAGTCACTGATTTGACATGG | 0.8716 | LOC400128-[TSC22] LOC400129 |
| 2128119 | rs17623690 | 13 | NC_000013.6 | 57602026 | A | C | TTCATAATCAGTAATTGTTTCTCAGACGTGCTTGATTCTGGAATCTCTTTG | 0.7394 | LOC341689-[ ]-DIAPH3 |
| 2129919 | rs9285228 | 13 | NC_000013.6 | 59162157 | T | C | TCAGTCACCAGCATGCCAAAACCATTCTTAGGAATTGCCCAACAGACCCTG | 0.7155 | LOC390407-[ ]-LOC387932 |
| 2129930 | rs9538922 | 13 | NC_000013.6 | 59164839 | T | C | ACCAGCTATGCTACCATTTCTACTCTGTTTATGCCTACTAAGAGGGCTTGA | 0.7194 | LOC390407-[ ]-LOC387932 |
| 2138144 | rs17084398 | 13 | NC_000013.6 | 67326957 | C | A | GATCTTTAGTACACAGTCTATTAATCCACTCAAAACCCCATGATTATCCTA | 0.7108 | LOC338862-[ ] LOC387906 |
| 2140871 | rs1543663 | 13 | NC_000013.6 | 69899703 | G | C | CCCTGAAAAGTTGGTATTTTCTTCCGTATCGGTCCTTCATCATTGAACATG | 0.0303 | KLHL1-[DACH]-FLJ22624 |
| 2142435 | rs9318170 | 13 | NC_000013.6 | 71738727 | C | G | CATTCACCTATTGTTCCTTGGAGACCAGTGACATTGATAGGGAAAATACTC | 0.0188 | LOC387934-[ ]-LOC122134 |
| 2147441 | rs9530848 | 13 | NC_000013.6 | 77397109 | C | T | AGCAAAAGGCAAAATCGACTAAGTGCCTTGCCTTCCTCTTAAGTTGGTAGA | 0.7698 | LOC390415-[ ]-C13orf10 |
| 2147936 | rs3861143 | 13 | NC_000013.6 | 78288603 | G | A | TATGCCACCTTCAAAAGCCTACCTCGTAAAAATTCTTCCTCTTTAGCCTGC | 0.818 | NDFIP2-[ ]-SPRY2 |
| 2147954 | rs3966634 | 13 | NC_000013.6 | 78381457 | A | G | TATAATGATAGTGATTGTTTCCTATATATCACAATTTGGAGATAAATAATC | 0.8333 | NDFIP2-[ ]-SPRY2 |
| 2153874 | rs9531941 | 13 | NC_000013.6 | 84511763 | T | C | ACTGTAATCATTCCAGTGAACAGTGTGGGTTTTTCTCAGAGACTGATGGGC | 0.2623 | SLITRK6-[ ]-LOC390417 |
| 2159712 | rs9516282 | 13 | NC_000013.6 | 92098288 | C | T | AATGTTTTAAAGTACAGTATTTGCTCGATGAAAATATGATGTTGTGACTTG | 0.655 | LOC400151-]GPC6]-DCT |
| 2163321 | rs17475472 | 13 | NC_000013.6 | 98981498 | A | G | GTAAATGCAAGAAAGTGAGAGTATCAGTTATGAAAGGCACCTTTAATACCA | 0.7929 | LOC400156-[FLJ14624]-LOC390423 |
| 2163423 | rs16957615 | 13 | NC_000013.6 | 99075795 | G | C | AGAAGGACAGGAATTTCCACCAGCAGAGCCCCTTAAGAAGGTTATCTGGGA | 0.9268 | FLJ14624-[ ]-LOC390423 |
| 2168345 | rs9519072 | 13 | NC_000013.6 | 101865262 | C | T | CTTATCATCCTTAGCATTTTTTAGACGGCCAGATTTGGAGTTTAATACTAG | 0.1917 | SLC10A2-[ ]-G30 |
| 2170305 | rs4771480 | 13 | NC_000013.6 | 102920777 | A | G | TATTCTCAACAGATCTCTCCTGACCATGCCGTTCATTGCCCTTGCTGTCTT | 0.7975 | SLC10A2-[ ]-G30 |
| 2172827 | rs9520083 | 13 | NC_000013.6 | 104818492 | G | A | AAAAGGCAGAGATTACAACCGAAGTGTTTTCCTTGACCCGGGAAGTGATCG | 0.3643 | LOC341604 [ ] EFNB2 |
| 2199587 | rs12355804 | 10 | NC_000010.5 | 3489854 | C | T | TAAATACTGTCTCAAGGTTGGTTAGCGCTTTCAGATGAAGCATCTCATTTG | 0.9184 | PITRM1-[ ] LOC387631 |
| 2201976 | rs1132293 | 10 | NC_000010.5 | 5685044 | C | T | CAGAACTGGTAACTAAGGCGGTGATCAAACAGGAATGCTTTTCTTCTCAGT | 0.6946 | CALML3-[ASB13] LOC399712 |
| 2203902 | rs2497469 | 10 | NC_000010.5 | 7165641 | C | G | AGACCTTGAGAAACGCTTCCAAAAACTGCAGGAATGATTTCTGAAGGCTGC | 0.7852 | PRKCQ-[ ] SFMBT2 |
| 2210865 | rs1556718 | 10 | NC_000010.5 | 13330966 | C | A | TAAGCTAATCATACCTCCCACTCTGCATCTGAGCAGGGTATCTGAGACTCC | 0.7265 | LOC221044-[PHYH] SEPHS1 |
| 2212327 | rs1041337 | 10 | NC_000010.5 | 14354736 | A | C | ATCACTATTTGTTTGAAAACAATGTCCTGATACCCTGCTGTCTTCCCAGTA | 0.4528 | PRPF18-[FRMD4]-C10orf45 |
| 2214222 | rs10737009 | 10 | NC_000010.5 | 16090179 | A | G | CCCTGATGACCTGGTCAATAGAAACAAATGAACACGTGACTTGCATTTGAG | 0.2466 | FLJ13397-[ ]-PTER |
| 2222231 | rs17440393 | 10 | NC_000010.5 | 23241574 | G | A | AAGACCATCAACATCAGACAATAAAGGGCAGTGACCACTGAGAGACGGGAA | 0.7748 | PIP5K2A-[ARMC3]-MSRB |
| 2223295 | rs10764444 | 10 | NC_000010.5 | 24304727 | G | A | GTTGGGTTGTTCCCGTGGGTTGACAGTCAGCATGTTTTTATTTATTAAATG | 0.9209 | LOC220213-[ ]-KIAA1217 |
| 2228591 | rs2250755 | 10 | NC_000010.5 | 29293136 | G | A | ACTTTGAGGCAGTAAATGTGAGGCAGTGACATACATACCTCCTTGTAATAG | 0.5157 | LOC256457-[ ]-MGC33408 |
| 2236226 | rs1192668 | 10 | NC_000010.5 | 37150953 | T | C | GCTGAGTTTGAGACTATGTTACAAATGTTTATCCTCAATGACATATGAGAA | 0.1346 | LOC387651-[ ]-LOC389948 |
| 2244319 | rs10826075 | 10 | NC_000010.5 | 50871973 | C | G | AGAAAGAGAGTATTTGGTTTTCCAGCCTCTTCAGGAGGAGGTTACTGGGGA | 0.7651 | LOC255319 [ ] MSMB, NCOA4 |
| 2246661 | rs293267 | 10 | NC_000010.5 | 52556116 | G | A | TGTGGATTAACTGTGCTCACAATAAGTATTTGTATCATCAATTGCATGACT | 0.6759 | ACF-[PRKG1]-CSTF2T |
| 2254679 | rs7913866 | 10 | NC_000010.5 | 58867791 | C | T | GATTTTGTAAGAAGCAAAAGTAATATGAGAACATGATCAGCTGATGGCTTA | 0.8559 | ZWINT-[ ]-MRPS35P3 |
| 2257769 | rs2127355 | 10 | NC_000010.5 | 61878819 | A | G | GATCCTCATTAGTCCCAAGGTTGACATATGAATCTCTTCTCCCTACACCAT | 0.5225 | LOC387684-[ ] CDC2 |
| 2267426 | rs1015193 | 10 | NC_000010.5 | 72929029 | T | C | CAAGAATATTCAATGGCCAACATATTTTTAGGCAAAAAATGCAAACTACAA | 0.5149 | CDH23 [PSAP]-CHST3 |
| 2274606 | rs4933449 | 10 | NC_000010.5 | 89051019 | G | T | TGGAAAACATTAGAAGAGGGTTCCTGTGTCATAGTTTCAAAATTCTTTTCA | 0.6675 | MINPP1-[ ] PAPSS2 |
| 2275497 | rs10788549 | 10 | NC_000010.5 | 81722565 | G | A | CCTTTCACCTGAGCAAGGCATAGACGATTCATTCTAAAAAGTGACACTGGG | 0.6063 | MAT1A, LOC143243 [ ] |
| | | | | | | | | | LOC143241, MGC16186 |
| 2287160 | rs7910115 | 10 | NC_000010.5 | 92858313 | G | A | CACTAAAGATAACTTTATAGAGAGGGGACTTTCAAATATTTCTATAACTAG | 0.9659 | MGC16202-[FLJ37306]-LOC387702 |
| 2298521 | rs11192851 | 10 | NC_000010.5 | 107790907 | A | C | AATTAGGAAAGGAAGAGGTCAATTAAAATTACTAAGTAAGGCATTAAGACA | 0.5947 | LOC86123-[ ]-SORCS1 |
| 2311188 | rs1937819 | 10 | NC_000010.5 | 122173898 | A | G | AGAATACACTTTTTATTTCAAAGACAAGTAAGGCAACACCCTCCTCCCAGC | 0.7381 | LOC196051-[ ]-WDR11 |
| 2312380 | rs10887014 | 10 | NC_000010.5 | 123316424 | T | C | CAACTGGCAGGTTAATCCCTGTTAATGAGCCCCAAATTTAATTATCTTACC | 0.8347 | FGFR2-[ATE1]-FLJ20003 |
| 2314337 | rs841009 | 10 | NC_000010.5 | 124934127 | C | T | TCAAGGAAGGTGTTTCAAACCAAGACTGATCAAGCAGCAGGAACTGCAAGG | 0.8254 | LOC390009-[ ]-GPR26 |
| 2316249 | rs11244755 | 10 | NC_000010.5 | 127261417 | C | T | TCATAGGTTGAGACTAAGCCATCAACTCCAAGTTTGAAAAGAAAAATAGCA | 0.6858 | DHX32-[FANK1] ADAM12 |
| 2317228 | rs10901612 | 10 | NC_000010.5 | 127719499 | C | T | TGTGAACTTGTAAGGATATTTGTTTCGGGCAAACACCACAGTTGAATGATA | 0.6154 | ADAM12-[FLJ32938]-DOCK1 |
| 2321879 | rs9831753 | 3 | NC_000003.6 | 1851192 | C | G | AACTACTCACCTATTATCCATAAAGCGCCCATGTATTCTACTTCTAATTGT | 0.5918 | LOC391504-[ ]-LOC391505 |
| 2324234 | rs1705820 | 3 | NC_000003.6 | 3201143 | C | A | TAAATCAGGGTAATTCTCAAGTTCCCAGTCTACAGAGGCTACAAACAGTTT | 0.2142 | TRNT1, LOC51185 [ ]-LRRN1 |
| 2328162 | rs17215933 | 3 | NC_000003.6 | 6174813 | G | A | GACATAGAGAAATGTTCAAGAGCTCGTTTGGAAAAGGAGATAGATAGTTCA | 0.9414 | MRPS35P1-[ ]-MRPS36P1 |
| 2328415 | rs155420 | 3 | NC_000003.6 | 6434012 | C | T | ATGGAGTTGGAAGCCCACAGTATTACGGCCTTGCAGTTCTAAGAGCGGGCC | 0.6342 | MRPS3SP1-[ ]-MRPS36P1 |
| 2338982 | rs1449533 | 3 | NC_000003.6 | 16754279 | T | G | GTGGTGGGTGGCGGATGGGACAGATTACTTAGACAGGGTTGCTGTAGACTT | 0.2189 | DAZL-[ ]-PLCL2 |
| 2340118 | rs17503281 | 3 | NC_000003.6 | 18617651 | C | A | CTCATGCTTGAGTTTTAATGGTGGACAAATTATGGGAGGGACATTTCCCCA | 0.8848 | LOC131185-[ ]-KCNH8 |
| 2340592 | rs2948099 | 3 | NC_000003.6 | 20027872 | G | T | AAAGCACCGCAACAGTGTGTTTTGATCTGAAGTCAATTACTTGTATCAGAA | 0.1734 | RAB5A [FLJ25449] PCAF |
| 2342573 | rs11914361 | 3 | NC_000003.6 | 22051102 | T | C | TAGATGTTGGAGCTATCAGATAACCTCATAATTGGTATAATAAAATGTTAA | 0.8106 | FLJ22419-[ ]-LOC389099 |
| 2343282 | rs978397 | 3 | NC_000003.6 | 22742532 | A | G | ACCAGCAAGTCCCCCTAACAAGGCCAACAGGTATTTGCCATAAGAACAGAC | 0.9874 | HMG1L5-[ ]-LOC391519 |
| 2345656 | rs2036429 | 3 | NC_000003.6 | 26506748 | T | C | TGATCATACCCAGTACTCGCTGGTTTTTATTTAATGTAGCTTTGAAAAAAA | 0.6354 | VENTX2P4-[ ]-LRP15 |
| 2345857 | rs4973783 | 3 | NC_000003.6 | 26526758 | A | T | CTCTGAGGCCAGGGCCAGGCCTGTGTCCCAGTGACCAGGTTCAGACTTTAG | 0.3611 | VENTX2P4-[ ]-LRP15 |
| 2349785 | rs17350388 | 3 | NC_000003.6 | 30804527 | A | G | GGCACGTATGCTCACAAAGTACCAGATGTTCTACATATGCCATTCTCTGAA | 0.7017 | TGFBR2-[LOC339896]-LOC339897 |
| 2351093 | rs6801121 | 3 | NC_000003.6 | 33507540 | C | T | AAGTTCCAGTGACATGCCCTAATGCCTCTTGCCATTTTATACATCGCTGCC | 0.6134 | UBP1-[ ] CLASP2 |
| 2354274 | rs17733640 | 3 | NC_000003.6 | 38407367 | A | G | TAATGACTGTATAATATTCTATCTGGGTAGACCAGGCACGGTGGCTCATAC | 0.5421 | SLC22A14-[XYLB] ACVR2B |
| 2355020 | rs1799423 | 3 | NC_000003.6 | 40166270 | G | A | AGCTACCCAAGTACCTGTCAGCTCTGTTTTCAATCTTACACTTTTCTTTCC | 0.5094 | MOBP-[MYRIP]-GC20 |
| 2358053 | rs11130066 | 3 | NC_000003.6 | 45489589 | C | T | GTGTAATACCCTTAGCTTTATATCTCTCAGTTTTCACACAATGTGTTGTAT | 0.3828 | RIS1-[LARS2]-LIMD1 |
| 2358096 | rs2578670 | 3 | NC_000003.6 | 45534058 | G | T | TTAAATTTTTATTTGCATATTTGTTTTCTATCCTAATTCCCTACTGATCTT | 0.6125 | RIS1-[LARS2]-LIMD1 |
| 2362171 | rs7426795 | 3 | NC_000003.6 | 54370745 | T | C | CTGTGTTTCTCCAAAGAAATGATCACCTTGATAGTTGGTGATTTATATGTG | 0.2785 | LOC266954-[CACNA2D3]-HT017 |
| 2362180 | rs7653648 | 3 | NC_000003.6 | 54364550 | G | C | CTGAGGACCCTGAAGCCACCAGTGTCGTATTAATCGAAACCAGGGACTTGT | 0.2975 | LOC266954-[CACNA2D3]-HT017 |
| 2368729 | rs9857754 | 3 | NC_000003.6 | 60718143 | A | C | AGACCAAATTCATGACATTGTTTCAACCAGTGCAGCTCCTTTCATATTAGT | 0.9087 | LOC391540, LOC391541 [ ]- |
| | | | | | | | | | PTPRG |
| 2369244 | rs9683298 | 3 | NC_000003.6 | 61224786 | C | T | GGGCAGTTGGTTATTACACAGAAAACATGTGCAACACTTAATTGACTCAAA | 0.9343 | LOC391541-[ ]-PTPRG |
| 2370749 | rs6807315 | 3 | NC_000003.6 | 62925331 | G | A | ACACCTTTTATTCATGCAGAGAATCGTGTTCCTCTATCAAAGACCCACAAC | 0.1915 | LOC389127-[ ]-LOC132205 |
| 2372263 | rs1561988 | 3 | NC_000003.6 | 64500596 | A | G | GTGCAGGCTGAGTCAAACTGCTGTCATCACCAGAAGCCTCCATCGGAAGGC | 0.7745 | PRICKLE2-[ADAMTS9]-BAIAP1 |
| 2378362 | rs1002200 | 3 | NC_000003.6 | 70792242 | A | T | TCATTCACAGATTTATTTATGGCCAAGCCACACACCTTTGTGTTCAACTTC | 0.5787 | LOC401072-[ ]-FOXP1 |
| 2379489 | rs12486652 | 3 | NC_000003.6 | 72615814 | A | G | TCAGGGCATGGTTAGTTTCAAATCAATTAAGCTCCTTATGACTTTATGATT | 0.4491 | LOC339875 [ ]-FLJ10539 |
| 2386150 | rs9866565 | 3 | NC_000003.6 | 79916897 | A | G | AGTGGTATATAAAACACAGTTGTTGACCACAATATAACTAAGTTACAGAGC | 0.4273 | ROBO1-[ ]-LOC391554 |
| 2386620 | rs3773220 | 3 | NC_000003.6 | 78622704 | C | T | CTCTGCATTAAAATAATAATCATGGCGAGCAACAGATAAAATAATGTTAAA | 0.7214 | MRPS17P3-[ROBO1]-LOC391554 |
| 2386633 | rs6788434 | 3 | NC_000003.6 | 78654588 | G | A | GTATTATACTTCAGTTTACGTAATCGGGAAAATAAGAGTGGTCTAGAGAAA | 0.7493 | MRPS17P3-[ROBO1]-LOC391554 |
| 2386656 | rs17016466 | 3 | NC_000003.6 | 78675379 | A | G | AAACAGTAACAACAACTGTATTTGCATAAGCACCCCATAATCCACACCCAC | 0.7495 | MRPS17P3-[ROBO1]-LOC391554 |
| 2386667 | rs3773240 | 3 | NC_000003.6 | 78703985 | C | T | CTGCTTTCTATGCTGGGGTGGCAACCTAATCCAAAATTCCTATTGCAGGTT | 0.7581 | MRPS17P3-[ROBO1]-LOC391554 |
| 2386700 | rs35077320 | 3 | NC_000003.6 | 78735232 | G | A | CCTTCTCTCGAAGTTTCTATATGCAGATCATGACTGAATATTGTTGTTTAA | 0.7103 | MRPS17P3-[ROBO1]-LOC391554 |
| 2387552 | rs6551427 | 3 | NC_000003.6 | 87126154 | C | G | CCATAGTGGTTAGTTCTACATTCAGCGAGTGGCTTAAAATTTATGCCAATG | 0.3167 | LOC285232-[ ]-DKFZP564O123 |
| 2392172 | rs10511181 | 3 | NC_000003.6 | 102010763 | T | G | CAAAGAGTTCTTAAAGCTGCTTTCTTGGTAATTATAACTGTTTCAAGGAAT | 0.8667 | TFG-[TARSH]-IMPG2 |
| 2392173 | rs2595894 | 3 | NC_000003.6 | 101998783 | G | C | AAGAACAACCATCTTGTTGCTCCAGGGCATAGGTGAAGATCCGCTGCAGCC | 0.1317 | TFG-[TARSH]-IMPG2 |
| 2393730 | rs9851200 | 3 | NC_000003.6 | 103893209 | T | G | TGAGATTCTTCCCTGCCTTCTTCCATAAATTCATTATATTCTAGCCCTAAT | 0.3579 | LOC131368-[LOC391561]-LOC391562 |
| 2393802 | rs345585 | 3 | NC_000003.6 | 103944914 | G | T | CTAGATAACACACAGCTACTAAATGGTAGGTCAAACTTTTTGATCTATTTA | 0.7189 | LOC131368-[LOC391561]-LOC391562 |
| 2393855 | rs344675 | 3 | NC_000003.6 | 103994849 | G | A | CCTTACATGTATTTCTTTAAATGGCGTAACTCCACCAAAGATGGTTTTGAT | 0.3051 | LOC131368-[LOC391561]-LOC391562 |
| 2393869 | rs344668 | 3 | NC_000003.6 | 104028539 | A | G | TTCTAAAAAATCACAAGCTGTGACAATCTTGACTATAAGTATTAAATTCAA | 0.3142 | LOC131368-[LOC391561]-LOC391562 |
| 2393872 | rs1692458 | 3 | NC_000003.6 | 104034085 | A | G | TTTCTACTTTTTCAACATCAGAAGGATAGATGTAGGACCTGCTGCTTTTTG | 0.3152 | LOC131368-[LOC391561]-LOC391562 |
| 2394342 | rs721778 | 3 | NC_000003.6 | 104592350 | T | C | CCCTTATTCTGTCTTCACGGAAAATTATTATGCATCCCTCAGTTAATACTG | 0.8029 | LOC391561-[ ]-LOC391562 |
| 2399557 | rs16856690 | 3 | NC_000003.6 | 111438775 | A | G | TGTAGAGGCCAGGAAAAGCTTGCTGAATTTAAAAGTCAAGTTTATTCCCCC | 0.9763 | LOC401083-[ ]-LOC389141 |
| 2402034 | rs16860899 | 3 | NC_000003.6 | 114438078 | A | G | AGGCCCTAACTCTACTTGAAGGTAGATTCATGGAGGGCTATGCAGAGATGA | 0.8125 | LOC402136 [FLJ11142] LOC152185 |
| 2402124 | rs2399481 | 3 | NC_000003.6 | 114555885 | G | C | GAAACTTACAGCATATTAACAGACAGTTATCAATGACAAAAACATTAAAAT | 0.8033 | LOC152185 [ ] FLJ20174 |
| 2402372 | rs324553 | 3 | NC_000003.6 | 115047652 | C | T | AAAAGATTAGTTTACTTAGTCTCTACAGGGTCTATTGTAGCCCTCTATTAT | 0.7178 | MGC42530 [KIAA1407] QTRTD1 |
| 2402892 | rs4399857 | 3 | NC_000003.6 | 115647494 | T | C | AAAATAATTTTTCCAAAGAGTTGATTATCTTAGACTAAACTAATGACTCAA | 0.7614 | FLJ39873-[ZNF288]-LOC344811 |
| 2405824 | rs10934442 | 3 | NC_000003.6 | 119230806 | C | G | TTCCCACAGTGGGCCCTGCAGAACCCTCAGATACGAAAAGTCAGCCCTTCG | 0.8361 | LOC285194-[LOC389142·-IGSF11 |
| 2410695 | rs2279988 | 9 | NC_000009.6 | 1032166 | G | T | AAGCAGCTGGGATCTAAGTTCATTTGCATCAGGGCCACTCCAGCTCAGTTC | 0.3248 | LOC401490 [LOC389702] DMRT2 |
| 2415217 | rs16926462 | 9 | NC_000009.6 | 7591812 | G | A | TTCACCCACAAGCTCCTGTTAAAATGCCCTCTTGTTAGACTCCATACCTCT | 0.8214 | LOC392285 [ ]-MGC4730 |
| 2420701 | rs1953021 | 9 | NC_000009.6 | 12904396 | G | T | TGAAATTAAATTATTTTTCTAGTTTGTAGAGGCTATATCCATGACCATTAT | 0.3172 | LOC286343-[ ]-TDPX2 |
| 2425790 | rs10963486 | 9 | NC_000009.6 | 18156934 | T | C | AGGACCTATTGGCGTGAATTAACTTTTGCATTGCGTTAGAAAAACTTTCTA | 0.7352 | SH3GL2-[ ]-ADAMTSL1 |
| 2426484 | rs7862714 | 9 | NC_000009.6 | 18588768 | G | C | GGCCAAGAGCTGGTGGCTGACTAGTGCTGTGTGAGCATGGGTTTATAAATG | 0.3772 | SH3GL2-[ADAMTSL1]-FLJ35283 |
| 2431351 | rs4977693 | 9 | NC_000009.6 | 26103846 | G | C | ATGAATAAGCTATTAACTAAACTGAGTGTGAGTATAGCAATGTATAATCTC | 0.6688 | LOC389708-[ ]-C9orf82 |
| 2437121 | rs831274 | 9 | NC_000009.6 | 33273286 | G | C | TCCTCCTCTCCTTCTCTCAACTCTCGCTGGCCTAGATGTCTCTCACATAGT | 0.1601 | BAG1, SPINK4 [C9orf83] NFX1 |
| 2437138 | rs706115 | 9 | NC_000009.6 | 33243605 | C | G | CTACCTCTACTACCTTGTCTGAGGTCTTAACCTGCCCAGTCCTGAATTCCT | 0.8381 | SPINK4 [ ] BAG1, C9orf83, NFX1 |
| 2446882 | rs17084935 | 9 | NC_000009.6 | 67806625 | T | A | TCTGGCCTATCTCATTTCCAAAAGTTGTGAGACCTGATATCGTACCTATAG | 0.8677 | APBA1-[LOC375743]-LOC138255 |
| 2448996 | rs7045535 | 9 | NC_000009.6 | 70667362 | T | C | TAGACCCCGACCAGCCTACATATTGTTTGCTCTAAGGTCTAGTGCCCTCTC | 0.7663 | ZNF216-[TMC1]-ALDH1A1 |
| 2450638 | rs10781268 | 9 | NC_000009.6 | 73266161 | C | A | TTGAGATATAAACTAAAATTCCCTCCCTATAGGTTTTGTATCTCTTCGTCA | 0.4729 | OSTF1 [ ]-LOC138932 |
| 2451656 | rs10869756 | 9 | NC_000009.6 | 74416460 | T | C | GTTCCCAGTCTGTGCCCATTCACAATTGACTCCAGCACATGAAAGAATTAG | 0.7672 | PCSK5-[ ] FLJ11149 |
| 2451662 | rs10869758 | 9 | NC_000009.6 | 74422495 | C | T | AAACTGTCTATTCATGATTTATGCACTGAGTATTGCAGTCCTCCAAACACT | 0.7741 | PCSK5-[ ] FLJ11149, LOC158473 |
| 2468866 | rs7029471 | 9 | NC_000009.6 | 101782027 | A | G | GTCCTTCCATTAGGAGGTCTGCAGAACAGAGATAAAAATGGAAGCCAGAGA | 0.2522 | LOC340511-[ ]-SMC2L1 |
| 2469246 | rs16923908 | 9 | NC_000009.6 | 102489899 | T | C | AGGCAATTCAGACAAAAAGTTGTGATAGCATAGAAAGCTCACTGGACAGAC | 0.983 | LOC347281-[ ]-LOC138805 |
| 2469920 | rs10761084 | 9 | NC_000009.6 | 102910990 | G | C | CTATTTTCCTCCCATAGATAATTTTGCTCATCGAGCTGAAGTTCGGAAAGC | 0.8024 | NIPSNAP3A, LOC402374 |
| | | | | | | | | | [NIPSNAP3B]ABCA1 |
| 2473163 | rs7850283 | 9 | NC_000009.6 | 107203002 | G | A | ATTTTAGGATGTTGGCTATAAACACGAATCCTTCCTCTATTTGCATGCTGA | 0.5256 | CTNNAL1 [C9orf5]-C9orf4 |
| 2473181 | rs7049112 | 9 | NC_000009.6 | 107225341 | C | G | GGAGGAAAAGATGAACCAACTCCCACAAAGACTGAATAGAAAAAGACTCAC | 0.5306 | CTNNAL1-[C9orf5]-C9orf4 |
| 2473479 | rs10512394 | 9 | NC_000009.6 | 107609061 | A | G | TCCAGGAAGAGAAGCACTTGGTCTCATATCATTATATTTAAGGAGCTCCAA | 0.6572 | PTPN3 [ ]-LOC402375 |
| 2474217 | rs1410051 | 9 | NC_000009.6 | 108397190 | T | C | AAAGATGACAGTGGCAATCAACATATGAGGAAAAAACACTGAGAGTCAATA | 0.7447 | LOC401546 [TXN] LOC255220 |
| 2475055 | rs947509 | 9 | NC_000009.6 | 128426973 | T | C | TGCATCCAGTGCAGCCCAAGAGGCGTCATGCATTCTCTTCCCCTTGTTTGC | 0.463 | FREQ-[LOC392395]-DKFZp434P0216 |
| 2478857 | rs12950376 | 17 | NC_000017.6 | 25687136 | A | T | CCAGACTACACAAGCACCCTGAGAAATGTGAAACCTCTCCCTTGGCCCCCT | 0.8119 | LOC388362-[ ]-LOC401876 |
| 2478983 | rs16965173 | 17 | NC_000017.6 | 25703114 | A | G | GATATCTGAAAACAATTTGTGAACTATAAAGCATTGTTAAAATAAAAGCCA | 0.6963 | LOC388362-[ ] LOC401876 |
| 2486070 | rs8077653 | 17 | NC_000017.6 | 62482336 | T | G | GCCCCCTTTCTGCCTTTGACCCTCATGGTGGCTTTGAGCAGGCATCTGAAT | 0.3286 | GH1, GH2, CSHL1, CSH1 [CD79B] |
| | | | | | | | | | SCN4A |
| 2488104 | rs1558875 | 17 | NC_000017.6 | 66941539 | T | C | TTGTTTCCTCCTTGGTCTTCAGTTTTCTCACTTGACAGTTGAGGGGTTGGA | 0.4765 | SLC16A6-[KIAA1001]-FLJ10055 |
| 2488188 | rs2909218 | 17 | NC_000017.6 | 67061669 | T | C | TGTGTCAGTTGGTAAAGAACTGGAACGAAATGTCCTGGTTTGACGTTTTGA | 0.7966 | KIAA1001, FLJ10055 [ ] PRKAR1A |
| 2490021 | rs9907514 | 17 | NC_000017.6 | 69328342 | G | A | AATCAAATCCATTTGCATGCCGCTAGTATTCTGTGGATTCTTATTATAACT | 0.4729 | LOC401887-[ ]-LOC124685 |
| 2492846 | rs9912666 | 17 | NC_000017.6 | 71624666 | G | A | ACATTACTTTAAGAAAGAAATCACCGATCAGTTCCTCTAATGCCTTGTTCT | 0.1504 | LOC400619-[SLC39A11]-L00390811 |
| 2522402 | rs727312 | 4 | NC_000004.6 | 80654964 | T | C | ATTCTGGTCATAAGAAAGGGGAAAATTGAGGCAGATTTTTTCGCCCTACTA | 0.4955 | PAQR3-[ ]-GK2 |
| 2528446 | rs955608 | 4 | NC_000004.6 | 120285975 | C | A | ACCCTGCTCTTTGAAAGTAGGAACCCCATCTTTTCATAGTTCTTGGCATAT | 0.126 | SEC24D-[SYNPO2]-MYOZ2 |
| 2563109 | rs711239 | 2 | NC_000002.6 | 30603678 | G | A | TCCTTCCACTTCTTCATCAATGACCGGCACCTGCAGGGTGTGAGTCACTGG | 0.7625 | LOC285043-[ ] UNQ1849 |
| 2583974 | rs1018139 | 4 | NC_000004.6 | 167425628 | G | A | TTCCTGGTTTGTGCAGTTCAACAACGGTGGTTTTGGACTGTATCTACTTGT | 0.8993 | LOC391715-[TLL1]-SPOCK3 |
| 2596560 | rs661197 | 15 | NC_000015.5 | 37812546 | T | C | AACTTGCTGACTTAGTACCATGAAATGAATAGCTATGAAGGAAGTGAGAGA | 0.9881 | FLJ35989 [GPR]-LOC388113 |
| 2603232 | rs256846 | 5 | NC_000005.5 | 155899663 | T | C | TAAATTCTTACCAAACTATGCCCAACATGTTTTCATTTCATTAAGCAGTTA | 0.2038 | LOC389340-[SGCD]-LOC153743 |
| 2606259 | rs245592 | 5 | NC_000005.5 | 162095447 | C | T | TAAAGCAATATTTTGCTAGTGCTAGCGTGTGTCCAAATGGAATTCTTTCCC | 0.7624 | GABRG2-[ ]-MRP63P6 |
| 2634363 | rs1035191 | 4 | NC_000004.6 | 88135652 | T | G | GTATCCCAATTCCTGAACGGTGGGTTATTAAATCTGAAACTTGAAGGAGGA | 0.7175 | MAPK10-[PTPN13]-SOAT |
| 2641152 | rs689695 | 15 | NC_000015.5 | 45068922 | A | C | TAAGATTTCACTTTGGTTAGTTAACACTTGAAAGTAAATACTCAGATACTA | 0.6289 | LOC145660-[ ]-SEMA6D |
| 2677683 | rs421628 | 4 | NC_000004.6 | 86748728 | G | A | CCTATTTTCTTGTTGTTGACAAGAAAGAAACATACAGCGTTTCAATGACAG | 0.4741 | LOC391675-[ ]-DKFZP564B1162 |
| 2678537 | rs1564138 | 4 | NC_000004.6 | 22202664 | T | C | AAATAAATACATAAATAAATAAAACTAAGAGGGAGTTGAGAATGACCAGAA | 0.1207 | FLJ30194-[GPR125]-GBA3 |
| 2684490 | rs1345439 | 16 | NC_000016.5 | 48528973 | G | A | TTTTCTGCCAATGACTTTCTTCACCATCTCTTTCCTGTGTCTCATTTGGCA | 0.3209 | N4BP1-[LOC388273]-CBLN1 |
| 2685247 | rs1346075 | 4 | NC_000004.6 | 68032302 | T | G | GCATGGAGAATATTGGTCATATTTGTTTACAGCATGTGTGACTTAATAAAT | 0.4612 | EPHA5-[ ]-CENPC1 |
| 2734519 | rs1403724 | 3 | NC_000003.6 | 150338303 | T | G | GCTTTTCTTCAAACATTTACTAGATTCTTATTTGTCAAATCTTTATGTGTG | 0.7577 | LOC389-160-[ ] LOC116441 |
| 2745240 | rs1417712 | 9 | NC_000009.6 | 106975923 | T | C | ATCCAATCCCTTCCCACCCGCATGATGACACTTTGCCAGAACTGGCAGCAA | 0.8561 | LOC347292-[ ] IKBKAP, |
| | | | | | | | | | ACTL7B, ACTL7A |
| 2751104 | rs1424148 | 16 | NC_000016.5 | 76976393 | C | G | ACTAAGTCAGGTGGGTAACACAGCAGTGAGGGCTGAGATCTTGCAGCTTAG | 0.1855 | CASPR4-[ ] HSRG1, LOC400547 |
| 2783077 | rs1460969 | 5 | NC_000005.5 | 7818388 | G | A | CAAATGATATAACGGCAGAAATACCGTATCTCGTATCTCTATTGACTGTGA | 0.5676 | LOC401174-[ADCY2]-LOC134121 |
| 2790279 | rs1484274 | 5 | NC_000005.5 | 154838009 | A | G | GGGAAAAATTAAGATGTTGGCCACAATTGCTATTAATTTGGCCTGAGCTGC | 0.5234 | LOC402234-[ ]-LOC389340 |
| 2814609 | rs349506 | 3 | NC_000003.6 | 141542451 | G | T | GGAGCTGGCCTTGGAAAGCTGATTGGTCGGGAGAAGTGGAAGAAGGAGATG | 0.8862 | NMNAT3-[CLSTN2]-TRIM42 |
| 2863248 | rs533159 | 4 | NC_000004.6 | 130600270 | A | T | AAGTCCCTGAGATTAAGAACACGGCAGAGGATTTGTGACTCTACTTGTGTT | 0.3094 | LOC391697-[ ]-LOC391698 |
| 2899607 | rs1927923 | 9 | NC_000009.6 | 115938436 | A | G | AGATGAGATCAAGTAAAATCTCCTTAGAAGCAGAATGGTTGATCAAATGTA | 0.8332 | TLR4-[ ]-LOC340477 |
| 2926674 | rs2045812 | 4 | NC_000004.6 | 22294777 | A | G | GACTATTTATGTGCTGTTTGCTAAGACAGTTCCTGAGTAGAACCCTGTCCC | 0.6426 | GPR125 [ ]-GBA3 |
| 2931858 | rs2054397 | 3 | NC_000003.6 | 179583353 | G | A | ACTAAATAGGGAAAATGTCAGCTGAGTCCCACATGTAGATTCCTTGTCATC | 0.5908 | LOC401101-[KCNMB2]-WIG1 |
| 2933057 | rs2056044 | 4 | NC_000004.6 | 184201330 | A | G | TCTTGCTGATGGGAACTATGACAACATGACATTGAAGAATTATGAGCTAAA | 0.6058 | LOC90768-[ ]-ODZ3 |
| 2946402 | rs2114438 | 15 | NC_000015.5 | 46415985 | T | C | CCATTTTAGATTTATTAGCAGTTAGTTGAAACAACAGATTTTGTGATTTTT | 0.2849 | DUT-[ ] FBN1 |
| 2946891 | rs2115828 | 15 | NC_000015.5 | 52183171 | A | G | TTCACAATCTTTGAATTTAAACTCAATCGGAAGGAACCTCACATGAGATTA | 0.9817 | LOC400376-[UNC13C]-C15orf15 |
| 2977230 | rs171649 | 5 | NC_000005.5 | 67585630 | G | A | GAGCTGTGTTTTGCATACATGGTCTGTGGTCTGTTTTGTGTCCTAGGTCTG | 0.6097 | EF1B3-[PIK3R1]-SLC30A5 |
| 2993728 | rs1295241 | 5 | NC_000005.5 | 154920028 | C | G | AAGTGAATTTTACTTTCTCCATGAAGCTTTCTAATCTCACAGAGTTACCCC | 0.4173 | LOC402234-[ ]-LOC389340 |
| 2997317 | rs2202308 | 4 | NC_000004.6 | 29577072 | A | G | AAATGTATGTGGTCATTAGGTTTTCAGCATTATTCAGTCATCCCCTCTTTT | 0.8236 | LOC391643-[ ]-PCDH7 |
| 3106665 | rs393973 | 5 | NC_000005.5 | 813578 | C | G | ACAGCATTTTACACCCCTAGCGTAACTGTGATTCAGATGAGGATTGCAACA | 0.6161 | LOC401167 [ ] ZDHHC11, LOC389261 |
| 3107121 | rs13171870 | 5 | NC_000005.5 | 1948745 | G | A | AACCGAGGACACAGACGCAGGGCTCGGGCAGAGTTCCCACGGTGGGCACAG | 0.5691 | LOC389267, IRX4 [ ]-IRX2 |
| 3111057 | rs2713575 | 3 | NC_000003.6 | 129615264 | A | G | CACAAGCTCATCATCTATTCGAGTCGAGAGTATTCAGTTATCAGCCTCAGA | 0.4955 | LOC391572 [GR6] RPN1, FLJ40473 |
| 3112389 | rs10512737 | 5 | NC_000005.5 | 40455544 | G | A | TAAATTCCTAGTGCCTTCGAGTAAAGCTGTTCATGGAGCATATTTAAGACA | 0.8709 | LOC285634-[LOC389285]-PTGER4 |
| 3114067 | rs6802103 | 3 | NC_000003.6 | 131858889 | T | C | CTTCTTGTGGTCATGTGAGACTCATTCCTTTTACTGTTTCTGTGCATACCT | 0.2047 | PIK3R4-[ ] GSTO3P1 |
| 3115306 | rs35097472 | 5 | NC_000005.5 | 6135712 | G | A | TCTCCAGTTGTTTCTTTTGTGTGAAGTAACTTCCAGAATCCATGACTCACA | 0.7383 | K1AA0947-[ ]-LOC401172 |
| 3115452 | rs4438885 | 5 | NC_000005.5 | 43466591 | A | C | TTTACTTGATTCTCCCAGTTGCTGGATATTTAGGTAGTTTGATTTTGTCTC | 0.9812 | CCL28 [ ] FLJ21657 |
| 3117549 | rs4854582 | 3 | NC_000003.6 | 134393392 | G | A | TCTGTCTCAACTCTATATCACCTGTATGCTTGTAGACTGGCAAAGAAAGTC | 0.1181 | NPHP3-[MGC3040] BFSP2 |
| 3117608 | rs1201674 | 3 | NC_000003.6 | 134405511 | T | C | CTCAACTCAGAGTCAGGGTTTGAACTATATCCCCGGCTGGAATGGGGGCCA | 0.8833 | NPHP3-[MGC3040] BFSP2 |
| 3118276 | rs10475362 | 5 | NC_000005.5 | 7331686 | C | T | TCTCCAAACAAAACCTCTCTCTTTCCGACTCTCCACCCTATGCTTTTCCAA | 0.3987 | POLS-[ ] LOC401174 |
| 3118987 | rs13189538 | 5 | NC_000005.5 | 7838724 | G | A | AATTCTCCCAACTTTGTTATTGGTCGTTGAGATGATACACATTCAGTACCA | 0.5736 | LOC401174-[ADCY2] LOC134121 |
| 3122355 | rs34286321 | 3 | NC_000003.6 | 138173434 | G | T | TACCTAGAAACTCCTTTAAATTTGGGTTTCTGAGAGTAGTAATGACTTGTA | 0.8018 | MGC34923-[ ]-NPM1P17 |
| 3122754 | rs7647718 | 3 | NC_000003.6 | 138188222 | A | G | TGTTTTTGGAGGGCAGAGATAAACTATGGAAAGATGGAATGCTGTATTTTT | 0.7963 | MGC34923-[ ]-NPM1P17 |
| 3123186 | rs6881925 | 5 | NC_000005.5 | 53710002 | T | C | CAAAGCACATGTGAGGCCCAGAAGGTGGTTTTTCATGCCTTTAATAACCAT | 0.3979 | ARFRP2-[ ]-HSPB3 |
| 3125188 | rs439893 | 5 | NC_000005.5 | 11335191 | T | C | CGAGTTTTTAAGTGCCCAGAGAAGATGTACTCAACAGGGAGAGTTAAGAGG | 0.621 | DAP-[CTNND2]-LOC401175 |
| 3127128 | rs26269 | 5 | NC_000005.5 | 14182336 | T | A | GGTTTCCAGCTATCATCCCTGTTTTACCTCTTCTTATTCCTGGGTTTGAGA | 0.522 | DNAH5-[ ] TRIO |
| 3134325 | rs2769187 | 9 | NC_000009.6 | 116309722 | A | G | TAAATTAAAGCATGAACTACGAGGAACCTGTATCTAAGTAAAGGGACTGAC | 0.5203 | LOC389787-[ ]-LOC347165 |
| 3134356 | rs2769192 | 9 | NC_000009.6 | 116321490 | A | G | GATGATGGTCATATAATTAAGTTTGACATCTTTACTGGAGAGCACATCAGT | 0.5378 | LOC389787-[ ]-LOC347165 |
| 3134360 | rs2806065 | 9 | NC_000009.6 | 116326118 | G | T | CACATTCTTTGTATCTATAACACATGCTTCTGAAATATCAGATACCCATCA | 0.5298 | LOC389787-[ ]-LOC347165 |
| 3141936 | rs7340627 | 3 | NC_000003.6 | 158153306 | A | G | CTCATTAGAAAGGACTTTGATGCACATTTTTATGCAAGAGGTGAATTCTTC | 0.6012 | LOC339892-[LOC339894] |
| | | | | | | | | | LOC391589, CCNL1 |
| 3142103 | rs16827384 | 3 | NC_000003.6 | 158306837 | A | C | TGGAGCATTCTTAAAGCTAGTAATGATCTTTGCAAATTTAAATTTTCCATA | 0.6842 | LOC391589-[FLJ12604]-PTX3 |
| 3150971 | rs7618373 | 3 | NC_000003.6 | 177657932 | G | A | GATAAGAGAGTAAAGGTAAGCTTTCGGGGCCCTTTTTCCACATGTCATATT | 0.6275 | LOC254827-[ ]-IRA1 |
| 3151597 | rs16829750 | 3 | NC_000003.6 | 179599636 | G | A | ACCACCCTATACCATCTCCTTAGGCGCAGCACTGTTTTGCTTCAGATGCTT | 0.5934 | LOC401101-[KCNMB2]-WIG1 |
| 3151603 | rs16829771 | 3 | NC_000003.6 | 179605903 | T | C | TAATCTGAGTTTCTGAATGTACATGTAGATGAGAGTACACATGTGTGAGAG | 0.5965 | LOC401101-[KCNMB2]-WIG1 |
| 3152573 | rs17188103 | 3 | NC_000003.6 | 183065384 | A | T | AAACAGAGAAGTCAATGGCAAGTTGAATTTTGTATGGTGACTTTACCAGAA | 0.8419 | LOC401102 [ ]-LOC401103 |
| 3152805 | rs1357451 | 3 | NC_000003.6 | 183806996 | G | A | TGATATTTCAAAGTTGTTTTCGCTCACATTCCTTTCATTACTGTCAAGAAT | 0.2736 | LOC402152-[ ] ATP11B |
| 3170195 | rs11661897 | 18 | NC_000018.5 | 69672542 | C | T | TGGGAAGAAGATCCAAACCACATCACCAGGATTCAGTTAACCATCCCCCTC | 0.3085 | LOC400655-[ ]-FBXO15 |
| 3174413 | rs12189352 | 5 | NC_000005.5 | 79619174 | A | C | TGAGACTTTCTCTGGCTGGCTGCCAACCAAAGACTTCCCACAATGGTTCTA | 0.5628 | THBS4-[C5orf12] LOC391803 |
| 3174758 | rs10514187 | 18 | NC_000018.5 | 71635777 | A | G | GGCTTGATCCTCTAATGACCCATGGACTTGAATTAGAACATCATCCTGGAT | 0.953 | LOC284274-[ ]-LOC284275 |
| 3181874 | rs601499 | 5 | NC_000005.5 | 88136760 | A | C | GCAAAGACTTCAGGGCCAGGCTCCAACTTTTACAAACACTCAAGATAGGAA | 0.4167 | MGC33214-[MEF2C]-CETN3 |
| 3187067 | rs6879703 | 5 | NC_000005.5 | 92529821 | G | A | ACTAACGCTGACAAGAGCAGACATCGTACAAGTGGGACAGGGGAAAAAAAT | 0.8922 | LOC391810-[ ]-LOC391811 |
| 3191406 | rs11149997 | 16 | NC_000016.5 | 77329233 | A | G | ATGTTGCATTTCAGATTTTTTTGAAAAAAAGCCAGAGCCCCAACAATTTAA | 0.3284 | ADAMTS18-[ ]-LOC388299 |
| 3193146 | rs9928690 | 16 | NC_000016.5 | 78378382 | A | G | GAAATCCTCCTTCCTTTTTAACAAGACAAGTAGAAAACGAAAACAACATCA | 0.6059 | LOC342419-[WWOX]-LOC401862 |
| 3194833 | rs441333 | 16 | NC_000016.5 | 79406333 | A | G | TGCAAATCAGCAAAGTGAAGCTGGTAAATCTCACTTCCTATTCCCTCTTAG | 0.5697 | WWOX-[ ] MAF |
| 3200432 | rs16957764 | 16 | NC_000016.5 | 82393722 | C | T | GCCACACACAAGCCTATTCTAAACCCGTCTTTCTTCCTTAAGTGTGCCAAT | 0.913 | MPHOSPH6-[ ] CDH13 |
| 3222073 | rs13032210 | 2 | NC_000002.6 | 147102514 | G | A | GTCAGATGTAGATACGTTTTTGAAGGACTGAAAACTAAATAATTCCATTTC | 0.985 | LOC402107-[ ]-LOC200583 |
| 3255514 | rs4679045 | 3 | NC_000003.6 | 33515931 | G | C | TCTTATCCATGAAATATTTTTGAATGAATTATCTGAATGAATATGCACATT | 0.6151 | UBP1-[CLASP2]-PDCD6IP |
| 3263451 | rs12053901 | 3 | NC_000003.6 | 111773106 | G | A | CAGATTGGCTTCATTCTTTAATTTCGATATGGACTCACTTACAAATGGACC | 0.9721 | LOC389141-[ ]-LOC151760 |
| 3263776 | rs16860850 | 3 | NC_000003.6 | 114372525 | G | A | TATATTAGTTTGTGAGCATTGAGTTGCCTAGAAAAGTTTGGAAGTCCTACA | 0.8123 | BOC [ ] LOC402136, FLJ11142 |
| 3271579 | rs6708711 | 2 | NC_000002.6 | 114619662 | A | G | GGAAAAAAAAAAGACTGAGGCCGTGATATGTGTAACATGTTATTAGATATG | 0.7258 | bA395L14.13-[SLC35F5]-ACTR3 |
| 3287599 | rs7218003 | 17 | NC_000017.6 | 49047130 | T | G | TTCTTGACTGTTGGTGAGGGAGTTGTATCAACCCCAGGTTCTTAAACTTGT | 0.3988 | CHAD, FLJ11164, FLJ20920, |
| | | | | | | | | | MRPS21P9 [ ] EPN3, MYCBPAP |
| 3288070 | rs2215290 | 17 | NC_000017.6 | 49698859 | C | T | ATCAAACTCAGTCTTCTGACTGGCTCCTTTGGTCTTCCCTATTTCTCTTAA | 0.8634 | SPAG9 [ ] NME1, NME2, MBTD1 |
| 3288843 | rs6504747 | 17 | NC_000017.6 | 50332654 | C | T | ACAAATGCAAGAGTAGAAGTACACACGGAGCAGTGGCAACTCAAGAGAGAA | 0.962 | LOC388401-[CA10]-LOC339209 |
| 3288850 | rs16950512 | 17 | NC_000017.6 | 50341788 | A | G | AACTCATTTTCCATTTTCCATGCATAGGATAGTAGGTCCACCTCACCTTTA | 0.931 | LOC388401-[CA10]-LOC339209 |
| 3290010 | rs4501302 | 5 | NC_000005.5 | 65974935 | A | G | CCCACCTCAGTGCATGTGTGTATACATGGATTGTTTTAGTATCTTAATCAC | 0.7552 | LOC202227-[LOC375449]-KIAA0303 |
| 3293153 | rs6870517 | 5 | NC_000005.5 | 67829547 | T | G | TTGTGACTGTCACCGGGAGACAGAATAAGTGAGGTGGCACAGGTGCAGTAT | 0.4793 | PIK3R1-[ ]-SLC30A5 |
| 3296481 | rs4794717 | 17 | NC_000017.6 | 55839242 | A | C | GAGTGTGGGAAATTAGTGCAGGCCCAAGGAAAGGAAGTGTGAAACTGCTTC | 0.1752 | FLJ11710 [MSI2]-MRPS23 |
| 3303532 | rs33934033 | 5 | NC_000005.5 | 96316939 | G | A | GTGTGGACTTTTACTGTTGAGCTAAGGTTTATGTTTATATATGTTTTATTC | 0.4718 | ARTS-1-[LRAP] LNPEP, FLJ39485 |
| 3318048 | rs6872948 | 5 | NC_000005.5 | 135601773 | T | C | GCTGCTTCACGCCTATAGCTCAGTTTTACAAATAGCTATAGTTTGGACGCT | 0.4334 | MADH5 [ ] TRPC7, LOC389332 |
| 3322140 | rs13155464 | 5 | NC_000005.5 | 142965137 | T | C | AGCTAGAACTCAAATCTGGCCTTGGTATGCAATCTTGACTATTTTAAGCTC | 0.872 | LOC389335-[ ]-LOC345537 |
| 3325587 | rs7732327 | 5 | NC_000005.5 | 146853063 | C | T | ATAACTCTAATGACTTTGTAGTTTACGTGCAAAGGCAGAAAGAGACCTCAA | 0.4176 | MGC22688-[DPYSL3]-KIAA0555 |
| 3326948 | rs17778143 | 5 | NC_000005.5 | 148206881 | T | C | AAGTGGCAACGAAATAAATGGTGTATGGACCTAAAGTTGTTAATAACTCAA | 0.7801 | HTR4-[ ] ADRB2 |
| 3328007 | rs10515637 | 5 | NC_000005.5 | 149270111 | A | G | CTACATT1GCAGCTGCCCATTTAGAATCAACCCACGCTGCCCAGAGCAGAG | 0.6198 | PPARGC1B [PDE6A]-SLC26A2 |
| 3329421 | rs9688110 | 5 | NC_000005.5 | 150992312 | G | A | GGGCATACCAGGGCTGTGGAAAACCGACACCTCAATGATTCCCTTTACCCT | 0.6614 | FAT2 [ ]-SPARC |
| 3332880 | rs12523643 | 5 | NC_000005.5 | 154831079 | T | G | CAGAAAAAATTTAGTGCTGTGGGAGTCATCATGATATCCCGGAAAGGCACA | 0.5465 | LOC402234-[ ]-LOC389340 |
| 3339124 | rs2431271 | 5 | NC_000005.5 | 162097173 | T | G | TAAGTAAACATTAAATGCTCATGAGGAAATATTTTACACCCAAATATTAAT | 0.7735 | GABRG2-[ ]-MRP63P6 |
| 3352050 | rs2014379 | X | NC_000023.5 | 105103239 | C | T | TGTAGGAAATTGGATGTTACTATTCCTTCAATTCGGGGCTTCTAATGGGTA | 0.8144 | ZCWCC2-[FLJ11016] FLJ20130 |
| 3373581 | rs17597288 | 18 | NC_000018.5 | 51326195 | A | G | AATGCCTGGGAAAATGATCTAAAGCAATAATCCTAAGAAATAACCAAGAGA | 0.8768 | SE57-1-[TCF4]-TXNL |
| 3386350 | rs17648108 | 5 | NC_000005.5 | 177945157 | T | C | GAAAATTTGAGGGAAATACAGCTGATTCCTTATGGTGAACCTGGCCCAGCC | 0.7125 | MRPL50P3-[COL23A1]-CLK4 |
| 3395544 | rs4380588 | 4 | NC_000004.6 | 6324430 | G | A | GCCATCTCTCCTCCAGGCTGGAGTCGGTGCTTCCCACAGTTACTTCTCACG | 0.763 | LOC285484, LOC389198 [ ] WFS1 |
| 3396622 | rs17178920 | 4 | NC_000004.6 | 8276158 | C | T | CAGCAGCAACTGGGACGGAGCAGGGCCCACTGAGTCAACCACCCAGGTGTG | 0.8043 | LOC389199-[ABLIM2]-LOC391618 |
| 3396741 | rs2280569 | 4 | NC_000004.6 | 8559677 | C | T | TCATAGACAAAACACATGTAACCCACGAAGAGTTTGTTTAAACCCAAGGAA | 0.782 | HTRA3-[ ] ACOX3 |
| 301101 | rs10006709 | 4 | NC_000004.6 | 17257440 | T | C | ACCCCAATCCAAAAATCCCTTTAACTGCAACTCTGAAAAAAATGCCAACGA | 0.9783 | LOC402173-[QDPR] LOC391639 |
| 3404095 | rs11933465 | 4 | NC_000004.6 | 22227454 | A | G | AAATGTAAATGGAGCATACCTCTGGATCAGAAGATTTTAGGTATTATTATT | 0.4132 | FLJ30194-[GPR125]-GBA3 |
| 3412405 | rs6829422 | 4 | NC_000004.6 | 100204996 | G | A | CTTGGTAGAAAATCTGGAAGCACTCGTCGCAAATGCCTTTACAACACTATG | 0.1709 | LOC132556-[ ]-EIF4E |
| 3414039 | rs10516493 | 4 | NC_000004.6 | 103743038 | A | T | GGCACAGCCTTGATGAACCTTACAAACTGCTCTCAGTGAGTGGTTCTCATC | 0.689 | SLC39A8 [ ]-NFKB1 |
| 3417519 | rs11098185 | 4 | NC_000004.6 | 114427028 | T | C | GAACATTAACAGCATCTCTCTCCTATAGATCCAGTTACGACAAAGGTTTTC | 0.2821 | LOC391689-[ ] ANK2 |
| 3419155 | rs7675219 | 4 | NC_000004.6 | 118148630 | A | C | CATAATTCTCAAGGACTTTTATTTAATAGGACTGGAGAGGGATCTGGACAT | 0.7392 | LOC344978-[ ]-TRAM1L1 |
| 3430918 | rs12643790 | 4 | NC_000004.6 | 91232542 | C | G | CCACTTCTATATTCTCTTCTTCCTTCATTTTTCAAATGTTTGCATTTAAAA | 0.9756 | SNCA [ ] MMRN |
| 3434449 | rs6824100 | 4 | NC_000004.6 | 94964127 | G | T | CTTTTTGTAAGAGGATACAATAAAAGTATGAGTCAAAGAATATATTGGGGA | 0.3755 | LOC133083-[GRID2]-ATOH1 |
| 3435351 | rs9942167 | 4 | NC_000004.6 | 96081870 | T | A | GAGGAGTCAGTTATCTCGGCGACTCTATTTCCTTTTCTGAATATACGATCA | 0.6103 | LIM [ ]-BMPR1B |
| 3443855 | rs11733446 | 4 | NC_000004.6 | 23339715 | T | C | CTCCAGGATCACTGTGATGATAGGTTCAGAATTCCTCCCTTCAATGCAACT | 0.6236 | GBA3-[ ]-PPARGC1A |
| 3444361 | rs12510610 | 4 | NC_000004.6 | 42420247 | T | A | ATCAACACCATCCCTTCTGCAGTCATTTCTTCACCATCCCTTCCACGCATG | 0.8414 | LOC389206-[ATP8A1]-LOC389207 |
| 3444374 | rs10517038 | 4 | NC_000004.6 | 42426784 | C | T | AAAGGCCATATAGAAGACAAAAATACCAACTGTATCTATCTCTTACATGAA | 0.8449 | LOC389206-[ATP8A1]-LOC389207 |
| 3447488 | s2348813 | 4 | NC_000004.6 | 45016728 | T | G | GGACTTTTAGCTTTTGAAATACAGGTTTTFTTTTTGGTAATCACATTAGAA | 0.6621 | GNPDA2-[ ]-LOC391648 |
| 3447858 | rs17669701 | 4 | NC_000004.6 | 27515699 | G | A | ATAACAAAATTGTAAGTGTTACTACGGAAAATTCCTTTTTACTACTCTTAG | 0.6594 | LOC391642-[ ]-LOC285509 |
| 3448879 | rs7662149 | 4 | NC_000004.6 | 28897386 | C | T | GAAATTACCATATCTCTGTCTTCTTCGCTGAGTCATGGAATCCCTTCAAAA | 0.7563 | LOC391643-[ ]-PCDH7 |
| 3449937 | rs17737935 | 4 | NC_000004.6 | 29650714 | T | C | TAATGGCCTGGGAGAGAGAAATACTTTGGACTTAAGCATTATGGTGTGTAG | 0.8361 | LOC391643-[ ]-PCDH7 |
| 3452760 | rs3113584 | 4 | NC_000004.6 | 33136093 | A | G | TATTTTTCTGAGCGACAAAATGTAGGAGGCATCATAAAATATGAGGAATGG | 0.7918 | LOC402174-[ ] LOC133185 |
| 3460224 | rs16994357 | 4 | NC_000004.6 | 38141804 | C | T | AGGTGGGTAGGACAGATGTCACCCTCTTTCAGGCAGGAATCTGAGAGGCTG | 0.9741 | TBC1D1-[ ]-FLJ13197 |
| 3475688 | rs13111373 | 4 | NC_000004.6 | 153691353 | G | C | TGCGTATGATCTCAATACTAAGAAAGATCAGAACTGCTTATACGTTACAGT | 0.608 | LOC389232-[ ]-LOC389233 |
| 3477251 | rs17032828 | 4 | NC_000004.6 | 156835329 | C | T | TACGTAGGAAAATATCTATGTTACACGATCTTGTGCAGTCACTGCTATTAA | 0.9123 | LOC389237 [ ] FLJ21159 |
| 3477963 | rs10049936 | 4 | NC_000004.6 | 157875784 | T | A | TCACAGAGACTTGCAGAAAAGAGATTAAATTTACCATAGGATATCCTTAGT | 0.8351 | FTHP2-[ ]-PDGFC |
| 3480149 | rs17332185 | 4 | NC_000004.6 | 161878565 | G | A | GCTGAACATTATTGTTCCTGAGACTGACTTCTTAGCCATTTATTTTACTTC | 0.6901 | PDZGEF1-[ ]- DKFZp566D234 |
| 3481741 | rs17460297 | 4 | NC_000004.6 | 163608108 | G | A | GGACTTGTACAACCCAGGATTCCTAGTCATTCCTGTCAAACATACACTTTC | 0.8208 | PDZGEF1-[DKFZp566D234]-LOC92345 |
| 3484509 | rs13340246 | 4 | NC_000004.6 | 167062794 | C | T | GCTTTGGGAATAGGGCTTACCAAAACTGGGGCATTTGGTAGGGATGTGGTA | 0.7445 | LOC402191 [ ]-LOC391715 |
| 3485444 | rs7657081 | 4 | NC_000004.6 | 167751275 | G | A | CCATCAGGATGCAATACATAAGCAAGTGAACCCACCCATAATTACTTAATA | 0.0064 | TLL1-[ ]-SPOCK3 |
| 3486976 | rs13119904 | 4 | NC_000004.6 | 66685700 | A | G | TTCATCTCCTGTCATCTCATCTTGTATTCTCACTGAACCTGTATGTTTAAA | 0.6912 | EPHA5-[ ]-CENPC1 |
| 3509817 | rs10415223 | 19 | NC_000019.6 | 61733990 | C | T | GTTTTGTCCTGCTGAATTGGCAGAGCAGTCCCAAAAGAACTGTAACTAAGG | 0.5385 | ZNF471, MGC9913 [ ] ZFP28, |
| | | | | | | | | | LOC388566 |
| 3510699 | rs10417951 | 19 | NC_000019.6 | 33967121 | G | A | AATCACTGCCAACAGTGGTGCCTGCGTTTGCAAGGGCAGAGGGGCTCTCAG | 0.8897 | LOC388526-[ ]-LOC148145 |
| 3511990 | rs879523 | 19 | NC_000019.6 | 61229262 | A | C | AGCAGAGCTATTTTGTAGATTGTTAAGAAGCTTTTGAGGCCTTTAAAGTAG | 0.5949 | NALP8 [NALP5]-LOC126208 |
| 3512057 | rs10518285 | 19 | NC_000019.6 | 36695508 | T | C | CTAGGCAGAGGTTGTTCTTTCTCGATAGCTGTTCCAAGAGTGCTATCCTGG | 0.4946 | LOC388529-[ ]-LOC339316 |
| 3522226 | rs13101469 | 4 | NC_000004.6 | 125280554 | A | T | TATTTTAAGTTTTTTTGTCTGATGGACTTCTGAGCTCTGCTGTCTGTCAAT | 0.806 | LOC402186-[ ]-LOC391694 |
| 3522237 | rs17492375 | 4 | NC_000004.6 | 125271718 | T | C | TGCAGTCGGTGTGTAATTAAGAGGATGTAATTAAGGATTATTGCTGGATGT | 0.8134 | LOC402186-[ ]-LOC391694 |
| 3524022 | rs17339087 | 4 | NC_000004.6 | 121861556 | C | A | CAGAATTAACGTGCTTCCAAACTGACAGCACCCATTAACAACTCACTCACA | 0.5696 | LOC344988-[ ]-PRDM5 |
| 3526374 | rs6659997 | 1 | NC_000001.5 | 89739285 | C | T | GGACAGTTCAAATGATTCTGGGTCTCATAATTGCTACATGATGTAAGGTGA | 0.7088 | AD158-[ ] LRRC5 |
| 3528443 | rs11352973 | 4 | NC_000004.6 | 128380774 | C | A | TTAAGGCTCCATTCAATCAAAAAAACATTTCATATCATCAATACCACTGTT | 0.7653 | LOC132817-[ ]-PDZK6 |
| 3530702 | rs12502992 | 4 | NC_000004.6 | 131910186 | G | A | ATGGTACCAGAAGACATGCTAGAGGGGATTGGAATAGGTGTGGAGAGCCAA | 0.7046 | HCP14-[ ]-LOC401155 |
| 3531432 | rs1705709 | 4 | NC_000004.6 | 132915159 | A | G | CTGTGCTGAATGCCAGGTAATTGTAACCAAACTCAGGTTCAACTGCTCACT | 0.7843 | HCP14-[ ]-LOC401155 |
| 3531816 | rs17049070 | 4 | NC_000004.6 | 133667612 | A | G | GATGTGTAAAACGTACGTGCCTAAGATCAATGACTGTGTCATCACAGAAGA | 0.8554 | LOC401155-[ ]-LOC402188 |
| 3548055 | rs12439488 | 15 | NC_000015.5 | 39610631 | G | A | AGACCCTTCTCTGGCCTTTTCACTAGAATACAACAAAGATTAAATCAGCTT | 0.8662 | TYRO3, LOC283747 [ ]-MGA |
| 3554315 | rs12904774 | 15 | NC_000015.5 | 55573969 | C | T | GCAGAGTTTTCACGAACAATGAAAACGCCTGGGAGTGTTCATTGCCAGTTA | 0.6293 | FLJ14957 [ ] FLJ30973 |
| 3554904 | rs4775005 | 15 | NC_000015.5 | 55924221 | G | A | GTGTAATCCTGTCTCAACATCTTGCGTAAGAACTCATTCTAGGAAAAAGAT | 0.4236 | GRINL1A-[ ] ALDH1A2 |
| 3556069 | rs34458502 | 15 | NC_000015.5 | 56413166 | G | A | GGTCTGTGGCCAAAGGCATATCCCAGTCACATGACCACTCCAGCAGCAAGG | 0.9825 | LOC145780-[ ] LIPC, |
| | | | | | | | | | LOC400377 |
| 3557017 | rs11634045 | 15 | NC_000015.5 | 71216435 | A | G | AGCAAAGAAGTTTGTGCCATTTCAAATAAGTGACATGTCAGACATGGAGTC | 0.6667 | ADP-GK-[NEO1]-LOC388134 |
| 3560562 | rs11853481 | 15 | NC_000015.5 | 45160455 | G | A | GCACAAAAATGCCTCAAAAAGTGTCGTAGCCTATATAGACCATATGATCTC | 0.6728 | LOC145660-[ ]-SEMA6D |
| 3561023 | rs16959379 | 15 | NC_000015.5 | 45394399 | G | A | GTTTTGATGCCATTAGGTTCAAGGGGCTCAACATCTGGTAGAAGAGATAAA | 0.9003 | LOC145660-[ ]-SEMA6D |
| 3562810 | rs2678425 | 15 | NC_000015.5 | 60240216 | A | G | AAGAGCTTGGAACAAAATTGCCACTGGGGACCATAAGACTAACTGCTGCGC | 0.6898 | LOC255177 [ ] FLJ38723 |
| 3563460 | rs12916536 | 15 | NC_000015.5 | 46422627 | A | G | AAGACTGCTACATTAATAATTTGGTATAAGTGTGGATGTGTGTGGGGGTGT | 0.2404 | DUT-[FBN1]-LOC400370 |
| 3563481 | rs9920570 | 15 | NC_000015.5 | 46431504 | A | G | CAACCACTATGCCCTCACCATCTGCACAACTGCTTTGGAGCTGCAGATCAG | 0.2485 | DUT-[FBN1]-LOC400370 |
| 3563492 | rs8034591 | 15 | NC_000015.5 | 46440824 | C | T | ATCTCTTCATATTGGGTTTTTTGAACGTGGGGAAATGTCTTITTCTTTCTA | 0.3002 | DUT-[FBN1]-LOC400370 |
| 3563516 | rs10519174 | 15 | NC_000015.5 | 46450680 | G | A | AACTAACGATATCATAGAAGATGACGCTCCTGATTTGTGGGTTAATCTTTC | 0.3027 | DUT-[FBN1]-LOC400370 |
| 3563548 | rs2555470 | 15 | NC_000015.5 | 46463496 | T | C | TAGTTATCTCAATATCCACAGAATGCTACCCTGACAGCAAAATGCCTTGCA | 0.7572 | DUT-[FBN1]-LOC400370 |
| 3563568 | rs4774517 | 15 | NC_000015.5 | 46475347 | G | T | GGGTGACAACTAGTGGAGTCCTAATGTGCACCAGGTGTGCTCTTTGTACCA | 0.7584 | DUT-[FBN1]-LOC400370 |
| 3563628 | rs11070644 | 15 | NC_000015.5 | 46500553 | T | C | GAAACTACAGTTGCTGCTTACTATTTGAAAGACTGTCAAAGGAGTGGCCAT | 0.7604 | DUT-[FBN1]-LOC400370 |
| 3565071 | rs12900106 | 15 | NC_000015.5 | 61731727 | G | T | TGGTCTTGTATAACAAAAAACCAACGCGTTCAAAATGGAATGATATCCTCT | 0.1262 | LOC400380-[HERC1]-DAPK2 |
| 3565203 | rs9972404 | 15 | NC_000015.5 | 61658726 | G | A | CTCAACAATTACTGATAACCAAATTGGCATAAGAAACTTACTTGCAGTTAA | 0.1246 | LOC400380-[HERC1]-DAPK2 |
| 3565214 | rs2228511 | 15 | NC_000015.5 | 61669846 | C | T | TGCAGGTGCCACATAACAGGTAGTACGGATTTCCACTCCCACATTCACCGC | 0.1251 | LOC400380-[HERC1]-DAPK2 |
| 3565239 | rs12906986 | 15 | NC_000015.5 | 61683798 | T | C | ACAAAATGTAATAAAAAGCTTTTGCTGGTATAAAGTTTTGTTGTTCTAACC | 0.1185 | LOC400380-[HERC1]-DAPK2 |
| 3565269 | rs4984318 | 15 | NC_000015.5 | 61800500 | C | T | AACCCTATCAGCAAAGCAGAATGAACGTGTGCTTCCAGGAGTTTGGAGTAT | 0.1324 | LOC400380-[HERC1]-DAPK2 |
| 3565281 | rs6494436 | 15 | NC_000015.5 | 61807121 | C | T | GTTAACTTCAAATCAGTCAATGACACGGAGTTGTTCAACAAAATTATAAAC | 0.1291 | LOC400380-[HERC1]-DAPK2 |
| 3565292 | rs10152453 | 15 | NC_000015.5 | 61814430 | C | A | CCCTACGCTCACCAACACAGATTTACTCACTTCCTCCTCTAACTCTCTTAC | 0.1293 | LOC400380-[HERC1]-DAPK2 |
| 3567213 | rs11637964 | 15 | NC_000015.5 | 62957552 | A | C | TATACAATTTGTGCATCATGGTACCACAACGATGAAAGTCAATTCTTTCTC | 0.9034 | pp9099-[LOC348094] ACP33 |
| 3567949 | rs3784308 | 15 | NC_000015.5 | 49243454 | A | G | TAGTCACTTAAATTTGGCTGACTAGAGCCTAATAGACCAGGAGAATTAAAA | 0.9481 | LOC388121-[CYP19A1]-FLJ11181 |
| 3592740 | rs26320 | 5 | NC_000005.5 | 115824481 | A | G | CATCTCTATGATTTTTGTTATTACAGTAGGTCACTGAGCTGTTATTTATTT | 0.5314 | COMMD10-[ ] SEMA6A |
| 3592834 | [NULL] | 5 | NC_000005.5 | 115854548 | G | C | AGGGAGCTTCATCAGTACTTTGTTCGTACCTCCAAATGGCAACTACAAGGG | 0.8375 | COMMD10-[ ] SEMA6A |
| 3594277 | rs2602234 | 4 | NC_000004.6 | 141114754 | A | G | CTTTAAATCAATTGAATTAGGGTTAGTGTGATCCCCAGATCATGAGGAAGC | 0.6336 | SET7-[ ] MGST2 |
| 3596962 | rs3909595 | 4 | NC_000004.6 | 142454652 | G | A | ATTCCAACATAGTTTAAATATCAATGTGCAATGCGTGCAAAAGAAATCAAA | 0.7105 | KIAA0882-[RNF150]-LOC389227 |
| 3605475 | rs7686137 | 4 | NC_000004.6 | 147784092 | T | C | GGCAGGAAGCAGGGAGCTCTAAGGGTCTAAAGAATCAGAGTGAAAACAGGA | 0.2941 | LOC345051-[ ]-DKFZP566M114 |
| 3609443 | rs6866678 | 5 | NC_000005.5 | 126844177 | A | G | TTCACCGGAAATGGTGTACCAATTAACAACATTCACATTATAGTTTGGGAC | 0.1961 | LOC345818-[MEGF10]-LOC389322 |
| 3611749 | rs17026183 | 4 | NC_000004.6 | 150882380 | T | C | AAGACTAGATCTTTTTGGTAAACTGCCTACTGAAGGGATCAGCTAAGGTCC | 0.452 | LOC389231-[ ] LOC285423 |
| 3612255 | rs2683090 | 15 | NC_000015.5 | 33824979 | A | G | CCAGTAACTTTTAAAATGGAGGCGGAATTAATAACAGCAGCAACAACCACA | 0.2717 | MGC14798-[ ]-HH114 |
| 3613803 | rs33985535 | 2 | NC_000002.6 | 11325911 | C | T | GCAGGTGTTCACTTGCGACCCTTTTCGTCCCTTCCAAATCCGGCAGGTTCT | 0.5104 | FLJ25143, FLJ33534 [C2orf22] |
| | | | | | | | | | ROCK2 |
| 3614176 | rs7668154 | 4 | NC_000004.6 | 152644037 | A | G | AATGCTTTATTGACATTATACGACTATGCTAAGAACTGTCAGGCCTCTGAG | 0.0458 | RPS3A, U736 [DKFZp434D0215]- |
| | | | | | | | | | ESSPL |
| 3614192 | rs11099788 | 4 | NC_000004.6 | 152663075 | G | A | CAGGCATAAATGAAATCAGATGCCCGCAGAGAAAGCTGTGTGATCACTCCA | 0.0465 | RPS3A-[DKFZp43400215]-ESSPL |
| 3616400 | rs8024166 | 15 | NC_000015.5 | 37731881 | T | C | ACATCCAGGAGCACTCAATATGTGTTGAATCAATTGTTCAAAAACAGAAAT | 0.9773 | THBS1-[FLJ35989]-GPR |
| 3641695 | rs17062188 | 4 | NC_000004.6 | 177488084 | T | C | AGAACCATTCATCTTCTTAATCCCATGCCTCCTGCTTGGTTTATTTGTATG | 0.6739 | GPM6A-[ ]-WDR17 |
| 3666604 | rs4779061 | 15 | NC_000015.5 | 81173449 | T | G | CACGTCTGTTTTTGTTTTCAGGAATTTCCCGAGTCTGTTGGTATAAAAAGA | 0.8355 | LOC283693 [LOC123722] KIAA1971 |
| 3666695 | rs17258343 | 15 | NC_000015.5 | 79933923 | A | G | TTAACCCTGTTATAGAAAGAATGCAAACAGAAAGAAAAGACTTAATGTAAT | 0.8224 | TMC3-[ ]-LOC390621 |
| 3666917 | rs4420497 | 15 | NC_000015.5 | 82516035 | C | T | TTTCTTCCATCCCTTTGGAAAATGCTGTTTAACCTTAACAATTGATATACC | 0.878 | LOC388158 [LOC388159] LOC388160 |
| 3668870 | rs17705887 | 15 | NC_000015.5 | 85624263 | G | C | GGGGACAGGAATCAAGGCGGGCCCAGGGTCGAGATAGAAGGAGCTATTTGA | 0.748 | LOC388169-[ ]-FLJ31461 |
| 3670712 | rs11631508 | 15 | NC_000015.5 | 86228234 | A | G | GGACACAGAGACGAGTATAAATTCAAAGGTCCTCCCTCCTTTAATAACACA | 0.6837 | LOC388170-[NTRK3]-MRPL46 |
| 3673009 | rs12437470 | 15 | NC_000015.5 | 89304173 | G | A | GTATCCTGACCATTCACAAAGTGTCGTGCCACAGCACTTGCATCATTAGGG | 0.8493 | PRC1, VPS33B [ ] LOC390638 |
| 3674864 | rs12443068 | 15 | NC_000015.5 | 91922674 | T | C | CACGAAGGCTACTTTCTCCGAGAAGCCCTCCTTACCCCCATGTTAAATCAG | 0.2732 | LOC390641-[ ]-LOC283682 |
| 3675147 | rs17711781 | 15 | NC_000015.5 | 92443188 | A | G | TTCCTTCAAACAATCACCAACTCCAGTTTCCTGATTTTAACTCAATTGTCA | 0.039 | LOC400455-[ ]-FLJ11175 |
| 3684385 | rs149511 | 5 | NC_000005.5 | 16522331 | T | C | CAAAAAAAATTACCAAATTGATCACTCTGAAGAATTTAAACTAAGATCACT | 0.6809 | ZPR9 [ ] FLJ20152 |
| 3686058 | rs16885644 | 5 | NC_000005.5 | 19694961 | A | T | GAAGCAAAATATATACAAGTTAAAGATATGTGTTCAGCTTCAGTCCAGTCT | 0.9724 | LOC391770-[CDH18]-LOC266786 |
| 3686735 | rs4492078 | 5 | NC_000005.5 | 21838097 | A | T | ATTTCCCATTTGTACACATGCAATATGATTAAAATAGATCTCTAAAGAAGA | 0.7458 | MGC22265-[CDH12]-PMCHL1 |
| 3686757 | rs13153198 | 5 | NC_000005.5 | 21860993 | T | C | GCTTCACTTTTCTGCCTTTACTTTGCTATTGGAAATTCCTATAATTTGCCT | 0.7935 | MGC22265-[CDH12]-PMCHL1 |
| 3686764 | rs6452004 | 5 | NC_000005.5 | 21869848 | G | T | TTTTCATCATCTCCTTTCCTGGGGTGTTTTCACCTCACCATTGGAGGCAGC | 0.8253 | MGC22265-[CDH12]-PMCHL1 |
| 3687650 | rs7726038 | 5 | NC_000005.5 | 23251375 | T | G | TTTGCGAAGATGTTTCCTATTGCCTTAAATACTTGCCTTGCACAGTAGCTT | 0.419 | CDH12-[ ]-LOC391771 |
| 3687661 | rs10038864 | 5 | NC_000005.5 | 23272263 | A | T | TTGATGGAATTGGAAAGGCAATTTCAGCTCTAAATCACCACAAATCTTCAG | 0.4564 | CDH12-[ ]-LOC391771 |
| 3692953 | rs13161116 | 5 | NC_000005.5 | 33827188 | T | G | ACAGCTGCAGCAGCTGCATATCAGATAATGGAATCTGCTGAGACACTGGGA | 0.6889 | LOC391776-[ADAMTS12]-SALPR |
| 3693100 | rs10077475 | 5 | NC_000005.5 | 34054510 | T | C | TAGAACCAGAAATGGGTGCCAGAGATATGCCTGCACTAATCTTAAGTGGGG | 0.9916 | AMACR, AAATP [ ] C1QTNF3 |
| 3700031 | rs8046048 | 16 | NC_000016.5 | 25455441 | A | G | GCTCCAGGTGAAAGAACAAAGCCACATGTCCTTTTCTCCACTCACCCCTGA | 0.3947 | HCP39 [ ] HS3ST4 |
| 3707022 | s16957821 | 17 | NC_000017.6 | 9148670 | C | G | GTCTTTAACATGTAACTCGATCTTCCACAGATTAAGTGACAAAGTCATTCA | 0.8711 | LOC388334-[NTN1]-LOC400572 |
| 3708771 | rs4785287 | 16 | NC_000016.5 | 49210946 | C | A | CAGCTCCATGGATGACTGGGAGAGACAGCCCCTTGCAGAGCACCCACACTT | 0.8226 | LOC400535 [MGC33367]-OAZ |
| 3713131 | rs17711876 | 17 | NC_000017.6 | 15044582 | A | G | TCCCAGTTCCTACAGGTTGTTCCTCAATGATGCCTGGATCTCACTTCAGGG | 0.6405 | LOC400576-[ ] LOC390765 |
| 3713139 | rs9914741 | 17 | NC_000017.6 | 15053614 | C | T | CACCCATATCTGCACTAAGATTTTCCGTAGGACTTTGGGGTCTTGTTTAAT | 0.6662 | LOC390765 [ ]-LOC388340 |
| 3715741 | rs7187684 | 16 | NC_000016.5 | 55573718 | T | C | GATAACGGATAGCGGCTCAGGAGAATATTAAGAGCATCGACTCTGGAGCCA | 0.1977 | LOC390732 [CESR] CES1 |
| 3748654 | rs34056793 | X | NC_000023.5 | 68207123 | T | C | GTTGGACAGCTTGGCCTCTGATTCATGTAAGCTTAAGAAGGTCTGCTACTA | 0.9843 | LOC139562-[ ] IGBP1 |
| 3751183 | rs5936560 | X | NC_000023.5 | 68565445 | A | T | TCTGCTCTCCTTGGGCCTTAGTGTCAGCTCTAAAGATTTATAAAGGTTGCA | 0.7368 | KIF4A, OBDPF [DLG3]-TEX11 |
| 3819011 | rs34963996 | X | NC_000023.5 | 85375129 | G | C | GTACAGTGTACAAACGTCAACAGAAGACAAAGATATGCCTAGAACTATCTA | 0.917 | DACH2-[ ]-KLHL4 |
| 3819892 | rs5949613 | X | NC_000023.5 | 92463278 | C | A | TGATTGCCGACCCCGTATATTGGTTCGAGGAGCTAGATTTCCTTACTGTTA | 0.5697 | LOC392503-[ ]-CALM1P1 |
| 3819894 | rs5949644 | X | NC_000023.5 | 92477379 | C | T | GTAAAATGTTTATTTTGCTCTACTACACCAGACTAATTAAACAAATCAGAA | 0.5656 | LOC392503-[ ]-CALM1P1 |
| 3820201 | rs5949835 | X | NC_000023.5 | 94424720 | T | C | TTATATGAAGCATTGCCCCATCTGCTCAATCCACAGACACACTTGCTTCTT | 0.6476 | LOC401606-[LOC392504]-DIAPH2 |
| 3820225 | rs6523008 | X | NC_000023.5 | 94480722 | A | G | CCCAGAAAACATTAGTAGTTGCCCCAAGTTATTCAAATACAGTTTTCCTGA | 0.641 | LOC401606-[LOC392504]-DIAPH2 |
| 3842849 | rs6610426 | X | NC_000023.5 | 39451157 | A | G | CTTAGTGGGTAACATCTTTCAACCCAGGTAAGATTTCTAATTAAGTTTAGA | 0.8499 | LOC402394-[ ] ATP6AP2 |
| 3848797 | rs17326689 | X | NC_000023.5 | 105624912 | G | A | CTTAAGGGGAGGATAGCTGGGACCCGTCTATTTCCGTGGATCTGGGCACAG | 0.898 | KIAA1817 [ ] PRPS1 |
| 3848852 | rs1407901 | X | NC_000023.5 | 106045348 | G | A | ATTCTCTGCTCTTGGAGACCTGAAAGGCCAGAGAACTCCAAAAGACAAAAG | 0.5627 | TEX13B [ ] MGC44287 |
| 3857566 | rs5952158 | X | NC_000023.5 | 114303633 | G | T | ACTAAACAACTGAAATGTTGCACTGGTGACCAATGGGCTGGCTGTCACCAG | 0.9717 | AGTR2-[ ] SLC6A14 |
| 3861184 | rs2238902 | X | NC_000023.5 | 10621952 | T | G | GCCCTAGAAGGATCTTTTACTTGCTTTCATTTTTCCACAGTCAAGTTATAT | 0.741 | HCCS-[ARHGAP6]-AMELX |
| 3862078 | rs5935311 | X | NC_000023.5 | 11739389 | T | C | TTTCCTCTTTCCTTTCTCACACTTATGCAGTTGGTTATAGTTCATCTATTG | 0.7273 | MSL3L1-[KIAA0316]-PRPS2 |
| 3862097 | rs7876995 | X | NC_000023.5 | 11756116 | C | G | TAACCAGATITCCCATTGACTCCTACGATGAGATTTACATAACTTGATATT | 0.7179 | MSL3L1-[KIAA0316]-PRPS2 |
| 3863206 | rs17322192 | X | NC_000023.5 | 12779533 | C | G | AATTTATAAAATGGATAGGCAATGCCTACTTGACATGATTGGTGAGGTGAA | 0.6033 | LOC389839 [ ]-EGFL6 |
| 3865731 | rs6632799 | X | NC_000023.5 | 15669009 | G | T | TTCTATTTACCACTGGACAAAGACCGAGCTAGAGGCAAATCTTGAGAATAA | 0.8404 | LOC139451-[LOC139452]-LOC392429 |
| 3866218 | rs12396950 | X | NC_000023.5 | 7489538 | G | T | CCCTTCATGGAGGACCTTCAGTTTTGGGGAGCTGACCTGTGCTGACTTAGC | 0.5739 | DXS1283E-VCX-2r |
| 3868546 | rs12833104 | X | NC_000023.5 | 112688913 | G | A | CAGCCTTTATCCTCAGAGCAATAACGATGATAGTGACAGTTCTTGACTTTT | 0.8004 | LOC286528-[HTR2C]-IL13RA2 |
| 3870054 | rs5956542 | X | NC_000023.5 | 121207687 | T | G | GAAAAATGGTCTCCTAATATTAACATTCCTAGCATACCACTGTCCCCTTCT | 0.5226 | LOC286423-[GRIA3]-LOC392533 |
| 3870185 | rs2473184 | X | NC_000023.5 | 121446469 | C | T | AAGCCATCTAGTCAGCTGAGGATGACTAAACAATTCAAAAGACTAAAAAAT | 0.274 | LOC402422-[THOC2]-BIRC4 |
| 3870505 | rs6608182 | X | NC_000023.5 | 122094859 | C | T | ATACTCTACAGCACATCTCATTCCCCTTATGAACCTGCCTCTAGTCAGTCA | 0.3758 | LOC392535-[ ]-LOC139116 |
| 3875859 | rs5931090 | X | NC_000023.5 | 134962628 | C | G | TGTATTT1ATCAGACTGTAAATAGGCCTGTTTAGAAGATTTGCTGCATTTT | 0.2881 | GPR101-[ ]-RAC4 |
| 3875872 | rs4829606 | X | NC_000023.5 | 134977294 | A | T | GTAAGAGAAAGACTTAAGAACCTGTAACAATATTGAATCCACAATCAGATA | 0.2731 | GPR101-[ ]-RAC4 |
| 3876840 | rs12556549 | X | NC_000023.5 | 136846343 | G | T | GTCCAATTTTCTATATCAGTTGTTAGAATTGACCATTAACTCTATAATATT | 0.8783 | FGF13-[ ]-SRD5AP1 |
| 3876937 | rs6528600 | X | NC_000023.5 | 137002181 | A | G | TATAACCATTTCTGTGGGTTCAGCAATCACCAACCAGAGAGAGGTAAATTG | 0.9805 | FGF13-[ ]-SRD5AP1 |
| 3881931 | rs5952057 | X | NC_000023.5 | 145491215 | C | G | CACTGTAAGGCCTTCTGTGTTAAAACAGCAGCCTTGCCTCCAGCACACATA | 0.0838 | LOC158813-[ ]-FMR1 |
| 3881989 | rs5905149 | X | NC_000023.5 | 145575900 | C | A | GGTCTTGTCTAGGGAGGAATGCACCCAATCCAAACAAATCATGACATGCCA | 0.8331 | LOC158813-[ ]-FMR1 |
| 3896084 | rs4828524 | X | NC_000023.5 | 16462614 | A | G | CTGGAATGCAGCTGAAATGACAGAAATGTTCCGAGCACTTTTTTTTTTTTT | 0.1917 | RNU4P6-[REPS2] PRO0386 |
| 3896625 | rs6418752 | X | NC_000023.5 | 21397671 | G | A | GTGGGCTGCTGTCTCCTGATGCTGCGTGGTGTGCCCCATGGACATTTGAAA | 0.3301 | SMS [ ] PHEX |
| 3901134 | rs6631192 | X | NC_000023.5 | 30223585 | G | A | TCACAAAAAAGCTGGTITTCATCAAGTCATGCACTGATTTGCATCTATTCT | 0.3481 | GK-[TAB3]-FTHL17 |
| 3901755 | rs5927030 | X | NC_000023.5 | 31066019 | G | A | TATGCTAATGATCTCTATTCCAGGCGAACAAATGTCCTCTGAATTTCCTTT | 0.6497 | FTHL17-[DMD]-LOC389843 |
| 3904418 | rs5928767 | X | NC_000023.5 | 34614015 | T | A | GACATACAGCAAAACACTTCTAAGCTTTTTTTTTAATAGCAAGTTTAAGTT | 0.8431 | LOC392440-[ ]-LOC340571 |
| 3904449 | rs5928811 | X | NC_000023.5 | 34696754 | C | T | CTTTTATTTTTTAAGTGAAGTGAAACGGTAACCTGTGCTAACAGGAAGCAA | 0.8347 | LOC392440-[ ]-LOC340571 |
| 3993569 | rs7569023 | 2 | NC_000002.6 | 10346596 | A | G | TGGGGCAGGCCCAGAATGGCCCTGAATCCAGTAACACCAACTGTCCCTGGA | 0.819 | RRM2-[FLJ25102]-HPCAL1 |
| 4008972 | rs777317 | 2 | NC_000002.6 | 215938741 | C | G | TATGGTATTTGGGGGACTAGAATTACAAATAGGCTCTGTGGTGCTTTTTTC | 0.0212 | LOC285176 [ ]-ABCA12 |
| 4027550 | rs4552392 | 3 | NC_000003.6 | 187030386 | C | T | AGAATCTGGGTTCAAGTCCAGGTGGCATGGCTTCCAGGCTGGCATGTGATG | 0.739 | SFRS10, LOC344887 [ ]-ETV5 |
| 4032210 | rs2651148 | 3 | NC_000003.6 | 194882597 | A | G | TGATAGGAAATGGGGCTTCAGGTTGAAATCAGATGAGAGCTGGCCTCTGAG | 0.1719 | OPA1-[ ]-LOC389186 |
| 4040872 | rs6835973 | 4 | NC_000004.6 | 187375123 | A | G | TGCTGTTCAGAGCATTGCTGACACCAGGGCATGAAACTAGTGACACAGTTA | 0.7602 | PRO0618-[ARGBP2]-TLR3 |
| 4044139 | rs4402996 | 4 | NC_000004.6 | 163806913 | G | C | CTTTTTTTGATATTATACAAAGAGTGAGATACAAAGGCATTTGTTTTATTA | 0.836 | DKFZp566D234-[ ]-LOC92345 |
| 4080468 | rs4617096 | 7 | NC_000007.8 | 118816377 | A | C | CAGGAAAGTAGTGAATTGTGGCACAAAGCTGGAATATGCTGTCTTTTCCTT | 0.8485 | ANKRD7-[ ]-KCND2 |
| 4092120 | rs7826501 | 8 | NC_000008.6 | 65726252 | C | A | AGTTTTCCTGGGGAAGCCGAGGCCGCCTAGAGGCAGAAAGCTGTCATCCCG | 0.793 | BHLHB5-[CYP7B1] LOC389665 |
| 4122930 | rs2904981 | 11 | NC_000011.5 | 64778386 | C | T | ATCATTTCCTTGACTGCTGTCCTAACGTTGGATGTTTGAATAGGGGTTTTG | 0.1365 | CAPN1, LOC387780 [ ] POLA2 |
| 4122932 | rs1633466 | 11 | NC_000011.5 | 64791533 | C | G | ATATTTTGGTGGCTTGGCTCTGGCTCGGACCCTGGCTGTGTAGGCCTGGTC | 0.1263 | CAPN1, LOC387780 [ ] POLA2 |
| 4139599 | rs1358221 | 12 | NC_000012.6 | 104428922 | T | C | CATTCTTCTCAGGACCTTTGCTCTTTGCCTTTCTCTGTCTGAAGGTTCACT | 0.5152 | LOC387882-[LOC390355]-LOC245718 |
| 4150772 | rs6496481 | 15 | NC_000015.5 | 86693066 | C | T | CCATGCTTACTCACTCTATGACCCACTGAGTAAAAGCTAGTTTGGAATAAA | 0.7881 | NTRK3-[ ] MRPL46, MRPS11 |
| 4171006 | rs2302845 | 18 | NC_000018.5 | 52597765 | G | A | TGTGGTCTTAGGTGAGAGAAGCCGCGCAGGCCCTGCTTCTGGCGGAACTGA | 0.363 | TXNL-[WDR7]-MGC33608 |
| 4213286 | rs2735801 | 14 | NC_000014.4 | 103569724 | T | A | TTATAAAATGATGATTCACTTTGTCTAATATCTCAGTTTTTATAATTAATC | 0.78 | GPR132-[ ] JAG2, NUDT14 |
| 4232561 | rs8084365 | 18 | NC_000018.5 | 12869737 | T | A | AAAATACTAAATTCATTTTTCCCGCTAGAATTTCAACAAGGTATCAAGTTC | 0.9583 | HCCA3-[PTPN2]-SEC13L |
| 4283222 | rs6466702 | 7 | NC_000007.8 | 118802003 | T | C | CTGGAGGGATCCGAGACCCACTCTCTTATTTTTCCTTCTGAACAAAAGCCT | 0.8463 | ANKRD7-[ ]-KCND2 |
| 4295305 | rs7591439 | 2 | NC_000002.6 | 113189939 | G | A | TCCAGACTTACAAGATTACTTCATGGTGAAAGTTTGGATTGATCAATAATT | 0.6101 | FLJ41410 [ ] LOC400998, |
| | | | | | | | | | LOC389020 |
| 4320535 | rs2651158 | 3 | NC_000003.6 | 194905895 | C | T | CAGTTCCAGCAGGGTTGGGTGAGACCTCAGGGTGATGCATGGGCACTGTTT | 0.7213 | OPA1-[ ]-LOC389186 |
| 4336765 | rs5912040 | X | NC_000023.5 | 115502230 | T | C | AGAGCTGAAGGAAAGGGTATTTATATATAAGCTGTTCTTGGTCCTAAAAAA | 0.8931 | LOC392527-[ ]-KLHL13 |
| 4353088 | rs4629907 | 8 | NC_000008.6 | 65800787 | T | C | GCTTGTACCTTGCTTACTAACATTGTGCTGCAATCACATGGCCAAGTCCAA | 0.7907 | LOC389665, CYP7B1 [ ]-LOC392227 |
| 4404676 | rs4240146 | X | NC_000023.5 | 11685568 | A | C | TAACCACCACCTTGGCTACATCTTCACTTTATACTTTCACAGGAGATTTTG | 0.8801 | MSL3L1-[KIAA0316]-PRPS2 |
| 4428163 | rs6001009 | 22 | NC_000022.5 | 36748395 | A | G | GCCCCAGACTTTGGGGGGCCCATCTACAAACCTAGGCTCACCCAGAATCCT | 0.6073 | PRKCABP, SLC16A8 [FLJ22582] |
| | | | | | | | | | PLA2G6 |
| 4431812 | rs6438666 | 3 | NC_000003.6 | 122836521 | T | C | ATGAATGAAACTGATGTGGCAGTATTTATAAACAGATGTAAATGGGAGATT | 0.3773 | GOLGB1 [KIAA0036] EAF2 |
| 4436593 | rs6505114 | 17 | NC_000017.6 | 27644703 | G | A | CTAAAGGCAAGCAACATAAAGATAAGTAAGCCCAGTGTCAGAGCTCAGGCT | 0.4524 | PIPOX-[LOC399700] LOC400589 |
| 4442429 | rs6589848 | 11 | NC_000011.5 | 120261670 | T | C | CTGTATTCAGTAAAGGAGTATTCGGTGAGGGGGAAGTTCATGATCAAATGT | 0.183 | ARHGEF12-[GRIK4]-MGC10233 |
| 4449341 | rs6825537 | 4 | NC_000004.6 | 189356525 | C | T | TTAAGACCAGTCTTTCTAACTAACCCAGTCAGACAAAAATAAAGAAAAAGT | 0.6902 | LOC389248-[ ] LOC391726 |
| 4453632 | rs6997149 | 8 | NC_000008.6 | 65773767 | G | A | ACAGGAATAGCTGAGGAAAAAGGCTGCTTATCATGATCTTCCTCTGCTGAG | 0.7908 | LOC389665, CYP7B1 [ ]-LOC392227 |
| 4476399 | rs8089593 | 18 | NC_000018.5 | 30053081 | A | T | GTCAAATCCATATGAGTATAAACTCATTGTACCAATATCCACGTGAAGCTA | 0.7146 | KIAA1713-[NOL4]-DTNA |
| 4478770 | rs4331673 | 3 | NC_000003.6 | 135030432 | C | A | TCCACTTCTCCATCGCACTGTCTGCCTTCACACAACTCATTCTTGGTCCTG | 0.8586 | FLJ22173-[SLCO2A1]-RYK |
| 4486048 | rs7825328 | 8 | NC_000008.6 | 74521967 | A | T | TCAAATCGAACAGGTAGTATTTGTAAGTGACTCCACCTCTTCTTTTACACA | 0.1819 | RDH10-[STAU2]-FLJ11011 |
| 4491310 | rs4131154 | 13 | NC_000013.6 | 106937076 | A | T | GGTAAGAGATACTGGTTATTATGATATAACAAGTGGTGGGATTCTTATGCT | 0.7505 | TNFSF13B-[ ] MYR8 |
| 4516175 | rs4129182 | 7 | NC_000007.8 | 118758986 | A | T | CAGGCAGGGCTAATGTGCAGCTCCCACTTGGATGGGAAGAACAGTGTGTGG | 0.1735 | ANKRD7-[ ]-KCND2 |
| 4545337 | rs4973591 | 2 | NC_000002.6 | 234048294 | G | A | TGGAGCCCTGCCATCTCTCGCTATCGTCTTCTGCCTCTCAACTAGGGGAAT | 0.6842 | L00389084-[NGEF]-NEU2 |
| 4551370 | rs6554742 | 5 | NC_000005.5 | 12714442 | G | A | TAGAGACTAGAGGAGGAGGTAATTGGTTTACATAGGGTCCAGGGGATTGGT | 0.8423 | CTNND2-[ ]-LOC401175 |
| 4552714 | rs4489033 | 5 | NC_000005.5 | 104895319 | A | T | AAGACATAATTGTATAATTTATTCCAATTGCTTTTTTTTAAACAAAAATCT | 0.0955 | RAB9P1-[ ]-LOC345571 |
| 4560172 | rs4302812 | 8 | NC_000008.6 | 103452803 | G | A | GCTGCTGGTCACTGACTCTGAACTGGGAGCCTGATCTTCTTCTCCAGCTCT | 0.3865 | DD5-[ ]-ODF1 |
| 4566219 | rs4421782 | 12 | NC_000012.6 | 128685945 | G | T | ACAGCGATTCTGTAAAGATTTCCAGGGCATATGGTCCCTAAAGGATCATCC | 0.7317 | N0D25-[KIAA1944]-LOC400088 |
| 4571614 | rs1539853 | 18 | NC_000018.5 | 33383431 | A | G | CTGGCAATATTTTTCCAGCTGCAAGACCAGTGTCACCCCAGGAAACTGTCC | 0.2537 | KIAA1328-[BRUNOL4]-LOC388474 |
| 4588473 | rs7721152 | 5 | NC_000005.5 | 117790836 | G | A | TAGTAGGGGAATTTTCTGTTGTCTTGTGTGGTTAACTTCTAGACTGTATTT | 0.83 | RPS17P2-[ ]-FLJ33977 |
| 4589662 | rs4534816 | 15 | NC_000015.5 | 86995087 | G | A | TAATCATTGGGTCTACAGAAACCACGTGCTTGAGAAATGGAAGCCCTGGAG | 0.8279 | ISG20-[ ]-AGC1 |
| 4592239 | rs6953246 | 7 | NC_000007.8 | 666S0912 | G | A | TGTCCACATAAGACAACCTCTGTTCGGAGCAATTAAAGGCGAATCTGGACC | 0.4296 | FLJ13195-[ ]-AUTS2 |
| 4662308 | rs4676753 | 3 | NC_000003.6 | 122849512 | T | C | TATTGGCCATGTAACTTTAGTGGAATGTTGGATAAAGGTCACTAATCTTAT | 0.3775 | GOLGB1-[KIAA0036] EAF2 |
| 4670519 | rs5904984 | X | NC_000023.5 | 144935838 | T | C | CTTTCCATGTTCCTGACTCCTAAGCTGAACACATTTTAATTTATTATATAT | 0.7116 | LOC158613-[ ]-FMR1 |
| 4672031 | rs1594887 | 18 | NC_000018.5 | 43144356 | G | A | GTTTAATTTCCATGTATCTTGTACCGTTTCCAGAGTTCCTCTCATTATTGT | 0.2817 | HSPC039-[ ]-LOC400650 |
| 10940 | rs2823731 | 21 | NC_000021.4 | 16566315 | A | G | ATACATTATATTTAAACATATCTCTATAGAGTCAACAAAATAAAATAAACA | 0.5986 | VDAC2P-[LOC388815]-LOC391270 |
| 12394 | rs2823937 | 21 | NC_000021.4 | 16947265 | A | C | ATGTAAGATCGTTTGGGAAAATGTTAAGACAGATATCTTGCTTTAATTTTT | 0.6183 | LOC388815 [ ]-LOC391270 |
| 17207 | rs2824839 | 21 | NC_000021.4 | 18746350 | C | A | GCTTATGTTTATGTGATGGCACCTGCGAGTACATAGAGGTTGGATATGTTA | 0.5494 | PRSS7 [ ]-LOC388816 |
| 17255 | rs2824848 | 21 | NC_000021.4 | 18775552 | T | C | ACTCCAACCCACAGCATTATTATTATTCAGTAGGTTATAGAGGTGTTATAC | 0.5109 | PRSS7-[ ]-LOC388816 |
| 20802 | rs2825756 | 21 | NC_000021.4 | 19999393 | A | G | ACCCCTTAAATTTTCATTTTCTCTCAAAGTCTCCTCTAAATTTAGTATATT | 0.8111 | SLC6A6P-[ ]-C1QBPP |
| 39641 | rs928261 | 21 | NC_000021.4 | 25378214 | T | C | TGATTAGCCTTCCATTTCATAAACCTTTTTTTCCCCTGGAATTGATAATGG | 0.3077 | LOC400860-[ ]-LOC284821 |
| 40257 | rs2829674 | 21 | NC_000021.4 | 25557670 | C | T | ATAAAGTTGGAATTTGGAGTCATGGCCTGAAAAATGTGAGCAAGTAAAGAA | 0.5755 | LOC400860-[ ]-LOC284821 |
| 54303 | rs2832046 | 21 | NC_000021.4 | 29038677 | T | C | TGGAAGGGTAGAACCTTAAGTAGTTTTTCATTCTCTGACTACTCAACTAGA | 0.8541 | C21orf100-[ ]-C21orf127 |
| 75560 | rs2226829 | 21 | NC_000021.4 | 36987160 | T | G | GATTTTGAGGCCATGTTTCCGTTAATCTGGACCGAGAGCCCTCTGGGAGAG | 0.311 | LOC388823-[ ] SIM2 |
| 76470 | rs2835628 | 21 | NC_000021.4 | 37439923 | A | G | AATGCGATTTGATGATTGTAACAGGACAAAATTTTGATTCTTTCGAAATTC | 0.2715 | DSCR5-[TTC3]-DSCR9 |
| 82434 | rs2836671 | 21 | NC_000021.4 | 39052714 | G | A | AGCTAGGTGGTGTTCTCGTGTACATGTTAGAGATGAGGAAACCCAATCTCT | 0.6101 | ERG-[LOC400866] ETS2 |
| 104324 | rs1296754 | 22 | NC_000022.5 | 16360114 | G | A | TCCTTGTTTCCCCCAGCCTTTTGTCGCTTAACATGTTTCTTTATGCTTATT | 0.3001 | CLCP1-[CECR2]-SLC25A18 |
| 120178 | rs465736 | 22 | NC_000022.5 | 28159320 | A | G | GTTCTAGAAGTGACAAAGCTGGGACACAATACCTTTATGCATGAAAAGGTT | 0.8434 | AP1B1-[REPL1] NEFH |
| 120634 | rs740041 | 22 | NC_000022.5 | 8524653 | A | G | CATCTCTCTTATCATGCTGCCTCCCAACATGCAGGGGAGAGTCCTGGCCTT | 0.601 | HSPC051, LOC55954 |
| | | | | | | | | | [ASC1p100]-MTMR3 |
| 120658 | rs2074707 | 22 | NC_000022.5 | 28534910 | G | A | CTGAATAAATGGCTCAATGAATAACGCACAAGTGAACATGTCAAACTGAAA | 0.6 | HSPC051 [ASC1p100]-MTMR3 |
| 120666 | rs17711377 | 22 | NC_000022.5 | 28537540 | C | T | TTTGGGCAGGTCTGTCCTTGGTTTCCTTATCGATGACCATGCAGCCCTTGC | 0.5875 | HSPC051 [ASC1p100]-MTMR3 |
| 120843 | rs2285667 | 22 | NC_000022.5 | 28708658 | T | A | AGCAAGAAAAGATTACTGTTCTGGCTCCCTTCAGCTTCTATGTCATTGCAT | 0.6244 | ASC1p100-[MTMR3] LOC400924 |
| 120880 | rs41157 | 22 | NC_000022.5 | 28729705 | T | C | CTTGGCCTTGGCTTTCATTTTGCATTGCTCTTAAATAATAAGTTTGCTTCT | 0.3814 | ASC1p100-[MTMR3] LOC391326, |
| | | | | | | | | | LOC400924 |
| 120906 | rs41168 | 22 | NC_000022.5 | 28742715 | A | C | CTCAGCCCCTGCTCTGAGTGCCATCAATTTAACTGTTTTGTGGTTCTTCTC | 0.3829 | ASC1p100-[MTMR3]LOC391326, |
| | | | | | | | | | LOC400924 |
| 121018 | rs1548389 | 22 | NC_000022.5 | 28898106 | T | C | AAAGGTATTGGACTTATATCCTTGATAGAATTGTAGACTGAGTCACTATAA | 0.5895 | MGC26710 [ ]-LIF |
| 133091 | rs34770535 | 22 | NC_000022.5 | 36928983 | T | A | TCCATCCGCTTCCCAGGCAGACCTATCAGCCAGACAGCTTCCGTCTTGCCT | 0.9712 | C22orf5 [ ] CSNK1E, LOC400927 |
| 137315 | rs926350 | 22 | NC_000022.5 | 41702889 | C | T | TAGAGGCAGCCATCAAATCACCACCCGGGAATGTTCAACTGCAAGTGTGCC | 0.7844 | PACSIN2-[TTLL1] BIK |
| 137829 | rs5996341 | 22 | NC_000022.5 | 42090418 | C | T | GGAGATTTCCTTGACTTCGTCTTCCCTCTTTTGGTCAAATTAAAAAATATC | 0.5363 | SCUBE1-[C22orf1]-FLJ23588 |
| 138564 | rs16991431 | 22 | NC_000022.5 | 42742499 | C | T | ACCCAGGAGGGCTTCTTGGAGGAGGCGGCCAGTAAGATGAGGTTGAAGATA | 0.7787 | CGI-51-[PARVB]-TRSPP1 |
| 145360 | rs6007770 | 22 | NC_000022.5 | 46571750 | A | G | CACCCCACACTGGACACATCCTTATAGGCACTGAGACACTTCTGGGAGCAC | 0.8837 | LOC400932-[ ]-LOC388914 |
| 159809 | rs4127784 | 14 | NC_000014.4 | 26997334 | C | T | CATCTTACAGAGTGAAGTGCCTGATCCTAAGATATGGTGGTCAAAGAGGAT | 0.5709 | RPL26P3 [ ]-BTF3P2 |
| 159815 | rs12882372 | 14 | NC_000014.4 | 27002793 | G | A | GTGCCTGGCCTACGATTTTAATTACGGTAGATTTATATTACACTTAAACCT | 0.6128 | RPL26P3-[ ]-BTF3P2 |
| 160343 | rs17114346 | 14 | NC_000014.4 | 27343901 | G | C | GTATTTTCTTTTAACTTTCAAAACTGTTTTTGCTCCAAAGAACAAAAGCAA | 0.9947 | LOC387978-[ ]-PRKCM |

TABLE 2

| | | | Binary | | Linear | | Time | |
|---|---|---|---|---|---|---|---|---|
| | | | Placebo (by | SSRI response (by | Placebo (by | SSRI response (by | Placebo (by | SSRI response (by |
| Gene | GeneID | Function | Genotype) | Interaction) | Genotype) | Interaction) | Genotype) | Interaction) |
| ADCY2 | 108 | adenylate cyclase 2 (expressed in brain) | | ANX | | | | |
| ALDH8A1 | 64577 | aldehyde dehydrogenase 8 family, member A1 | | | | ANX | | |
| ALDH9A1 | 223 | aldehyde dehydrogenase 9 family, member A1. The | | | | | | ANX, INSOM |
| | | enzyme catalyzes the dehydrogenation of gamma- | | | | | | |
| | | aminobutyraldehyde to gamma-aminobutyric acid (GABA). | | | | | | |
| AUTS2 | 26053 | autism susceptibility candidate 2 | INSOM | | CLILLY, HAMDT | | | |
| CDH12 | 1010 | cadherin 12, type 2 (N-cadherin 2). This particular cadherin | | | HAMDT, INSOM | | | |
| | | appears to be expressed specifically in the brain and its | | | | | | |
| | | temporal pattern of expression would be consistent with a | | | | | | |
| | | role during a critical period of neuronal development, | | | | | | |
| | | perhaps specifically during synaptogenesis. | | | | | | |
| CDH18 | 1016 | cadherin 18, type 2. This particular cadherin is expressed | | INSOM | | HAMDT | | |
| | | specifically in the central nervous system and is putatively | | | | | | |
| | | involved in synaptic adhesion, exon outgrowth and guidance. | | | | | | |
| DAT1 | 55885 | neuronal specific transcription factor DAT1. (aka LIM domain only | CLILLY | | HAMDT, INSOM | | | |
| | | 3 (rhombotin-like 2)) | | | | | | |
| DMD | 1756 | dystrophin (muscular dystrophy, Duchenne and Becker types) | | | | CLILLY | | |
| DRD2 | 1813 | dopamine receptor D2. A missense mutation in this gene | | HAMDT | | | | |
| | | causes myoclonus dystonia; other mutations have been | | | | | | |
| | | associated with schizophrenia | | | | | | |
| GRID2 | 2895 | glutamate receptor, ionotropic, delta 2. Predominant | | CLILLY | | CLILLY | | |
| | | excitatory neurotransmitter receptors in the mammalian | | | | | | |
| | | brain. GRID2 is a predicted 1,007 amino acid protein that | | | | | | |
| | | shares 97% identity with the mouse homolog, which is | | | | | | |
| | | expressed selectively in cerebellar Purkinje cells. | | | | | | |
| GRM8 | 2918 | glutamate receptor, metabotropic 8. The major excitatory | | | | | CLILLY | |
| | | neurotransmitter in the central nervous system and | | | | | | |
| | | activates both ionotropic and metabotropic glutamate | | | | | | |
| | | receptors. Glutamatergic neurotransmission is involved in | | | | | | |
| | | most aspects of normal brain function and can be perturbed | | | | | | |
| | | in many neuropathologic conditions. | | | | | | |
| HTR2C | 3358 | 5-hydroxytryptamine (serotonin) receptor 2C. Higher | | | | CLILLY | | |
| | | distribution of the -759T allele of the 5HT2C receptor in | | | | | | |
| | | normal controls compared with in patients with | | | | | | |
| | | schizophrenia. Involvement of the -759C/T polymorphism of | | | | | | |
| | | the 5-HT2CR in clozapine-induced weight gain in German | | | | | | |
| | | patients with schizophrenia. 5HTR2C Cys23Ser | | | | | | |
| | | polymorphism may be associated with migraine with aura in | | | | | | |
| | | a Japanese population. | | | | | | |
| HTR3A | 3359 | 5-hydroxytryptamine (serotonin) receptor 3A. This gene | | | | | | ANX, INSOM |
| | | encodes subunit A of the type 3 receptor for 5- | | | | | | |
| | | hydroxytryptamine (serotonin), a biogenic hormone that | | | | | | |
| | | functions as a neurotransmitter. This receptor causes fast, | | | | | | |
| | | depolarizing responses in neurons after activation. | | | | | | |
| LAMA2 | 3908 | laminin, alpha 2 (merosin, congenital muscular dystrophy). | | | | CLILLY | | CLILLY |
| | | Mutations in this gene have been identified as the cause of | | | | | | |
| | | congenital merosin-deficient muscular dystrophy. | | | | | | |
| LARS2 | 23395 | leucyl-tRNA synthase 2, mitochondrial. Upregulation of | | INSOM | | INSOM | | |
| | | LARS2 is a hallmark of 324A > G mutation. The | | | | | | |
| | | accumulation of 324A > G mutation in the brain may havea | | | | | | |
| | | pathophysiologic role in bipolar disorder and schizophrenia. | | | | | | |
| LOC399921 | 399921 | (similar to SHANK2) | | | | | | INSOM |
| NCAM1 | 4684 | neural cell adhesion molecule 1. Genetic variations in | | | INSOM | | | |
| | | neural cell adhesion molecule 1 or nearby genes could | | | | | | |
| | | confer risks associated with bipolar affective disorder in | | | | | | |
| | | Japanese individuals. | | | | | | |
| PHYH | 5264 | phytanoyl-CoA 2-hydroxylase. Genetic variations underlie | | | | | | INSOM |
| | | Refsum disease, an autosomal recessive disorder | | | | | | |
| | | characterized clinically by a tetrad of abnormalities: retinitis | | | | | | |
| | | pigmentosa, peripheral neuropathy, cerebellar ataxia, and | | | | | | |
| | | elevated protein levels in the cerebrospinal fluid (CSF) | | | | | | |
| | | without an increase in the number of cells in the CSF. | | | | | | |
| PNR | 9038 | putative neurotransmitter receptor | | CLILLY | | | | |
| ROBO1 | 6091 | roundabout, axon guidance receptor, homolog 1. This | HAMDT | | ANX, CLILLY, | | | |
| | | receptor is involved in the decision by axons to cross the | | | HAMDT | | | |
| | | central nervous system midline. | | | | | | |
| SEMA5A | 9037 | sema domain, seven thrombospondin repeats (type 1 and | | | ANX | | | INSOM |
| | | type 1-like), transmembrane domain (TM) and short | | | | | | |
| | | cytoplasmic domain, (semaphorin) 5A. Involved in axonal | | | | | | |
| | | guidance during neural development. | | | | | | |
| SHANK2 | 22941 | SH3 and multiple ankyrin repeat domains 2. This gene | | | | | | ANX, INSOM |
| | | encodes a protein that is a member of the Shank family of | | | | | | |
| | | synaptic proteins that may function as molecular scaffolds | | | | | | |
| | | in the postsynaptic density (PSD). The alternative splicing | | | | | | |
| | | demonstrated in Shank genes has been suggested as a | | | | | | |
| | | mechanism for regulating the molecular structure of Shank | | | | | | |
| | | and the spectrum of Shank-interacting proteins in the PSDs | | | | | | |
| | | of adult and developing brain. | | | | | | |
| SLC1A1 | 6505 | solute carrier family 1 (neuronal/epithelial high affinity | | ANX | | | | |
| | | glutamate transporter) | | | | | | |
| SLC5A7 | 60482 | solute carrier family 5 (choline transporter), member 7. | | | | HAMDT | | |
| | | Neurotransmitter of the central and peripheral nervous | | | | | | |
| | | system that regulates a variety of autonomic, cognitive, and | | | | | | |
| | | motor functions. SLC5A7 is a Na(+)- and Cl(−)-dependent | | | | | | |
| | | high-affinity transporter that mediates the uptake of choline | | | | | | |
| | | for acetylcholine synthesis in cholinergic neurons. | | | | | | |
| SLC6A14 | 11254 | solute carrier family 6 (neurotransmitter transporter), | | CLILLY | | | | |
| | | member 14. Transports both neutral and cationic amino | | | | | | |
| | | acids in an Na(+)- and Cl(−)-dependent manner. | | | | | | |
| WFS1 | 7466 | Wolfram syndrome 1 (wolframin). Diverse neurologic | | INSOM | | | | |
| | | symptoms, including a predisposition to psychiatric illness, | | | | | | |
| | | may also be associated with this disorder. A large number | | | | | | |
| | | and variety of mutations in this gene, particularly in exon 8, | | | | | | |
| | | can be associated with this syndrome. Mutations in this | | | | | | |
| | | gene can also cause autosomal dominant deafness 6 | | | | | | |
| | | (DFNA6), also known as DFNA14 or DFNA38. | | | | | | |
| WNT2 | 7472 | wingless-type MMTV integration site family member 2. A | | | | CLILLY | | |
| | | strong candidate gene for autism. | | | | | | |

TABLE 3

| | | | | | Alleles | | | | |
|---|---|---|---|---|---|---|---|---|---|
| SNP | SNP | | | | reference | alternate | | | |
| PerI ID | dbSNP rsID | Chrom. | Accession | Position | base | base | Flanking Sequence | Gene | Analysis |

| | | | | | | | | | |
|---|---|---|---|---|---|---|---|---|---|
| 2783077 | rs1460969 | 5 | NC_000005.5 | 7818388 | G | A | CAAATGATATAACGGCAGAAATACCGTATCTCGTATCTCTATTGACTGTGA | ADCY2 | BIA |
| 3118987 | n/a | 5 | NC_000005.5 | 7838724 | G | A | AATTCTCCCAACTTTGTTATTGGTCGTTGAGATGATACACATTCAGTACCA | ADCY2 | BIA |
| 280097 | rs728030 | 6 | NC_000006.6 | 135226864 | C | A | GATTTGTATACTATTGAGGTATTAACGATCCATATTTAACCAAGTGTTTTC | ALDH8A1 | LIA |
| 1128559 | rs4578194 | 1 | NC_000001.5 | 162837993 | C | T | TGACATTGAAGACCAGAATGGTTCACTTGATGAGAGTCCCCAAAGCTAGTG | ALDH9A1 | TIA |
| 555251 | rs1003404 | 7 | NC_000007.8 | 68467077 | T | C | TGTCTTTATCTGCACTATAAAATACTGCAGCCTAGCTGGATGAGACGGTTA | AUTS2 | LGC, LGH |
| 555297 | rs10487947 | 7 | NC_000007.8 | 68416498 | G | A | GTGCCCAGCCCCTGGTGATTTTATGGAGAACTTACTCTGTGCCCTTGGATA | AUTS2 | Genotype |
| 555302 | n/a | 7 | NC_000007.8 | 68407681 | G | A | TACAAGTATGATAGCATCAAACACAGGGCTTAGTTTGCATGCCCTCTTATA | AUTS2 | Genotype |
| 1572691 | rs17762851 | 7 | NC_000007.8 | 69630695 | C | T | CAAGTAATTGAATCTTCTAATGGAACAAACTGGTCTCTGCTTAATGATTTG | AUTS2 | BGI |
| 4592239 | rs6953246 | 7 | NC_000007.8 | 66650912 | G | A | TGTCCACATAAGACAACCTCTGTTCGGAGCAATTAAAGGCGAATCTGGACC | AUTS2 | Genotype |
| 3686735 | rs4492078 | 5 | NC_000005.5 | 21838097 | A | T | ATTTCCCATTTGTACACATGCAATATGATTAAAATAGATCTCTAAAGAAGA | CDH12 | LGI |
| 3686757 | rs13153198 | 5 | NC_000005.5 | 21860993 | T | C | GCTTCACTTTTCTGCCTTTACTTTGCTATTGGAAATTCCTATAATTTGCCT | CDH12 | LGI, LGH |
| 3686764 | rs6452004 | 5 | NC_000005.5 | 21869848 | G | T | TTTTCATCATCTCCTTTCCTGGGGTGTTTTCACCTCACCATTGGAGGCAGC | CDH12 | LGI |
| 3687650 | rs7726038 | 5 | NC_000005.5 | 23251375 | T | G | TTTGCGAAGATGTTTCCTATTGCCTTAAATACTTGCCTTGCACAGTAGCTT | CDH12 | Linear, |
| | | | | | | | | | Genotype |
| 3687661 | rs10038864 | 5 | NC_000005.5 | 23272263 | A | T | TTGATGGAATTGGAAAGGCAATTTCAGCTCTAAATCACCACAAATCTTCAG | CDH12 | Linear, |
| | | | | | | | | | Genotype |
| 3686058 | rs16885644 | 5 | NC_000005.5 | 19694961 | A | T | GAAGCAAAATATATACAAGTTAAAGATATGTGTTCAGCTTCAGTCCAGTCT | CDH18 | BII, LIH |
| 906272 | rs7304507 | 12 | NC_000012.6 | 16545580 | G | A | CACAACTGTATCTGAACAGATTCTCGITACATAAAACCGCACACACAGTGT | DAT1 | LGH, LGI |
| 3901755 | rs5927030 | X | NC_000023.5 | 31066019 | G | A | TATGCTAATGATCTCTATTCCAGGCGAACAAATGTCCTCTGAATTTCCTTT | DMD | LIC |
| 612631 | rs1107162 | 11 | NC_000011.5 | 112826688 | A | G | TGGGTGTCTGAGGCCCTTGCCCCTCGCTTATCTTCTCCCAGATACATAAGA | DRD2 | BIH |
| 1752273 | rs10891539 | 11 | NC_000011.5 | 112774141 | G | C | GTAACCCCGGGAGCTGAGTGAGAGAGGCTCCTTCCCTTACATCCACATGCC | DRD2 | BIH |
| 1752293 | rs754672 | 11 | NC_000011.5 | 112786785 | C | T | TCCTGGGCCACTGAATTGCCAACTGCGTGACCCAAGGCTCCTCTAAACCTG | DRD2 | BIH |
| 3434449 | rs6824100 | 4 | NC_000004.6 | 94964127 | G | T | CTTTTTGTAAGAGGATACAATAAAAGTATGAGTCAAAGAATATATTGGGGA | GRID2 | BIC, LIC |
| 528496 | rs2237794 | 7 | NC_000007.8 | 126346076 | G | C | TCAAATTAAGGGGATCATCAACAACGTTTTCTACAGTTCACATAGGAGGCG | GRM8 | TGC |
| 3868546 | n/a | X | NC_000023.5 | 112688913 | G | A | CAGCCTTTATCCTCAGAGCAATAACGATGATAGTGACAGTTCTTGACTTTT | HTR2C | LIC |
| 1752882 | rs17626940 | 11 | NC_000011.5 | 113430360 | G | A | CACTGAGTAAGCAGGTGCCTCCAAAGGTCTTACTAAGCCACAGGTAGGAAG | HTR3A | TIA |
| 1529845 | rs17056873 | 6 | NC_000006.6 | 129456505 | C | G | TTGAAAGCTTCTGTAAACAGTTGAACTTCAAATTAAAAGGTAAGTAGGAAC | LAMA2 | LIC |
| 1529998 | rs265326 | 6 | NC_000006.6 | 129570746 | C | G | GTTTATTTTTCATGGTTTTAACCCAGCATTAAGTAGCATGGTTTTTAGCAT | LAMA2 | TIC |
| 1530007 | rs265392 | 6 | NC_000006.6 | 129576597 | A | T | AATATGAAAGAGACATGTGAATCTCTGCCTTTGAATACTTAGGATGTGTTT | LAMA2 | TIC |
| 355828 | rs9375582 | 6 | NC_000006.6 | 128938976 | T | G | TTTGTAACTTCCTTGAAGGCAGAGTTTCTTCTTCGGGTTTGTATTATCTAT | LAMA2 | Interaction, |
| | | | | | | | | | CLilly |
| 2358053 | rs11130066 | 3 | NC_000003.6 | 45489589 | C | T | GTGTAATACCCTTAGCTTTATATCTCTCAGTTTTCACACAATGTGTTGTAT | LARS2 | BII, LII |
| 2358096 | rs2578670 | 3 | NC_000003.6 | 45534058 | G | T | TTAAATTTTTATTTGCATATTTGTTTTCTATCCTAATTCCCTACTGATCTT | LARS2 | BII, LII |
| 1703484 | rs11236931 | 11 | NC_000011.5 | 70195793 | C | G | CAGCTATTGCTTATGCTCCACGCACCATTTGCCCTTTTGGAGGATCATCGT | LOC399921 | TII |
| 1752125 | rs605843 | 11 | NC_000011.5 | 112662883 | T | C | GGTGATCAGCATGCTGCTGGCCCTATGATGATAAGTAGTGGGCTCTTCCTT | NCAM1 | LGI |
| 1752273 | rs10891539 | 11 | NC_000011.5 | 112774141 | G | C | GTAACCCCGGGAGCTGAGTGAGAGAGGCTCCTTCCCTTACATCCACATGCC | NCAM1 | LGI |
| 2210865 | rs1556718 | 10 | NC_000010.5 | 13330966 | C | A | TAAGCTAATCATACCTCCCACTCTGCATCTGAGCAGGGTATCTGAGACTCC | PHYH | TII |
| 1532522 | rs6924201 | 6 | NC_000006.6 | 132877599 | C | T | GTTCCATAACCTTTGGGGCCAATTACAGGTCATGGATACACTGTTCCTAAG | PNR | BIC |
| 2386150 | rs9866565 | 3 | NC_000003.6 | 79916897 | A | G | AGTGGTATATAAAACACAGTTGTTGACCACAATATAACTAAGTTACAGAGC | ROBO1 | Genotype |
| 2386620 | rs3773220 | 3 | NC_000003.6 | 78622704 | C | T | CTCTGCATTAAAATAATAATCATGGCGAGCAACAGATAAAATAATGTTAAA | ROBO1 | LGH |
| 2386633 | rs6788434 | 3 | NC_000003.6 | 78654588 | G | A | GTATTATACTTCAGTTTACGTAATCGGGAAAATAAGAGTGGTCTAGAGAAA | ROBO1 | BGH, LGC, |
| | | | | | | | | | LGH |
| 2386656 | rs17016466 | 3 | NC_000003.6 | 78675379 | A | G | AAACAGTAACAACAACTGTATTTGCATAAGCACCCCATAATCCACACCCAC | ROBO1 | BGH, LGC, |
| | | | | | | | | | LGH |
| 2386667 | rs3773240 | 3 | NC_000003.6 | 78703985 | C | T | CTGCTTTCTATGCTGGGGTGGCAACCTAATCCAAAATTCCTATTGCAGGTT | ROBO1 | LGA, LGC, |
| | | | | | | | | | LGH |
| 2386700 | n/a | 3 | NC_000003.6 | 78735232 | G | A | CCTTCTCTCGAAGTTTCTATATGCAGATCATGACTGAATATTGTTGTTTAA | ROBO1 | BGH |
| 829556 | rs3822787 | 5 | NC_000005.5 | 9345951 | A | G | TTTCATATCCCACACTGAATACCTTGTGATGGCACTGCCACTACCACTGTT | SEMA5A | LGA |
| 829565 | rs6874451 | 5 | NC_000005.5 | 9339456 | C | A | CCCTTCAAGAGCTGACTGACCAGGGCTGGACAGTTAACTCACTCCTCCAGT | SEMA5A | |
| 1703484 | rs11236931 | 11 | NC_000011.5 | 70195793 | C | G | CAGCTATTGCTTATGCTCCACGCACCATTTGCCCTTTTGGAGGATCATCGT | SHANK2 | TIA |
| 863475 | rs6476875 | 9 | NC_000009.6 | 4519671 | T | C | ATTAGATAATTAAAAGCCTCTGCCATCAGTCAAAATGAAACTTTTTTTGTG | SLC1A1 | BIA |
| 1293364 | rs2630505 | 2 | NC_000002.6 | 108260966 | C | T | TTTCTTTGCAAACCTGTCTTGCCTATTTTTCCTTAGGTTGAAAGGATTCTG | SLC5A7 | LIH |
| 3857566 | rs5952158 | X | NC_000023.5 | 114303633 | G | T | ACTAAACAACTGAAATGTTGCACTGGTGACCAATGGGCTGGCTGTCACCAG | SLC6A14 | BIC |
| 3395544 | rs4380588 | 4 | NC_000004.6 | 6324430 | G | A | GCCATCTCTCCTCCAGGCTGGAGTCGGTGCTTCCCACAGTTACTTCTCACG | WFS1 | BII |
| 523650 | rs39311 | 7 | NC_000007.8 | 116508620 | T | G | CCCAGGGACCTTTCAATTTTATGCTTATCTTTCTTTATATATTAATATCAA | WNT2 | LIC |

TABLE 4
CNS Relevant Genes

| Model | Subscale | Perlegen_SNP_ID | FisherPval | FisherQval |
|---|---|---|---|---|
| BinGeno | Hamd T | 2386656 | 0.000779837 | 0.828149458 |
| BinGeno | Hamd T | 2386633 | 0.000789689 | 0.828149458 |
| BinGeno | Hamd T | 2386700 | 0.001988906 | 0.928914363 |
| BinGeno | INSOM | 1572691 | 0.000422659 | 0.730598063 |
| BinInteract | ANX | 3123186 | 0.00000185 | 0.172688059 |
| BinInteract | ANX | 2783077 | 0.0000939 | 0.884946863 |
| BinInteract | ANX | 918719 | 0.000127152 | 0.884946863 |
| BinInteract | ANX | 3118987 | 0.000141478 | 0.884946863 |
| BinInteract | ANX | 863475 | 0.002422978 | 0.894646315 |
| BinInteract | CLILLY | 3857566 | 0.001116038 | 0.885573423 |
| BinInteract | CLILLY | 3434449 | 0.001582873 | 0.891698664 |
| BinInteract | CLILLY | 1532522 | 0.001134751 | 0.885573423 |
| BinInteract | HamdT | 918719 | 1.66E−08 | 0.003031537 |
| BinInteract | HamdT | 1752273 | 0.0000244 | 0.60757736 |
| BinInteract | HamdT | 1752293 | 0.001136911 | 0.809078132 |
| BinInteract | HamdT | 612631 | 0.001603207 | 0.825277365 |
| BinInteract | INSOM | 2358096 | 0.001386535 | 0.907862841 |
| BinInteract | INSOM | 3686058 | 0.001461721 | 0.907862841 |
| BinInteract | INSOM | 2358053 | 0.00147295 | 0.907862841 |
| BinInteract | INSOM | 3395544 | 0.0001657 | 0.769877609 |
| LinearGeno | ANX | 2386667 | 0.000217924 | 0.942712636 |
| LinearGeno | ANX | 829556 | 0.000455991 | 0.942712636 |
| LinearGeno | CLILLY | 2386667 | 0.0000301 | 0.904680398 |
| LinearGeno | CLILLY | 2386633 | 0.0000993 | 0.904680398 |
| LinearGeno | CLILLY | 2386656 | 0.000102605 | 0.904680398 |
| LinearGeno | CLILLY | 555251 | 0.000287397 | 0.904680398 |
| LinearGeno | HamdT | 2386667 | 0.000018 | 0.607031989 |
| LinearGeno | HamdT | 2386633 | 0.0000782 | 0.896847022 |
| LinearGeno | HamdT | 2386656 | 0.0000811 | 0.896847022 |
| LinearGeno | HamdT | 555251 | 0.000475397 | 0.896847022 |
| LinearGeno | HamdT | 906272 | 0.000661222 | 0.896847022 |
| LinearGeno | HamdT | 3686757 | 0.001523578 | 0.896847022 |
| LinearGeno | HamdT | 2386620 | 0.000595901 | 0.896847022 |
| LinearGeno | INSOM | 3686764 | 0.0000394 | 0.597735223 |
| LinearGeno | INSOM | 3686757 | 0.000292836 | 0.750338675 |
| LinearGeno | INSOM | 906272 | 0.000934093 | 0.892544184 |
| LinearGeno | INSOM | 1752125 | 0.001057331 | 0.892544184 |
| LinearGeno | INSOM | 3686735 | 0.001622323 | 0.905792619 |
| LinearInteract | ANX | 918719 | 0.000156764 | 0.831437921 |
| LinearInteract | ANX | 280097 | 0.000185873 | 0.831437921 |
| LinearInteract | CLILLY | 3434449 | 0.000251456 | 0.694668025 |
| LinearInteract | CLILLY | 1529845 | 0.000366086 | 0.782412669 |
| LinearInteract | CLILLY | 523650 | 0.0006434 | 0.853208783 |
| LinearInteract | CLILLY | 3868546 | 0.001672875 | 0.853208783 |
| LinearInteract | CLILLY | 3901755 | 0.003165907 | 0.853208783 |
| LinearInteract | HamdT | 918719 | 0.00000454 | 0.465870671 |
| LinearInteract | HamdT | 3686058 | 0.000696406 | 0.828221269 |
| LinearInteract | HamdT | 1293364 | 0.000848603 | 0.83215468 |
| LinearInteract | Insom | 2358096 | 0.000278621 | 0.843864781 |
| LinearInteract | Insom | 2358053 | 0.000420189 | 0.903324249 |
| TimeGeno | CLILLY | 528496 | 0.000978517 | 0.459526693 |
| TimeInteract | INSOM | 3484509 | 0.0000476 | 0.217953355 |
| TimeInteract | INSOM | 1527003 | 0.0000854 | 0.239113003 |
| TimeInteract | INSOM | 3435351 | 5.66E−05 | 0.229827322 |
| TimeInteract | INSOM | 829565 | 0.000702387 | 0.256026918 |
| TimeInteract | INSOM | 2210865 | 0.000253873 | 0.24564659 |
| BinGeno | Hamd T | 2386656 | 0.000779837 | 0.828149458 |
| BinGeno | Hamd T | 2386633 | 0.000789689 | 0.828149458 |
| BinGeno | Hamd T | 2386700 | 0.001988906 | 0.928914363 |
| BinGeno | INSOM | 1572691 | 0.000422659 | 0.730598063 |
| BinInteract | ANX | 3123186 | 0.00000185 | 0.172688059 |
| BinInteract | ANX | 2783077 | 0.0000939 | 0.884946863 |
| BinInteract | ANX | 918719 | 0.000127152 | 0.884946863 |
| BinInteract | ANX | 3118987 | 0.000141478 | 0.884946863 |
| BinInteract | ANX | 863475 | 0.002422978 | 0.894646315 |
| BinInteract | CLILLY | 3857566 | 0.001116038 | 0.885573423 |
| BinInteract | CLILLY | 3434449 | 0.001582873 | 0.891698664 |
| BinInteract | CLILLY | 1532522 | 0.001134751 | 0.885573423 |
| BinInteract | HamdT | 918719 | 1.66E−08 | 0.003031537 |

TABLE 5A
Novel Linear GenotypeANX

| Perlegen_SNP | FisherPval | FisherQval |
|---|---|---|
| 3480149 | 7.60E−07 | 0.14672334 |
| 1203638 | 4.34E−05 | 0.909972399 |
| 1552540 | 0.000166206 | 0.942712636 |
| 4589662 | 0.000265204 | 0.942712636 |
| 2349785 | 0.000357934 | 0.942712636 |
| 2236226 | 0.00036501 | 0.942712636 |
| 3565269 | 0.000500392 | 0.942712636 |
| 1871489 | 0.000505634 | 0.942712636 |
| 670526 | 0.00050872 | 0.942712636 |
| 3865731 | 0.000618448 | 0.942712636 |
| 1711184 | 0.000682355 | 0.942712636 |
| 1871506 | 0.000688648 | 0.942712636 |
| 3122355 | 0.000691856 | 0.942712636 |
| 622215 | 0.000773498 | 0.942712636 |
| 3565203 | 0.000901856 | 0.942712636 |
| 1203600 | 0.000994258 | 0.942712636 |
| 3565214 | 0.001005416 | 0.942712636 |
| 3693100 | 0.001042893 | 0.942712636 |
| 1800398 | 0.001106212 | 0.942712636 |
| 2338982 | 0.001330951 | 0.942712636 |
| 1363739 | 0.00155695 | 0.942712636 |
| 1234410 | 0.001614595 | 0.942712636 |
| 507206 | 0.002048899 | 0.942712636 |
| 3401101 | 0.002060611 | 0.942712636 |
| 1581736 | 0.002626153 | 0.942712636 |
| 4171006 | 0.002636135 | 0.942712636 |
| 160343 | 0.002707919 | 0.942712636 |
| 10940 | 0.003258375 | 0.942712636 |
| 3444374 | 9.56E−05 | 0.909972399 |
| 3444361 | 0.000100994 | 0.909972399 |
| 1709425 | 0.002144821 | 0.942712636 |

TABLE 5B
CLILLY

| Perlegen_SNP | FisherPval | FisherQval |
|---|---|---|
| 3819892 | 4.91E−06 | 0.54809676 |
| 1614288 | 7.09E−06 | 0.54809676 |
| 3819894 | 0.0000085 | 0.54809676 |
| 3174413 | 0.000117562 | 0.904680398 |
| 1586214 | 0.000230335 | 0.904680398 |
| 848514 | 0.000255731 | 0.904680398 |
| 526026 | 0.000294614 | 0.904680398 |
| 1586226 | 0.000311848 | 0.904680398 |
| 4080468 | 0.000344132 | 0.904680398 |
| 3122754 | 0.000346132 | 0.904680398 |
| 1586261 | 0.00035993 | 0.915854528 |
| 1586311 | 0.0003752 | 0.919303147 |
| 4283222 | 0.000393001 | 0.919303147 |
| 497179 | 0.00039329 | 0.919303147 |
| 4516175 | 0.000397545 | 0.919303147 |
| 1586171 | 0.000404069 | 0.919303147 |
| 3612255 | 0.000537438 | 0.931747307 |
| 891440 | 0.000590668 | 0.931747307 |
| 1325111 | 0.000590691 | 0.931747307 |
| 770139 | 0.000621158 | 0.931747307 |
| 4040872 | 0.000654293 | 0.931747307 |
| 2369244 | 0.000722202 | 0.931747307 |
| 1406391 | 0.000726591 | 0.931747307 |
| 1553131 | 0.000743514 | 0.931747307 |
| 1476779 | 0.000756724 | 0.931747307 |
| 303552 | 0.000949059 | 0.931747307 |
| 1686926 | 1.12E−03 | 0.931747307 |
| 770116 | 0.001175479 | 0.931747307 |
| 1124752 | 0.001192117 | 0.931747307 |
| 3170195 | 1.20E−03 | 0.931747307 |
| 1778519 | 1.22E−03 | 0.931747307 |
| 2368729 | 1.27E−03 | 0.931747307 |
| 1868867 | 1.40E−03 | 0.931747307 |
| 2343282 | 1.42E−03 | 0.931747307 |
| 3485444 | 1.42E−03 | 0.931747307 |
| 3870505 | 1.43E−03 | 0.931747307 |
| 1385352 | 1.48E−03 | 0.931747307 |
| 3509817 | 1.57E−03 | 0.931747307 |
| 1754883 | 1.93E−03 | 0.931747307 |
| 2378362 | 2.36E−03 | 0.931747307 |
| 2386700 | 2.44E−03 | 0.931747307 |
| 3715741 | 2.48E−03 | 0.931747307 |
| 770100 | 2.49E−03 | 0.931747307 |
| 3127128 | 0.002508585 | 0.931747307 |
| 145360 | 0.002603226 | 0.931747307 |
| 3111057 | 0.002733873 | 0.931747307 |
| 1507256 | 0.003177268 | 0.931747307 |
| 1988046 | 0.0000266 | 0.904680398 |
| 214385 | 0.000163538 | 0.904680398 |
| 1988073 | 0.000215354 | 0.904680398 |
| 2386620 | 0.000226369 | 0.904680398 |
| 944388 | 0.000311437 | 0.904680398 |
| 736610 | 0.000542892 | 0.931747307 |
| 3554315 | 0.000999035 | 0.931747307 |
| 2034022 | 0.001075507 | 0.931747307 |
| 3560562 | 0.001293959 | 0.931747307 |

TABLE 5C
HMDT

| Perlegen_SNP | FisherPval | FisherQval |
|---|---|---|
| 3122754 | 0.0000872 | 0.896847022 |
| 2392173 | 0.000231798 | 0.896847022 |
| 552812 | 0.000248879 | 0.896847022 |
| 3820225 | 0.00029218 | 0.896847022 |
| 1744345 | 0.000384995 | 0.896847022 |
| 2056363 | 0.000397117 | 0.896847022 |
| 3326948 | 0.000420049 | 0.896847022 |
| 3904449 | 0.000553595 | 0.896847022 |
| 2469920 | 0.000576937 | 0.896847022 |
| 2056311 | 0.000631026 | 0.896847022 |
| 3820201 | 0.000718243 | 0.896847022 |
| 3904418 | 0.000981211 | 0.896847022 |
| 2342573 | 0.001130165 | 0.896847022 |
| 982183 | 0.001166563 | 0.896847022 |
| 2343282 | 0.001182526 | 0.896847022 |
| 1800398 | 0.00138105 | 0.896847022 |
| 811022 | 0.001558082 | 0.896847022 |
| 2392172 | 0.001580955 | 0.896847022 |
| 3560562 | 0.001996024 | 0.896847022 |
| 4122932 | 0.002016422 | 0.896847022 |
| 3715741 | 0.002053633 | 0.896847022 |
| 3127128 | 0.002080509 | 0.896847022 |
| 704088 | 0.002308886 | 0.896847022 |
| 3819892 | 0.00000513 | 0.527880571 |
| 3819894 | 6.50E−06 | 0.527880571 |
| 214385 | 0.000127198 | 0.896847022 |
| 3352050 | 0.000215055 | 0.896847022 |
| 3170195 | 5.19E−04 | 0.896847022 |
| 4670519 | 1.38E−03 | 0.896847022 |

TABLE 5D
INSOM

| Perlegen_SNP | FisherPval | FisherQval |
|---|---|---|
| 3255514 | 1.75E−05 | 0.597735223 |
| 1059228 | 0.000042 | 0.597735223 |
| 2267426 | 0.0000729 | 0.597735223 |
| 3875859 | 0.0000753 | 0.597735223 |
| 3875872 | 0.000111592 | 0.64432005 |
| 302732 | 0.000175396 | 0.679570313 |
| 617225 | 0.000221704 | 0.679570313 |
| 2351093 | 0.000279014 | 0.738377687 |
| 617266 | 0.000335036 | 0.753411245 |
| 1445906 | 0.000399251 | 0.792425957 |
| 3142103 | 0.000614577 | 0.887129335 |
| 1661334 | 0.000702605 | 0.892544184 |
| 4122932 | 0.000732945 | 0.892544184 |
| 2437138 | 0.000767675 | 0.892544184 |
| 3263451 | 0.000820484 | 0.892544184 |
| 4122930 | 0.000835698 | 0.892544184 |
| 617297 | 0.000841362 | 0.892544184 |
| 632286 | 0.000947842 | 0.892544184 |
| 2492846 | 0.000953843 | 0.892544184 |
| 1096046 | 0.001007114 | 0.892544184 |
| 2437121 | 0.001117106 | 0.892544184 |
| 555297 | 0.001230793 | 0.896794961 |
| 555302 | 0.001428932 | 0.905792619 |
| 2345656 | 0.001549704 | 0.905792619 |
| 1390626 | 0.001640637 | 0.905792619 |
| 3666917 | 0.001840013 | 0.905792619 |
| 54303 | 2.12E−03 | 0.905792619 |
| 1205283 | 0.002162269 | 0.905792619 |
| 617524 | 0.002284142 | 0.905792619 |
| 3861184 | 2.37E−03 | 0.905792619 |
| 4428163 | 2.78E−03 | 0.905792619 |
| 1166312 | 3.30E−03 | 0.905792619 |
| 2450638 | 3.41E−03 | 0.905792619 |
| 555380 | 1.40E−05 | 0.597735223 |
| 2490021 | 4.63E−05 | 0.597735223 |
| 272259 | 2.05E−04 | 0.679570313 |
| 2387552 | 2.19E−04 | 0.679570313 |
| 343816 | 2.56E−04 | 0.689832893 |
| 2274606 | 6.58E−04 | 0.892544184 |

TABLE 6A
Novel Binary Genotype
ANX

| Perlegen_SNP | FisherPval | FisherQval |
|---|---|---|
| 3565214 | 3.74E−05 | 0.50542105 |
| 1552540 | 5.78E−05 | 0.50542105 |
| 3565203 | 5.83E−05 | 0.50542105 |
| 3565239 | 6.06E−05 | 0.50542105 |
| 3565269 | 6.82E−05 | 0.50542105 |
| 3565292 | 8.03E−05 | 0.50542105 |
| 3565281 | 8.13E−05 | 0.50542105 |
| 2993728 | 8.35E−05 | 0.50542105 |
| 1085506 | 8.99E−05 | 0.51524345 |
| 3565071 | 0.000111338 | 0.569136379 |
| 1800398 | 0.000131748 | 0.622957831 |
| 3444361 | 0.000169552 | 0.681000056 |
| 1085178 | 0.000170831 | 0.681000056 |
| 1760288 | 0.000204967 | 0.682043477 |
| 2790279 | 0.000211394 | 0.682043477 |
| 2056363 | 0.000215427 | 0.682043477 |
| 3510699 | 0.000218452 | 0.682043477 |
| 3332880 | 0.000225065 | 0.683003986 |
| 3444374 | 0.000232693 | 0.683003986 |
| 1760335 | 0.000235817 | 0.683003986 |
| 1660150 | 0.000271029 | 0.702213033 |
| 4571614 | 0.00032878 | 0.721089018 |
| 358756 | 0.000344218 | 0.731510011 |
| 3870185 | 0.000384177 | 0.754459136 |
| 2099207 | 0.000402239 | 0.754459136 |
| 3674864 | 0.000424343 | 0.754459136 |
| 755186 | 0.000430845 | 0.754459136 |
| 670526 | 0.000486446 | 0.793975608 |
| 552812 | 0.000514299 | 0.812041175 |
| 3122754 | 0.000564623 | 0.818752203 |
| 3443855 | 0.00066048 | 0.821882069 |
| 3865731 | 0.00083574 | 0.821882069 |
| 1334452 | 0.000845319 | 0.821882069 |
| 3303532 | 0.000963663 | 0.821882069 |
| 2488188 | 0.000983659 | 0.821882069 |
| 17207 | 0.000990907 | 0.821882069 |
| 17255 | 0.001029423 | 0.821882069 |
| 2684490 | 0.001088913 | 0.821882069 |
| 552869 | 0.001225311 | 0.821882069 |
| 3708771 | 0.001311459 | 0.825432856 |
| 3611749 | 0.00131821 | 0.825432856 |
| 4320535 | 0.001362934 | 0.825432856 |
| 3122355 | 0.001641419 | 0.859979826 |
| 1226296 | 0.001687938 | 0.865896663 |
| 979327 | 0.001694382 | 0.866135458 |
| 972242 | 0.002417108 | 0.883359242 |
| 2634363 | 0.00296125 | 0.88406624 |
| 904811 | 6.80E−05 | 0.50542105 |
| 163068 | 0.000434797 | 0.754459136 |
| 3114067 | 0.001013389 | 0.821882069 |
| 2324234 | 0.001798169 | 0.871763249 |

TABLE 6B
LILLY

| Perlegen_SNP | FisherPval | FisherQval |
|---|---|---|
| 1912900 | 3.18E−06 | 0.596175312 |
| 303552 | 3.40E−05 | 0.695989982 |
| 727193 | 8.59E−05 | 0.734767541 |
| 2369244 | 9.39E−05 | 0.736100904 |
| 2048484 | 1.74E−04 | 0.76187412 |
| 3326948 | 1.78E−04 | 0.76187412 |
| 3554315 | 2.01E−04 | 0.76187412 |
| 979327 | 2.02E−04 | 0.76187412 |
| 552812 | 2.27E−04 | 0.809544088 |
| 1678333 | 0.000233957 | 0.809544088 |
| 348386 | 0.000373255 | 0.866621002 |
| 4476399 | 0.000381287 | 0.866621002 |
| 3318048 | 0.000395318 | 0.866621002 |
| 4336765 | 0.000521725 | 0.872932395 |
| 358303 | 0.000531893 | 0.872932395 |
| 2153874 | 0.000562685 | 0.872932395 |
| 532395 | 0.000738205 | 0.872932395 |
| 2138144 | 0.000791168 | 0.872932395 |
| 2340118 | 0.000804415 | 0.872932395 |
| 2314337 | 0.000812532 | 0.872932395 |
| 769719 | 0.000838837 | 0.875967022 |
| 1923923 | 0.000976584 | 0.892714143 |
| 1811416 | 0.000983074 | 0.892714143 |
| 1613798 | 0.00100905 | 0.892714143 |
| 256781 | 0.001115052 | 0.893838388 |
| 1085506 | 0.001138308 | 0.893838388 |
| 3111057 | 0.001186916 | 0.893838388 |
| 2147441 | 0.001299697 | 0.893838388 |
| 906272 | 0.001324264 | 0.893838388 |
| 1476779 | 0.001376456 | 0.893838388 |
| 3141936 | 0.001405744 | 0.893838388 |
| 784172 | 0.001456013 | 0.893838388 |
| 3666917 | 0.001539025 | 0.893838388 |
| 472520 | 0.001565519 | 0.893838388 |
| 516338 | 0.001745923 | 0.899583403 |
| 2203902 | 0.00194163 | 0.899583403 |
| 1811405 | 0.001974277 | 0.899583403 |
| 2405824 | 0.002197354 | 0.899583403 |
| 411305 | 0.002328438 | 0.899583403 |
| 2583974 | 0.002511038 | 0.899583403 |
| 1811387 | 0.002520753 | 0.899583403 |
| 690495 | 0.002576258 | 0.899583403 |
| 1001406 | 0.003106124 | 0.899583403 |
| 138564 | 0.003294954 | 0.899583403 |
| 159809 | 0.000405794 | 0.866621002 |
| 159815 | 0.000421702 | 0.869490807 |
| 739904 | 0.000467182 | 0.872932395 |
| 3444361 | 5.83E−04 | 0.872932395 |
| 1689616 | 0.000627178 | 0.872932395 |
| 1215253 | 0.000657292 | 0.872932395 |
| 3444374 | 0.000695425 | 0.872932395 |
| 12394 | 0.001941884 | 0.899583403 |
| 1912900 | 3.18E−06 | 0.596175312 |

TABLE 6C
HMDT

| Perlegen_SNP | FisherPval | FisherQval |
|---|---|---|
| 797205 | 7.86E−05 | 0.709314361 |
| 3303532 | 8.32E−05 | 0.709314361 |
| 3904418 | 1.24E−04 | 0.795496029 |
| 2468866 | 1.51E−04 | 0.814566784 |
| 1337252 | 1.58E−04 | 0.814566784 |
| 3819894 | 2.50E−04 | 0.814566784 |
| 945100 | 2.67E−04 | 0.822165733 |
| 3122754 | 2.71E−04 | 0.822165733 |
| 20802 | 3.80E−04 | 0.828149458 |
| 894518 | 0.000393786 | 0.828149458 |
| 1756543 | 0.000469699 | 0.828149458 |
| 409576 | 0.000480102 | 0.828149458 |
| 1905516 | 0.000504668 | 0.828149458 |
| 1611770 | 0.000644481 | 0.828149458 |
| 3901134 | 0.000661764 | 0.828149458 |
| 1085506 | 0.000663327 | 0.828149458 |
| 1614322 | 0.000730175 | 0.828149458 |
| 2342573 | 0.000745396 | 0.828149458 |
| 3748654 | 0.000765359 | 0.828149458 |
| 104324 | 0.000786264 | 0.828149458 |
| 3592834 | 0.000845061 | 0.828149458 |
| 2603232 | 0.000952835 | 0.834137568 |
| 773759 | 0.001057905 | 0.850070392 |
| 2084108 | 0.001078411 | 0.861045508 |
| 3396741 | 0.001367631 | 0.899405123 |
| 1085178 | 0.00139558 | 0.899405123 |
| 3554904 | 0.0014199 | 0.899405123 |
| 2386150 | 0.001491836 | 0.899405123 |
| 2641152 | 0.001734342 | 0.910370919 |
| 1614316 | 0.001789176 | 0.910793817 |
| 1614294 | 0.00192105 | 0.922748439 |
| 3193146 | 0.003011683 | 0.949612679 |
| 739904 | 0.0000213 | 0.511169381 |
| 1919024 | 0.0000731 | 0.709314361 |
| 3819892 | 0.000282295 | 0.822165733 |
| 1614290 | 0.000365771 | 0.828149458 |
| 1369909 | 0.000406079 | 0.828149458 |
| 3106665 | 0.000895662 | 0.828149458 |
| 2172827 | 0.002347173 | 0.941963678 |

TABLE 6D
INSOM

| Perlegen_SNP | FisherPval | FisherQval |
|---|---|---|
| 2001950 | 4.07E−05 | 0.357795756 |
| 4232561 | 7.04E−05 | 0.461502025 |
| 2267426 | 1.53E−04 | 0.554223547 |
| 3875872 | 1.56E−04 | 0.554223547 |
| 1953297 | 1.69E−04 | 0.582369298 |
| 1579845 | 2.17E−04 | 0.640392906 |
| 2677683 | 2.34E−04 | 0.640392906 |
| 365290 | 2.82E−04 | 0.665911054 |
| 3477963 | 2.94E−04 | 0.665911054 |
| 3531432 | 0.000390416 | 0.730598063 |
| 1534880 | 0.000497346 | 0.743456859 |
| 3692953 | 0.000746627 | 0.78171834 |
| 2345857 | 0.000838902 | 0.78171834 |
| 2420701 | 0.000862483 | 0.78171834 |
| 1872350 | 0.000876414 | 0.78171834 |
| 2001971 | 0.00100183 | 0.78171834 |
| 2450638 | 0.001022434 | 0.78171834 |
| 1150093 | 0.001046691 | 0.784376782 |
| 2522402 | 0.00107975 | 0.784376782 |
| 269181 | 0.001315011 | 0.792261726 |
| 2475055 | 0.001343751 | 0.792261726 |
| 3866218 | 0.001538641 | 0.809869609 |
| 1534840 | 0.001753581 | 0.824147879 |
| 3263451 | 0.001896905 | 0.824147879 |
| 3447858 | 0.002543217 | 0.824147879 |
| 2399557 | 0.002597211 | 0.824147879 |
| 3181874 | 0.0026232 | 0.824147879 |
| 4551370 | 0.002777384 | 0.824147879 |
| 1059228 | 0.000017 | 0.282347423 |
| 653852 | 0.000106972 | 0.53369726 |
| 1386738 | 0.000113385 | 0.536680775 |
| 3271579 | 0.000133604 | 0.540105954 |
| 272259 | 0.000227256 | 0.640392906 |
| 883568 | 0.000230191 | 0.640392906 |
| 2355020 | 0.001056604 | 0.784376782 |
| 910353 | 0.001507947 | 0.809869609 |

TABLE 7A
Novel Binary Interaction
ANX

| Perlegen_SNP | FisherPval | FisherQval |
|---|---|---|
| 3123186 | 1.85E−06 | 0.172688059 |
| 3673009 | 0.0000496 | 0.884946863 |
| 2039518 | 9.15E−05 | 0.884946863 |
| 2147954 | 0.000106072 | 0.884946863 |
| 766107 | 0.000159418 | 0.884946863 |
| 2394342 | 0.000178761 | 0.884946863 |
| 679867 | 0.000219445 | 0.884946863 |
| 1268020 | 0.000246353 | 0.884946863 |
| 3460224 | 0.000246582 | 0.884946863 |
| 3296481 | 0.000295821 | 0.884946863 |
| 1918812 | 0.000330117 | 0.884946863 |
| 1054104 | 0.000330847 | 0.884946863 |
| 814339 | 0.000367622 | 0.893358301 |
| 2009288 | 0.000457655 | 0.894646315 |
| 2478983 | 5.58E−04 | 0.894646315 |
| 1410980 | 0.000569958 | 0.894646315 |
| 668040 | 0.000631952 | 0.894646315 |
| 2039526 | 0.000655166 | 0.894646315 |
| 279774 | 0.000661047 | 0.894646315 |
| 2118459 | 0.000672331 | 0.894646315 |
| 2118498 | 0.000682401 | 0.894646315 |
| 814347 | 0.000684825 | 0.894646315 |
| 3713131 | 0.000765601 | 0.894646315 |
| 2606259 | 0.00084099 | 0.894646315 |
| 3876937 | 0.000861585 | 0.894646315 |
| 1260720 | 0.000898008 | 0.894646315 |
| 2118500 | 0.000909127 | 0.894646315 |
| 1816467 | 0.000953432 | 0.894646315 |
| 499954 | 0.001012624 | 0.894646315 |
| 3430918 | 0.001027032 | 0.894646315 |
| 3200432 | 0.00103623 | 0.894646315 |
| 1054144 | 0.001144291 | 0.894646315 |
| 2379489 | 0.001164992 | 0.894646315 |
| 1555949 | 0.001174928 | 0.894646315 |
| 1921315 | 0.001376591 | 0.894646315 |
| 3512057 | 0.001476664 | 0.894646315 |
| 1268046 | 0.001495458 | 0.894646315 |
| 3339124 | 0.00161942 | 0.894646315 |
| 3605475 | 0.001730173 | 0.894646315 |
| 2321879 | 0.001776967 | 0.894646315 |
| 4672031 | 0.001803243 | 0.894646315 |
| 3666695 | 0.002116898 | 0.894646315 |
| 3863206 | 0.002390844 | 0.894646315 |
| 280907 | 0.002392077 | 0.894646315 |
| 3107121 | 0.002535831 | 0.894646315 |
| 3666604 | 0.002566629 | 0.894646315 |
| 2316249 | 0.0000784 | 0.884946863 |
| 1812844 | 0.0000944 | 0.884946863 |
| 522335 | 0.000160729 | 0.884946863 |

TABLE 7B
CLILLY

| Perlegen_SNP | FisherPval | FisherQval |
|---|---|---|
| 1585755 | 4.22E−07 | 0.077530634 |
| 1829453 | 1.40E−05 | 0.517313148 |
| 1276639 | 1.74E−05 | 0.517313148 |
| 121018 | 0.0000443 | 0.626268241 |
| 1456212 | 0.0000589 | 0.676045239 |
| 3596962 | 0.000091 | 0.796361322 |
| 2147954 | 0.0000916 | 0.796361322 |
| 1982441 | 0.000114012 | 0.805532947 |
| 1162438 | 0.000213482 | 0.814796791 |
| 3328007 | 0.000242756 | 0.814796791 |
| 120906 | 2.71E−04 | 0.814796791 |
| 1276713 | 3.08E−04 | 0.814796791 |
| 120880 | 0.000373024 | 0.814796791 |
| 2410695 | 0.000447794 | 0.814796791 |
| 1899724 | 0.000470189 | 0.824954655 |
| 3417519 | 0.000485803 | 0.827452887 |
| 1208424 | 0.000564644 | 0.850429771 |
| 120634 | 0.000653797 | 0.885573423 |
| 3993569 | 0.000662617 | 0.885573423 |
| 2474217 | 0.000719272 | 0.885573423 |
| 120658 | 0.000750656 | 0.885573423 |
| 2037647 | 0.00080789 | 0.885573423 |
| 40257 | 0.000876468 | 0.885573423 |
| 1899741 | 0.000890829 | 0.885573423 |
| 120843 | 0.000925325 | 0.885573423 |
| 2931858 | 0.001095026 | 0.885573423 |
| 3151597 | 0.001196332 | 0.885573423 |
| 1654367 | 0.001295452 | 0.885573423 |
| 120666 | 0.001355155 | 0.885573423 |
| 1468451 | 0.001382791 | 0.885573423 |
| 3151603 | 0.001428266 | 0.885573423 |
| 39641 | 0.001469412 | 0.885573423 |
| 1899780 | 0.001497294 | 0.885573423 |
| 1616405 | 0.001568294 | 0.891698664 |
| 3896084 | 0.001581456 | 0.891698664 |
| 3174758 | 0.001637129 | 0.891698664 |
| 542193 | 0.001726239 | 0.891698664 |
| 2997317 | 0.001768637 | 0.891698664 |
| 2257769 | 0.00183541 | 0.892192954 |
| 2312380 | 0.001871729 | 0.892192954 |
| 507284 | 0.001941043 | 0.892192954 |
| 3848797 | 0.002284974 | 0.892192954 |
| 1597461 | 0.002434229 | 0.892192954 |
| 3449937 | 1.97E−05 | 0.517313148 |
| 75560 | 0.000174256 | 0.814796791 |
| 4213286 | 3.43E−04 | 0.814796791 |
| 552991 | 0.000422671 | 0.814796791 |
| 1585729 | 0.000427575 | 0.814796791 |

TABLE 7C
HMDT

| Perlegen_SNP | FisherPval | FisherQval |
|---|---|---|
| 1918812 | 3.72E−05 | 0.60757736 |
| 1918804 | 4.89E−05 | 0.60757736 |
| 2009288 | 5.20E−05 | 0.60757736 |
| 1718389 | 5.52E−05 | 0.60757736 |
| 1276573 | 8.44E−05 | 0.60757736 |
| 1974444 | 0.000113195 | 0.615572839 |
| 3322140 | 0.000121191 | 0.615572839 |
| 1974401 | 0.00013132 | 0.630594089 |
| 1974421 | 0.00017814 | 0.69686637 |
| 3700031 | 0.000188579 | 0.69686637 |
| 1974303 | 0.000218152 | 0.69686637 |
| 1974527 | 0.000242629 | 0.69686637 |
| 1971980 | 0.000255884 | 0.69686637 |
| 3475688 | 0.000306172 | 0.69686637 |
| 1974476 | 0.000307586 | 0.69686637 |
| 594176 | 0.000309842 | 0.69686637 |
| 1974329 | 0.000327942 | 0.69686637 |
| 1618767 | 0.000465081 | 0.781912236 |
| 3673009 | 0.000507853 | 0.792004627 |
| 3386350 | 0.000520291 | 0.792004627 |
| 1654367 | 0.00053906 | 0.792004627 |
| 3417519 | 0.000604733 | 0.792004627 |
| 313699 | 0.000653236 | 0.792004627 |
| 3594277 | 0.000672211 | 0.792004627 |
| 4139599 | 0.000683342 | 0.794218849 |
| 1718430 | 0.000752184 | 0.80418697 |
| 1119230 | 0.000801378 | 0.804943919 |
| 1095559 | 0.000958354 | 0.804943919 |
| 3288843 | 0.000965382 | 0.804943919 |
| 2486070 | 0.000997419 | 0.804943919 |
| 2116144 | 0.001054683 | 0.804943919 |
| 1804744 | 0.001072266 | 0.804943919 |
| 2009302 | 0.001085174 | 0.804943919 |
| 2039518 | 0.001174992 | 0.809078132 |
| 3325587 | 0.001235909 | 0.814899938 |
| 2041414 | 0.001471288 | 0.825277365 |
| 898321 | 0.001523322 | 0.825277365 |
| 3522226 | 0.001577137 | 0.825277365 |
| 962652 | 0.001642249 | 0.825277365 |
| 1208424 | 0.001949287 | 0.825277365 |
| 3522237 | 0.002101045 | 0.834350889 |
| 2049385 | 0.002102726 | 0.834350889 |
| 2147936 | 0.002488053 | 0.857485245 |
| 821871 | 0.002655072 | 0.857485245 |
| 313717 | 0.000150857 | 0.688189146 |
| 1645177 | 0.000232364 | 0.69686637 |
| 3713131 | 0.000551873 | 0.792004627 |

TABLE 7D
INSOM

| Perlegen_SNP | FisherPval | FisherQval |
|---|---|---|
| 1506595 | 0.0000512 | 0.769877609 |
| 2022927 | 0.0000777 | 0.769877609 |
| 492423 | 0.000125609 | 0.769877609 |
| 1790542 | 0.00013433 | 0.769877609 |
| 3684385 | 0.000176913 | 0.769877609 |
| 2022897 | 0.000189485 | 0.779065024 |
| 2032297 | 0.00022658 | 0.81484458 |
| 2022921 | 0.000277437 | 0.81484458 |
| 882595 | 0.000291303 | 0.81484458 |
| 2118459 | 0.000301345 | 0.81484458 |
| 2201976 | 0.000340408 | 0.81484458 |
| 2118498 | 0.000378114 | 0.81484458 |
| 4442429 | 0.000464368 | 0.81484458 |
| 2118500 | 0.000504471 | 0.820508228 |
| 1004017 | 0.000549364 | 0.822644008 |
| 3594277 | 5.53E−04 | 0.822644008 |
| 1801860 | 0.000666931 | 0.867795214 |
| 3288850 | 0.000678021 | 0.870222616 |
| 903569 | 0.000749141 | 0.874873059 |
| 3548055 | 0.000781836 | 0.874873059 |
| 4436593 | 0.000820021 | 0.874873059 |
| 3511990 | 0.000843385 | 0.874873059 |
| 813112 | 0.000945285 | 0.874873059 |
| 368144 | 0.000972484 | 0.874873059 |
| 1798659 | 0.000997355 | 0.88044959 |
| 2244319 | 0.001088076 | 0.890815319 |
| 1646094 | 0.001167786 | 0.890815319 |
| 3486976 | 0.001291877 | 0.907862841 |
| 383165 | 0.001379008 | 0.907862841 |
| 1626238 | 0.001420778 | 0.907862841 |
| 383212 | 0.001456677 | 0.907862841 |
| 383214 | 0.001527116 | 0.907862841 |
| 3288843 | 0.001861693 | 0.929507725 |
| 334643 | 0.002154937 | 0.929507725 |
| 4027550 | 0.002344654 | 0.929507725 |
| 882560 | 0.002762401 | 0.929507725 |
| 1999459 | 0.0000743 | 0.769877609 |
| 446808 | 0.000123552 | 0.769877609 |
| 4588473 | 2.60E−04 | 0.81484458 |

TABLE 8A
Novel Linear Interaction
ANX

| Perlegen_SNP | FisherPval | FisherQval |
|---|---|---|
| 1276573 | 5.54E−06 | 0.690054875 |
| 3123186 | 7.69E−06 | 0.690054875 |
| 1276558 | 0.0000109 | 0.690054875 |
| 679867 | 0.0000297 | 0.802428017 |
| 1966373 | 0.000104802 | 0.831437921 |
| 1276697 | 0.000143427 | 0.831437921 |
| 2147954 | 0.000147486 | 0.831437921 |
| 1009275 | 0.000162966 | 0.831437921 |
| 542193 | 0.000230103 | 0.831437921 |
| 2451656 | 0.000250362 | 0.831437921 |
| 2394342 | 0.000299991 | 0.831437921 |
| 1705437 | 0.00030737 | 0.831437921 |
| 1276713 | 0.00032498 | 0.831437921 |
| 1405831 | 0.000427174 | 0.831437921 |
| 2451662 | 0.000443097 | 0.831437921 |
| 1276683 | 0.000450169 | 0.831437921 |
| 1276648 | 0.000497576 | 0.831437921 |
| 1293364 | 0.000499701 | 0.831437921 |
| 3296481 | 0.000511291 | 0.831437921 |
| 1405790 | 0.000531537 | 0.831437921 |
| 1276639 | 0.000556925 | 0.831437921 |
| 1953110 | 0.000579846 | 0.831437921 |
| 3863206 | 0.00069296 | 0.846614455 |
| 2020226 | 0.000900507 | 0.861520912 |
| 1276632 | 0.000924432 | 0.861520912 |
| 3562810 | 0.000951699 | 0.861520912 |
| 2222231 | 0.000956645 | 0.861520912 |
| 4404676 | 0.001016381 | 0.861520912 |
| 1966361 | 1.07E−03 | 0.861520912 |
| 1067961 | 0.00111909 | 0.861520912 |
| 2009288 | 0.001170816 | 0.861520912 |
| 1327662 | 0.001195037 | 0.861520912 |
| 340241 | 0.001215751 | 0.861520912 |
| 1943237 | 0.001262444 | 0.861520912 |
| 1953067 | 0.001280055 | 0.861520912 |
| 2317228 | 0.001319994 | 0.872365434 |
| 607843 | 0.001440926 | 0.879626447 |
| 2328415 | 0.001460599 | 0.879626447 |
| 1260720 | 0.00150703 | 0.890470959 |
| 1737980 | 0.001590802 | 0.890843761 |
| 3670712 | 0.001726221 | 0.910379216 |
| 342860 | 0.001793934 | 0.914302351 |
| 1966340 | 0.00191288 | 0.914302351 |
| 3614176 | 0.002350182 | 0.920669311 |
| 3614192 | 0.002644713 | 0.920669311 |
| 2448996 | 0.000178558 | 0.831437921 |
| 3848852 | 0.000511764 | 0.831437921 |
| 3713131 | 0.000531864 | 0.831437921 |
| 120178 | 0.000662362 | 0.846614455 |

TABLE 8B
CLILLY

| Perlegen_SNP | FisherPval | FisherQval |
|---|---|---|
| 2946891 | 5.12E−05 | 0.638207215 |
| 4453632 | 6.97E−05 | 0.638207215 |
| 1125283 | 0.0000702 | 0.638207215 |
| 75560 | 0.0000928 | 0.638207215 |
| 4353088 | 0.000110719 | 0.664647994 |
| 1431533 | 0.000122898 | 0.664647994 |
| 1829453 | 0.000128244 | 0.664647994 |
| 2009288 | 0.000142893 | 0.664647994 |
| 3673009 | 0.000146588 | 0.664647994 |
| 2147954 | 0.000154918 | 0.664647994 |
| 3700031 | 0.000158351 | 0.664647994 |
| 1637827 | 0.00017212 | 0.664647994 |
| 4092120 | 0.000194946 | 0.664647994 |
| 3848797 | 0.000212959 | 0.664647994 |
| 3567213 | 0.000214566 | 0.664647994 |
| 3862078 | 0.000297168 | 0.754714194 |
| 3118276 | 0.00032455 | 0.782412669 |
| 539841 | 0.000353884 | 0.782412669 |
| 3386350 | 0.000510755 | 0.853208783 |
| 1992318 | 0.000742658 | 0.853208783 |
| 120178 | 0.000810091 | 0.853208783 |
| 1899724 | 0.00107746 | 0.853208783 |
| 3328007 | 0.001168322 | 0.853208783 |
| 3862097 | 0.001170505 | 0.853208783 |
| 3481741 | 0.001230609 | 0.853208783 |
| 3896625 | 0.001298284 | 0.853208783 |
| 3641695 | 0.001416178 | 0.853208783 |
| 3452760 | 1.54E−03 | 0.853208783 |
| 1276639 | 0.001801397 | 0.853208783 |
| 441242 | 0.001823328 | 0.853208783 |
| 1257281 | 0.001887601 | 0.853208783 |
| 3857566 | 0.002132232 | 0.853208783 |
| 2009302 | 0.002205987 | 0.853208783 |
| 295720 | 0.002448359 | 0.853208783 |
| 1616405 | 0.002788514 | 0.853208783 |
| 1718389 | 0.0000241 | 0.638207215 |
| 1718318 | 0.0000813 | 0.638207215 |
| 1718464 | 0.0000906 | 0.638207215 |
| 3668870 | 0.000188479 | 0.664647994 |
| 1585729 | 0.00025874 | 0.694668025 |
| 2163321 | 0.000896254 | 0.853208783 |
| 407964 | 0.001173731 | 0.853208783 |

TABLE 8C
HMDT

| Perlegen_SNP | FisherPval | FisherQval |
|---|---|---|
| 1276573 | 1.56E−05 | 0.587718388 |
| 2147954 | 0.0000683 | 0.808590884 |
| 1431533 | 0.000083 | 0.808590884 |
| 3673009 | 0.000138062 | 0.808590884 |
| 492423 | 0.000246481 | 0.808590884 |
| 2022927 | 0.00024776 | 0.808590884 |
| 3751183 | 0.000268238 | 0.808590884 |
| 2039518 | 0.000279019 | 0.808590884 |
| 3713131 | 0.000288195 | 0.808590884 |
| 1966373 | 0.000346958 | 0.808590884 |
| 2009288 | 0.000381859 | 0.808590884 |
| 3862078 | 0.000414353 | 0.808590884 |
| 3322140 | 0.000428216 | 0.808590884 |
| 238656 | 0.000471915 | 0.809584162 |
| 2394342 | 0.000484275 | 0.809584162 |
| 342860 | 0.000529174 | 0.814916867 |
| 1966340 | 0.000600408 | 0.815837689 |
| 3700031 | 0.000624746 | 0.815837689 |
| 3288843 | 0.000738017 | 0.83215468 |
| 3862097 | 0.000847967 | 0.83215468 |
| 75560 | 0.000869617 | 0.83215468 |
| 2163321 | 0.000873453 | 0.83215468 |
| 1228577 | 0.00088711 | 0.83215468 |
| 280097 | 0.000993832 | 0.834108886 |
| 4295305 | 0.001020798 | 0.834108886 |
| 1276713 | 0.001116771 | 0.834108886 |
| 920234 | 0.001169028 | 0.834108886 |
| 355828 | 0.001173625 | 0.834108886 |
| 2022921 | 1.24E−03 | 0.834108886 |
| 3296481 | 0.001455371 | 0.834108886 |
| 1327662 | 0.001626338 | 0.834108886 |
| 1255229 | 0.001683553 | 0.834108886 |
| 2022897 | 0.001830583 | 0.834108886 |
| 1674227 | 0.002575817 | 0.834108886 |
| 1276697 | 0.002758631 | 0.834108886 |
| 1718389 | 0.0000284 | 0.710281555 |
| 3594277 | 0.000132887 | 0.808590884 |
| 120178 | 0.000216002 | 0.808590884 |
| 1637827 | 0.000309853 | 0.808590884 |
| 456564 | 0.000768785 | 0.83215468 |
| 340241 | 0.001083959 | 0.834108886 |
| 3848852 | 0.001195067 | 0.834108886 |

TABLE 8D
INSOM

| Perlegen_SNP | FisherPval | FisherQval |
|---|---|---|
| 2022927 | 3.69E−06 | 0.352035264 |
| 2022921 | 9.33E−06 | 0.41157311 |
| 2022897 | 0.0000137 | 0.435168041 |
| 882595 | 0.0000582 | 0.813843552 |
| 3288850 | 0.0000698 | 0.813843552 |
| 2402372 | 0.000102337 | 0.813843552 |
| 4552714 | 0.000137926 | 0.822423426 |
| 1835296 | 0.000166361 | 0.843864781 |
| 882560 | 0.000198517 | 0.843864781 |
| 3152805 | 0.000328147 | 0.850132495 |
| 1933844 | 0.00035106 | 0.850132495 |
| 806436 | 0.00043081 | 0.903324249 |
| 1443883 | 0.000445147 | 0.92324168 |
| 2201976 | 0.000459083 | 0.928580435 |
| 4592239 | 0.000488364 | 0.928580435 |
| 1793653 | 0.000506956 | 0.928580435 |
| 2362180 | 0.000514903 | 0.928580435 |
| 3288843 | 0.000734393 | 0.928580435 |
| 2163423 | 0.000742812 | 0.928580435 |
| 509722 | 0.000756721 | 0.928580435 |
| 2425790 | 0.00079793 | 0.928580435 |
| 3687650 | 0.000921659 | 0.928580435 |
| 1487452 | 0.000921934 | 0.928580435 |
| 2118500 | 0.000987775 | 0.928580435 |
| 389068 | 0.000988607 | 0.928580435 |
| 2118459 | 0.000990178 | 0.928580435 |
| 1801860 | 0.001034554 | 0.928580435 |
| 2118498 | 0.001069126 | 0.93771496 |
| 2734519 | 1.09E−03 | 0.93771496 |
| 4150772 | 0.001193171 | 0.945922473 |
| 3881989 | 0.001491892 | 0.945922473 |
| 3687661 | 0.001568211 | 0.947507343 |
| 805214 | 0.001805088 | 0.947507343 |
| 707407 | 0.001831411 | 0.947507343 |
| 1443605 | 0.001846362 | 0.947507343 |
| 162110 | 0.001938188 | 0.947507343 |
| 2362171 | 0.001993684 | 0.947507343 |
| 3414039 | 0.002452871 | 0.947507343 |
| 1178707 | 0.002703093 | 0.947507343 |
| 2011187 | 0.000261546 | 0.843864781 |
| 2298521 | 0.000841374 | 0.928580435 |

TABLE 9A
Novel Time Genotype
ANX

| Perlegen_SNP | FisherPval | FisherQval |
|---|---|---|
| 3707022 | 4.62E−06 | 0.235784691 |
| 2926674 | 0.000163201 | 0.424519262 |
| 4560172 | 0.000248978 | 0.44842821 |
| 2899607 | 0.000249158 | 0.44842821 |
| 2814609 | 0.000310263 | 0.46001297 |
| 1778519 | 0.000721743 | 0.484505028 |
| 428043 | 0.000778985 | 0.484505028 |
| 470618 | 0.000783983 | 0.484505028 |
| 3528443 | 0.000865545 | 0.485779518 |
| 1871685 | 0.000976687 | 0.497142424 |
| 3609443 | 0.001219375 | 0.506356705 |
| 195161 | 0.001291125 | 0.506356705 |
| 1273590 | 0.001551024 | 0.506356705 |
| 1871599 | 0.001572185 | 0.506356705 |
| 3526374 | 0.001972571 | 0.515833505 |
| 1097038 | 0.002002389 | 0.517482115 |
| 1994975 | 0.002167276 | 0.517482115 |
| 2393872 | 0.002184268 | 0.517482115 |
| 1953274 | 0.0022123 | 0.517482115 |
| 1016164 | 0.002560161 | 0.517482115 |
| 1871356 | 0.003353166 | 0.517482115 |
| 2024965 | 0.0000238 | 0.393476267 |
| 3477251 | 4.66E−05 | 0.412075398 |
| 1442327 | 1.24E−04 | 0.424519262 |
| 2393802 | 1.42E−04 | 0.424519262 |
| 664783 | 0.00021314 | 0.424519262 |
| 2393869 | 0.000329567 | 0.46001297 |
| 133091 | 0.000354042 | 0.46001297 |
| 4449341 | 0.000413976 | 0.46001297 |
| 1068351 | 0.000420015 | 0.46001297 |
| 82434 | 0.000448337 | 0.46001297 |
| 2168345 | 0.000497556 | 0.46001297 |
| 3290010 | 0.000506156 | 0.46001297 |
| 2393855 | 0.000637224 | 0.476997044 |
| 772518 | 0.000964571 | 0.497142424 |
| 799054 | 0.000981067 | 0.497142424 |
| 2287160 | 0.001150663 | 0.506356705 |
| 1387272 | 0.001168355 | 0.506356705 |
| 921310 | 0.001171036 | 0.506356705 |

TABLE 9B
CLILLY

| Perlegen_SNP | FisherPval | FisherQval |
|---|---|---|
| 897732 | 2.45E−04 | 0.41318456 |
| 1751158 | 0.000371169 | 0.41318456 |
| 2129930 | 0.000644294 | 0.437981223 |
| 1542474 | 0.000688814 | 0.437981223 |
| 3842849 | 0.000706702 | 0.437981223 |
| 3293153 | 0.000757452 | 0.439831724 |
| 872845 | 0.000850228 | 0.457229241 |
| 3194833 | 0.000897274 | 0.457733935 |
| 2402892 | 0.001141571 | 0.459526693 |
| 2129919 | 0.001319595 | 0.459526693 |
| 2685247 | 0.001862107 | 0.489835882 |
| 1508781 | 0.00194803 | 0.489835882 |
| 2528446 | 0.001987446 | 0.490336675 |
| 1812391 | 0.002395617 | 0.497908003 |
| 2083902 | 0.003224027 | 0.523486735 |
| 3112389 | 0.0000354 | 0.376972281 |
| 1962816 | 0.0000614 | 0.41318456 |
| 1064263 | 0.000114319 | 0.41318456 |
| 1507420 | 0.00013244 | 0.41318456 |
| 2393730 | 0.000146501 | 0.41318456 |
| 1975561 | 0.000171247 | 0.41318456 |
| 872816 | 1.92E−04 | 0.41318456 |
| 2933057 | 2.12E−04 | 0.41318456 |
| 2016686 | 2.70E−04 | 0.41318456 |
| 2041419 | 0.000312286 | 0.41318456 |
| 289615 | 0.000358779 | 0.41318456 |
| 1110134 | 0.00055928 | 0.430111 |
| 1863938 | 0.000962527 | 0.459526693 |
| 3616400 | 0.001516401 | 0.481894275 |

TABLE 9C
HMDT

| Perlegen_SNP | FisherPval | FisherQval |
|---|---|---|
| 3563568 | 2.25E−05 | 0.249391178 |
| 2678537 | 0.0000827 | 0.332822531 |
| 3134360 | 0.000213409 | 0.390773007 |
| 1139328 | 0.000372266 | 0.390773007 |
| 3563516 | 0.000394451 | 0.390773007 |
| 1962816 | 0.000471096 | 0.395307587 |
| 583166 | 0.000565653 | 0.402688916 |
| 1139305 | 0.000593291 | 0.406019506 |
| 3563492 | 0.000612684 | 0.406019506 |
| 3524022 | 0.00063592 | 0.413665189 |
| 652898 | 0.000752324 | 0.413665189 |
| 4008972 | 0.000789429 | 0.413665189 |
| 3556069 | 0.000929596 | 0.413665189 |
| 3134325 | 0.000953244 | 0.413665189 |
| 3115306 | 0.001058728 | 0.413665189 |
| 1525920 | 0.001204693 | 0.420174108 |
| 3152573 | 0.00123702 | 0.420174108 |
| 4478770 | 0.001280074 | 0.423209962 |
| 3531816 | 0.001294216 | 0.423261022 |
| 2946402 | 0.001356481 | 0.423261022 |
| 3125188 | 0.001514607 | 0.423261022 |
| 3557017 | 0.001590062 | 0.423261022 |
| 1062376 | 2.22E−03 | 0.436828936 |
| 3373581 | 2.42E−03 | 0.438404385 |
| 2159712 | 2.39E−05 | 0.249391178 |
| 3563548 | 0.0000252 | 0.249391178 |
| 3819011 | 0.0000269 | 0.249391178 |
| 3563481 | 0.0000292 | 0.249391178 |
| 3563460 | 0.0000318 | 0.249872575 |
| 3613803 | 0.0000428 | 0.292000884 |
| 3530702 | 0.000099 | 0.361446136 |
| 2393730 | 0.000155764 | 0.390181686 |
| 396080 | 0.000221694 | 0.390773007 |
| 3563628 | 0.000307602 | 0.390773007 |
| 697891 | 0.000333853 | 0.390773007 |
| 2275497 | 0.00034491 | 0.390773007 |
| 2426484 | 0.00038266 | 0.390773007 |
| 3134356 | 0.000393166 | 0.390773007 |
| 2372263 | 0.000406511 | 0.394068758 |
| 1057517 | 5.01E−04 | 0.402681881 |
| 2128119 | 5.23E−04 | 0.402688916 |
| 1609848 | 5.34E−04 | 0.402688916 |
| 765032 | 5.67E−04 | 0.402688916 |
| 1542978 | 8.16E−04 | 0.413665189 |
| 3870054 | 8.75E−04 | 0.413665189 |
| 583132 | 1.23E−03 | 0.420174108 |
| 1043573 | 1.91E−03 | 0.430398146 |
| 625834 | 1.92E−03 | 0.430398146 |

TABLE 9D
INSOM

| Perlegen_SNP | FisherPval | FisherQval |
|---|---|---|
| 2170305 | 5.32E−05 | 0.293434184 |
| 1266537 | 0.000094 | 0.32358147 |
| 1266507 | 0.0000984 | 0.326256158 |
| 1676930 | 0.000106647 | 0.328494305 |
| 619434 | 0.000114647 | 0.328494305 |
| 1266553 | 0.000177716 | 0.328494305 |
| 1266544 | 0.000204458 | 0.328494305 |
| 137315 | 0.000327011 | 0.337109803 |
| 4566219 | 0.000342467 | 0.337109803 |
| 1266518 | 0.000347196 | 0.337109803 |
| 1912331 | 0.000556345 | 0.351426097 |
| 2054953 | 0.000568621 | 0.351426097 |
| 1266440 | 0.000679701 | 0.356828226 |
| 502021 | 0.000858376 | 0.356828226 |
| 918677 | 0.001103536 | 0.371891524 |
| 2214222 | 0.001239894 | 0.371891524 |
| 2563109 | 0.001469077 | 0.371891524 |
| 3222073 | 0.001611094 | 0.371891524 |
| 3592740 | 0.001848532 | 0.371891524 |
| 3187067 | 0.0020983 | 0.378644893 |
| 3876840 | 0.002335442 | 0.382583156 |
| 350246 | 0.00242566 | 0.382583156 |
| 3447488 | 3.02E−05 | 0.255898146 |
| 1067713 | 3.55E−05 | 0.255898146 |
| 1538591 | 5.16E−05 | 0.293434184 |
| 2473163 | 0.000119953 | 0.328494305 |
| 3881931 | 0.000138905 | 0.328494305 |
| 1749997 | 0.000142746 | 0.328494305 |
| 1311548 | 0.000156803 | 0.328494305 |
| 849011 | 0.000231891 | 0.334958773 |
| 1356550 | 0.000480317 | 0.349469535 |
| 885372 | 0.000569351 | 0.351426097 |
| 222093 | 0.00068797 | 0.356828226 |
| 3567949 | 0.000809638 | 0.356828226 |
| 1219347 | 0.001545233 | 0.371891524 |
| 4491310 | 0.001647392 | 0.371891524 |
| 2473181 | 0.001684149 | 0.371891524 |
| 283793 | 0.001764281 | 0.371891524 |
| 3561023 | 0.002865393 | 0.390706869 |

TABLE 10A
Novel Time Interaction
ANX

| Perlegen_SNP | FisherPval | FisherQval |
|---|---|---|
| 3713131 | 9.15E−06 | 0.306866978 |
| 1142006 | 5.46E−05 | 0.306866978 |
| 764102 | 0.000536158 | 0.377200974 |
| 814713 | 0.000588304 | 0.377200974 |
| 2328162 | 0.000621617 | 0.379589409 |
| 1084766 | 0.000702356 | 0.38405202 |
| 3329421 | 0.000824696 | 0.38405202 |
| 1813965 | 0.000961741 | 0.38405202 |
| 3713139 | 0.001229152 | 0.389198483 |
| 2246661 | 0.001277413 | 0.389198483 |
| 2473479 | 0.001331496 | 0.389198483 |
| 627822 | 0.001372247 | 0.390932515 |
| 1970899 | 0.001565757 | 0.397278593 |
| 519293 | 0.001606167 | 0.397278593 |
| 331584 | 0.001958863 | 0.402125056 |
| 137829 | 0.002155473 | 0.405909615 |
| 3191406 | 0.002689128 | 0.413865805 |
| 2311188 | 3.26E−05 | 0.306866978 |
| 363483 | 3.98E−05 | 0.306866978 |
| 2370749 | 6.75E−05 | 0.306866978 |
| 2063822 | 6.92E−05 | 0.306866978 |
| 3404095 | 7.81E−05 | 0.306866978 |
| 2478857 | 0.000110399 | 0.306866978 |
| 2402034 | 0.00011311 | 0.306866978 |
| 2140871 | 0.000136662 | 0.318402541 |
| 1276506 | 0.000147878 | 0.334250414 |
| 2469246 | 0.000166279 | 0.349741651 |
| 1221183 | 0.00017339 | 0.35139515 |
| 3263776 | 0.000183931 | 0.356186695 |
| 803273 | 0.000193952 | 0.356186695 |
| 800376 | 0.000219992 | 0.363391865 |
| 1205110 | 0.000231023 | 0.363391865 |
| 3412405 | 0.000288464 | 0.363391865 |
| 1614525 | 0.00036689 | 0.36985778 |
| 3288070 | 0.000394113 | 0.36985778 |
| 2402124 | 0.000427615 | 0.369944554 |
| 320863 | 0.000722421 | 0.38405202 |
| 3419155 | 0.001147717 | 0.386608801 |
| 4486048 | 0.001244998 | 0.389198483 |

TABLE 10B
CLILLY

| Perlegen_SNP | FisherPval | FisherQval |
|---|---|---|
| 2021615 | 4.15E−07 | 0.03756741 |
| 2013955 | 4.81E−07 | 0.03756741 |
| 2863248 | 0.000804611 | 0.41730746 |
| 908741 | 0.000832835 | 0.417435445 |
| 4044139 | 0.001156522 | 0.418863405 |
| 1447578 | 0.001313825 | 0.422175319 |
| 406464 | 0.001716397 | 0.429118827 |
| 1929382 | 0.002010338 | 0.434304601 |
| 842261 | 0.002027941 | 0.434304601 |
| 2596560 | 0.002334375 | 0.436213574 |
| 2446882 | 0.002455637 | 0.436213574 |
| 3117549 | 0.0000226 | 0.314577976 |
| 3117608 | 0.000023 | 0.314577976 |
| 2751104 | 0.0000286 | 0.318800029 |
| 2415217 | 0.000133376 | 0.390483336 |
| 2977230 | 0.000160487 | 0.390483336 |
| 1401365 | 0.000192711 | 0.390483336 |
| 1102760 | 0.000255908 | 0.394940024 |
| 3118276 | 2.79E−04 | 0.394940024 |
| 1059350 | 2.94E−04 | 0.394940024 |
| 208445 | 3.41E−04 | 0.394940024 |
| 2745240 | 3.45E−04 | 0.394940024 |
| 2199587 | 4.81E−04 | 0.409390172 |
| 3448879 | 0.000540829 | 0.409390172 |
| 519293 | 0.000546219 | 0.409390172 |
| 1206778 | 0.001074808 | 0.418863405 |
| 1066286 | 0.001445325 | 0.422175319 |

TABLE 10C
HMDT

| Perlegen_SNP | FisherPval | FisherQval |
|---|---|---|
| 2142435 | 1.02E−06 | 0.132093615 |
| 2223295 | 2.34E−06 | 0.132093615 |
| 2431351 | 0.00000257 | 0.132093615 |
| 1586038 | 0.0000511 | 0.31980934 |
| 1586042 | 0.000135644 | 0.31980934 |
| 3150971 | 0.000281239 | 0.32594106 |
| 1839162 | 0.000480641 | 0.35099799 |
| 414780 | 0.00056594 | 0.35099799 |
| 561917 | 0.000736895 | 0.35099799 |
| 674238 | 0.000795846 | 0.353567555 |
| 1136710 | 0.001195291 | 0.37398024 |
| 1771665 | 0.00127509 | 0.378016308 |
| 169827 | 0.001377927 | 0.37915258 |
| 1136732 | 0.001775531 | 0.387152393 |
| 1667424 | 0.001921795 | 0.391942669 |
| 486447 | 0.000036 | 0.31980934 |
| 1596162 | 0.0000562 | 0.31980934 |
| 4662308 | 0.0000625 | 0.31980934 |
| 1208382 | 0.000125181 | 0.31980934 |
| 437310 | 0.000175495 | 0.31980934 |
| 2228591 | 1.98E−04 | 0.31980934 |
| 4431812 | 2.60E−04 | 0.325592215 |
| 2354274 | 2.97E−04 | 0.326814783 |
| 2040404 | 5.12E−04 | 0.35099799 |
| 1329983 | 1.31E−03 | 0.37915258 |
| 4032210 | 0.001350039 | 0.37915258 |
| 1224387 | 0.001798156 | 0.389879921 |

TABLE 10D
INSOM

| Perlegen_SNP | FisherPval | FisherQval |
|---|---|---|
| 3484509 | 4.76E−05 | 0.217953355 |
| 1527003 | 0.0000854 | 0.239113003 |
| 2340592 | 1.37E−06 | 0.121551406 |
| 1246865 | 6.06E−06 | 0.177647402 |
| 2488104 | 1.95E−05 | 0.202246478 |
| 2027772 | 0.0000887 | 0.240564576 |
| 76470 | 0.000107824 | 0.24564659 |
| 3675147 | 0.000129775 | 0.24564659 |
| 2030091 | 0.000204348 | 0.24564659 |
| 4545337 | 0.000225941 | 0.24564659 |
| 481459 | 0.000309842 | 0.24564659 |
| 2097268 | 0.000558842 | 0.249236468 |
| 1148525 | 0.000697271 | 0.256026918 |
| 1232320 | 0.000748209 | 0.256370115 |
| 761347 | 0.000923 | 0.262408796 |
| 761361 | 0.001003284 | 0.262481603 |
| 1071708 | 0.001310358 | 0.274344974 |
| 2038957 | 0.001339653 | 0.274344974 |
| 2254679 | 0.001384269 | 0.278146832 |
| 603200 | 0.001429538 | 0.279575331 |
| 1803884 | 0.002026076 | 0.292569879 |
| 1132867 | 0.002219513 | 0.293495601 |
| 2027435 | 0.002306692 | 0.295707052 |
| 2060561 | 0.002870454 | 0.308997794 |
| 594210 | 0.002925541 | 0.309239015 |
| 2212327 | 4.38E−05 | 0.214668503 |
| 1064904 | 0.000104007 | 0.24564659 |
| 3396622 | 0.000104718 | 0.24564659 |
| 663947 | 0.000257256 | 0.24564659 |
| 1071700 | 0.000338154 | 0.24564659 |
| 1046450 | 0.000357938 | 0.24564659 |
| 1833522 | 0.000360144 | 0.24564659 |
| 502183 | 0.000524599 | 0.249236468 |
| 3115452 | 0.000545186 | 0.249236468 |
| 178191 | 0.00093653 | 0.262408796 |
| 3287599 | 0.000977578 | 0.262408796 |
| 882230 | 0.001633093 | 0.281881007 |

1. A method of polymorphic profiling an individual comprising:
determining a polymorphic profile in at least two but no more than 1000 polymorphic sites, the polymorphic sites including at least two sites shown in Table 1 or in linkage disequilibrium therewith.

2. The method of claim 1, wherein the polymorphic profile is determined in at least two polymorphic sites shown in Table 3.

3. (canceled)

4. The method of claim 1, wherein the polymorphic profile is determined in at least 2 and no more than 50 different polymorphic sites shown in Table 3.

5. (canceled)

6. The method of claim 1, wherein the polymorphic profile is determined in at least 5 polymorphic sites shown in Table 1 or 3.

7-9. (canceled)

10. The method of claim 1, wherein the polymorphic profile is determined in at least two polymorphic sites in or within 10 kb of the at least two genes shown in Table 1.

11. The method of claim 1, wherein the polymorphic profile is determined in at least two polymorphic sites in or within 10 kb of at least two genes shown in Table 2.

12. (canceled)

13. The method of claim 1, wherein the polymorphic profile is determined at polymorphic sites in at least 5 genes shown in Table 1 or Table 2.

14. The method of claim 1, wherein the polymorphic profile is determined in at least two polymorphic sites shown in Table 1 or 3.

15. (canceled)

16. The method of claim 1, wherein one of the polymorphic sites is in the TTC12 gene or in linkage disequilibrium therewith.

17. The method of claim 16, wherein one of the polymorphic sites is SNP No. 1752273.

18. A method of determining whether a patient with depression is suitable for treatment with an SSRI or inclusion in a clinical trial for testing an SSRI, comprising:
determining the presence of a polymorphic profile in at least one polymorphic site shown in Table 1 or 3 or in linkage disequilibrium therewith; and
determining whether to treat the patient with the SSRI or include the patient in a clinical trial based on the polymorphic profile.

19. (canceled)

20. The method of claim 18, further comprising determining the total number of alleles in the polymorphic profile associated with a positive response to SSRIs and the total number of alleles in the polymorphic profile associated with a negative (or lack of) response to SSRIs, whereby a higher number of alleles associated with the positive response than alleles associated with a negative response is an indication of whether a patient with depression is amenable to treatment with SSRIs or should be included in a clinical trial for testing an SSRI.

21. The method of claim 18, further comprising determining the total number of alleles in the polymorphic profile associated with a positive response to placebo and the total number of alleles in the polymorphic profile associated with a negative response (or lack of) to placebo, whereby a higher number of alleles associated with the positive response than alleles associated with a negative response is an indication of whether a patient is susceptible to a placebo effect or should be excluded from a clinical trial for testing an SSRI.

22. The method of claim 18, wherein the method determines which polymorphic forms are present in at least 10 polymorphic sites shown in Table 1 or Table 3.

23. (canceled)

24. The method of claim 18, further comprising treating the patient with an SSRI.

25. The method of claim 18, further comprising treating the patient with a treatment for depression other than treatment with an SSRI.

26. The method of claim 20, further comprising performing a clinical trial to test an SSRI on a population including the patient.

27. The method of claim 21, further comprising performing a clinical trial to test the SSRI on a population not including the patient.

28. The method of claim 18, wherein one of the polymorphic sites is in the gene TTC12 or in linkage disequilibrium therewith.

29. The method of claim 18, wherein the polymorphism is SNP No.

30. A method of expression profiling, comprising:
determining expression levels of at least 2 and no more than 10,000 genes in a subject, wherein at least two of the genes are from Table 1 or 2, the expression levels forming an expression profile.

31. (canceled)

32. The method of claim 1, wherein the subject has depression.

33-41. (canceled)

42. A method of screening a compound activity in modulating depression, comprising:
determining whether a compound binds to, modulates the expression of, or modulates the activity of a polypeptide encoded by a gene shown in Table 1 or Table 2.

43-46. (canceled)

47. A method of effecting treatment or prophylaxis of depression, comprising:
administering to a subject having or at risk of depression a compound that modulates the expression or activity of a gene shown in Table 1 or 2.

48-54. (canceled)
